(19) United States
(12) Patent Application Publication
(10) Pub. No.: US 2026/0297093 A1
(43) Pub. Date: Oct. 1, 2026

(54) Sulfoximine compound having FGFR inhibitory effect, pharmaceutical composition comprising same, and use thereof

(71) Applicant: KINOTECK THERAPEUTICS CO., LTD, Guangzhou (CN)

(72) Inventors: Shaohua CHANG, Guangzhou (CN); Xiaofei CHEN, Guangzhou (CN); Jiaguo LI, Guangzhou (CN); Huiqiong LI, Guangzhou (CN); Pinglian WU, Guangzhou (CN); Yinhui WU, Guangzhou (CN); Xiaomei REN, Guangzhou (CN); Dawei MA, Guangzhou (CN)

(21) Appl. No.: 18/881,170

(22) PCT Filed: Jul. 5, 2023
(86) PCT No.: PCT/CN2023/105960

§ 371 Date: Jan. 3, 2025

(30) Foreign Application Priority Data
Jul. 6, 2022 (CN) 202210798720.1

(51) Int. Cl. C07D 487/04; A61K 31/437; A61K 31/519; A61K 31/536; A61K 31/541; A61P 35/00; C07B 59/00; C07D 471/04; C07D 519/00

(52) U.S. Cl. CPC C07D 487/04; A61K 31/437; A61K 31/519; A61K 31/536; A61K 31/541; A61P 35/00; C07B 59/002; C07D 471/04; C07D 519/00

(57) ABSTRACT

Provided are a sulfoximine compound having an FGFR inhibitory effect, a pharmaceutical composition comprising said sulfoximine compound, and a use thereof. Specifically provided are a sulfoximine compound as represented by formula (I) which can be used as a fibroblast growth factor receptor (FGFR) inhibitor, a tautomer, a stereoisomer, a hydrate, a solvate, a pharmaceutically acceptable salt thereof, a pharmaceutical composition comprising the compound, and a use thereof in the preparation of a pharmaceutical composition; wherein R₁, R₂, R₃, R₄, R₅, Cy^{A}, Cy^{B}, Cy^{C}, etc., are as defined in the description.

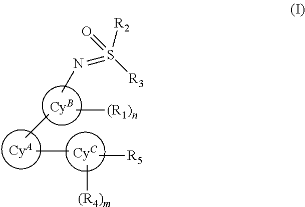

### TECHNICAL FIELD

The present invention relates to the field of medicinal chemistry, and specifically provides a structurally novel FGFR inhibitor.

### BACKGROUND

Fibroblast growth factor receptor (FGFR) is a receptor for fibroblast growth factor (FGF) signaling, and its family consists of four members (FGFR1, FGFR2, FGFR3, FGFR4), which are glycoproteins consisting of extracellular immunoglobulin (Ig) like domains, hydrophobic transmembrane regions, and intracellular portion including tyrosine kinase regions. The binding of FGF ligands induces receptor dimerization and conformational changes in the intracellular domain, which cause intermolecular phosphorylation of the kinase domain and intracellular tail. Phosphorylated residues act as docking sites for attachment proteins, which promote downstream signaling cascades that lead to cellular behavior including proliferation, survival, differentiation, migration, and angiogenesis. FGFRs signal abnormalities involve a variety of cancer types, including liver cancer, intrahepatic cholangiocarcinoma, bladder cancer, endometrial cancer, breast cancer and lung cancer, and disease progression occurs through overexpression, point mutation and/or chromosomal translocation.

With the continuous deepening of research, pan FGFR1-3 inhibitors have shown clinical responses to various cancers with altered FGFR, but also exhibit target-limited toxicity, which lead to adverse side effects such as hyperphosphatemia and tissue mineralization, which originate from the fact that the regulation of phosphate reabsorption is mediated by FGFR1 and FGFR3. Some studies have summarized that FGFR2 translocation occurred in 14% of intrahepatic cholangiocarcinoma; FGFR2 mutations occurred in 12-14% of endometrial cancers and 5% of squamous non-small cell lung cancers; FGFR2 was amplified in 12-14% of gastric cancer and 4% of breast cancer. FGFR2 plays a role in promoting acquired resistance to human epidermal growth factor receptor 2 (HER2) targeted therapy by indirectly overactivating FGFR2 in tumor associated fibroblasts.

Therefore, based on the clinical needs to be met, the development of FGFR2 selective inhibitors for treatment has great value and prospects.

### SUMMARY OF THE INVENTION

The purpose of the present invention is to develop a small molecule compound as FGFR inhibitor.

In the first aspect of the present invention, provided is a compound as shown in formula (I), a tautomer, a stereoisomer, a hydrate, a solvate, or a pharmaceutically acceptable salt thereof:

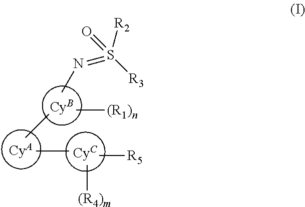

wherein, n and m are each independently 0, 1, 2, or 3;
Cy^{A} is selected from the group consisting of:

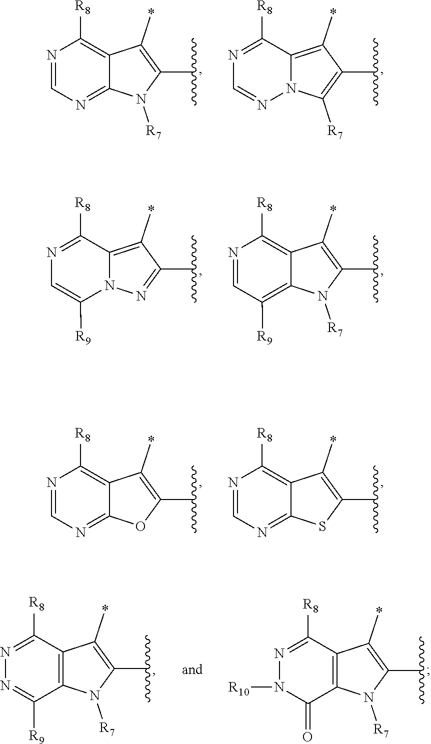

wherein [symbol] denotes a bond connected to Cy^{B}, and

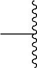

denotes a bond connected to Cy^{C};
Cy^{B} is selected from the group consisting of: C₆₋₁₀ aryl and 5-12 membered heteroaryl;
Cy^{C} is selected from the group consisting of: C₆₋₁₀ aryl, 5-12-membered heteroaryl, saturated or partially unsaturated C₃₋₆ carbocyclyl, saturated or partially unsaturated 4-12-membered heterocyclyl;
R₁ is selected from the group consisting of: H, D, halogen, CN, OH, NH₂, C₁₋₆ alkyl, C₁₋₆ alkoxy, C₁₋₆ haloalkyl, C₁₋₆ haloalkoxy, C₁₋₆ alkylamino, C₂₋₄ alkenyl, C₁₋₄ haloalkenyl, C₂₋₄ alkynyl, C₁₋₄ haloalkynyl, C₃₋₆ saturated or partially unsaturated carbocyclyl, C₃₋₆ halogenated saturated or partially unsaturated carbocyclyl, C₃₋₆ saturated or partially unsaturated carbocyclyl-O—, SF₅ and 4-12 membered heterocyclyl;
R₂ and R₃ are each independently selected from the group consisting of: C₁₋₆ alkyl, C₁₋₆ alkoxy, C₁₋₆ haloalkyl, C₁₋₆ haloalkoxy, C₁₋₆ alkylamino, C₃₋₆ saturated or partially unsaturated carbocyclyl, and 4-12-membered heterocyclyl; wherein R₂ and R₃ can be the same or different; or R₂ and R₃ together with the sulfur atom to which they are connected form a 4-12-membered heterocyclyl;
R₄ is selected from the group consisting of: H, D, halogen, CN, OH, NH₂, C₁₋₆ alkyl, C₁₋₆ alkoxy, C₁₋₆ haloalkyl, C₁₋₆ haloalkoxy, C₁₋₆ alkylamino, C₂₋₄ alkenyl, C₁₋₄ haloalkenyl, C₂₋₄ alkynyl, C₁₋₄ haloalkynyl, C₃₋₆ saturated or partially unsaturated carbocyclyl, C₃₋₆ halogenated saturated or partially unsaturated carbocyclyl, C₃₋₆ carbocyclyl-O—, SF₅ and 4-12-membered heterocyclyl;
R₅ is -L-Rw, wherein L is selected from the group consisting of: covalent bond, —C₁₋₄ alkyl-, —NR₆—, and —C₁₋₄ alkyl NR₆—;
R₆ is selected from the group consisting of: H, C₁₋₆ alkyl, C₁₋₆ alkoxy, C₁₋₆ haloalkyl and
Rw is selected from the group consisting of: halogen, cyano,

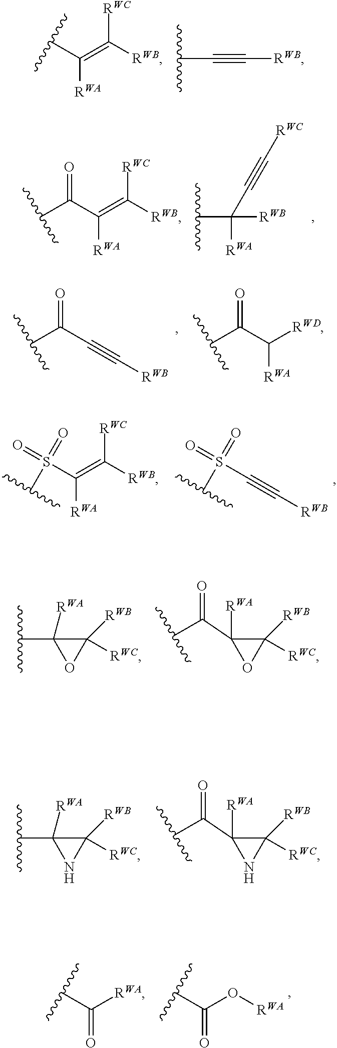

and 5-12-membered nitrogen-containing heterocyclyl;
R^{WA}, R^{WB}, and R^{WC} are each independently selected from the group consisting of: H, D, halogen, CN, C(O)Ra, C(O)ORa, NRaRb, C(O)NRaRb, C(O)NRaORb, or selected from the group consisting of: C₁₋₆ alkyl, C₁₋₆ alkoxy, C₁₋₆ haloalkyl, C₁₋₆ haloalkoxy, phenyl, 3 to 7-membered saturated or partially unsaturated heterocyclyl with 1 to 2 heteroatoms independently selected from nitrogen, oxygen and sulfur, and 5 to 6-membered heteroaryl with 1 to 4 heteroatoms independently selected from nitrogen, oxygen and sulfur; and R^{WA}, R^{WB}, and R^{WC} can each be independently substituted with 1, 2, or 3 substituents selected from the group consisting of: C₁₋₆ alkylamino and C₁₋₆ alkoxy;
or R₆ and Rw together form a ring; and the ring is selected from the group consisting of: 5-12 membered heteroaryl and 4-12 membered heterocyclyl;
R₇ is selected from the group consisting of: H, C₁₋₆ alkyl, C₁₋₆ alkoxy, C₁₋₆ haloalkyl, C₁₋₆ haloalkoxy, C₁₋₆ alkylamino, C₂₋₄ alkenyl, C₁₋₄ haloalkenyl, C₂₋₄ alkynyl, C₁₋₄ haloalkynyl, C₃₋₆ saturated or partially unsaturated carbocyclyl, C₃₋₆ halogenated saturated or partially unsaturated carbocyclyl, C₆₋₁₀ aryl, 5-12 membered heteroaryl and 4-12-membered heterocyclyl; or R₄ and R₇, as well as the atoms between them, together form a ring, and the ring is selected from the group consisting of: C₆₋₈ saturated or partially unsaturated carbocyclyl, C₆₋₁₀ aryl, 6-12 membered heteroaryl and 6-12 membered saturated or partially unsaturated heterocyclyl;
R₈ is selected from the group consisting of: H, OH, NH₂, C₁₋₆ alkyl, C₁₋₆ alkoxy, C₁₋₆ haloalkyl, C₁₋₆ haloalkoxy and C₁₋₆ alkylamino;
R₉ is selected from the group consisting of: H, D, halogen, CN, NH₂, C₁₋₆ alkyl, C₁₋₆ alkoxy, C₁₋₆ haloalkyl, C₁₋₆ haloalkoxy and C₁₋₆ alkylamino;
unless otherwise specified, each of the above-mentioned alkyl, alkoxy, haloalkyl, haloalkoxy, alkylamino, alkenyl, haloalkenyl, alkynyl, haloalkynyl, saturated or partially unsaturated carbocyclyl, halogenated saturated or partially unsaturated carbocyclyl, saturated or partially unsaturated heterocyclyl, aryl and heteroaryl can be substituted with one or more Ra;
Ra and Rb are each independently selected from H, D, halogen, CN, oxo (═O), OH, NH₂, C₁₋₆ alkyl, C₁₋₆ alkoxy, C₁₋₆ haloalkyl, C₁₋₆ haloalkoxy, C₁₋₆ alkylamino, C₂₋₄ alkyenyl, C₁₋₄ haloalkenyl, C₂₋₄ alkynyl, C₁₄ haloalkynyl, C₃₋₆ saturated or partially unsaturated carbocyclyl, C₃₋₆ halogenated saturated or partially unsaturated carbocyclyl, C₃₋₆ saturated or partially unsaturated carbocyclyl-O— and 4-12 membered heterocyclyl;
wherein, the heterocyclyl is a saturated or partially unsaturated non-aromatic group; the carbocyclyl or heterocyclyl can be optionally in the form of a monocycle, bridged ring, spirocycle, or fused ring.

In some embodiments, the compound has a structure as shown in formula (I-1):

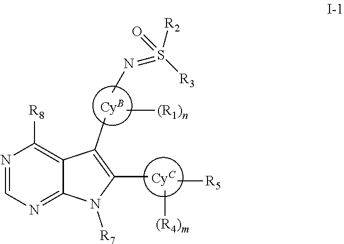

wherein, R₁, R₂, R₃, R₄, R₅, R₇, R₈, Cy^{B}, and Cy^{C} have the definitions as claimed in claim 1;
or R₇, R₄, and atoms to which they connected together form a 5-12 membered carbocyclyl or heterocyclyl.

In some embodiments, the compound has a structure as shown in formula (I-2):

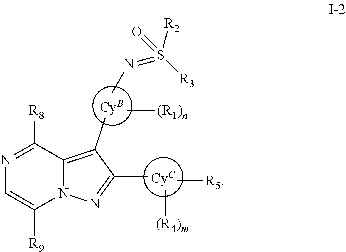

In another preferred embodiment, R₅ is selected from the group consisting of: H, NH₂, C₁₋₆ alkyl, and C₁₋₆ alkylamino.

In another preferred embodiment, R₉ is selected from the group consisting of: H, D, halogen, CN, C₁₋₆ alkyl and C₁₋₆ haloalkyl.

In some embodiments, Cy^{B} is selected from the group consisting of: phenyl and 5-7 membered heteroaryl.

In another preferred embodiment, Cy^{B} is selected from the group consisting of: phenyl, pyridyl, pyrimidinyl, pyridazinyl, pyrrolyl, furanyl, thiophenyl, imidazolyl, pyrazolyl, thiazolyl, triazolyl, and oxazolyl.

In some embodiments, Cy^{C} is selected from the group consisting of: phenyl, 5-7 membered heteroaryl, saturated or partially unsaturated C₃₋₆ saturated or partially unsaturated carbocyclyl, and saturated or partially unsaturated 4-7 membered heterocyclyl.

In another preferred embodiment, Cy^{C} is selected from the group consisting of: phenyl, pyridyl, pyrimidinyl, pyridazinyl, pyrrolyl, furanyl, thiophenyl, imidazolyl, pyrazolyl, thiazolyl, triazolyl, oxazolyl, 5-7 membered saturated or partially unsaturated heterocyclyl.

In some embodiments, R₅ is -L-Rw, wherein L is selected from the group consisting of: covalent bonds, —C₁₄ alkyl, —NR₆—;
R₆ is selected from the group consisting of: H, C₁₋₆ alkyl;
Rw is selected from the group consisting of: cyano,

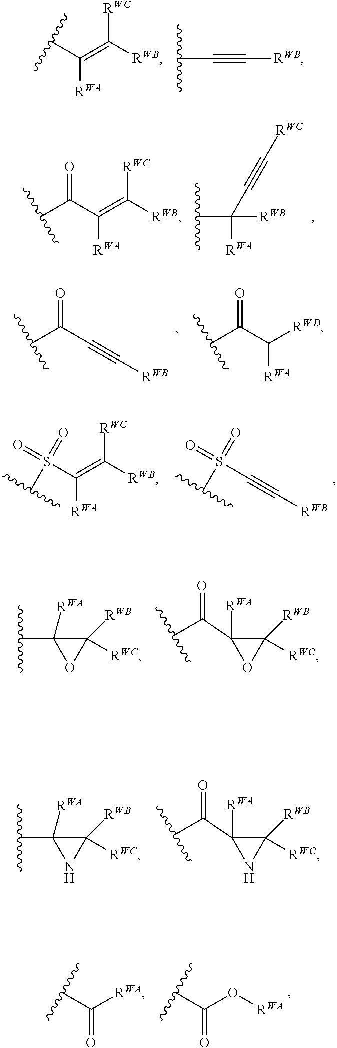

and 5-7 membered nitrogen-containing heterocyclyl;
R^{WA}, R^{WB}, and R^{WC} are each independently selected from the group consisting of: H, D, halogen, C₁₋₆ alkyl and C₁₋₆ haloalkyl; and R^{WA}, R^{WB}, and R^{WC} can each be independently substituted with 1, 2, or 3 substituents selected from the group consisting of: C₁₋₆ alkylamino, and C₁₋₆ alkoxy;
or R₆ and Rw together form a ring; and the ring is selected from the group consisting of: 5-7 membered heteroaryl and 4-7 membered heterocyclyl.

In some embodiment,
R₁ is selected from the group consisting of: H, D, halogen, CN, OH, NH₂, C₁₋₆ alkyl, C₁₋₆ alkoxy, C₁₋₆ haloalkyl, C₁₋₆ haloalkoxy, C₁₋₆ alkylamino, C₂₋₄ alkenyl, C₁₋₄ haloalkenyl, C₂₋₄ alkynyl, C₁₋₄ haloalkynyl and SF₅;
R₂ and R₃ are each independently selected from the group consisting of: C₁₋₆ alkyl, C₁₋₆ alkoxy, C₁₋₆ haloalkyl, C₁₋₆ haloalkoxy and C₁₋₆ alkylamino; wherein, R₂ and R₃ can be the same or different;
or R₂ and R₃ together with the sulfur atom to which they are connected form a 3-7 membered heterocyclyl (preferably a 4-6 membered heterocyclyl);
R₄ is selected from the group consisting of: H, D, halogen, CN, OH, NH₂, C₁₋₆ alkyl, C₁₋₆ alkoxy, C₁₋₆ haloalkyl, C₁₋₆ haloalkoxy and C₁₋₆ alkylamino.

In another preferred embodiment, R₁ is selected from the group consisting of: H, D, halogen, C₁₋₆ alkyl, C₁₋₆ alkoxy, C₁₋₆ haloalkyl and C₁₋₆ haloalkoxy;
R₂ and R₃ are each independently selected from the group consisting of: C₁₋₆ alkyl, C₁₋₆ haloalkyl, C₁₋₆ alkylamino and C₁₋₆ haloalkylamino; wherein, R₂ and R₃ can be the same or different; or R₂ and R₃ together with the sulfur atom to which they are connected form a 4-8 membered heterocyclyl;
R₄ is selected from the group consisting of H, D, halogen, C₁₋₆ alkyl, C₁₋₆ alkoxy, C₁₋₆ haloalkyl and C₁₋₆ haloalkoxy.

In some embodiments, the compound is selected from the group consisting of

[chemical structure]

[chemical structure]

[chemical structure]

[chemical structure]

[chemical structure]

[chemical structure]

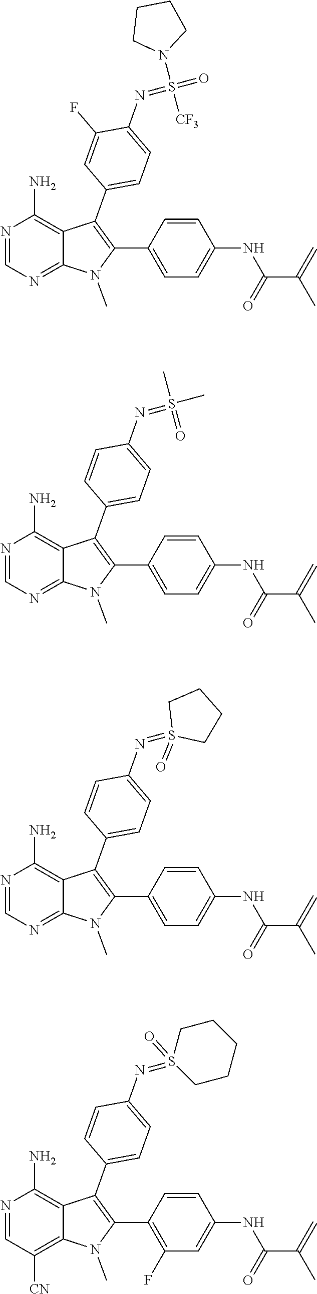 [SMILES: C=C(C)C(=O)Nc1ccc(-c2c(-c3ccc(N=S(=O)(N4CCCC4)C(F)(F)F)c(F)c3)c3c(N)ncnc3n2C)cc1 ; C=C(C)C(=O)Nc1ccc(-c2c(-c3ccc(N=S(C)(C)=O)cc3)c3c(N)ncnc3n2C)cc1 ; C=C(C)C(=O)Nc1ccc(-c2c(-c3ccc(N=S4(=O)CCCC4)cc3)c3c(N)ncnc3n2C)cc1 ; C=C(C)C(=O)Nc1ccc(-c2c(-c3ccc(N=S4(=O)CCCCC4)cc3)c3c(N)ncc(C#N)c3n2C)c(F)c1]

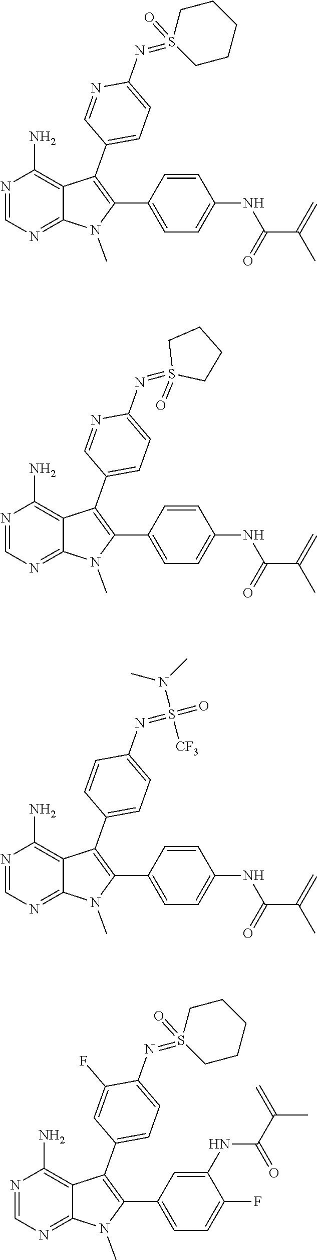 [SMILES: C=C(C)C(=O)Nc1ccc(-c2c(-c3ccc(N=S4(=O)CCCCC4)nc3)c3c(N)ncnc3n2C)cc1 ; C=C(C)C(=O)Nc1ccc(-c2c(-c3ccc(N=S4(=O)CCCC4)nc3)c3c(N)ncnc3n2C)cc1 ; C=C(C)C(=O)Nc1ccc(-c2c(-c3ccc(N=S(=O)(N(C)C)C(F)(F)F)cc3)c3c(N)ncnc3n2C)cc1 ; C=C(C)C(=O)Nc1cc(-c2c(-c3ccc(N=S4(=O)CCCCC4)c(F)c3)c3c(N)ncnc3n2C)ccc1F]

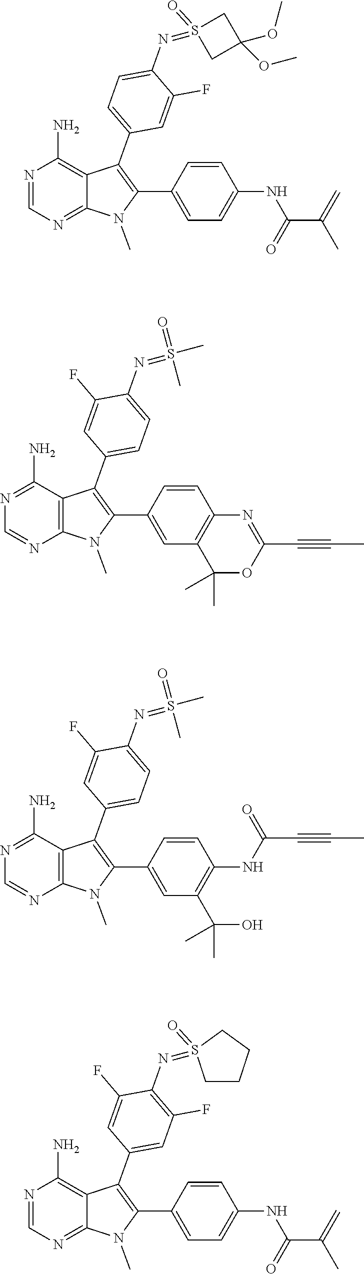 [SMILES: C=C(C)C(=O)Nc1ccc(-c2c(-c3ccc(N=S4(=O)CC(OC)(OC)C4)c(F)c3)c3c(N)ncnc3n2C)cc1 ; CC#CC1=Nc2ccc(-c3c(-c4ccc(N=S(C)(C)=O)c(F)c4)c4c(N)ncnc4n3C)cc2C(C)(C)O1 ; CC#CC(=O)Nc1ccc(-c2c(-c3ccc(N=S(C)(C)=O)c(F)c3)c3c(N)ncnc3n2C)cc1C(C)(C)O ; C=C(C)C(=O)Nc1ccc(-c2c(-c3cc(F)c(N=S4(=O)CCCC4)c(F)c3)c3c(N)ncnc3n2C)cc1]

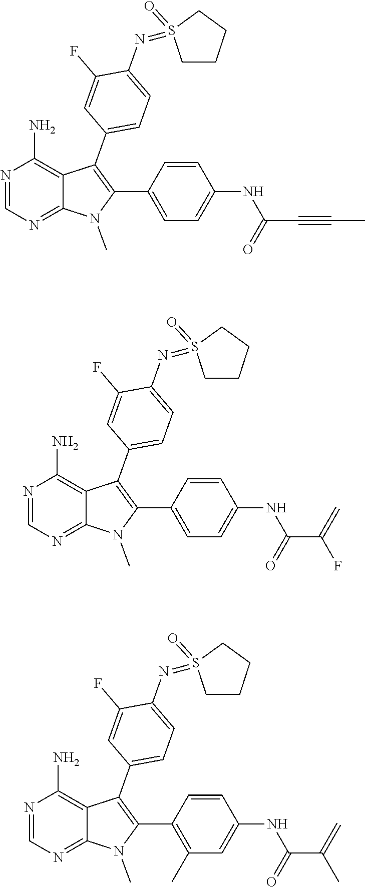 [SMILES: CC#CC(=O)Nc1ccc(-c2c(-c3ccc(N=S4(=O)CCCC4)c(F)c3)c3c(N)ncnc3n2C)cc1 ; C=C(F)C(=O)Nc1ccc(-c2c(-c3ccc(N=S4(=O)CCCC4)c(F)c3)c3c(N)ncnc3n2C)cc1 ; C=C(C)C(=O)Nc1ccc(-c2c(-c3ccc(N=S4(=O)CCCC4)c(F)c3)c3c(N)ncnc3n2C)c(C)c1]

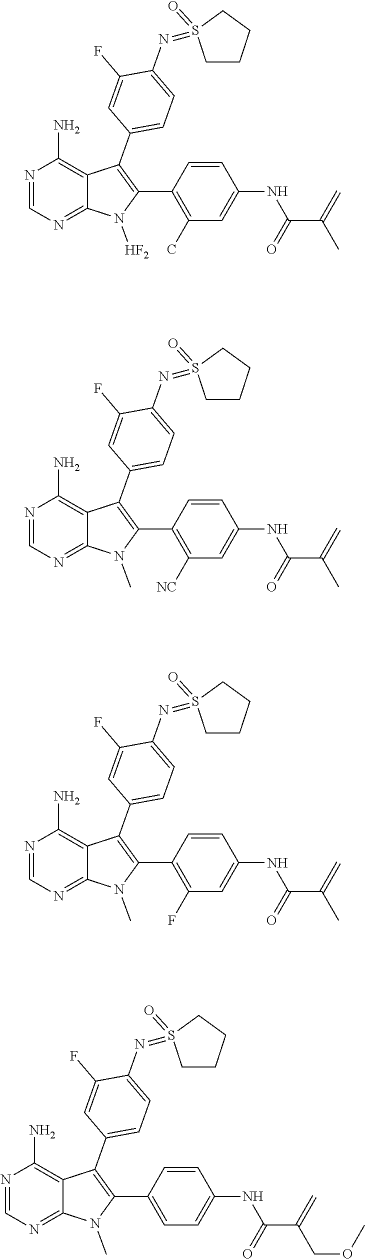

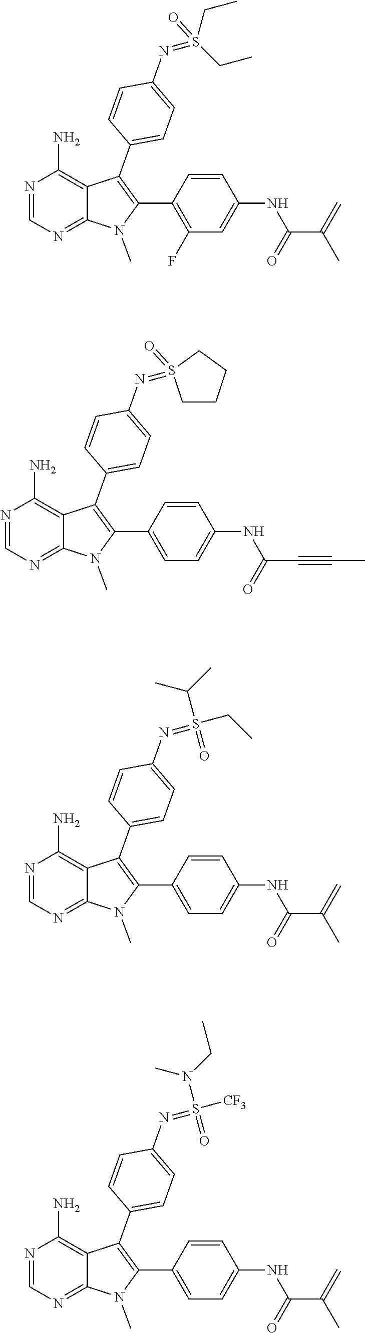 [SMILES: C=C(C)C(=O)Nc1ccc(-c2c(-c3ccc(N=S(=O)(CC)CC)cc3)c3c(N)ncnc3n2C)c(F)c1 ; CC#CC(=O)Nc1ccc(-c2c(-c3ccc(N=S4(=O)CCCC4)cc3)c3c(N)ncnc3n2C)cc1 ; C=C(C)C(=O)Nc1ccc(-c2c(-c3ccc(N=S(=O)(CC)C(C)C)cc3)c3c(N)ncnc3n2C)cc1 ; C=C(C)C(=O)Nc1ccc(-c2c(-c3ccc(N=S(=O)(N(C)CC)C(F)(F)F)cc3)c3c(N)ncnc3n2C)cc1]

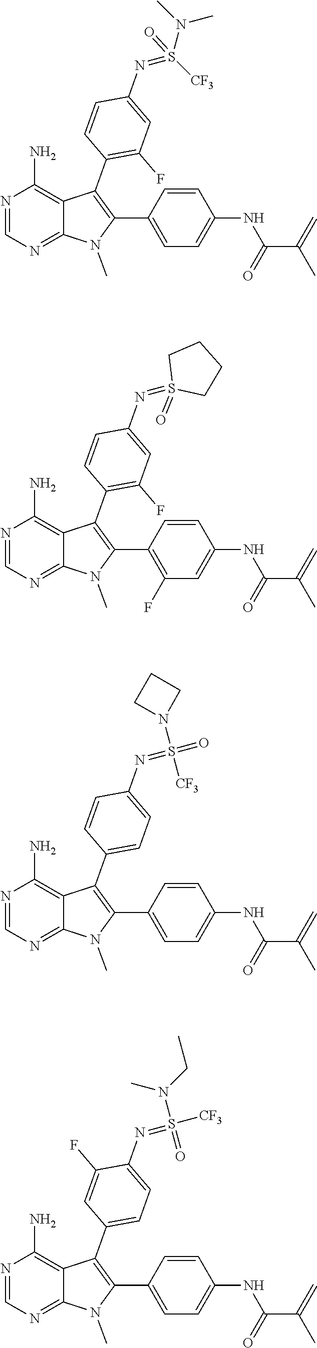 [SMILES: C=C(C)C(=O)Nc1ccc(-c2c(-c3ccc(N=S(=O)(N(C)C)C(F)(F)F)cc3F)c3c(N)ncnc3n2C)cc1 ; C=C(C)C(=O)Nc1ccc(-c2c(-c3ccc(N=S4(=O)CCCC4)cc3F)c3c(N)ncnc3n2C)c(F)c1 ; C=C(C)C(=O)Nc1ccc(-c2c(-c3ccc(N=S(=O)(N4CCC4)C(F)(F)F)cc3)c3c(N)ncnc3n2C)cc1 ; C=C(C)C(=O)Nc1ccc(-c2c(-c3ccc(N=S(=O)(N(C)CC)C(F)(F)F)c(F)c3)c3c(N)ncnc3n2C)cc1]

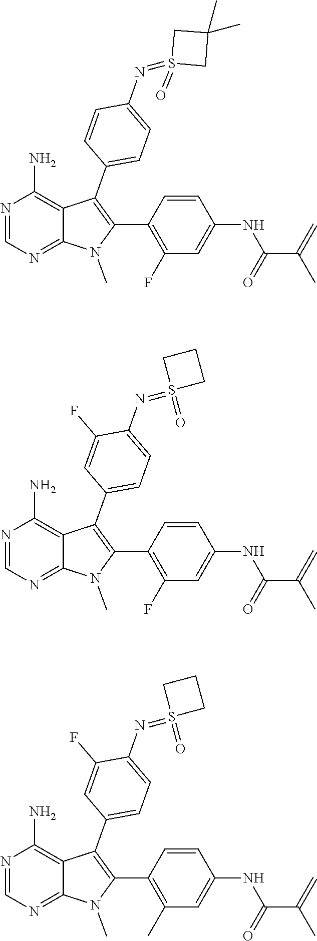 [SMILES: C=C(C)C(=O)Nc1ccc(-c2c(-c3ccc(N=S4(=O)CC(C)(C)C4)cc3)c3c(N)ncnc3n2C)c(F)c1 ; C=C(C)C(=O)Nc1ccc(-c2c(-c3ccc(N=S4(=O)CCC4)c(F)c3)c3c(N)ncnc3n2C)c(F)c1 ; C=C(C)C(=O)Nc1ccc(-c2c(-c3ccc(N=S4(=O)CCC4)c(F)c3)c3c(N)ncnc3n2C)c(C)c1]

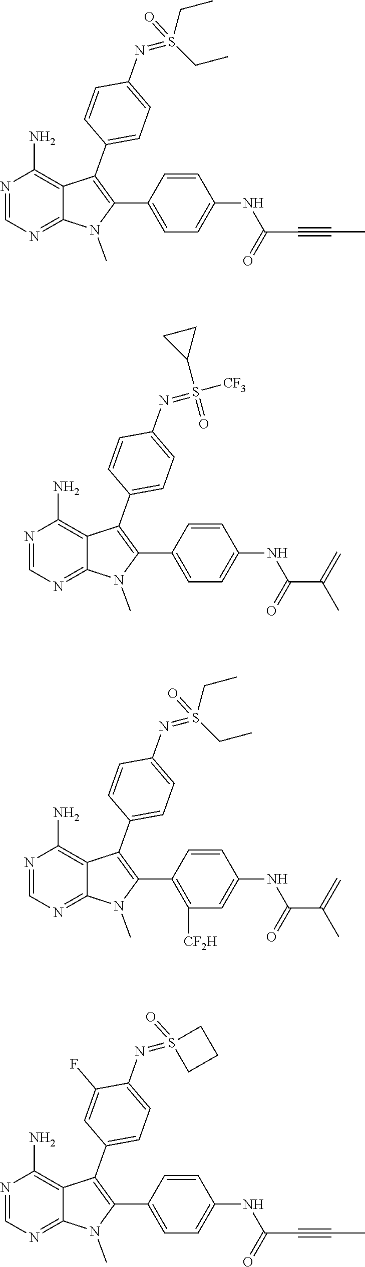 [SMILES: CC#CC(=O)Nc1ccc(-c2c(-c3ccc(N=S(=O)(CC)CC)cc3)c3c(N)ncnc3n2C)cc1 ; C=C(C)C(=O)Nc1ccc(-c2c(-c3ccc(N=S(=O)(C4CC4)C(F)(F)F)cc3)c3c(N)ncnc3n2C)cc1 ; C=C(C)C(=O)Nc1ccc(-c2c(-c3ccc(N=S(=O)(CC)CC)cc3)c3c(N)ncnc3n2C)c(C(F)F)c1 ; CC#CC(=O)Nc1ccc(-c2c(-c3ccc(N=S4(=O)CCC4)c(F)c3)c3c(N)ncnc3n2C)cc1]

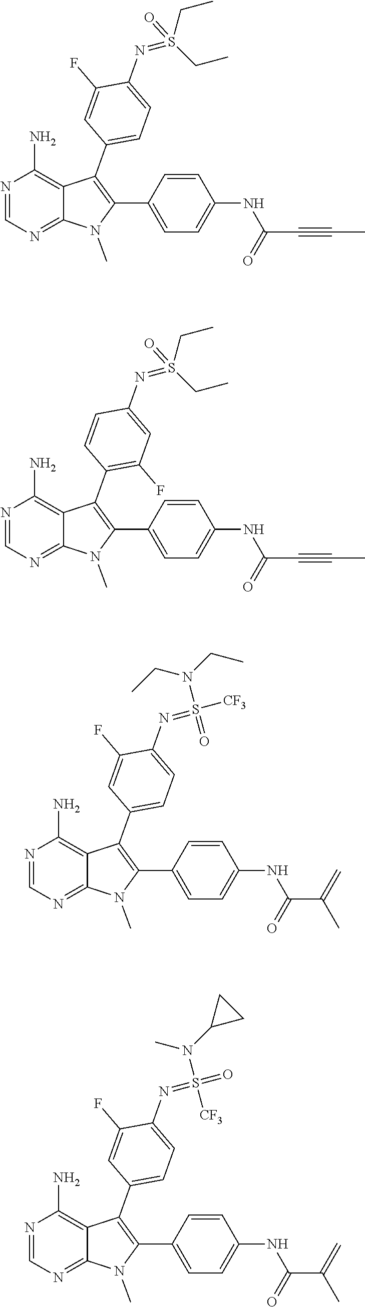 [SMILES: CC#CC(=O)Nc1ccc(-c2c(-c3ccc(N=S(=O)(CC)CC)c(F)c3)c3c(N)ncnc3n2C)cc1 ; CC#CC(=O)Nc1ccc(-c2c(-c3ccc(N=S(=O)(CC)CC)cc3F)c3c(N)ncnc3n2C)cc1 ; C=C(C)C(=O)Nc1ccc(-c2c(-c3ccc(N=S(=O)(N(CC)CC)C(F)(F)F)c(F)c3)c3c(N)ncnc3n2C)cc1 ; C=C(C)C(=O)Nc1ccc(-c2c(-c3ccc(N=S(=O)(N(C)C4CC4)C(F)(F)F)c(F)c3)c3c(N)ncnc3n2C)cc1]

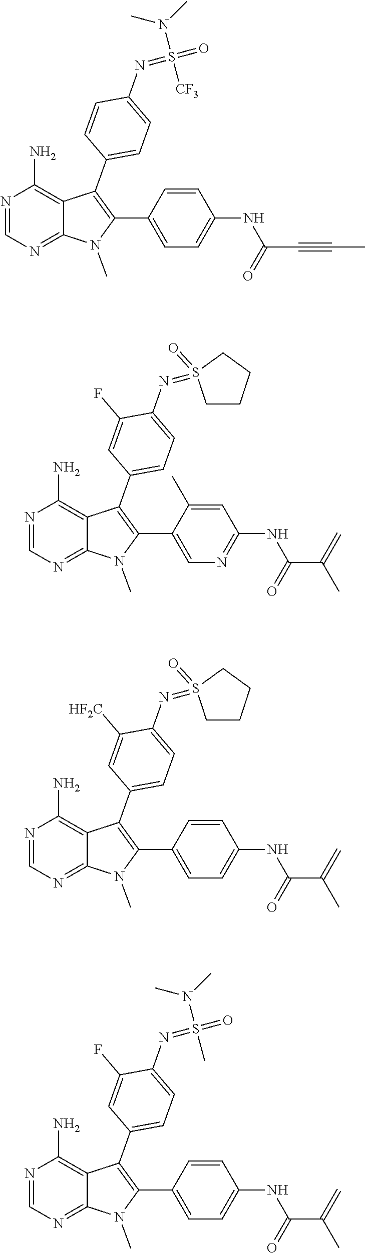

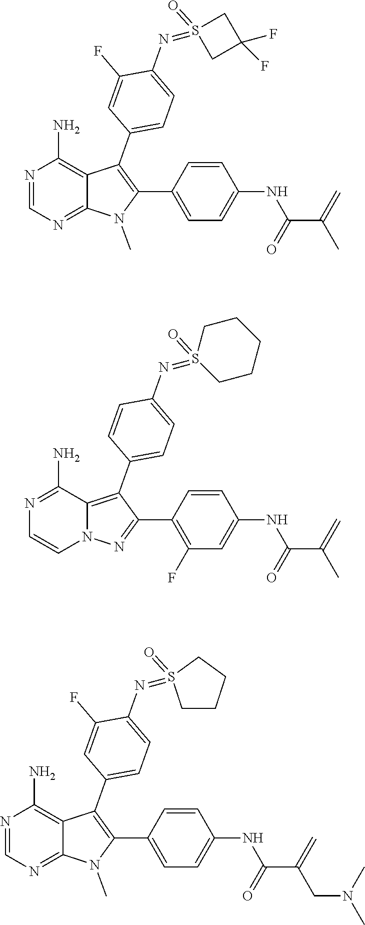 [SMILES: C=C(C)C(=O)Nc1ccc(-c2c(-c3ccc(N=S4(=O)CC(F)(F)C4)c(F)c3)c3c(N)ncnc3n2C)cc1 ; C=C(C)C(=O)Nc1ccc(-c2nn3ccnc(N)c3c2-c2ccc(N=S3(=O)CCCCC3)cc2)c(F)c1 ; C=C(CN(C)C)C(=O)Nc1ccc(-c2c(-c3ccc(N=S4(=O)CCCC4)c(F)c3)c3c(N)ncnc3n2C)cc1]

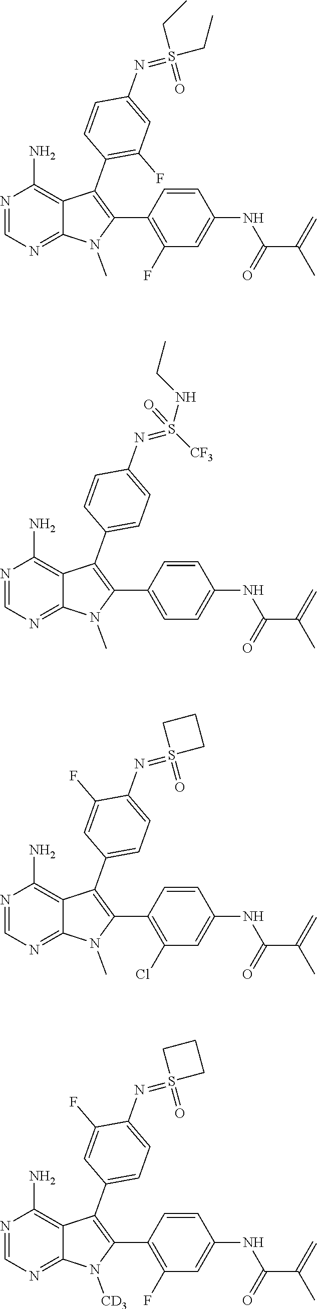 [SMILES: C=C(C)C(=O)Nc1ccc(-c2c(-c3ccc(N=S(=O)(CC)CC)cc3F)c3c(N)ncnc3n2C)c(F)c1 ; C=C(C)C(=O)Nc1ccc(-c2c(-c3ccc(N=S(=O)(NCC)C(F)(F)F)cc3)c3c(N)ncnc3n2C)cc1 ; C=C(C)C(=O)Nc1ccc(-c2c(-c3ccc(N=S4(=O)CCC4)c(F)c3)c3c(N)ncnc3n2C)c(Cl)c1 ; [2H]C([2H])([2H])n1c(-c2ccc(NC(=O)C(=C)C)cc2F)c(-c2ccc(N=S3(=O)CCC3)c(F)c2)c2c(N)ncnc21]

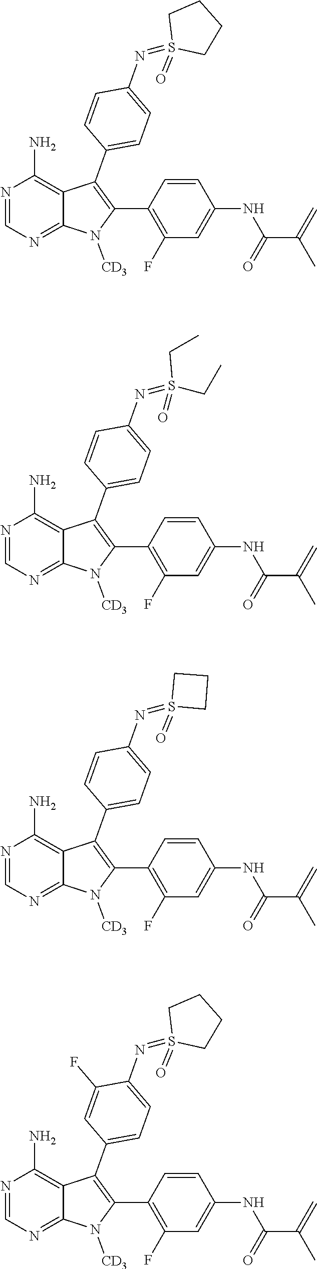 [SMILES: [2H]C([2H])([2H])n1c(-c2ccc(NC(=O)C(=C)C)cc2F)c(-c2ccc(N=S3(=O)CCCC3)cc2)c2c(N)ncnc21 ; [2H]C([2H])([2H])n1c(-c2ccc(NC(=O)C(=C)C)cc2F)c(-c2ccc(N=S(=O)(CC)CC)cc2)c2c(N)ncnc21 ; [2H]C([2H])([2H])n1c(-c2ccc(NC(=O)C(=C)C)cc2F)c(-c2ccc(N=S3(=O)CCC3)cc2)c2c(N)ncnc21 ; [2H]C([2H])([2H])n1c(-c2ccc(NC(=O)C(=C)C)cc2F)c(-c2ccc(N=S3(=O)CCCC3)c(F)c2)c2c(N)ncnc21]

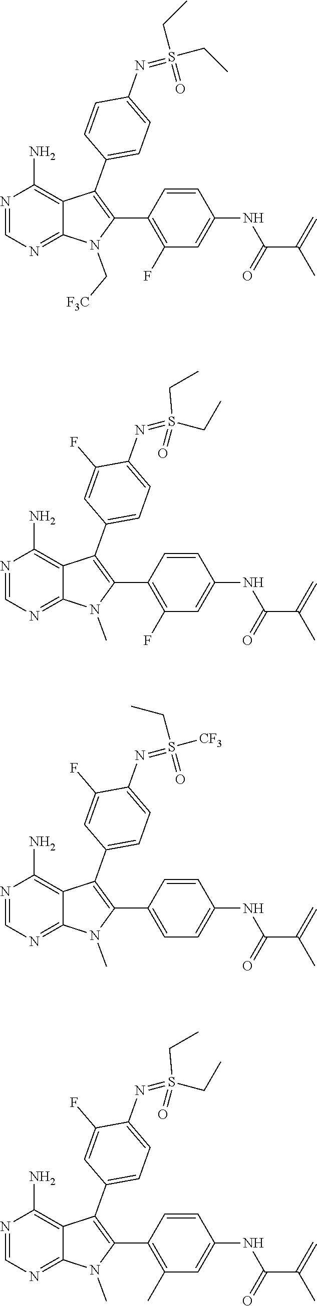 [SMILES: C=C(C)C(=O)Nc1ccc(-c2c(-c3ccc(N=S(=O)(CC)CC)cc3)c3c(N)ncnc3n2CC(F)(F)F)c(F)c1 ; C=C(C)C(=O)Nc1ccc(-c2c(-c3ccc(N=S(=O)(CC)CC)c(F)c3)c3c(N)ncnc3n2C)c(F)c1 ; C=C(C)C(=O)Nc1ccc(-c2c(-c3ccc(N=S(=O)(CC)C(F)(F)F)c(F)c3)c3c(N)ncnc3n2C)cc1 ; C=C(C)C(=O)Nc1ccc(-c2c(-c3ccc(N=S(=O)(CC)CC)c(F)c3)c3c(N)ncnc3n2C)c(C)c1]

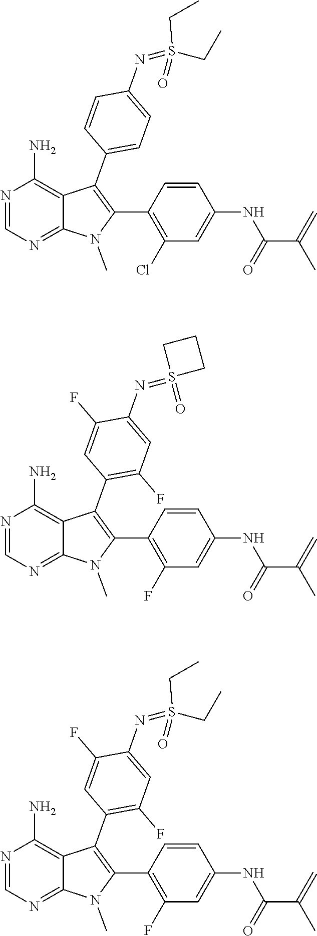 [SMILES: C=C(C)C(=O)Nc1ccc(-c2c(-c3ccc(N=S(=O)(CC)CC)cc3)c3c(N)ncnc3n2C)c(Cl)c1 ; C=C(C)C(=O)Nc1ccc(-c2c(-c3cc(F)c(N=S4(=O)CCC4)cc3F)c3c(N)ncnc3n2C)c(F)c1 ; C=C(C)C(=O)Nc1ccc(-c2c(-c3cc(F)c(N=S(=O)(CC)CC)cc3F)c3c(N)ncnc3n2C)c(F)c1]

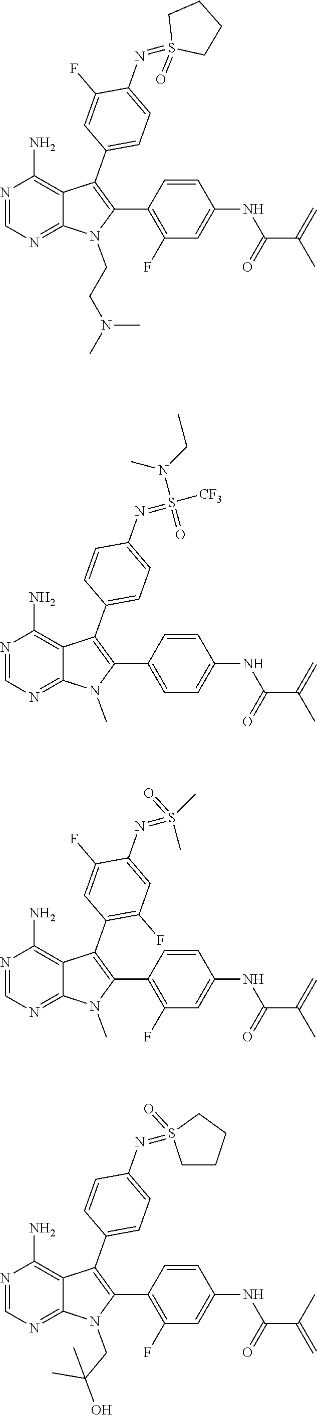 [SMILES: C=C(C)C(=O)Nc1ccc(-c2c(-c3ccc(N=S4(=O)CCCC4)c(F)c3)c3c(N)ncnc3n2CCN(C)C)c(F)c1 ; C=C(C)C(=O)Nc1ccc(-c2c(-c3ccc(N=S(=O)(N(C)CC)C(F)(F)F)cc3)c3c(N)ncnc3n2C)cc1 ; C=C(C)C(=O)Nc1ccc(-c2c(-c3cc(F)c(N=S(C)(C)=O)cc3F)c3c(N)ncnc3n2C)c(F)c1 ; C=C(C)C(=O)Nc1ccc(-c2c(-c3ccc(N=S4(=O)CCCC4)cc3)c3c(N)ncnc3n2CC(C)(C)O)c(F)c1]

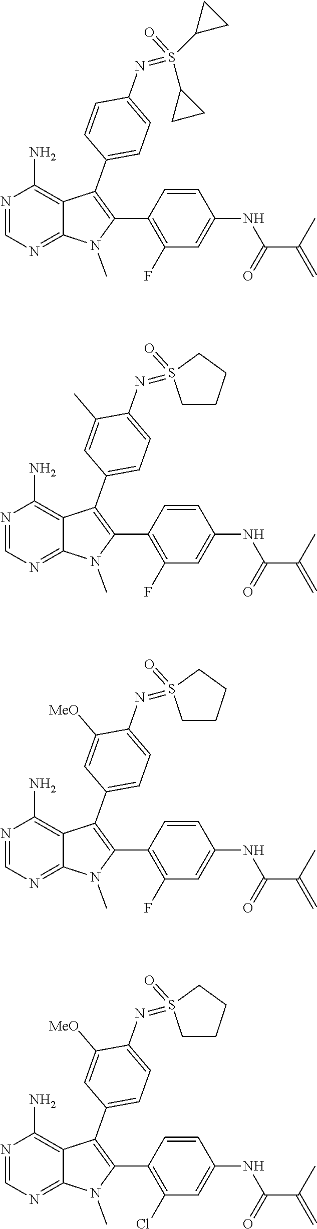 [SMILES: C=C(C)C(=O)Nc1ccc(-c2c(-c3ccc(N=S(=O)(C4CC4)C4CC4)cc3)c3c(N)ncnc3n2C)c(F)c1 ; C=C(C)C(=O)Nc1ccc(-c2c(-c3ccc(N=S4(=O)CCCC4)c(C)c3)c3c(N)ncnc3n2C)c(F)c1 ; C=C(C)C(=O)Nc1ccc(-c2c(-c3ccc(N=S4(=O)CCCC4)c(OC)c3)c3c(N)ncnc3n2C)c(F)c1 ; C=C(C)C(=O)Nc1ccc(-c2c(-c3ccc(N=S4(=O)CCCC4)c(OC)c3)c3c(N)ncnc3n2C)c(Cl)c1]

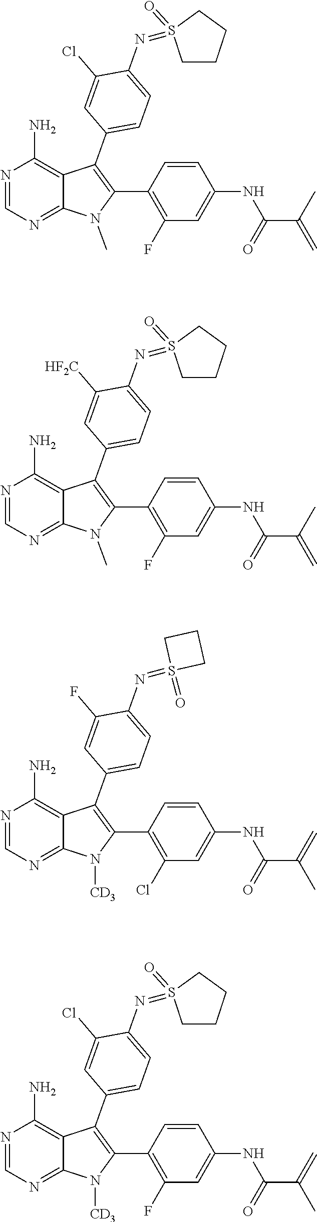

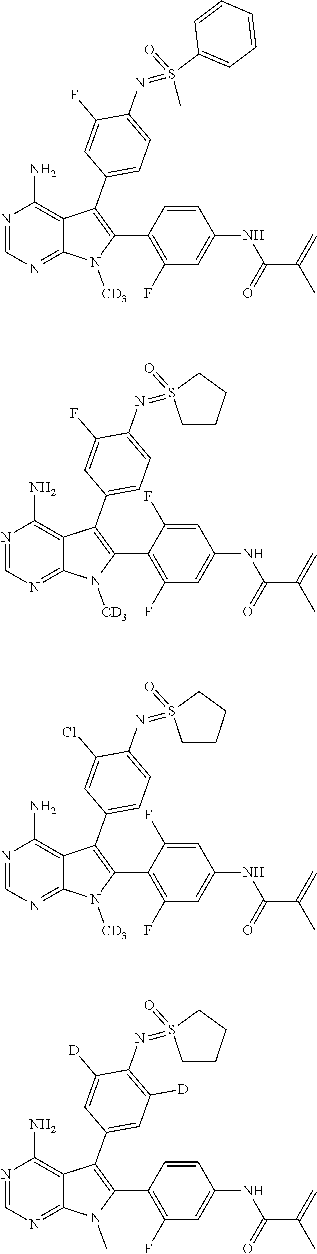 [SMILES: [2H]C([2H])([2H])n1c(-c2ccc(NC(=O)C(=C)C)cc2F)c(-c2ccc(N=S(C)(=O)c3ccccc3)c(F)c2)c2c(N)ncnc21 ; [2H]C([2H])([2H])n1c(-c2c(F)cc(NC(=O)C(=C)C)cc2F)c(-c2ccc(N=S3(=O)CCCC3)c(F)c2)c2c(N)ncnc21 ; [2H]C([2H])([2H])n1c(-c2c(F)cc(NC(=O)C(=C)C)cc2F)c(-c2ccc(N=S3(=O)CCCC3)c(Cl)c2)c2c(N)ncnc21 ; [2H]c1cc(-c2c(-c3ccc(NC(=O)C(=C)C)cc3F)n(C)c3ncnc(N)c23)cc([2H])c1N=S1(=O)CCCC1]

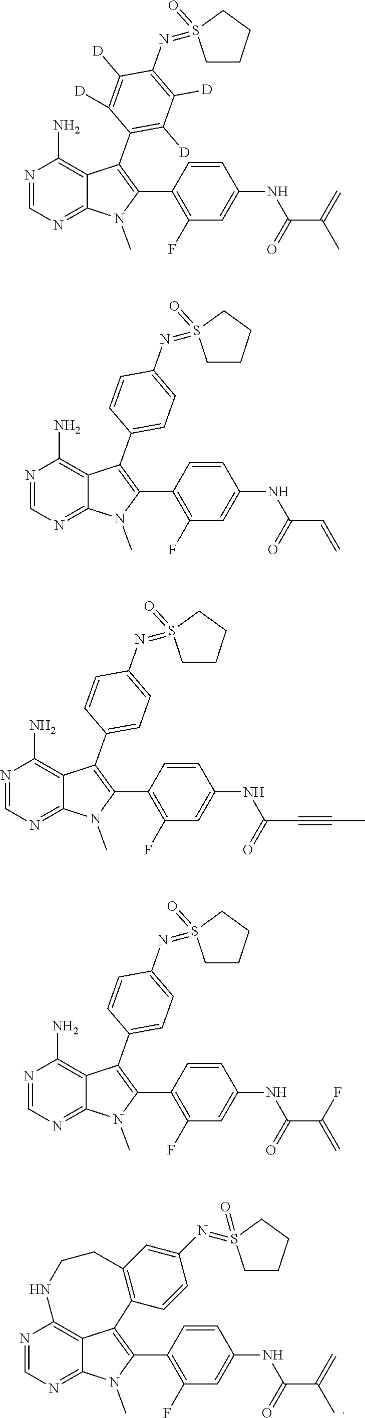 [SMILES: [2H]c1c([2H])c(-c2c(-c3ccc(NC(=O)C(=C)C)cc3F)n(C)c3ncnc(N)c23)c([2H])c([2H])c1N=S1(=O)CCCC1 ; C=CC(=O)Nc1ccc(-c2c(-c3ccc(N=S4(=O)CCCC4)cc3)c3c(N)ncnc3n2C)c(F)c1 ; CC#CC(=O)Nc1ccc(-c2c(-c3ccc(N=S4(=O)CCCC4)cc3)c3c(N)ncnc3n2C)c(F)c1 ; C=C(F)C(=O)Nc1ccc(-c2c(-c3ccc(N=S4(=O)CCCC4)cc3)c3c(N)ncnc3n2C)c(F)c1 ; C=C(C)C(=O)Nc1ccc(-c2c3c4c(ncnc4n2C)NCCc2cc(N=S4(=O)CCCC4)ccc2-3)c(F)c1]

In the second aspect of the present invention, provided is a pharmaceutical composition comprising:
(1) one or more the compound as described in the first aspect of the present invention, a tautomer, a stereoisomer, a hydrate, a solvate, or a pharmaceutically acceptable salt thereof,
(2) one or more pharmaceutically acceptable carriers, excipients, adjuvants, excipients, and/or diluents.

In another preferred embodiment, the pharmaceutical composition is used for treating or preventing diseases or condition associated with abnormal activity or expression levels of FGFR; preferably, the disease or condition is selected from the group consisting of: cholangiocarcinoma, hepatic carcinoma, breast carcinoma, prostate carcinoma, lung carcinoma, thyroid carcinoma, gastric carcinoma, ovarian carcinoma, rectal carcinoma, endometrial carcinoma and uroepithelial carcinoma.

In another preferred embodiment, the cholangiocarcinoma is intrahepatic cholangiocarcinoma.

In another preferred embodiment, the hepatic carcinoma is hepatocellular carcinoma.

In another preferred embodiment, the lung carcinoma is squamous cell carcinoma or non-small cell lung carcinoma.

In the third aspect of the present invention, provided is a use of the compound as described in the first aspect of the present invention, a tautomer, a stereoisomer, a hydrate, a solvate, or a pharmaceutically acceptable salt thereof, or mixture thereof, or a pharmaceutical composition as described in the second aspect of the present invention in the preparation a pharmaceutical composition for treating or preventing diseases or conditions associated with abnormal activity or expression levels of FGFR.

In another preferred embodiment, the disease or condition is specifically associated with a subtype of FGFR; preferably, the FGFR subtype is selected from the group consisting of: FGFR2.

It should be understood that, within the scope of the present invention, each of the above-mentioned technical features of the present invention and each of the technical features specifically described in the following (such as the examples) can be combined with each other to form new or preferred technical solutions. Due to space limitations, it will not be elaborated herein.

### EMBODIMENTS FOR CARRYING OUT THE INVENTION

After long-term and intensive research, the inventors provided a sulfoximine derivative with FGFR inhibitory activity, which has good FGFR inhibitory activity and can be used for preventing, treating, and/or alleviating indications associated with FGFR dysregulation. Based on the above discoveries, the inventors have completed the present invention.

### Definition

As used herein, the term “alkyl” includes straight or branched alkyl. For example, a C₁-C₈ alkyl refers to a straight or branched alkyl with 1 to 8 carbon atoms, such as methyl, ethyl, propyl, isopropyl, butyl, isobutyl, tert-butyl, etc.

As used herein, the term “alkenyl” includes straight or branched alkenyl. For example, C₂-C₆ alkenyl refers to a straight or branched alkenyl with 2-6 carbon atoms, such as vinyl, allyl, 1-propenyl, isopropenyl, 1-butenyl, 2-butenyl, or similar groups.

As used herein, the term “alkynyl” includes straight or branched alkynyl. For example, C₂-C₆ alkynyl refers to a straight or branched alkynyl with 2-6 carbon atoms, such as ethynyl, propynyl, butynyl, or similar groups.

As used herein, the term “carbocyclyl” refers to a cyclic saturated or partially unsaturated aliphatic hydrocarbyl with a specific number of carbon atoms. For example, C₃₋₁₀ carbocyclyl refers to a cyclic saturated or partially unsaturated aliphatic hydrocarbyl with 3-10 carbon atoms. It can be a monocyclic group, such as cyclopropyl, cyclobutyl, cyclopentyl, cyclohexyl, or similar groups. It can also be in the form of a bicyclic group, such, bridged ring or a spiro.

As used herein, the term “alkylamino” refers to an amine group substituted with alkyl. For example, “C₁-C₈ alkylamino” refers to an amino group substituted with a C₁-C₈ alkyl, which can be monosubstituted or disubstituted; for example, methylamino, ethylamino, propylamino, isopropylamino, butylamino, isobutyramimino, tert-butylamino, dimethylamino, diethylamino, dipropylamino, diisopropylamino, dibutylamino, diisobutylamino, di tert-butylamino, etc.

As used herein, the term “alkoxy” refers to a group with a structure of alkyl-O—. For example, “C₁-C₈ alkoxy” refers to a straight or branched alkoxy with 1-8 carbon atoms, including methoxy, ethoxy, propoxy, isopropoxy, butoxy, isobutoxy, tert-butoxy, etc.

As used herein, the term “haloalkyl” refers to an alkyl in which one or more hydrogen atoms are substituted with halogens, wherein, the definition of alkyl is described above.

As used herein, the term “haloalkoxy” refers to an alkoxy in which one or more hydrogen atoms are substituted with halogens, wherein, the definition of alkoxy is described above.

As used herein, the term “heterocyclyl” or “heterocyclic alkyl” refers to a saturated or partially saturated cyclic group with a specific number of ring atoms (such as 3-10 ring atoms), wherein 1-3 atoms are heteroatoms selected from N, S, and O. It can be in the form of a monocyclic ring, a bicyclic ring, or a multiple ring, such, bridged ring or a spirocycle. Specific examples can include oxetanyl, azetidinyl, tetrahydro-2H-pyranyl, piperidinyl, tetrahydrofuranyl, morpholinyl, and pyrrolidinyl.

As used herein, the term “aryl” refers to an aromatic ring group with a specific number of carbon atoms, such as C₆-C₁₀ aryl refers to an aromatic ring group with 6-10 carbon atoms, such as phenyl, naphthyl, and the like.

As used herein, the term “heteroaryl” refers to a cyclic aromatic group with a specific number of atoms, wherein 1-3 atoms are heteroatoms selected from N, S, and O. For example, a 5-12 membered heteroaryl refers to an aromatic ring group with 5-12 carbon atoms. It can be in the form of a monocyclic ring or a fused ring. Specific examples can include pyridyl, pyridazinyl, pyrimidinyl, pyrazinyl, triazinyl, pyrrolyl, pyrazolyl, imidazolyl, (1,2,3)-triazolyl, and (1,2,4)-triazolyl, tetrazolyl, furanyl, thiophenyl, imidazolyl, thiazolyl, oxazolyl, etc.

Unless otherwise specified as “substituted or unsubstituted”, the groups of the present invention may be substituted with substituents selected from the group consisting of: halogen, cyano, nitro, hydroxyl, amino, C₁-C₆ alkyl-amino, C₁-C₆ alkyl, C₂-C₆ alkenyl, C₂-C₆ alkynyl, C₁-C₆ alkoxy, halogenated C₁-C₆ alkyl, halogenated C₂-C₆ alkenyl, halogenated C₂-C₆ alkynyl, halogenated C₁-C₆ alkoxy, allyl, benzyl, C₆-C₁₂ aryl, C₁-C₆ alkoxy-C₁-C₆ alkyl, C₁-C₆-alkoxy carbonyl, phenoxycarbonyl, C₂-C₆ alkynyl-carbonyl, C₂-C₆ alkynyl carbonyl, C₃-C₆ carbocyclyl-carbonyl, C₁-C₆ alkyl-sulfonyl, etc.

As used herein, “halogen” or “halogen atom” refers to F Cl, Br, and I. More preferably, the halogen or halogen atom is selected from F, Cl, and Br. “Halogenated” refers to being substituted with atoms selected from F Cl, Br, and I.

Unless otherwise specified, the structural formulas described in the present invention are intended to include all isomeric forms (such as enantiomers, diastereomers, and geometric isomers (or conformational isomers)), such as R and S configurations with asymmetric centers, (Z) and (E) isomers with double bonds, etc. Therefore, the individual stereoisomers or enantiomers, diastereomers, or geometric isomers (or conformational isomers) of the compounds of the present invention, or the combination thereof are within the scope of the present invention.

As used herein, the term “tautomer” refers to structural isomers with different energies that can be transformed into each other by exceed the low energy barrier. For example, proton tautomers (i.e. proton shift) include interconversion through proton transfer, such as 1H-indazole and 2H-indazole. Valence tautomers include interconversion through recombination of some bonding electrons.

As used herein, the term “solvate” refers to a complex formed by the coordination of a compound of the present invention with a solvent molecule in a specific ratio.

As used herein, the term “hydrate” refers to a complex formed by the coordination of a compound of the present invention with water.

### Active Ingredient

In the present invention, an active ingredient that can effectively inhibit FGFR is provided. The active ingredient is a compound represented by formula (I), which can effectively prevent, treat, and/or alleviate FGFR related diseases.

Experiments have shown that the active ingredients of the present invention can effectively inhibit FGFR kinase proteins, thereby preventing, treating, and/or alleviating FGFR related diseases.

It should be understood that the active ingredients of the present invention include compounds represented by formula (I), or pharmaceutically acceptable salts, or prodrugs thereof. It should be understood that the active ingredients of the present invention also include crystal forms, amorphous compounds, and deuterated compounds of compounds of formula (I).

The term “pharmaceutically acceptable salt” refers to a salt formed by the compound of the present invention with an acid or base that is suitable for pharmaceutical use. Pharmaceutically acceptable salts include inorganic salts and organic salts. A preferred type of salt is the salt formed by the compound of the present invention with an acid. Suitable acids for salt formation include but are not limited to inorganic acids such as hydrochloric acid, hydrobromic acid, hydrofluoric acid, sulfuric acid, nitric acid, phosphoric acid, etc; organic acids such as formic acid, acetic acid, trifluoroacetic acid, propionic acid, oxalic acid, malonic acid, succinic acid, fumaric acid, maleic acid, lactic acid, malic acid, tartaric acid, citric acid, picric acid, benzoic acid, methanesulfonic acid, ethanesulfonic acid, p-toluenesulfonic acid, benzenesulfonic acid, naphthalene sulfonic acid, etc; and amino acids such as proline, phenylalanine, aspartic acid, glutamic acid, etc. Another preferred type of salt is the salt formed by the compound of the present invention with a base, such, alkali metal salts (e.g. sodium or potassium salts), alkaline earth metal salts (e.g. magnesium or calcium salts), ammonium salts (e.g. lower alkyl alcohol ammonium salts and other pharmaceutically acceptable amine salts), such as methylamine salt, ethylamine salt, propylamine salt, dimethylamine salt, trimethylamine salt, diethylamine salt, triethylamine salt, tert-butylamine salt, ethylenediamine salt, hydroxyethyl amine salt, dihydroxyethylamine salt, trihydroxyethylamine salt and amine salts formed by morpholine, piperazine, and lysine, respectively.

### Pharmaceutical Composition and Mode of Administration

Due to the excellent FGFR kinase inhibitory activity of the compounds of the present invention, the compounds of the present invention and various crystal forms, pharmaceutically acceptable inorganic or organic salts, hydrates or solvates thereof, as well as pharmaceutical compositions containing the compounds of the present invention as the main active ingredient, can be used for preventing, treating and/or alleviating FGFR related diseases, such as treating cancer.

The pharmaceutical composition of the present invention comprises a safe and effective amount of the compound of the present invention and a pharmaceutically acceptable excipient or carrier. The term “safe and effective amount” refers to the amount of a compound that is sufficient to significantly improve the condition without causing serious side effects. Generally, pharmaceutical compositions contain 1-2000 mg of the compound of the present invention per dose, and more preferably 10-200 mg of the compound of the present invention per dose.

Preferably, the “one dose” is a capsule or tablet. “Pharmaceutically acceptable carrier” refers to one or more compatible solid or liquid fillers or gel substances, which are suitable for human use and must have sufficient purity and low toxicity. “Compatible” here refers to each component in the composition can be blended with the compound of the present invention and with each other without significantly reducing the efficacy of the compound. Examples of pharmaceutically acceptable carriers include cellulose and derivatives thereof (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agents (such as sodium dodecyl sulfate), coloring agents, flavoring agents, stabilizers, antioxidants, preservatives, pyrogen-free water, etc.

There is no special limitation of mode of administration of the compound or pharmaceutical combination of the present invention, and representative mode of administration include (but are not limited to): oral, parenteral (intravenous, intramuscular, or subcutaneous).

Solid dosage forms for oral administration include capsules, tablets, pills, powders, and granules. In these solid dosage forms, the active compound is mixed with at least one conventional inert excipient (or carrier), such as sodium citrate or dicalcium phosphate, or with the following components: (a) fillers or compatibilizers, such as starch, lactose, sucrose, glucose, mannitol, and silica; (b) binders, such as hydroxymethylcellulose, alginate, gelatin, polyvinylpyrrolidone, sucrose, and arabic gum; (c) humectants, such as glycerin; (d) disintegrants, such,gar, calcium carbonate, potato starch or cassava starch, alginic acid, certain composite silicates, and sodium carbonate; (e) dissolution-retarding agents, such as paraffin; (f) absorption accelerators, such as quaternary ammonium salt; (g) wetting agents, such as cetyl alcohol and glycerol monostearate; (h) adsorbents, such as kaolin; and (i) lubricants, such as talc, calcium stearate, magnesium stearate, solid polyethylene glycol, sodium dodecyl sulfate, or mixtures thereof. In capsules, tablets, and pills, the dosage form may also contain buffering agents.

Solid dosage forms such as tablets, sugar pills, capsules, pills, and granules can be prepared by coating and shell materials, such as enteric coatings and other materials known in the art. They may contain opaque agents, and the active compounds or compounds in this combination can be released in a delayed mode in a certain part of the digestive tract. Examples of usable embedding components are polymeric substances and wax based substances. When necessary, the active compound and one or more of the above-mentioned excipients can form microcapsule.

Liquid dosage forms for oral administration include pharmaceutically acceptable emulsion, solutions, suspensions, syrups or tinctures. In addition to active compounds, liquid formulations may include conventional inert diluents used in this art, such as water or other solvents, solubilizers, and emulsifiers, such as ethanol, isopropanol, ethyl carbonate, ethyl acetate, propylene glycol, 1,3-butanediol, dimethylformamide, as well as oils, particularly cottonseed oil, peanut oil, corn germ oil, olive oil, castor oil, and sesame oil, or the combination thereof.

In addition to these inert diluents, the composition may also contain adjuvants such as humectants, emulsifiers and suspensions, sweeteners, corrigens, and spices.

In addition to the active compounds, the suspensions may contain suspensions, such as ethoxylated isooctadecanol, polyoxyethylene sorbitol and dehydrated sorbitol esters, microcrystalline cellulose, methanol aluminum and agar, or the combination thereof.

The compositions for parenteral injection may include physiologically acceptable sterile aqueous or anhydrous solutions, dispersions, suspensions or emulsions, and sterile powders for being re-dissolved into sterile injectable solutions or dispersions. Suitable aqueous and non-aqueous carriers, diluents, solvents, or excipients include water, ethanol, polyols, and suitable combination thereof.

The compound of the present invention can be administered alone or in combination with other pharmaceutically acceptable therapeutic agents.

When administered in combination, the pharmaceutical composition further comprises one or more (2, 3, 4, or more) other pharmaceutically acceptable therapeutic agents. One or more of the other pharmaceutically acceptable therapeutic agents (2, 3, 4, or more) can be used simultaneously, separately, or sequentially with the compound of the present invention for preventing, treating, and/or alleviating FGFR mediated diseases.

When using a pharmaceutical composition, a safe and effective dosage of the compound of the present invention is applied to a mammal (such, human) in need of treatment, wherein the dosage of administration is a pharmaceutically effective dosage, for a person weighed 60 kg, the daily dosage is usually 1-2000 mg, preferably 20-500 mg. Of course, the specific dosage should also consider factors such as the route of administration and the patient's health condition, which are within the skill range of skilled physicians.

### Beneficial Effects

According to embodiments of the present invention, the present invention provides novel structurally, metabolically stable, and highly effective FGFR inhibitors, which can be used for effectively treating FGFR related diseases and conditions.

The compound of the present invention has a good inhibitory effect on FGFR and good in vitro efficacy. In addition, the results of mouse experiments showed that the compounds of the present invention exhibited excellent pharmacokinetic properties and good druggability.

The following will explain the solution of the present invention in conjunction with the examples. Technicians in this art will understand that the following examples are only used to illustrate the present invention and should not be considered as limiting the scope of the present invention. The specific technology or conditions with no description in the examples are performed under the technology or conditions described in the literature in this art or according to manufacturer's instructions. The reagents or instruments used without specifying the manufacturer are conventional products that can be obtained through commercial purchase.

Unless otherwise specified, the structures of the compounds of the present invention are determined by nuclear magnetic resonance (NMR) and/or mass spectrometry (MS). The unit of NMR shift is 10-6 ppm. The solvents used for NMR determination are deuterated dimethyl sulfoxide, deuterated chloroform, deuterated methanol, etc. The internal standard is tetramethylsilane (TMS).

The abbreviation definition of the present invention is as follows:
M: molar concentration, such as 1M hydrochloric acid refers to 1 mol/L hydrochloric acid solution
HATU: 0-(7-azabenzotriazole-1-yl)-N,N,N,N-tetramethylurea hexafluorophosphate
T3P: 1-propylphosphamide
DIPEA: also known as DIEA, diisopropylethylamine, i.e. N,N-diisopropylethylamine
NIS: N-iodosuccinimide
NBS: N-bromosuccinimide
EA: ethyl acetate
TFA: trifluoroacetic acid
DMF: N,N-dimethylformamide
THF: tetrahydrofuran
PE: petroleum ether
LC-MS: liquid chromatography-mass spectrometry
DMSO: dimethyl sulfoxide
Xantphos: 4,5-diphenylphosphine-9,9-dimethyloxaanthracene
TLC: thin layer chromatography
IC50: half inhibitory concentration, which refers to the concentration at which half of the maximum inhibitory effect is achieved.

Unless otherwise indicated, the compounds listed herein are named and numbered using ChemBioDraw Ultra 14.0.

### Intermediate 1g 5-iodo-7-methyl-7H-pyrrolo [2,3-d] pyrimidin-4-amine

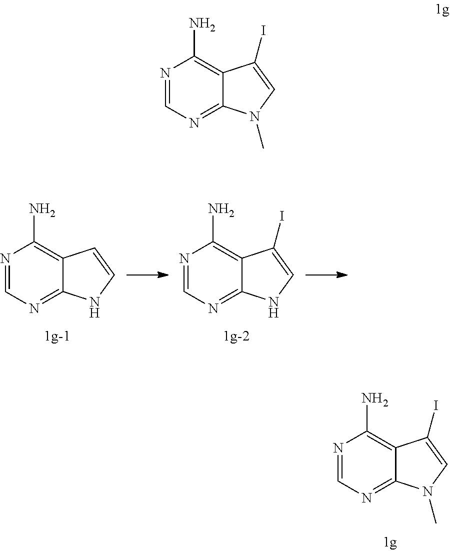

### Step 1 5-iodo-7H-pyrrolo [2,3-d] pyrimidin-4-amine 1g-2

Compound 1g-1 (1.0 g, 7.46 mmol) was dissolved in dichloromethane (15 mL), sodium hydroxide (329 mg, 8.21 mmol) and NIS (1.75 g, 7.84 mmol) were added in sequence at room temperature and reacted for 18 h. The reaction solution was then concentrated added water (20 mL), and stirred for 30 min, filtered, dried to obtain compound 1g-2 (1.85g), gray solid.

MS (ESI) m/z 261 [M+H]⁺.

¹H NMR (400 MHz, DMSO-d₆) δ 11.97 (s, 1H), 8.06 (s, 1H), 7.37 (s, 1H), 6.55 (s, 2H).

### Step 2: 5-iodo-7-methyl-7H-pyrrolo [2,3-d] pyrimidin-4-amine 1g

The 1g-2 (1.0 g, 3.85 mmol) was dissolved in DMF (20 mL), cesium carbonate (2.5 g, 7.69 mmol) and iodomethane (665 mg, 4.62 mmol) were added. The reaction solution was heated up to 80° C. and reacted for 18 h. After cooled down to room temperature, the reaction solution was filtered and concentrated to obtain a crude product, the crude product was purified by column chromatography (PE/EtOAc=0/1) to obtain compound 1g (0.78 g), brown solid.

MS (ESI) m/z 275 [M+H]⁺.

¹H NMR (400 MHz, CDCl₃) δ 8.32 (s, 1H), 7.05 (s, 1H), 5.73 (s, 2H), 3.81 (s, 3H).

### Intermediate 1j N-(3-fluoro-4-(4,4,5,5-tetramethyl-1,3,2-dioxaborolan-2-yl)phenyl)

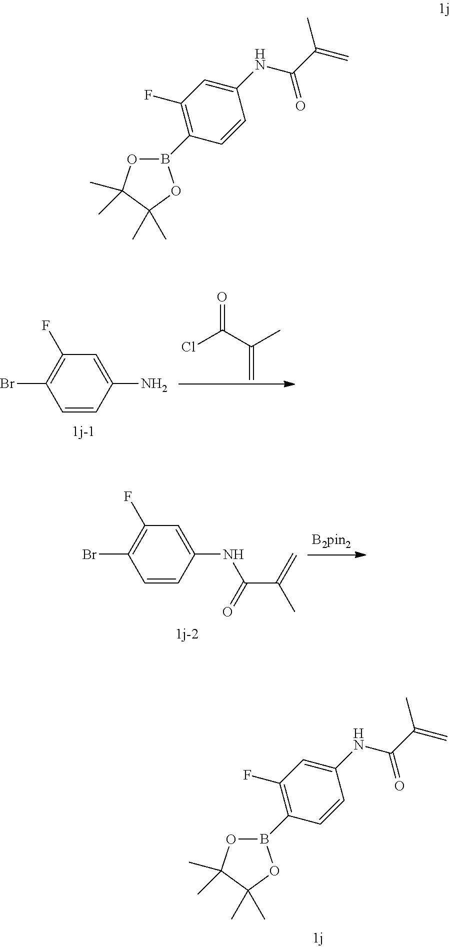

### Step 1: N-(4-bromo-3-fluorophenyl) methacrylamide 1j-2

Compound 1j-1 (5.0 g, 26.3 mmol) and triethylamine (8.0 g, 11 ml) were dissolved in DCM (50 ml), at 0° C., 2-methylacryloyl chloride (3.03 g, 2.8 ml) was added dropwise to the reaction solution, and stirred for 1 h. The reaction solution was diluted with water (100 ml), and extracted with DCM (100 ml) three times. The organic phase was washed with saturated saline solution, dried over anhydrous Na₂SO₄, and concentrated under reduced pressure, the obtained crude product was purified by silica gel column chromatography (EA/PE=1/4) to obtain compound 1j-2 (4.9 g, 72%), pale yellow white solid.

MS (ESI) m/z 258, 260 [M+H]⁺.

### Step 2: N-(3-fluoro-4-(4,4,5,5-tetramethyl-1,3,2-dioxoborolan-2-yl)phenyl) methacrylamide 1j

To a sealed tube was added Compound 1j-2 (2.0 g, 7.75 mmol), bis(pinacolato)diboron (3.94 g, 15.5 mmol), Pd(dppf)Cl₂ (567 mg, 0.77 mmol), AcOK (2.28 g, 23.25 mmol), and 1,4-dioxane (20 ml), and replaced with argon gas three times. The mixture reacted at 90° C. for 5 h. The reaction was then quenched with water and extracted with DCM. The organic phase was washed with saturated saline solution, dried over anhydrous Na₂SO₄, and concentrated under reduced pressure. The obtained crude product was purified by silica gel column chromatography (EA/PE=1/8 to 1/4) to obtain compound 1j (1.52 g, 64%), yellow solid.

MS (ESI) m/z 306 [M+H]⁺.

¹H NMR (500 MHz, CDCl₃) δ 7.70-7.65 (m, 2H), 7.53 (dd, J 11.5, 1.9 Hz, 1H), 7.20 (dd, J 8.2, 1.9 Hz, 1H), 5.78 (s, 1H), 5.48 (d, J 1.7 Hz, 1H), 2.04 (s, 3H), 1.34 (s, 12H).

### Intermediate 4a N-(4-(4,4,5,5-tetramethyl-1,3,2-dioxaborolan-2-yl)phenyl) methacrylamide

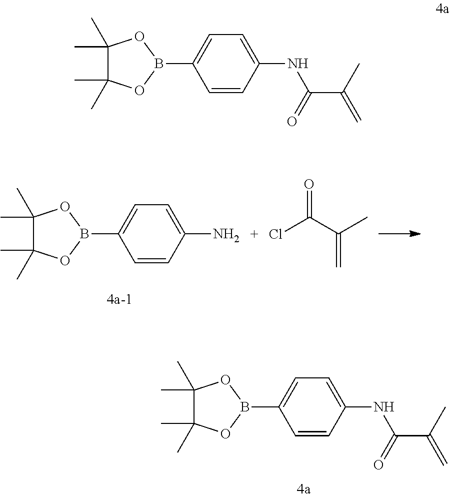

Compound 4a-1 (2.0 g, 9.12 mmol) was dissolved in ultra dry DCM (50 mL), triethylamine (2.77 g, 27.36 mmol) was added, and stirred at 0° C. for 5 min. The 2-methylacrylamide was diluted with ultra dry DCM (30 mL), slowly dropwise added to the reaction solution and reacted at 0° C. for 2 h. The reaction solution was poured into water, and extracted with DCM three times. The organic layers were combined and washed with saturated salt once, the organic phase was dried over anhydrous sodium sulfate, concentrated under reduced pressure, and purified by column chromatography (PE/EA=2/1) to obtain compound 4a (2.15 g), white solid.

MS (ESI) m/z 288 [M+H]⁺.

¹H NMR (400 MHz, DMSO-d₆) δ 9.87 (s, 1H), 7.71 (d, J=8.5 Hz, 2H), 7.61 (d, J=8.6 Hz, 2H), 5.81 (t, J=1.0 Hz, 1H), 5.55-5.51 (m, 1H), 1.95 (s, 3H), 1.28 (s, 12H).

### Intermediate 8e N-(4-(4-amino-5-bromo-7-methyl-7H-pyrrolo [2,3-d] pyrimidin-6-yl)-3-fluorophenyl) methacrylamide

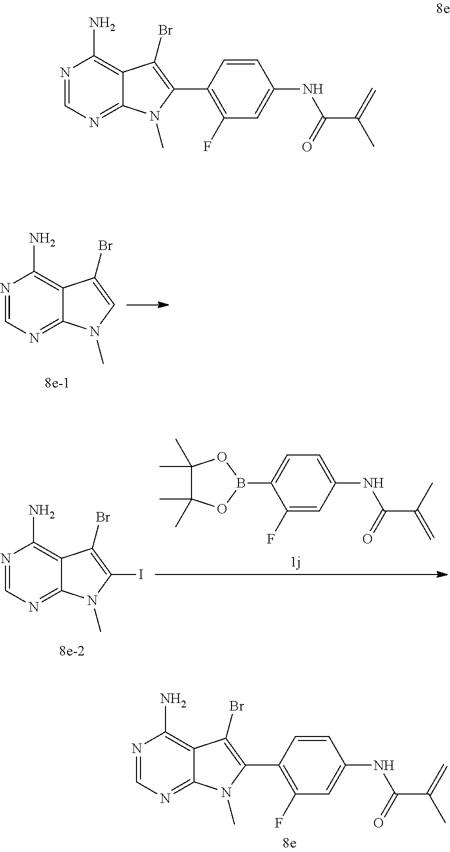

### Step 1: 5-bromo-6-iodo-7-methyl-7H-pyrrolo [2,3-d] pyrimidin-4-amino 8e-2

Compound 8e-1 (10.6 g 0.0466 mol) was dissolved in dichloromethane (100 mL) and trifluoroacetic acid (26.7 g 0.2334 mol), at 10° C., N-iodosuccinimide (10.5 g 0.0466 mol) was added in batches to react for 4 h. After the reaction was completed, saturated sodium bicarbonate aqueous solution (150 mL) was added slowly at 10° C., then a solid was precipitated, filtered and dried to obtain compound 8e-2 (13.4 g), purple solid.

MS (ESI) m/z 352.6 [M+H]⁺.

¹H NMR (400 MHz, CDCl₃) δ 8.22 (s, 1H), 5.61 (s, 2H), 3.80 (s, 3H).

### Step 2: N-(4-(4-amino-5-bromo-7-methyl-7H-pyrrolo [2,3-d] pyrimidin-6-yl)-3-fluorophenyl) methacrylamide 8e

Compound 1j (2 g, 6.5 mmol), compound 8e-2 (2.3 g, 6.5 mmol), tetrakis (triphenylphosphine) palladium (0.65 g, 0.56 mmol), and potassium phosphate (3.6 g, 0.017 mol) were added into a reaction flask, and replaced with nitrogen gas three times, then DMF (35 mL) and water (5 mL) were added, and stirred at 50° C. for 16 h. After the reaction was completed, water (150 mL) and dichloromethane (200 mL) were added to extract the solution, the organic phase was washed with saturated saline and concentrated to obtain a crude product. The crude product was purified by column chromatography to obtain compound 8e (1.1 g), light yellow solid.

MS (ESI) m/z 404.2 [M+H]⁺.

¹H NMR (400 MHz, CDCl₃) δ 8.35 (s, 1H), 7.82 (dd, J 11.7, 2.1 Hz, 1H), 7.69 (s, 1H), 7.44-7.33 (m, 2H), 5.87 (s, 1H), 5.66 (s, 2H), 5.58 (q, J 1.5 Hz, 1H), 3.66 (d, J 1.1 Hz, 3H), 2.12 (dd, J 1.6, 0.9 Hz, 3H).

### Intermediate 9e N-(4-(4-amino-5-bromo-7-methyl-7H-pyrrolo [2,3-d] pyrimidin-6-yl) phenyl) methacrylamide

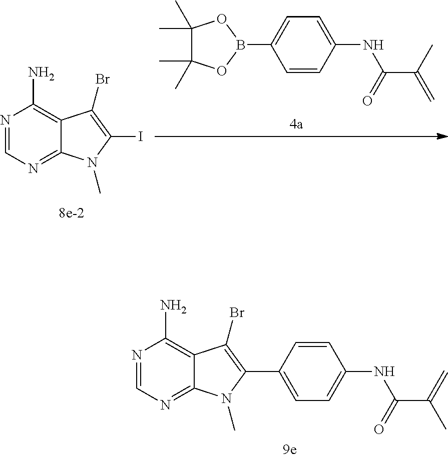 [SMILES: Cn1c(I)c(Br)c2c(N)ncnc21 ; C=C(C)C(=O)Nc1ccc(B2OC(C)(C)C(C)(C)O2)cc1 ; C=C(C)C(=O)Nc1ccc(-c2c(Br)c3c(N)ncnc3n2C)cc1]

Intermediate 9e was synthesized with reference to intermediate 8e. MS (ESI) m/z 386.32 [M+H]⁺.

¹H NMR (400 MHz, CDCl₃) δ 8.32 (s, 1H), 7.77-7.71 (m, 2H), 7.62 (s, 1H), 7.48-7.41 (m, 2H), 5.90-5.82 (m, 1H), 5.63 (s, 2H), 5.53 (q, J 1.6 Hz, 1H), 3.67 (s, 3H), 2.10 (dd, J=1.6, 0.9 Hz, 3H).

### Intermediate 12a 1-(4-(4-amino-6-iodo-7-methyl-7H-pyrrolo [2,3-d] pyrimidin-5-yl)-2-fluorophenyl) imino) hexahydro-1λ⁶-thiopyran-1-oxide

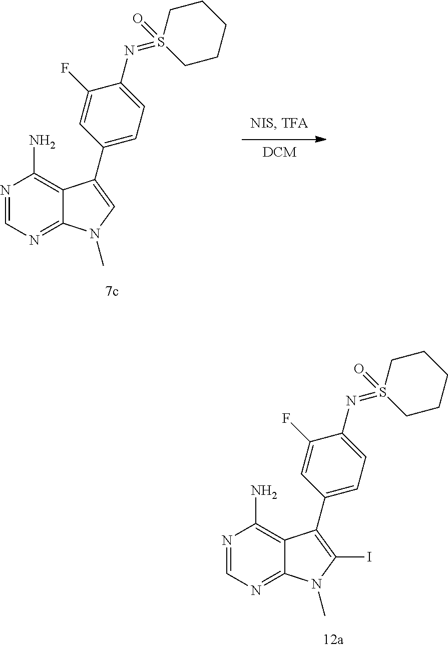

Intermediate 12a was synthesized with reference to intermediate 84b, MS (ESI) m/z 500 [M+H]⁺.

### Intermediate 12b N-(4-fluoro-3-(4,4,5,5-tetramethyl-1,3,2-dioxaborolan-2-yl) phenyl) methacrylamide

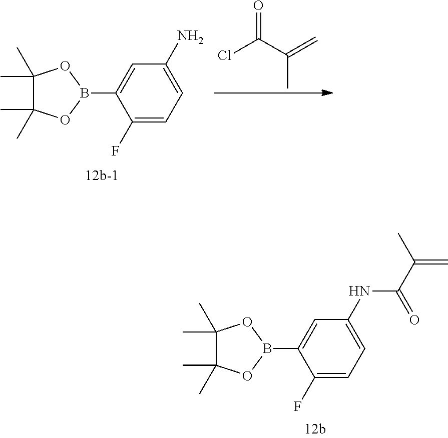

Intermediate 12b was synthesized with reference to compound 4a, MS (ESI) m/z 306 [M+H]⁺.

### Intermediate 14a N-(3-methoxy-4-(4,4,5,5-tetramethyl-1,3,2-dioxaborolan-2-yl) phenyl) methacrylamide

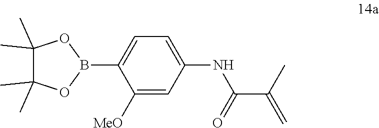 [SMILES: C=C(C)C(=O)Nc1ccc(B2OC(C)(C)C(C)(C)O2)c(OC)c1]

Intermediate 14a was synthesized with reference to WO2020231990A1.

### Intermediate 15a (E)-N-(4-(4,4,5,5-tetramethyl-1,3,2-dioxaborolan-2-yl)phenyl)but-2-enamide

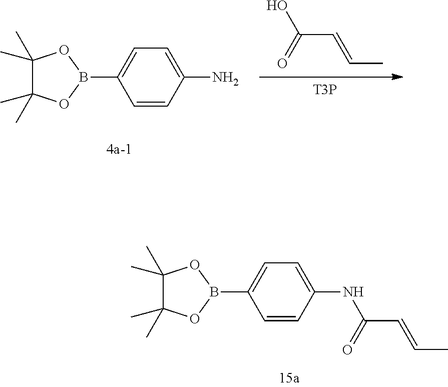

Intermediate 15a was synthesized with reference to compound 21a, MS (ESI) m/z 289 [M+H]⁺.

¹H NMR (400 MHz, CDCl₃) δ 7.80-7.73 (m, 2H), 7.57 (d, J 8.1 Hz, 2H), 7.28 (s, 1H), 6.99 (dq, J 15.2, 6.9 Hz, 1H), 5.94 (dq, J 15.0, 1.7 Hz, 1H), 1.90 (dd, J 6.9, 1.7 Hz, 3H), 1.33 (s, 12H).

### Intermediate 16a (E)-4-(dimethylamino)-N-(4-(4,4,5,5-tetramethyl-1,3,2-dioxaborolane-2-yl)phenyl) but-2-enamide

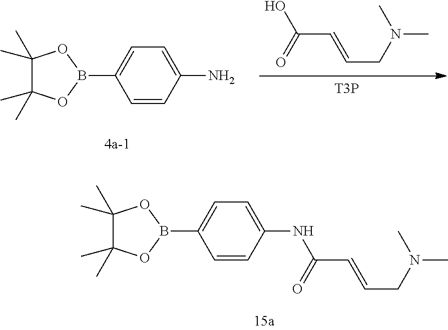

Intermediate 16a was synthesized with reference to compound 21a, MS (ESI) m/z 331 [M+H]⁺.

### Intermediate 18c 1-((4-(4-amino-6-iodo-7-methyl-7H-pyrrolo [2,3-d] pyrimidin-5-yl)-2-fluorophenyl) imino) tetrahydro-1H-1λ⁶-thiophene-1-oxide

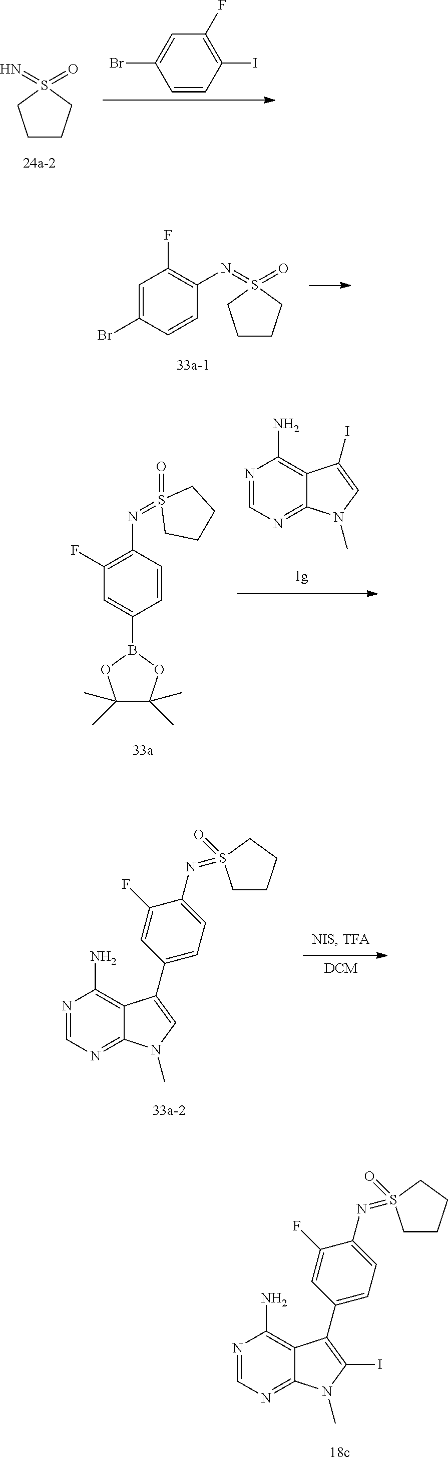

Intermediate 18c was synthesized with reference to intermediate 77d, MS (ESI) m/z 486

[M+H]⁺.

### Intermediate 19f ((4-(4-amino-6-bromo-7-methyl-7H-pyrrolo [2,3-d] pyrimidin-5-yl)-2-fluorophenyl) imino) dimethyl-λ⁶-sulfone

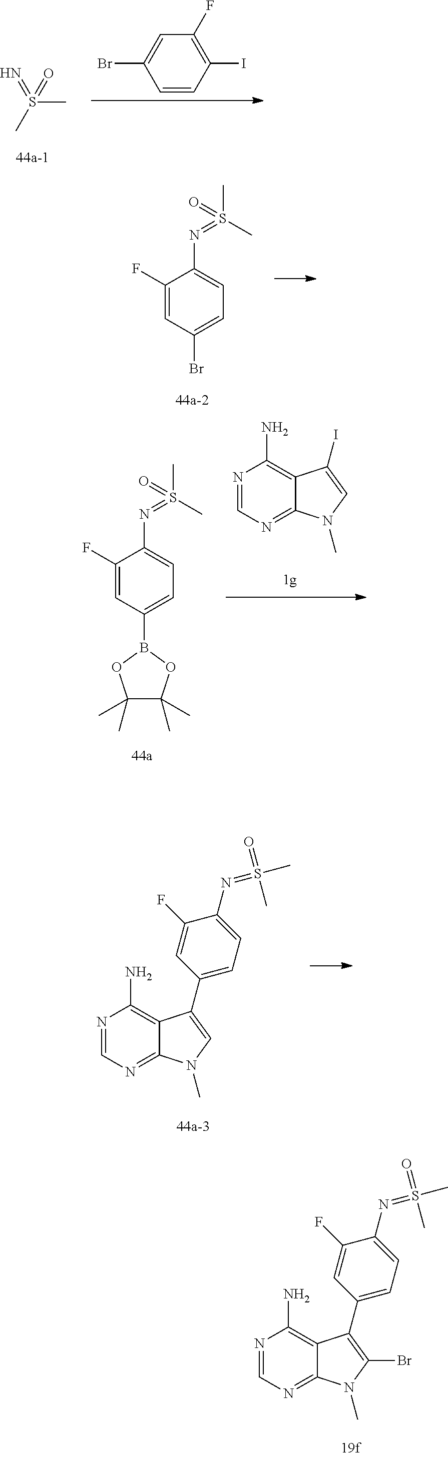

Intermediate 19f was synthesized with reference to compound 5d, MS (ESI) m/z 413 [M+H]⁺.

### Intermediate 21a 2-fluoro-N-(4-(4,4,5,5-tetramethyl-1,3,2-dioxaborolan-2-yl) phenyl) acrylamide

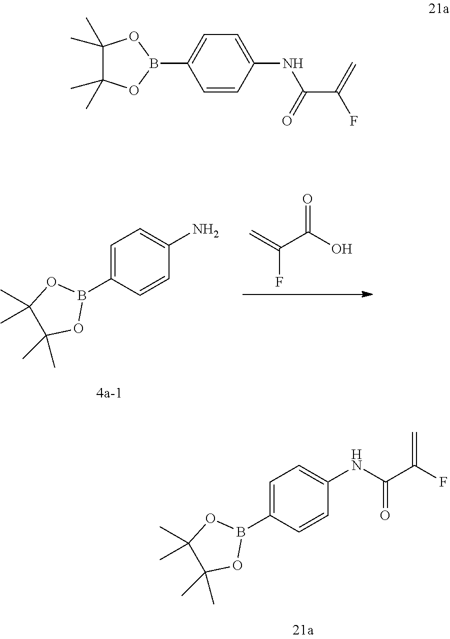

Compound 4a-1 (500 mg, 2.28 mmol), 2-fluoroacrylic acid (246.6 mg, 2.74 mmol), and triethylamine (1.38 g, 13.68 mmol) were dissolved in DMF (20 mL), and T3P (1.09 g, 3.42 mmol) was added dropwise at 0° C. to react overnight. After the reaction was completed, H₂O (100 mL) was added and extracted with ethyl acetate (50 mL×3) three times. The organic layers were combined, washed with saturated saline solution, the organic layer was dried over anhydrous sodium sulfate, concentrated under reduced pressure, and purified by column chromatography (PE/EA=3/1) to obtain compound 21a (480 mg), yellow solid.

MS (ESI) m/z 292.1 [M+H]⁺.

¹H NMR (400 MHz, CDCl₃) δ 7.97 (s, 1H), 7.81 (d, J 8.5 Hz, 2H), 7.61 (d, J=8.5 Hz, 2H), 5.83 (dd, J 47.9, 3.4 Hz, 1H), 5.26 (dd, J 15.3, 3.4 Hz, 1H), 1.34 (s, 12H).

### Intermediate 22a N-(3-methyl-4-(4,4,5,5-tetramethyl-1,3,2-dioxaborolan-2-yl) phenyl) methacrylamide

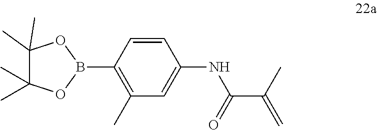 [SMILES: C=C(C)C(=O)Nc1ccc(B2OC(C)(C)C(C)(C)O2)c(C)c1]

Intermediate 22a was synthesized with reference to WO2022109577 A1.

### Intermediate 24a 1-((4-bromophenyl)imino)tetrahydro-1H-1λ⁶-thiophene-1-oxide

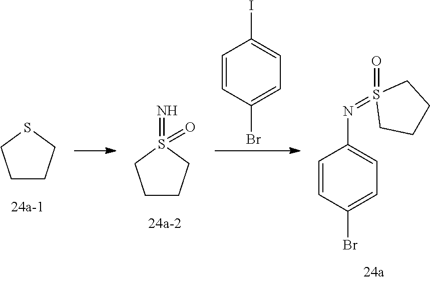 [SMILES: C1CCSC1 ; N=S1(=O)CCCC1 ; Brc1ccc(I)cc1 ; O=S1(=Nc2ccc(Br)cc2)CCCC1]

### Step 1: 1-iminotetrahydro-1H-1λ⁶-thiophene-1-oxide 24a-2

At room temperature, tetrahydrothiophene (1 g, 11.34 mmol) and ammonium carbamate (1.33 g, 17.01 mmol) were added to a round bottom flask and dissolved in methanol (10 mL). It was cooled to 0° C. in an ice water mixture, and iodobenzene diethyl ester (7.67 g, 23.82 mmol) was added in batches. After the addition was completed, the temperature was raised up slowly to room temperature, and the reaction was stirred for 16 h. After the reaction was completed, the mixture was concentrated under reduced pressure to obtain a crude product. The crude product was filtered under reduced pressure, the filtrate was diluted with water (3 mL), and then sodium bicarbonate solid was added slowly to the diluted filtrate to adjust the pH to 7. The mixture was filtered under reduced pressure, and the filtrate was concentrated under reduced pressure to obtain a crude product. The crude product was purified by column chromatography (DCM/MeOH=20/1) to obtain compound 24a-2 (0.7 g), orange yellow oil.

MS (ESI) m/z 120 [M+H]⁺.

¹H NMR (400 MHz, DMSO-d₆) δ 3.00-2.92 (m, 4H), 2.14-2.01 (m, 4H).

### Step 2 1-((4-bromophenyl) imino) tetrahydro-1H-1λ⁶-thiophene-1-oxide 24a

At room temperature, compound 24a-2 (3 g, 25.2 mmol), p-bromoiodobenzene (7.83 g, 27.72 mmol), tris (dibenzylideneacetone) dipalladium (0.46 g, 0.5 mmol), 4,5-bisdiphenyl phosphine-9,9-dimethylxanthene (0.875 g, 1.51 mmol), cesium carbonate (9.86 g, 30.24 mmol), and 1,4-dioxane (30 mL) were sequentially added to a round bottom flask, followed by vacuum pumping and nitrogen gas purging three times, then heated up to 100° C. and stirred for 16 h. After the reaction was completed, the mixture was cooled down to room temperature and filtered under reduced pressure. The filter cake was washed with dichloromethane three times, and all filtrates were combined and concentrated under reduced pressure to obtain a crude product. The crude product was purified by column chromatography (PE/EtOAc=4/1) to obtain compound 24a (5.3 g), yellow solid.

MS (ESI) m/z 273 [M+H]⁺.

¹H NMR (400 MHz, DMSO-d₆) δ 7.35 (d, J=8.0 Hz, 2H), 6.87 (d, J=8.0 Hz, 2H), 3.33-3.17 (m, 4H), 2.14-2.04 (m, 4H).

### Intermediate 25a N-(3-cyano-4-(4,4,5,5-tetramethyl-1,3,2-dioxaborolan-2-yl) phenyl) methacrylamide

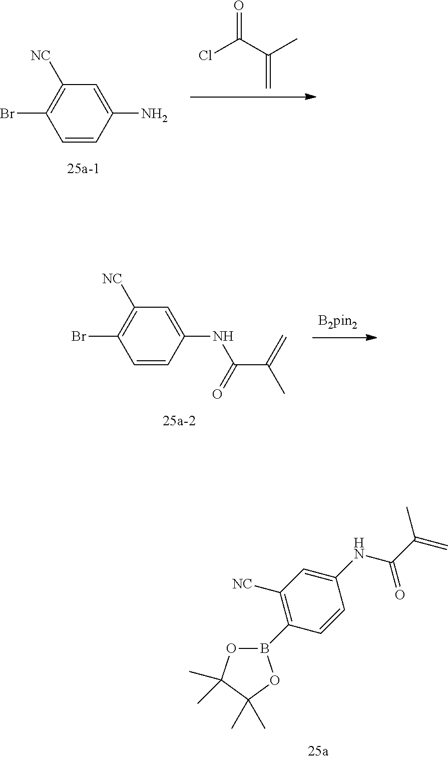

Intermediate 25a was synthesized with reference to compound 1j, MS (ESI) m/z 313 [M+H]⁺.

### Intermediate 16a N-(4-(4,4,5,5-tetramethyl-1,3,2-dioxaborolan-2-yl)phenyl)but-2-ynamide

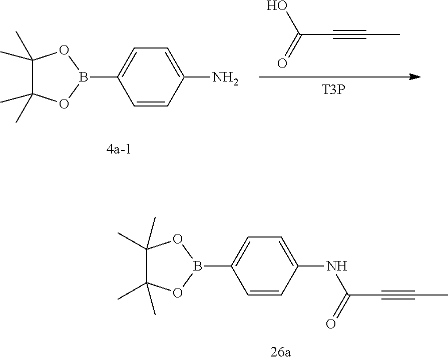

Intermediate 26a was synthesized with reference to compound 1a, MS (ESI) m/z 286 [M+H]⁺.

### Intermediate 27a 1-((2,6-difluoro-4-(4,4,5,5-tetramethyl-1,3,2-dioxaborolan-2-yl) phenyl) imino) tetrahydro-1H-1λ⁶-thiophene-1-oxide

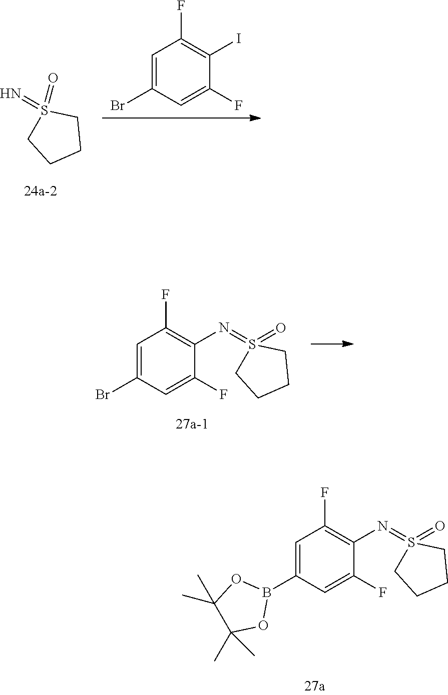 [SMILES: N=S1(=O)CCCC1 ; Fc1cc(Br)cc(F)c1I ; O=S1(=Nc2c(F)cc(Br)cc2F)CCCC1 ; CC1(C)OB(c2cc(F)c(N=S3(=O)CCCC3)c(F)c2)OC1(C)C]

Intermediate 27a was synthesized with reference to compound 1f, MS (ESI) m/z 358 [M+H]⁺.

### Intermediate 29a 1-((5-bromopyridin-2-yl) imino) tetrahydro-1H-1λ⁶-thiophene-1-oxide

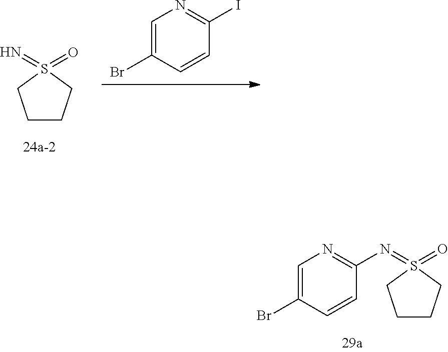 [SMILES: N=S1(=O)CCCC1 ; Brc1ccc(I)nc1 ; O=S1(=Nc2ccc(Br)cn2)CCCC1]

Intermediate 29a was synthesized with reference to compound 1d, MS (ESI) m/z 276 [M+H]⁺.

### Intermediate 33a 1-((2-fluoro-4-(4,4,5,5-tetramethyl-1,3,2-dioxaborolan-2-yl) phenyl) imino) tetrahydro-1H-1λ⁶-thiophene-1-oxide

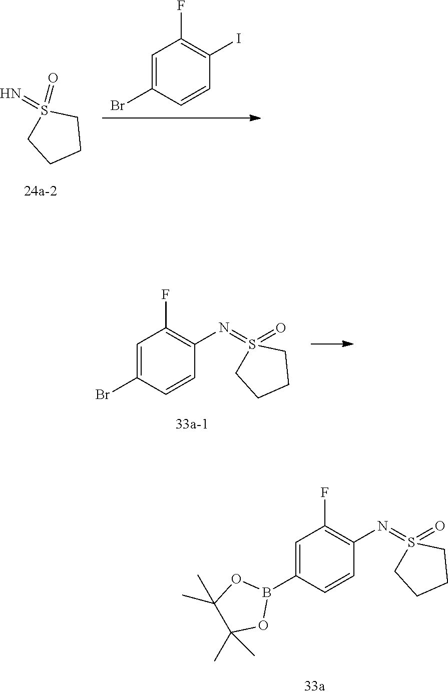 [SMILES: N=S1(=O)CCCC1 ; Fc1cc(Br)ccc1I ; O=S1(=Nc2ccc(Br)cc2F)CCCC1 ; CC1(C)OB(c2ccc(N=S3(=O)CCCC3)c(F)c2)OC1(C)C]

Intermediate 33a was synthesized with reference to compound 1f, MS (ESI) m/z 340 [M+H]⁺

### Intermediate 34a 1-((2-methyl-4-(4,4,5,5-tetramethyl-1,3,2-dioxaborolan-2-yl) phenyl) imino) hexahydro-1λ⁶-thiopyran-1-oxide

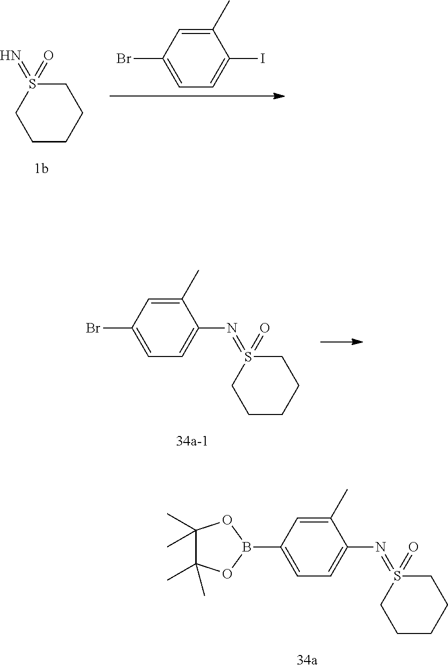

Intermediate 34a was synthesized with reference to compound 1f, MS (ESI) m/z 350 [M+H]⁺.

### Intermediate 60d 1-(4-(4-amino-6-iodo-7-methyl-7H-pyrrolo [2,3-d] pyrimidin-5-yl) phenyl) imino) tetrahydro-1H-1λ⁶-thiophene-1-oxide

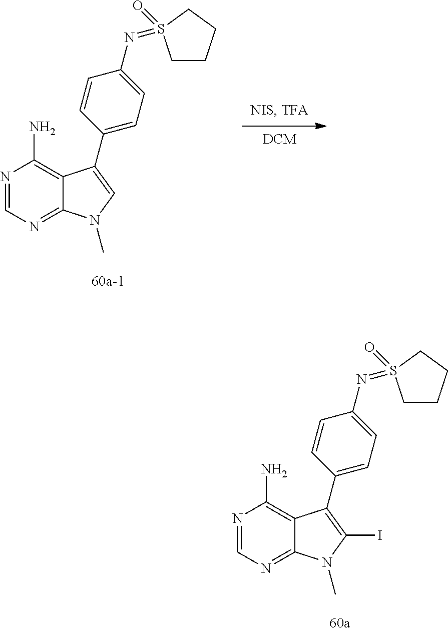

Intermediate 60a was synthesized with reference to intermediate 84b, MS (ESI) m/z 468 [M+H]⁺.

### Intermediate 61a 1-(4-(4-amino-6-iodo-7-methyl-7H-pyrrolo [2,3-d] pyrimidin-5-yl)-3-fluorophenyl) imino) tetrahydro-1H-1λ-thiophene-1-oxide

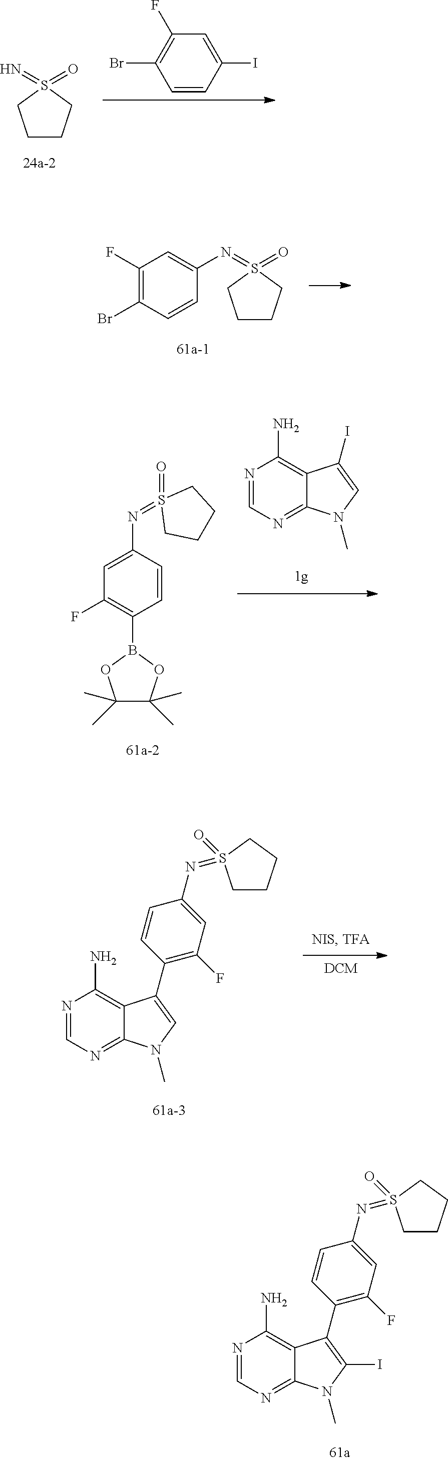

Intermediate 61a was synthesized with reference to intermediate 77d, MS (ESI) m/z 486 [M+H]⁺.

### Intermediate 74a N-(3-chloro-4-(4,4,5,5-tetramethyl-1,3,2-dioxaborolan-2-yl) phenyl) methacrylamide

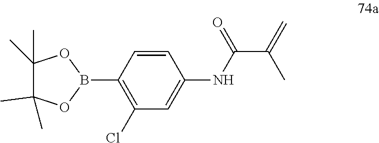 [SMILES: C=C(C)C(=O)Nc1ccc(B2OC(C)(C)C(C)(C)O2)c(Cl)c1]

Intermediate 74a was synthesized with reference to WO2023046117 A1.

### Intermediate 84a 1-((4-(4-amino-7-(methyl-d₃)-7H-pyrrolo [2,3-d] pyrimidin-5-yl)-2-fluorophenyl) imino)-1λ⁶-thiophene-1-oxide

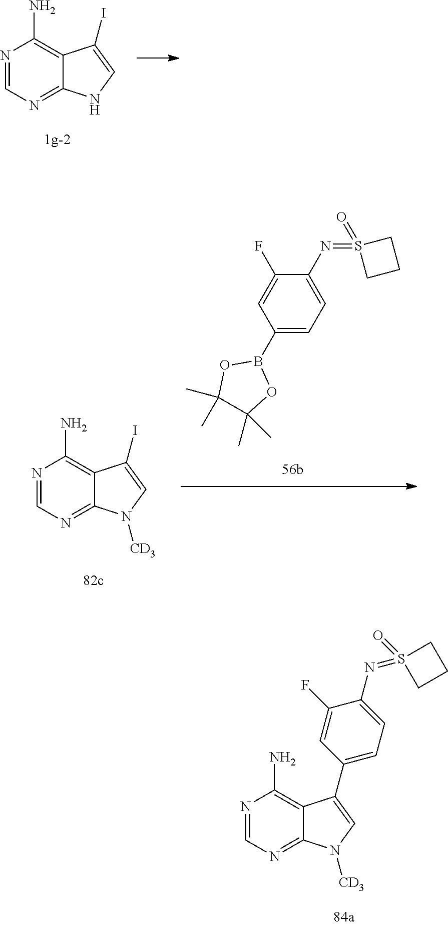 [SMILES: Nc1ncnc2[nH]cc(I)c12 ; [2H]C([2H])([2H])n1cc(I)c2c(N)ncnc21 ; CC1(C)OB(c2ccc(N=S3(=O)CCC3)c(F)c2)OC1(C)C ; [2H]C([2H])([2H])n1cc(-c2ccc(N=S3(=O)CCC3)c(F)c2)c2c(N)ncnc21]

### Step 1 5-iodo-7-(methyl-d₃)-7H-pyrrole [2,3-d] pyrimidin-4-amine 82c

Compound 1g-2 (10.0 g, 38.5 mmol) was dissolved in N,N-dimethylformamide (50 mL), cesium carbonate (18.8 g, 57.8 mmol) was added at room temperature, then cooled down to 0° C., deuterated iodomethane (6.70 g, 46.1 mmol) was added to react at room temperature for 1 h. After the reaction was completed, it was extracted with dichloromethane (100 mL×3), and the organic phases were combined and concentrated under reduced pressure to obtain a crude product. To the crude product was added water (100 mL×3), a brown solid was precipitated. The brown solid was collected by filtration to obtain compound 82c (5.80 g), brown solid.

MS (ESI) m/z 277.70 [M+H]⁺.

¹H NMR (400 MHz, DMSO) δ 8.10 (s, 1H), 7.45 (s, 1H), 6.85-6.35 (m, 2H).

### Step 2 1-((4-(4-amino-7-(methyl-d₃)-7H-pyrrolo [2,3-d] pyrimidin-5-yl)-2-fluorophenyl) imino)-1λ⁶ thiophene-1-oxide 84a

Compound 82c (0.50 g, 1.80 mmol) was dissolved in 1,4-dioxane (10 mL) and H₂O (2 mL), compound 56b (0.65 g, 2.00 mmol), potassium phosphate (1.15 g, 5.40 mmol), and tetrakis (triphenylphosphine) palladium (0.21 g, 0.18 mmol) were added, and reacted at 90° C. for 14 h under N₂ protection. After TLC monitored the completion of the reaction, it was extracted with ethyl acetate (50 mL×3), and the organic phases were combined and concentrated under reduced pressure to obtain a crude product. The crude product was purified by column chromatography (DCM/MeOH=10/1) to obtain compound 84a (420 mg).

MS (ESI) m/z 349.10 [M+H]⁺.

### Intermediate 85a diethyl ((3-fluoro-4-(4,4,5,5-tetramethyl-1,3,2-dioxaborolan-2-1) phenyl) imino)-λ⁶-sulfone

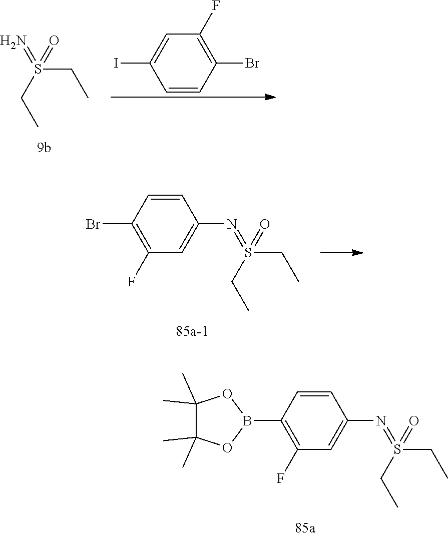

Intermediate 85a was synthesized with reference to compound 1f, MS (ESI) m/z 342 [M+H]⁺.

### Intermediate 90c N-(5-chloro-2-methyl-4-(4,4,5,5-tetramethyl-1,3,2-dioxaborolan-2-yl) phenyl) methacrylamide

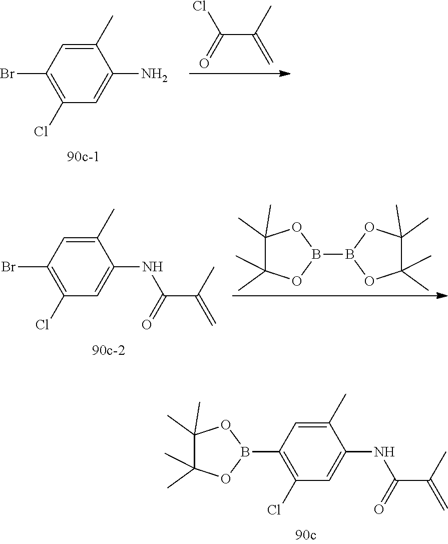

### Step 1: N-(4-bromo-5-chloro-2-methylphenyl) methacrylamide 90c-2

The 90c-1 (500 mg, 2.27 mmol) and Et₃N(459 mg, 4.54 mmol) were dissolved in DCM (5 mL), methacryloyl chloride (357 mg, 3.41 mmol) was added to the reaction solution at 0° C. to react for 1 h at 0° C., then concentrated under reduced pressure, extracted with added H₂O (20 mL) and dichloromethane, the organic phase was washed with saturated saline solution, dried over anhydrous sodium sulfate, concentrated under reduced pressure, and purified by column chromatography to obtain compound 90c-2 (509 mg, 2.27), white solid.

MS (ESI) m/z 288 [M+H]+

### Step 2: N-(5-chloro-2-methyl-4-(4,4,5,5-tetramethyl-1,3,2-dioxaborolan-2-yl) phenyl) methacrylamide 90c

Compound 90c-2 (457 mg, 1.29 mmol), anhydrous 1,4-dioxane (6 mL), bis(pinacolato)diboron (606 mg, 2.39 mmol), potassium acetate (468 mg, 4.77 mmol), and Pd(dppf)Cl₂ (115 mg, 0.16 mmol) were added to the reaction flask in sequence, after replaced with nitrogen three times, heated up to 90° C. to react for 4 h. TLC showed the formation of new dots. After the reaction was completed, the reaction solution was cooled down to room temperature, diluted with EA and H₂O, filtered through diatomaceous earth, extracted with ethyl acetate, and the organic phase was washed with saturated saline solution, dried over anhydrous sodium sulfate, concentrated under reduced pressure, and purified by column chromatography (PE/EA=1/1) to obtain compound 90c (350 mg), light yellow oil.

MS (ESI) m/z 336 [M+H]⁺

### Intermediate 92a 2-((dimethylamino)methyl)-N-(4-(4,4,5,5-tetramethyl-1,3,2-dioxaborolan-2-yl) phenyl) acrylamide

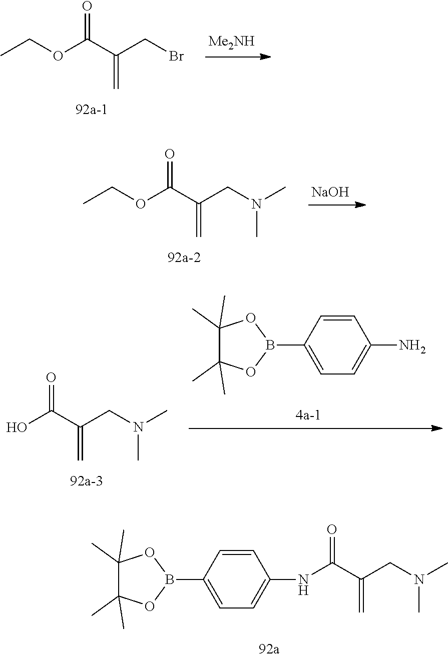

### Step 1 2-((dimethylamino) methyl) ethyl acrylate 92a-2

Compound 92a (5 g, 26.04 mmol) was dissolved in tetrahydrofuran (20 mL) at room temperature. After it was cooled down to 0° C., the dimethylamine (1.40 g, 31.2 mmol) dissolved in tetrahydrofuran (10 mL) was added slowly dropwise to the above solution to react at room temperature for 6 h. After TLC monitored the completion of the reaction, the mixture was filtered through a Buchner funnel, and the filtrate was collected and concentrated under reduced pressure to obtain crude product compound 92a-2 (2 g), yellow oily liquid.

MS (ESI) m/z 158 [M+H]⁺.

### Step 2 2-((dimethylamino) methyl) acrylic acid 92a-3

At room temperature, compound 92a-2 (2 g, 12.7 mmol) was dissolved in tetrahydrofuran (5 mL) and water (5 mL), and lithium hydroxide (365.76 mg, 15.24 mmol) was added in batches. The reaction was stirred at room temperature for additional 4 h. After TLC monitored the completion of the reaction, the mixture was concentrated under reduced pressure to obtain crude product compound 92a-3 (1.2 g), yellow solid.

MS (ESI) m/z 130 [M+H]⁺.

### Step 3 2-(dimethylamino)methyl)-N-(4-(4,4,5,5-tetramethyl-1,3,2-dioxaborolan-2-yl) phenyl) acrylamide 92a

At room temperature, compound 92a-3 (500 mg, 3.88 mmol) and 4-aminophenylboronic acid pinacol ester (679.34 mg, 3.10 mmol) were dissolved in dichloromethane (5 ml), and triethylamine (1.18 g, 11.64 mmol) was added. After it was cooled down to 0° C., a solution of 1-propylphosphonic anhydride in 50% ethyl acetate (1.85 g, 5.82 mmol) was added slowly dropwise to react at room temperature for 4 h. The reaction solution was diluted with water, extracted with dichloromethane (5 mL×3), washed with saturated saline solution (10 mL), and the organic phases were combined, dried over anhydrous sodium sulfate, and concentrated under reduced pressure to obtain a crude product. The crude product was purified by column chromatography (DCM/MeOH=15/1) to obtain compound 92a (130 mg), light yellow solid.

MS (ESI) m/z 331 [M+H]⁺.

### Intermediate 93a ((4-bromophenyl) imino) dicyclopropyl-λ⁶-sulfone

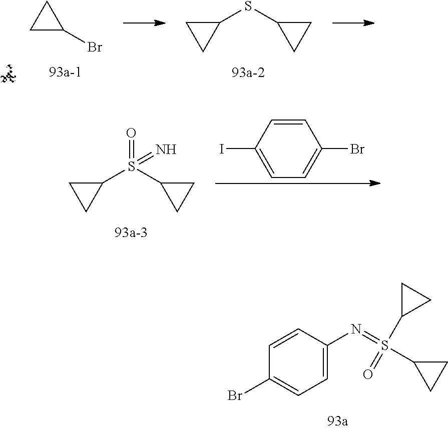

### Step 1 dicyclopropyl sulfide 93a-2

Compound 93a-1 (2.0 g, 16.7 mmol) was dissolved in DMF (5 mL), Na₂S (640 mg, 8.2 mmol) was added to react in a sealed tube at 100° C. for 16 h. After the reaction was completed, 30 mL of ethyl acetate was added, and the organic phase was washed with saline solution (10 mL×3), concentrated under reduced pressure to obtain a brown crude product 93a-2, which was directly used in the next step.

### Step 2 dicyclopropyl sulfoximine 93a-3

The crude product 93a-2 from the previous step was dissolved in methanol (15 mL), NH₄CO₂NH₂ (1.3 g, 16.7 mmol) and PhI(OAc)₂ (6.7 g, 20.8 mmol) was added in sequence. The reaction solution was left to react at room temperature for 16 h. After TLC monitored the completion of the reaction, it was cooled down to room temperature, the spun-dried crude product was purified by column chromatography (DCM/MeOH=10/1) to obtain compound 93a-3 (264 mg), brown oily substance.

MS (ESI) m/z 146.1 [M+H]⁺.

¹H NMR (400 MHz, CDCl₃) δ 2.54 (m, 2H), 1.20 (m, 4H), 1.02 (m, 4H).

### Step 3 ((4-bromophenyl) imino) dicyclopropyl-λ⁶-sulfone 93a

Compound 93a-3 (264.0 mg, 1.82 mmol) was dissolved in 1,4-dioxane (5 mL), p-bromoiodobenzene (566 mg, 2.0 mmol), Pd₂(dba)₃ (50 mg, 0.05 mmol), xantphos (60 mg, 0.10 mmol), and cesium carbonate (1180 mg, 3.64 mmol) were added at room temperature. The reaction was heated up to 90° C. to react for 4 h, after TLC monitored the completion of the reaction, it was cooled down to room temperature, ethyl acetate (30 mL) and saturated sodium bicarbonate solution (10 mL) was added, and it was extracted with ethyl acetate (20 mL×3), and the organic phases were combined, washed with saturated saline solution (50 mL), concentrated under reduced pressure to obtain a crude product, and the crude product was purified by column chromatography (PE/EtOAc=3/1) to obtain compound 93a (60 mg).

MS (ESI) m/z 300.0 [M+H]⁺.

¹H NMR (400 MHz, CDCl₃) δ 7.30-7.25 (m, 2H), 6.97-6.92 (m, 2H), 2.54-2.44 (m, 2H), 1.38-1.24 (m, 4H), 1.09-0.95 (m, 4H).

### Intermediate 103a 5-iodo-7-(2,2,2-trifluoroethyl)-7H-pyrrolo [2,3-d] pyrimidin-4-amine

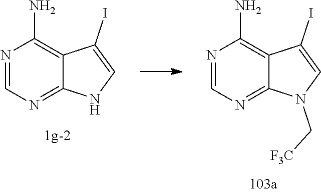 [SMILES: Nc1ncnc2[nH]cc(I)c12 ; Nc1ncnc2c1c(I)cn2CC(F)(F)F]

Compound 1g-2 (1.0 g, 3.85 mmol), 2,2,2-trifluoroethyl trifluoromethane sulfonate (1.34 g, 5.78 mmol), and cesium carbonate (2.5 g, 7.7 mmol) was dissolved in DMF (10 mL) to react at room temperature for 3 h, and it was concentrated under reduced pressure to remove the solvent, extracted with H₂O (100 mL) and DCM three times, the organic layers were combined, washed with saturated sodium chloride once, dried over anhydrous sodium sulfate, concentrated under reduced pressure, and purified by column chromatography (PE/EA=1/2) to obtain compound 103a (850 mg), white solid.

MS (ESI) m/z 343.1 [M+H]⁺.

¹H NMR (400 MHz, DMSO-d₆) δ 8.16 (s, 1H), 7.50 (s, 1H), 6.79 (s, 2H), 5.03 (q, J 9.3 Hz, 2H).

### Intermediate 112a 1-(4-(4-amino-6-(4-amino-2-fluorophenyl)-7-methyl-7H−pyrrolo [2,3-d] pyrimidin 3-5-yl) phenyl) imino) tetrahydro-1H-1λ⁶-thiophene-1-oxide

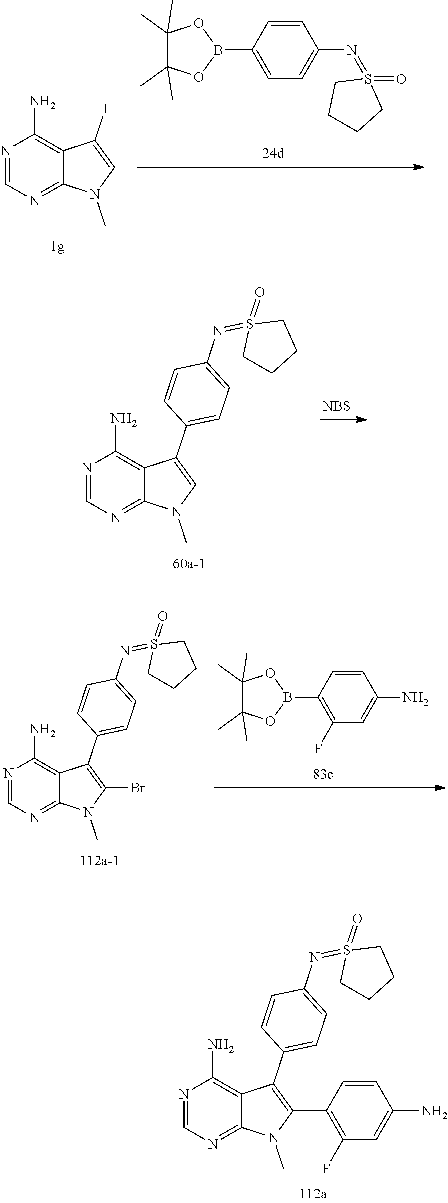

Compound 112a was synthesized with reference to the similar synthetic route and steps of compound 83d, wherein the compound 82c was replaced with compound 1g in first step, MS (ESI) m/z 451.2 [M+H]⁺.

### Intermediate 113a 1-bromo-4-iodobenzene-3,5-d₂

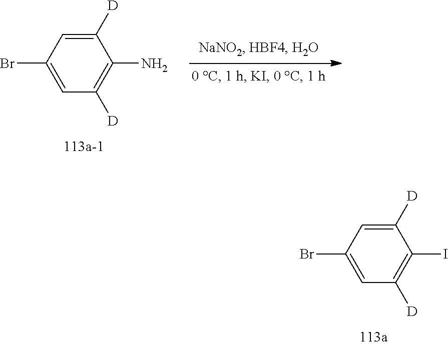 [SMILES: [2H]c1cc(Br)cc([2H])c1N ; [2H]c1cc(Br)cc([2H])c1I]

Compound 113a-1 was synthesized with reference to ACS Catalysis (2016), 6 (11), 7814-7823. Compound 113a-1 (724 mg, 4.16 mmol), H₂O (33 mL), and 40% HBF₄ (2.28 g, 913 mg, 10.40 mmol) were added into the reaction flask in sequence, and it was cooled in an ice salt water bath for 10 min. NaNO₂ (344 mg, 4.99 mmol)-H₂O (5.7 mmol) solution was added in the ice salt water bath to react in the ice salt water bath for 1 h. NaI (1.24 g, 8.24 mmol)-H₂O (5.8 mmol) solution was added slowly to the above reaction solution to react in the ice salt water bath for additional 1 h. Then saturated NaHSO₃ aqueous solution (20 mL) was added and stirred for additional 10 min, and H₂O and EA were added for extraction, the organic phase was washed with saturated saline solution, dried over anhydrous sodium sulfate, concentrated under reduced pressure, and purified by column chromatography to obtain compound 113a (225 mg), yellow solid.

MS (GC) m/z 284.1 [M]+

### Example 1 N-(4-(4-amino-7-methyl-5-(4-(1-oxotetrahydro-2H-1λ⁶-thiopyran-1-imino) phenyl)-7H-pyrrolo [2,3-d] pyrimidin-6-yl)-3-fluorophenyl) methacrylamide

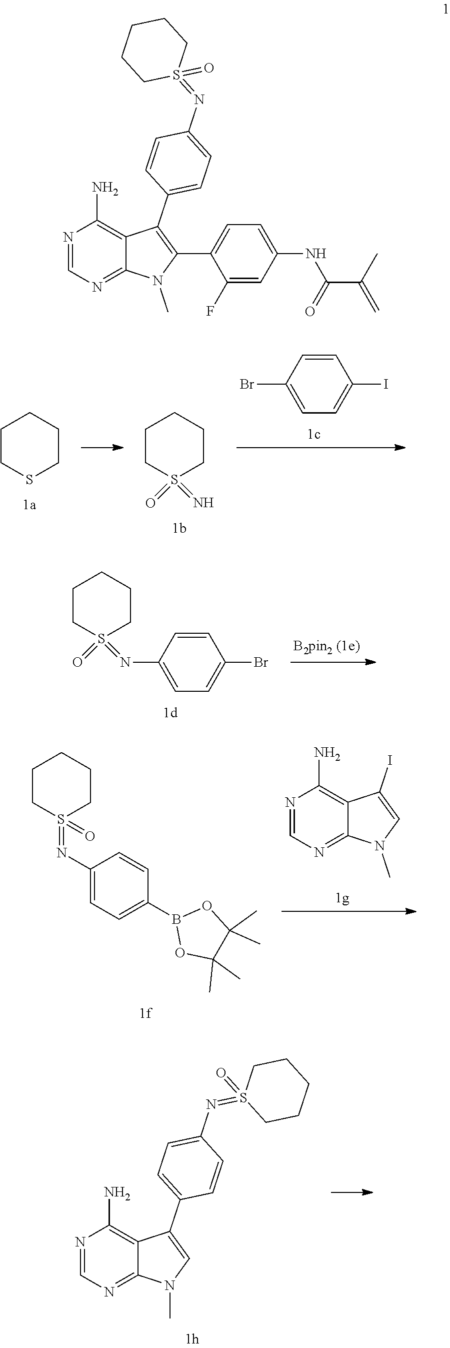

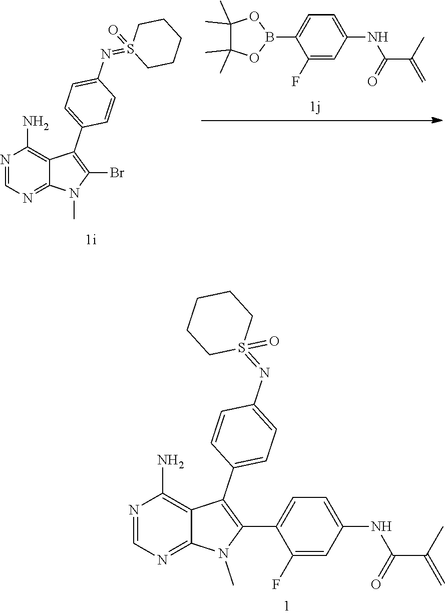

### Step 1 1-iminohexahydro-1λ⁶-thiopyran-1-oxide 1b

Tetrahydro-2H-pyran 1a (2.00 g, 19.6 mmol) and ammonium carbamate (2.29 g, 29.4 mmol) were added into a flask in sequence, then MeOH (40 mL) and iodobenzene diethyl ester (13.24 g, 41.1 mmol) were added. The reaction solution was stirred open at room temperature for 30 min, and then it was concentrated under reduced pressure to remove the solvent to obtain a crude product. The crude product was purified by column chromatography (MeOH/DCM=1/80) to obtain compound 1b (1.62 g), white solid.

MS (ESI) m/z 134 [M+H]⁺.

### Step 2 1-((4-bromophenyl) imino) hexahydro-1λ⁶-thiopyran-1-oxide 1d

Compound 1b (282 mg, 2.12 mmol), cesium carbonate (806 mg, 2.47 mmol), 1-bromo-4-iodobenzene 1c (500 mg, 1.77 mmol), Pd₂(dba)₃ (40 mg, 0.05 mmol), and Xantphos (77 mg, 0.13 mmol) were added to 1,4-dioxane (10 mL), and the reaction solution was heated up to 105° C. for 13 h. The reaction solution was filtered through diatomaceous earth, the filter cake was washed with ethyl acetate (50 mL), and saturated saline solution (50 mL) was used for washing. The organic phase was dried over anhydrous sodium sulfate and concentrated under reduced pressure to obtain a crude product. The crude product was purified by column chromatography (PE/EtOAc=4/1 to 1/1) to obtain compound 1d (410 mg), yellow oily liquid.

MS (ESI) m/z 288 [M+H]⁺.

### Step 3 1-(4-(4,4,5,5-methyl-1,3,2-dioxaborolan-2-yl)phenyl)imino)hexahydro-1λ⁶-thiopyran-1-oxide 1f

Compound 1d (350 mg, 1.22 mmol), bis(pinacolato)diboron 1e (372 mg, 1.46 mmol), Pd(dppf)Cl₂ (179 mg, 0.24 mmol), potassium acetate (359 mg, 3.66 mmol), and 1,4-dioxane (10 mL) was added into a sealed tube, after replaced with argon gas three times, sealed, and the reaction solution was heated up to 90° C. and stirred for 12 h. Water (10 mL) was added, and it was extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=4/1) to obtain compound 1f (356 mg), yellow solid.

MS (ESI) m/z 336 [M+H]⁺.

### Step 4 1-(4-(4-amino-7-methyl-7H-pyrrolo [2,3-d] pyrimidin-5-yl)phenyl)imino)hexahydro-1λ⁶-thiopyran-1-oxide 1h

Compound 1f (302 mg, 0.9 mmol), 5-iodo-7-methyl-7H-pyrrolo [2,3-d] pyrimidin-4-amine 1g (206 mg, 0.75 mmol), Pd(PPh₃)₄ (66 mg, 0.075 mmol), and K₃PO₄ (302 mg, 1.88 mmol) were added into a sealed tube, after replaced with argon gas three times, and 1,4-dioxane (5 mL) and H₂O (0.5 mL) were added. The reaction solution was heated up to 90° C. and stirred for 5 h. Then water (30 mL) was added, and extracted with DCM (60 mL×3). The organic phase was washed with saturated saline solution (50 mL), dried over anhydrous sodium sulfate, and concentrated under reduced pressure to obtain a crude product. The crude product was purified by column chromatography (MeOH/DCM=1/50) to obtain compound 1h (184 mg), light yellow solid.

MS (ESI) m/z 356 [M+H]⁺.

¹H NMR (500 MHz, CDCl₃) δ 8.31 (s, 1H), 7.33-7.29 (m, 2H), 7.21-7.17 (m, 2H), 6.91 (s, 1H), 5.50 (s, 2H), 3.83 (s, 3H), 3.43-3.35 (m, 2H), 3.14 (ddd, J=13.9, 8.9, 5.0 Hz, 2H), 2.15-2.04 (m, 4H), 1.78 (ddd, J=12.3, 6.5, 3.8 Hz, 1H), 1.62 (dtt, J=13.6, 9.0, 4.6 Hz, 1H).

### Step 5 1-(4-(4-amino-6-bromo-7-methyl-7H-pyrrolo[2,3-d] pyrimidin-5-yl) phenyl) imino) hexahydro-1λ⁶-thiopyran-1-oxide 1i

Compound 1h (150 mg, 0.42 mmol) was dissolved in DMF (3 mL), NBS (83 mg, 0.46 mmol) was added at 0° C., and the reaction solution was stirred at 0° C. for 1 h. Then DCM (30 mL) was added, and washed with saturated saline solution (10 mL×3), the organic phase was dried over anhydrous sodium sulfate, and concentrated under reduced pressure to obtain a crude product. The crude product was purified by column chromatography (MeOH/DCM=1/50) to obtain compound 1i (99 mg), white solid.

MS (ESI) m/z 434 [M+H]⁺.

### Step 6 N-(4-(4-amino-7-methyl-5-(4-(1-oxotetrahydro-2H-1λ⁶-thiopyran-1-imino)phenyl)-7H-pyrrolo [2,3-d] pyrimidin-6-yl) 3-fluorophenyl) methacrylamide 1

Compound 1i (50 mg, 0.12 mmol), N-(3-fluoro-4-(4,4,5,5-tetramethyl-1,3,2-dioxaborolan-2-yl) methacrylamide 1j (42 mg, 0.14 mmol), Pd(PPh₃)₄ (28 mg, 0.024 mmol), and K₃PO₄ (76 mg, 0.36 mmol) were added into a sealed tube, after replaced with argon gas three times, and 1,4-dioxane (1 mL) and H₂O (0.2 mL) were added, sealed, and the reaction solution was heated up to 100° C. and stirred for 12 h. Then ethyl acetate (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/40) to obtain compound 1 (24 mg), light yellow solid.

MS (ESI) m/z 356 [M+H]⁺.

¹H NMR (500 MHz, CDCl₃) δ 8.30 (s, 1H), 7.95 (s, 1H), 7.71 (dd, J=11.6, 2.1 Hz, 1H), 7.12-6.97 (m, 6H), 5.79 (s, 1H), 5.47 (s, 1H), 5.37 (s, 2H), 3.63 (s, 3H), 3.31 (dt, J=11.7, 5.0 Hz, 2H), 3.15-3.07 (m, 2H), 2.10-2.03 (m, 4H), 2.03 (s, 3H), 1.72 (dp, J=15.2, 5.2 Hz, 1H), 1.60 (ddt, J=15.2, 11.9, 5.6 Hz, 1H).

### Example 2 1-(4-(4-amino-7-methyl-5-(4-(1-oxotetrahydro-2H-1λ⁶-thiopyran-1-imino) phenyl)-7H-pyrrolo [2,3-d] pyrimidin-6-yl)-3,6-dihydropyridine-1(2H)-yl)prop-2-enyl-1-one

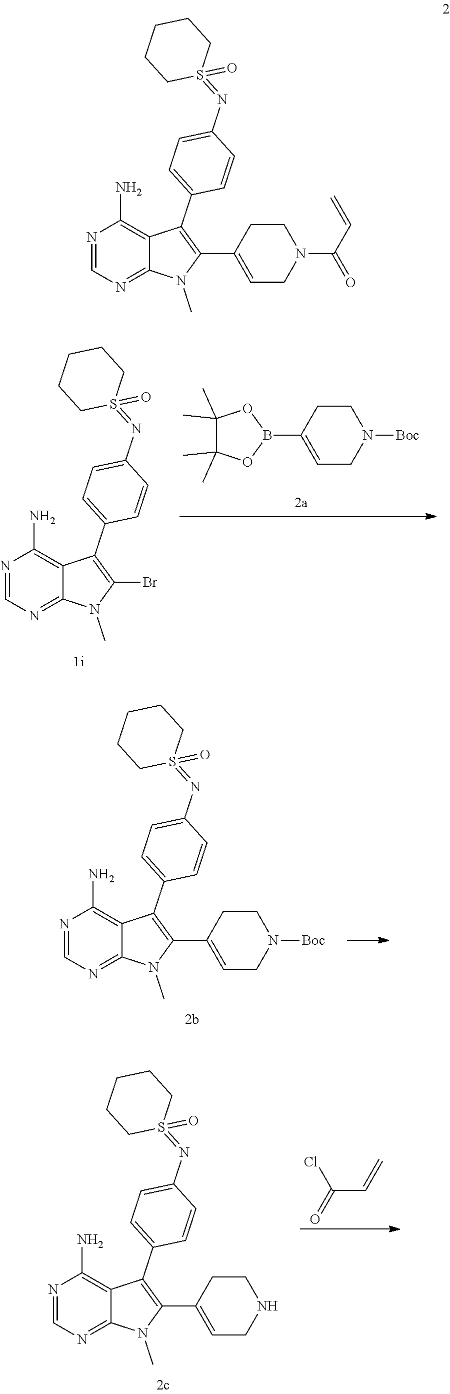

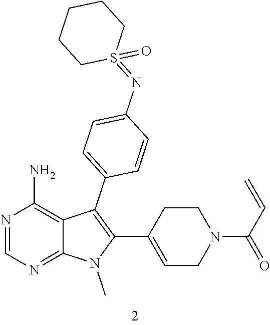 [SMILES: C=CC(=O)N1CC=C(c2c(-c3ccc(N=S4(=O)CCCCC4)cc3)c3c(N)ncnc3n2C)CC1]

### Step 1 4-(4-amino-7-methyl-5-(4-((1-oxotetrahydro-2H-1λ⁶-thiopyran-1-imino)phenyl)-7H-pyrrolo [2,3-d] pyrimidin-6-yl)-3,6-dihydropyridine-1 (2H)-tert-butyl carbonate 2b

Compound 1i (345 mg, 0.8 mmol), compound 2a (295.6 mg, 0.96 mmol), Pd(PPh₃)₄ (184 mg, 0.16 mmol), and potassium phosphate (509.5 mg, 2.4 mmol) were dissolved in anhydrous 1,4-dioxane (8 mL) and H₂O (2 mL), after replaced with nitrogen gas three times, the reaction was refluxed at 100° C. for 12 h. After the reaction was completed, the reaction solution was cooled down to room temperature, filtered through diatomaceous earth, concentrated under reduced pressure to remove the solvent, and it was extracted with EA/H₂O three times, the organic layers were combined, washed with saturated saline solution once, dried over anhydrous sodium sulfate, concentrated under reduced pressure, and purified by column chromatography (DCM/MEOH=40/1) to obtain compound 2b (312 mg), yellow brown solid.

MS (ESI) m/z 537 [M+H]⁺.

### Step 2 1-(4-(4-amino-7-methyl-6-(1,2,3,6-tetrahydropyridine-4-yl)-7H-pyrrolo[2,3-d]pyrimidin-5-yl)phenyl)imino)hexahydro-1λ⁶-thiopyran-1-oxide 2c

Compound 2b (310 mg, 0.58 mmol) was dissolved in dichloromethane (10 mL), trifluoroacetic acid (2 mL) was added dropwise at room temperature to react for 5 h. After the reaction was completed, it was concentrated under reduced pressure to obtain a crude product 2c (250 mg).

MS (ESI) m/z 437 [M+H]⁺.

### Step 3 1-(4-(4-amino-7-methyl-5-(4-(1-oxotetrahydro-2H-1λ⁶-thiopyran-1-imino) phenyl)-7H-pyrrolo[2,3-d] pyrimidin-6-yl)-3,6-dihydropyridine-1(2H)-yl)prop-2-enyl-1-one 2

Compound 2c (125 mg, 0.29 mmol) was dissolved in ultra dry DCM (3 mL), TEA (232 mg, 2.29 mmol) was added at 0° C. and stirred for 5 min. A solution of acryloyl chloride (28.5 mg, 0.32 mmol) in dichloromethane (2 mL) was added slowly dropwise to the above reaction solution to react at 0° C. for 1 h. After the reaction was completed, it was extracted with DCM/H₂O three times, the organic layers were combined, washed with saturated saline solution, dried over anhydrous sodium sulfate, concentrated under reduced pressure, and purified by column chromatography (DCM/MeOH=30/1) to obtain compound 2 (103 mg), white powdery solid.

MS (ESI) m/z 491 [M+H]⁺.

¹H NMR (400 MHz, MeOD-d4) δ 8.13 (s, 1H), 7.26 (dd, J=8.5, 2.9 Hz, 2H), 7.16 (dd, J=8.3, 1.3 Hz, 2H), 6.84-6.65 (m, 1H), 6.29-6.14 (m, 1H), 6.04-5.88 (m, 1H), 5.86-5.67 (m, 1H), 4.32-4.19 (m, 2H), 3.72 (s, 3H), 3.70 (t, J=5.3 Hz, 2H), 3.47-3.36 (m, 2H), 3.30-3.21 (m, 2H), 2.22 8-2.14 (m, 2H), 2.12-2.01 (m, 4H), 1.81-1.73 (m, 1H), 1.72-1.61 (m, 1H).

### Example 3 1-(4-(4-amino-7-methyl-5-(4-(1-oxotetrahydro-2H-1λ⁶-thiopyran-1-imino) phenyl)-7H-pyrrolo[2,3-d] pyrimidin-6-yl)-3,6-dihydropyridine-1(2H)-yl)2-methylprop-2-enyl-1-one

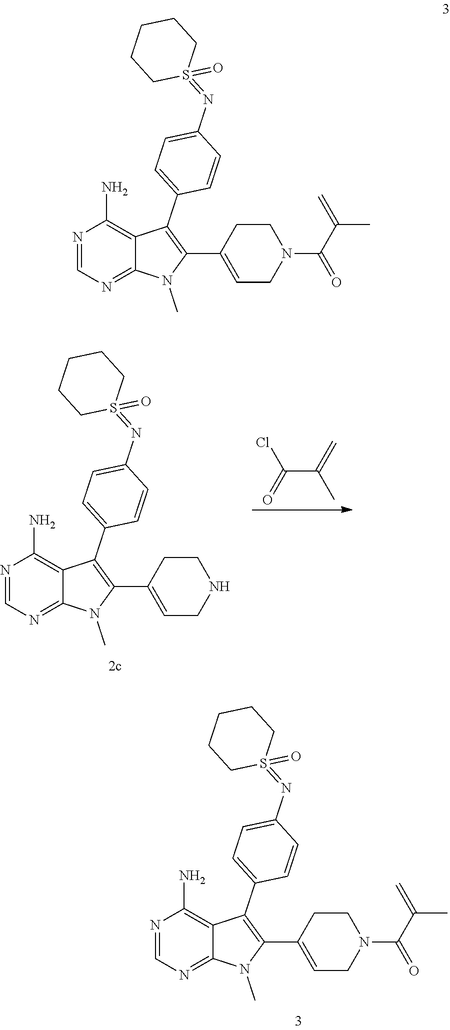

### Step 1 1-(4-(4-amino-7-methyl-5-(4-(1-oxotetrahydro-2H-1λ⁶-thiopyran-1-imino) phenyl)-7H-pyrrolo[2,3-d] pyrimidin-6-yl)-3,6-dihydropyridine-1(2H)-yl)2-methylprop-2-enyl-1-one

Compound 2c (125 mg, 0.29 mmol) was dissolved in ultra dry DCM (3 mL), TEA (232 mg, 2.29 mmol) was added at 0° C. and stirred for 5 min. A solution of methacryloyl chloride (32.8 mg, 0.32 mmol) in dichloromethane (2 mL) was added dropwise to the above reaction solution to react at 0° C. for 1 h. After the reaction was completed, it was extracted with DCM/H₂O three times, the organic layers were combined, washed with saturated saline solution, dried over anhydrous sodium sulfate, concentrated under reduced pressure, and purified by column chromatography (DCM/MeOH=30/1) to obtain compound 3 (67.8 mg), white powdery solid.

MS (ESI) m/z 505 [M+H]⁺.

¹H NMR (400 MHz, MeOD-d4) δ 8.13 (s, 1H), 7.27 (d, J=8.0 Hz, 2H), 7.16 (d, J=8.4 Hz, 2H), 5.93 (d, J=31.5 Hz, 1H), 5.24 (d, J=13.4 Hz, 1H), 5.02 (s, 1H), 4.20 (d, J=3.5 Hz, 2H), 3.77-3.6 (m, 5H), 3.48-3.37 (m, 2H), 3.29-3.22 (m, 2H), 2.25-2.14 (m, 2H), 2.13-1.99 (m, 4H) 1.93 (t, J=1.4 Hz, 3H), 1.82-1.72 (m, 1H), 1.71-1.61 (m, 1H).

### Example 4 N-(4-(4-amino-7-methyl-5-(4-(1-oxotetrahydro-2H-1λ⁶-thiopyran-1-imino) phenyl)-7H-pyrrolo [2,3-d] pyrimidin-6-yl) phenyl) methacrylamide

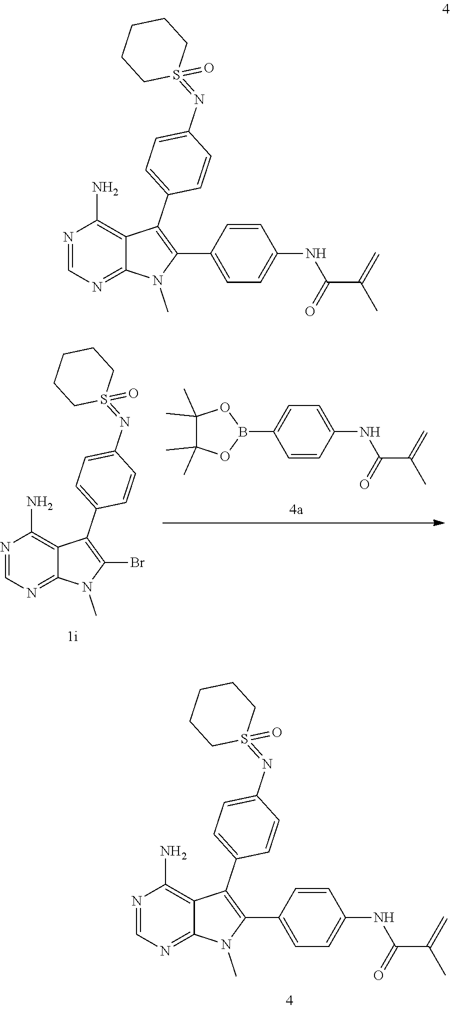

### Step 1 N-(4-(4-amino-7-methyl-5-(4-(1-oxotetrahydro-2H-1λ⁶-thiopyran-1-imino) phenyl)-7H-pyrrolo [2,3-d] pyrimidin-6-yl) phenyl) methacrylamide 4

Compound 1i (180 mg, 0.42 mmol), compound 4a (143.2 mg, 0.5 mmol), Pd(PPh₃)₄ (97.1 mg, 0.08 mmol), and potassium phosphate (267.5 mg, 1.26 mmol) were dissolved in anhydrous 1,4-dioxane (4 mL) and H₂O (1 mL), after replaced with nitrogen gas three times, heated up to 100° C., and the reaction was refluxed for 12 h. After the reaction was completed, the reaction solution was cooled down to room temperature, filtered through diatomaceous earth, concentrated under reduced pressure to remove the solvent, and it was extracted with EA/H₂O three times, the organic layers were combined, washed with saturated salt once, dried over anhydrous sodium sulfate, concentrated under reduced pressure, and purified by column chromatography (DCM/MeOH=40/1) to obtain compound 4 (128 mg), light yellow solid.

MS (ESI) m/z 515 [M+H]⁺.

¹H NMR (400 MHz, MeOD-d4) δ 8.17 (s, 1H), 7.67-7.61 (m, 2H), 7.29-7.24 (m, 2H), 7.17-7.11 (m, 2H), 7.08-7.03 (m, 2H), 5.80 (s, 1H), 5.52 (dd, J=1.7, 0.8 Hz, 1H), 3.68 (s, 3H), 3.43-3.33 (m, 2H), 3.28-3.17 (m, 2H), 2.07-1.99 (m, 7H), 1.78-1.69 (m, 1H), 1.68-1.59 (m, 1H)).

### Example 5 N-(4-(4-amino-5-(3-methoxy-4-(1-oxotetrahydro-2H-1λ⁶-thiopyran-1-imino) phenyl)-7-methyl-7H-pyrrolo [2,3-d] pyrimidin-6-yl)-3-fluorophenyl) methacrylamide

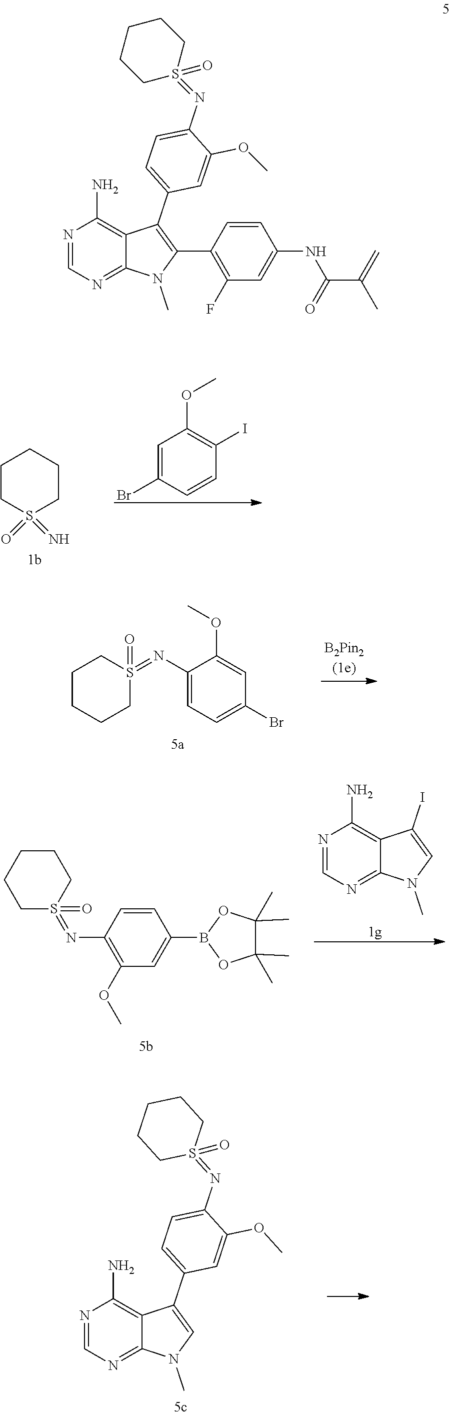

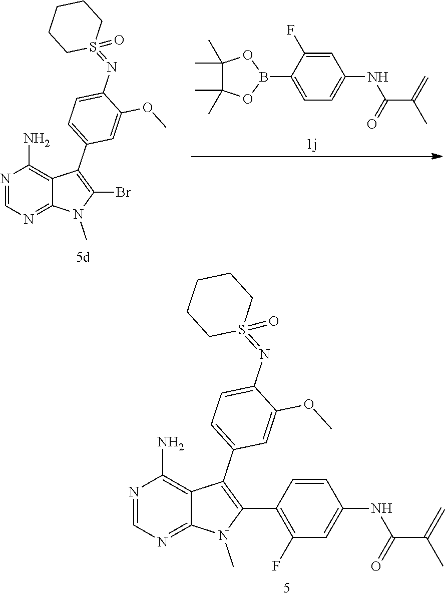 [SMILES: COc1cc(-c2c(Br)n(C)c3ncnc(N)c23)ccc1N=S1(=O)CCCCC1 ; C=C(C)C(=O)Nc1ccc(B2OC(C)(C)C(C)(C)O2)c(F)c1 ; C=C(C)C(=O)Nc1ccc(-c2c(-c3ccc(N=S4(=O)CCCCC4)c(OC)c3)c3c(N)ncnc3n2C)c(F)c1]

### Step 1 1-((4-bromo-2-methoxyphenyl) imino) hexahydro-1λ⁶-thiopyran-1-oxide 5a

Compound 1b (638 mg, 4.79 mmol), 4-bromo-1-iodo-2-methoxybenzene (1.79 g, 5.75 mmol), Pd₂(dba)₃ (109.7 mg, 0.12 mmol), Xantphos (208 mg, 0.36 mmol), cesium carbonate (2.3 g, 7.19 mmol) were sequentially added to the reaction flask, after replaced with nitrogen gas three times, anhydrous 1,4-dioxane (25 mL) was added, the temperature was raised to 100° C., and the reaction was refluxed for 6 h. After the reaction was completed, the reaction solution was filtered through diatomaceous earth, concentrated under reduced pressure, and purified by column chromatography (PE/EA=5/1) to obtain compound 5a (650 mg), light yellow oil.

MS (ESI) m/z 318 [M+H]⁺.

### Step 2 1-((2-methoxy-4-(4,4,5,5-tetramethyl-1,3,2-dioxaborolan-2-yl) phenyl) imino) hexahydro-1λ⁶-thiopyran-1-oxide 5b

Compound 5a (650 mg, 2.05 mmol), bis(pinacolato)diboron 1e (624.8 mg, 2.46 mmol), potassium acetate (603.6 mg, 6.15 mmol), and Pd(dppf)Cl₂ (300 mg, 0.41 mmol) were added into a reaction flask in sequence, after replaced with nitrogen gas three times, anhydrous 1,4-dioxane (15 mL) was added to dissolve, and the reaction solution reacted overnight at 90° C. After the reaction was completed, the reaction solution was cooled down to room temperature, filtered through diatomaceous earth, concentrated under reduced pressure, extracted with DCM/H₂O three times, the organic layers were combined, washed with saturated salt once, dried over anhydrous sodium sulfate, concentrated and purified by silica gel column chromatography (PE/EA=4/1) to obtain compound 5b (405 mg), yellow solid.

MS (ESI) m/z 366 [M+H]⁺.

### Step 3 1-(4-(4-amino-7-methyl-7H-pyrrolo [2,3-d] pyrimidin-5-yl)-2-methoxyphenyl) imino) hexahydro-1λ⁶-thiopyran-1-oxide 5c

Compound 5b (401 mg, 1.1 mmol), compound 1g (274 mg, 1.0 mmol), Pd(PPh₃)₄ (116 mg, 0.1 mmol), and potassium phosphate (530.7 mg, 2.5 mmol) were dissolved in 1,4-dioxane (12 mL) and water (3 mL), after replaced with nitrogen gas three times, the reaction was carried out overnight at 90° C. It was filtered through diatomaceous earth, concentrated under reduced pressure to remove the solvent, extracted with EA/H₂O three times, the organic layers were combined and washed with saturated salt water once, dried over anhydrous sodium sulfate, concentrated under reduced pressure and purified by column chromatography (DCM/MEOH=30/1) to obtain compound 5c (180 mg), light yellow solid.

MS (ESI) m/z 382 [M+H]⁺.

### Step 4 1-(4-(4-amino-6-bromo-7-methyl-7H-pyrrolo [2,3-d] pyrimidin-5-yl)-2-methoxyphenyl) imino) hexahydro-1λ⁶-thiopyran-1-oxide 5d

Compound 5c (174 mg, 0.45 mmol) was dissolved in anhydrous DMF (4 mL), NBS (88 mg, 0.50 mmol) was added at 0° C. to react for 1 h. After the reaction was completde, sodium thiosulfate solution was added to quench the reaction, and it was extracted with EA/H₂O 4 times, the organic layers were combined, washed with saturated salt water once, dried over anhydrous sodium sulfate, concentrated under reduced pressure, and purified by column chromatography (DCM/MEOH=30/1) to obtain compound 5d (107 mg), yellow solid.

MS (ESI) m/z 464 [M+H]⁺.

¹H NMR (400 MHz, CDCl₃) δ 8.28 (s, 1H), 7.31 (d, J=7.7 Hz, 1H), 6.92 (d, J=8.4 Hz, 2H), 5.53 (s, 2H), 3.87 (s, 3H), 3.85 (s, 3H), 3.43-3.34 (m, 2H), 3.28-3.16 (m, 2H), 2.16-2.03 (m, 4H), 1.79-1.62 (m, 2H).

### Step 5 N-(4-(4-amino-5-(3-methoxy-4-(1-oxotetrahydro-2H-1λ⁶-thiopyran-1-imino) phenyl)-7-methyl-7H-pyrrolo [2,3-d] pyrimidin-6-yl)-3-fluorophenyl) methacrylamide 5

Compound 5d (101 mg, 0.22 mmol), compound 1j (99.8 mg, 0.33 mmol), Pd(PPh₃)₄ (50.4 mg, 0.04 mmol), and potassium phosphate (138.9 mg, 0.65 mmol) were dissolved in 1,4-dioxane (8 mL) and water (2 mL), after replaced with nitrogen gas three times, the reaction was refluxed at 100° C. for 12 h. After the reaction was completed, the reaction solution was cooled down to room temperature, filtered through diatomaceous earth, concentrated under reduced pressure to remove the solvent, and it was extracted with DCM/H₂O three times, the organic layers were combined, washed with saturated salt water once, dried over anhydrous sodium sulfate, concentrated under reduced pressure, and purified by column chromatography (DCM/MEOH=40/1) to obtain compound 5 (63 mg), light yellow solid.

MS (ESI) m/z 563 [M+H]⁺.

¹H NMR (400 MHz, Methanol-d4) δ 8.40 (s, 1H), 7.77 (dd, J=12.2, 2.1 Hz, 1H), 7.44 (dd, J=8.5, 2.1 Hz, 1H), 7.25 (dd, J=9.9, 8.1 Hz, 2H), 6.98-6.82 (m, 2H), 5.82 (s, 1H), 5.57 (q, J=1.6 Hz, 1H), 3.75 (s, 3H), 3.73 (s, 3H), 3.69-3.59 (m, 2H), 3.57-3.43 (m, 2H), 2.18-2.07 (m, 4H), 2.02 (s, 3H), 1.84-1.65 (m, 2H).

### Example 6 N-(4-(4-amino-5-(4-(4,4-difluoro-1-oxotetrahydro-2H-1λ⁶-thiopyran-1-imino) phenyl)-7-methyl-7H-pyrrolo [2,3-d] pyrimidin-6-yl)-3-fluorophenyl) methacrylamide

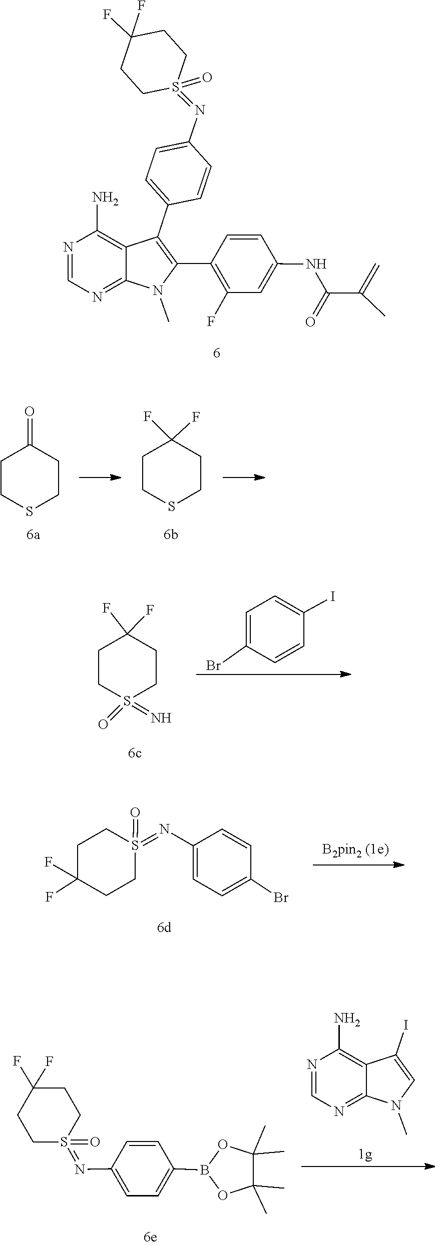

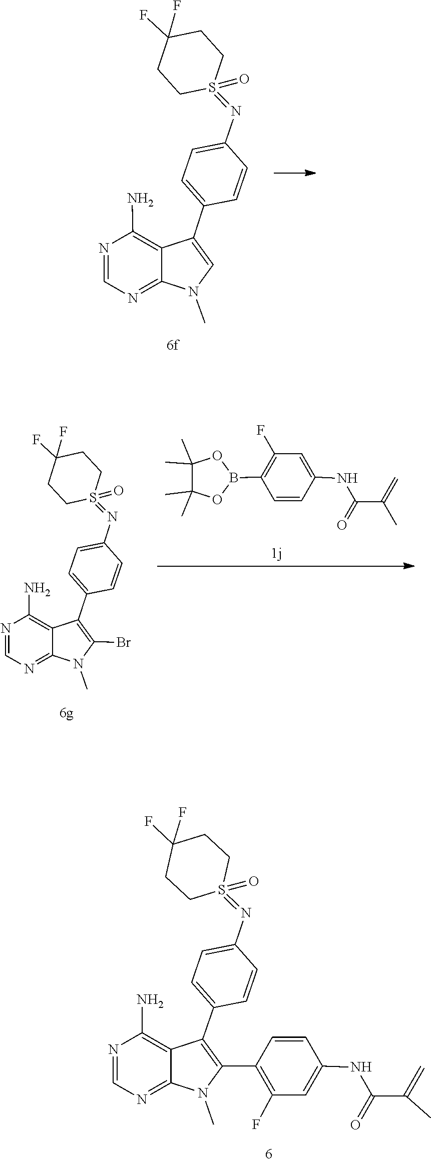

### Step 1 4,4-difluorotetrahydro-2H-pyran 6b

Compound 6a (2.0 g, 17.24 mmol) was dissolved in ultra dry DCM (100 mL), stirred at 0° C. for 5 min, BAST (5.6 g, 34.47 mmol) was added to react for additional 4 h. After the reaction was completed, the reaction solution was poured into a cold NaHCO₃ aqueous solution, and it was extracted with DCM three times, the organic layers were combined, and washed with saturated saltwater once, dried over anhydrous sodium sulfate, concentrated and dried to obtain compound 6b (2.38 g), oily liquid, which were used directly for the next reaction step without purification.

MS (ESI) m/z 139 [M+H]⁺.

### Step 2 4,4-difluoro-1-imino-tetrahydro-1λ⁶-thiopyran-1-oxide 6c

Compound 6b (2.38 g, 17.24 mmol) was dissolved in anhydrous methanol (100 mL), ammonium carbonate (2.49 g, 25.86 mmol) and iodobenzoic acid (13.9 g, 43.1 mmol) were added to the above reaction solution sequentially, and reacted at room temperature for 4 h. It was concentrated under reduced pressure to remove the solvent, and extracted with DCM/H₂O three times, the organic layers were combined, washed with saturated salt water once, dried over anhydrous sodium sulfate, concentrated and purified by column chromatography (DCM/MEOH=40/1) to obtain compound 6c (1.07 g), yellow liquid.

MS (ESI) m/z 170 [M+H]⁺.

### Step 3 1-((4-bromophenyl)imino)-4,4-difluorotetrahydro-1λ⁶-thiopyran-1-oxide 6d

Compound 6c (1.07 g, 6.33 mmol), p-bromoiodobenzene (2.14 g, 7.60 mmol), Pd₂(dba)₃ (144.9 mg, 0.16 mmol), Xantphos (274.7 mg, 0.47 mmol), and cesium carbonate (3.09 g, 9.49 mmol) were added to a reaction flask sequentially, after replaced with nitrogen gas three times, anhydrous 1,4-dioxane (35 mL) was added, the temperature was raised up to 100° C., and the reaction was refluxed for 6 h. The reaction solution was filtered through diatomaceous earth, concentrated under reduced pressure, and purified by column chromatography (PE/EA=5/1) to obtain compound 6d (228 mg), yellow solid.

MS (ESI) m/z 324 [M+H]⁺.

### Step 4 4,4-difluoro-1-((4-(4,4,5,5-tetramethyl-1,3,2-dioxaborolan-2-yl)phenyl)imino) hexahydro-1λ⁶-thiopyran-1-oxide 6e

Compound 6d (224 mg, 0.69 mmol), compound 1e (211.3 mg, 0.83 mmol), potassium acetate (203 mg, 2.07 mmol), and Pd(dppf)Cl₂ (101 mg, 0.14 mmol) were added to the reaction flask in sequence, after replaced with nitrogen three times, anhydrous 1,4-dioxane (8 mL) was added to dissolve, and then heated up to 90° C. and reacted overnight. It was filtered through diatomaceous earth, concentrated under reduced pressure, extracted with DCM/H₂O three times, the organic layers were combined, washed with saturated salt once, dried over anhydrous sodium sulfate, concentrated and purified by column chromatography (PE/EA=3/1) to obtain compound 6e (164 mg), yellow solid.

MS (ESI) m/z 372 [M+H]⁺.

### Step 5 1-(4-(4-amino-7-methyl-7H-pyrrolo [2,3-d] pyrimidin-5-yl) phenyl) imino)-4,4-difluorohexahydro-1λ⁶-thiopyran-1-oxide 6f

Compound 6e (158 mg, 0.43 mmol), compound 1g (106 mg, 0.39 mmol), Pd(PPh₃)₄ (45 mg, 0.039 mmol), and potassium phosphate (207 mg, 0.88 mmol) were dissolved in 1,4-dioxane (4 mL) and water (1 mL), after replaced with nitrogen three times, heated up to 90° C. and reacted overnight. It was filtered through diatomaceous earth, concentrated under reduced pressure to remove the solvent, extracted three times with DCM/H₂O, the organic layers were combined, and washed with saturated sodium chloride once, dried over anhydrous sodium sulfate, concentrated under reduced pressure and purified by column chromatography (DCM/MEOH=30/1) to obtain compound 6f (155 mg), light yellow solid.

MS (ESI) m/z 392 [M+H]⁺.

¹H NMR (400 MHz, CDCl₃) δ 8.31 (s, 1H), 7.34 (d, J=8.4 Hz, 2H), 7.19 (d, J=8.4 Hz, 2H), 6.93 (s, 1H), 5.61 (s, 2H), 3.85 (s, 3H), 3.56-3.46 (m, 2H), 3.45-3.35 (m, 2H), 2.65-2.45 (m, 4H).

### Step 6 1-(4-(4-amino-6-bromo-7-methyl-7H-pyrrolo [2,3-d] pyrimidin-5-yl) phenyl) imino)-4,4-difluorohexahydro-1λ⁶-thiopyran-1-oxide 6g

Compound 6f (150 mg, 0.38 mmol) was dissolved in anhydrous DMF (4 mL), NBS (75.1 mg, 0.42 mmol) was added at 0° C. to react for 1 h. Sodium thiosulfate solution was added to quench the reaction, and it was extracted with EA/H₂O 4 times, the organic layers were combined, and washed with saturated salt water once, the organic phase was dried over anhydrous sodium sulfate, concentrated under reduced pressure, and purified by column chromatography (DCM/MEOH=30/1) to obtain compound 6g (52 mg), yellow solid.

MS (ESI) m/z 470, 472 [M+H]⁺.

¹H NMR (400 MHz, CDCl₃) δ 8.28 (s, 1H), 7.35 (d, J=8.4 Hz, 2H), 7.20 (d, J=8.4 Hz, 2H), 5.12 (s, 2H), 3.83 (s, 3H), 3.56-3.48 (m, 2H), 3.46-3.38 (m, 2H), 2.65-2.52 (m, 4H).

### Step 7 N-(4-(4-amino-5-(4-(4,4-difluoro-1-oxotetrahydro-2H-1λ⁶-thiopyran-1-imino) phenyl)-7-methyl-7H-pyrrolo [2,3-d] pyrimidin-6-yl)-3-fluorophenyl) methacrylamide 6

Compound 6g (40 mg, 0.09 mmol), compound 1j (39 mg, 0.13 mmol), Pd(PPh₃)₄ (19.7 mg, 0.02 mmol), and potassium phosphate (54.3 mg, 0.26 mmol) were dissolved in 1,4-dioxane (4 mL) and water (1 mL), after replaced with nitrogen three times, the reaction was refluxed at 100° C. for 12 h. After the reaction solution was cooled down to room temperature, it was filtered through diatomaceous earth, concentrated under reduced pressure to remove the solvent, and extracted with DCM/H₂O three times, the organic layers were combined, washed with saturated salt water once, dried over anhydrous sodium sulfate, concentrated under reduced pressure, and purified by column chromatography (DCM/MEOH=40/1) to obtain compound 6 (18 mg), white solid.

MS (ESI) m/z 569 [M+H]⁺.

¹H NMR (400 MHz, MeOD-d4) δ 8.37 (s, 1H), 7.76 (dd, J=12.3, 2.0 Hz, 1H), 7.41 (dd, J=8.5, 2.1 Hz, 1H), 7.26-7.15 (m, 3H), 7.10 (d, J=8.8 Hz, 2H), 5.82 (s, 1H), 5.57 (q, J=1.5 Hz, 1H), 3.73 (s, 3H), 3.61-3.41 (m, 4H), 2.61-2.40 (m, 4H), 2.02 (dd, J=1.6, 1.0 Hz, 3H).

### Example 7 N-(4-(4-amino-5-(3-fluoro-4-((1-oxotetrahydro-2H-1λ⁶-thiopyran-1-imino) phenyl)-7-methyl-7H-pyrrolo [2,3-d] pyrimidin-6-yl)-3-fluorophenyl) methacrylamide

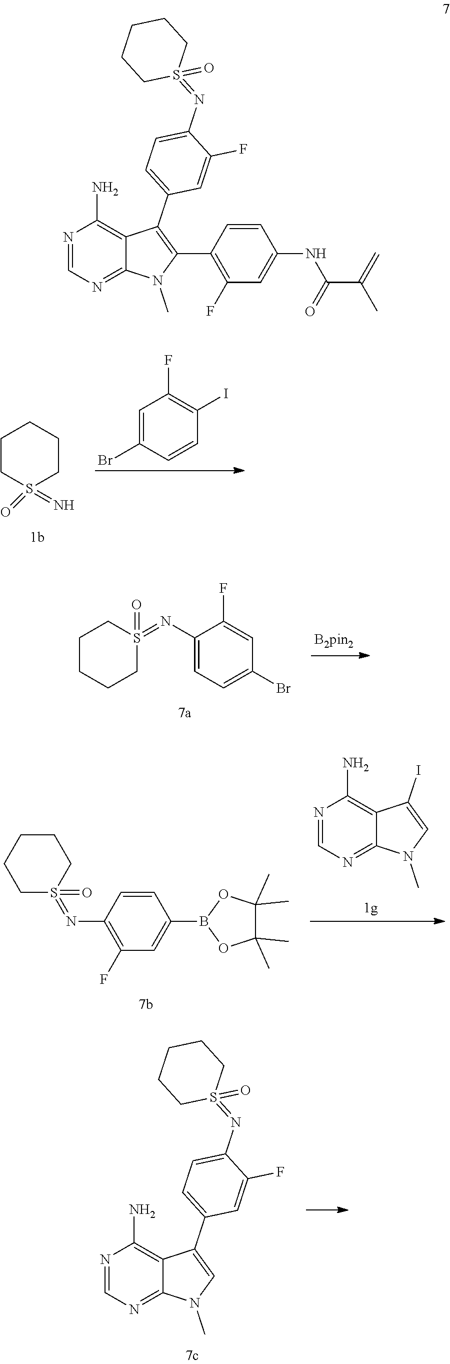

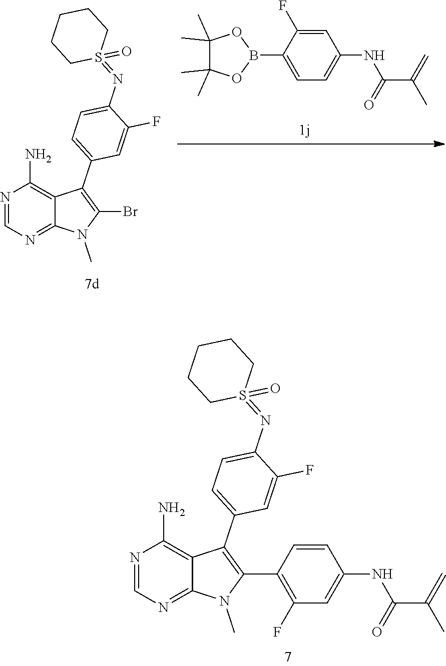 [SMILES: Cn1c(Br)c(-c2ccc(N=S3(=O)CCCCC3)c(F)c2)c2c(N)ncnc21 ; C=C(C)C(=O)Nc1ccc(B2OC(C)(C)C(C)(C)O2)c(F)c1 ; C=C(C)C(=O)Nc1ccc(-c2c(-c3ccc(N=S4(=O)CCCCC4)c(F)c3)c3c(N)ncnc3n2C)c(F)c1]

### Step 1-((4-bromo-2-fluorophenyl) imino) hexahydro-1λ⁶-thiopyran-1-oxide 7a

Compound 1b (104 mg, 0.78 mmol), 1-bromo-3-fluoro-4-iodobenzene (281.2 mg, 0.94 mmol), Pd₂(dba)₃ (17.9 mg, 0.02 mmol), Xantphos (33.8 mg, 0.06 mmol), and cesium carbonate (381.2 mg, 1.2 mmol) were added to a reaction flask sequentially, after replaced with nitrogen three times, anhydrous 1,4-dioxane (8 mL) was added, the temperature was raised up to 100° C., and the reaction was refluxed for 6 h. It was filtrated through diatomaceous earth, concentrated under reduced pressure, and purified by column chromatography (PE/EA=5/1) to obtain compound 7a (181 mg), light yellow oil.

MS (ESI) m/z 305 [M+H]⁺.

### Step 2 1-((2-Fluoro-4-(4,4,5,5-tetramethyl-1,3,2-dioxaborolan-2-yl) phenyl) imino) hexahydro-1λ⁶-thiopyran-1-oxide 7b

Compound 7a (181 mg, 0.59 mmol), bis(pinacolato)diboron (181 mg, 0.71 mmol), potassium acetate (173.7 mg, 1.77 mmol), and Pd(dppf)Cl₂ (86.3 mg, 0.12 mmol) were added to the reaction flask in sequence, after replaced with nitrogen three times, anhydrous 1,4-dioxane (8 mL) was added to dissolve, and heated up to 90° C. to react overnight. After the reaction was completed, the reaction solution was cooled down to room temperature, filtered through diatomaceous earth, concentrated under reduced pressure, extracted with DCM/H₂O three times, the organic layers were combined, washed with saturated salt once, the organic phase was dried over anhydrous sodium sulfate, concentrated and purified by column chromatography (PE/EA=4/1) to obtain compound 7b (231 mg), yellow liquid.

MS (ESI) m/z 354 [M+H]⁺.

¹H NMR (400 MHz, CDCl₃) δ 7.20 (dd, J=10.1, 2.1 Hz, 1H), 7.14 (t, J=8.3 Hz, 1H), 7.10 (dd, J=8.7, 2.1 Hz, 1H), 3.39-3.29 (m, 2H), 3.18-3.07 (m, 2H), 2.18-2.01 (m, 4H), 1.79-1.71 (m, 1H), 1.68-1.53 (m, 1H).

### Step 3 1-(4-(4-amino-7-methyl-7H-pyrrolo [2,3-d] pyrimidin-5-yl)-2-fluorophenyl) imino) hexahydro-1λ⁶-thiopyran-1-oxide 7c

Compound 7b (231 mg, 0.65 mmol), compound 1g (149 mg, 0.55 mmol), Pd(PPh₃)₄ (62.8 mg, 0.06 mmol), and potassium phosphate (292 mg, 1.38 mmol) were dissolved in 1,4-dioxane (8 mL) and water (2 mL), after replaced with nitrogen three times, the reaction was carried out at 90° C. overnight. After the reaction was completed, it was cooled down to room temperature, filtered through diatomaceous earth, concentrated under reduced pressure to remove the solvent, extracted with DCM/H₂O three times, the organic layers were combined, washed with saturated sodium chloride once, dried over anhydrous sodium sulfate, concentrated under reduced pressure and purified by column chromatography (DCM/MEOH=30/1) to obtain compound 7c (132 mg), yellow solid.

MS (ESI) m/z 374 [M+H]⁺.

¹H NMR (400 MHz, CDCl₃) δ 8.32 (s, 1H), 7.33 (t, J=8.4 Hz, 1H), 7.16 (dd, J=11.3, 2.1 Hz, 1H), 7.09 (dd, J=8.3, 1.9 Hz, 1H), 6.92 (s, 1H), 5.37 (s, 2H), 3.83 (s, 3H), 3.45-3.33 (m, 2H), 3.23-3.08 (m, 2H), 2.22-2.13 (m, 4H), 1.86-1.73 (m, 1H), 1.70-1.57 (m, 1H).

### Step 4 1-(4-(4-amino-6-bromo-7-methyl-7H-pyrrolo [2,3-d] pyrimidin-5-yl)-2-fluorophenyl) imino) hexahydro-1λ⁶-thiopyran-1-oxide 7d

Compound 7c (132 mg, 0.35 mmol) was dissolved in anhydrous DMF (4 mL), NBS (69.2 mg, 0.39 mmol) was added at 0° C., and the reaction was quenched with sodium thiosulfate solution after 2 h of reaction, and it was extracted with EA/H₂O 4 times, the organic layers were combined, washed with saturated salt water once, the organic phase was dried over anhydrous sodium sulfate, concentrated under reduced pressure, and purified by column chromatography (DCM/MEOH=30/1) to obtain compound 7d (60 mg), yellow solid.

MS (ESI) m/z 452 [M+H]⁺.

¹H NMR (400 MHz, CDCl₃) δ 8.28 (s, 1H), 7.36 (t, J=8.5 Hz, 1H), 7.17 (dd, J=11.3, 2.1 Hz, 1H), 7.07 (dd, J=8.2, 2.2 Hz, 1H), 5.30 (s, 2H), 3.84 (s, 3H), 3.47-3.34 (m, 2H), 3.25-3.15 (m, 2H), 2.23-2.08 (m, 4H), 1.82-1.73 (m, 1H), 1.72-1.60 (m, 1H).

### Step 5 N-(4-(4-amino-5-(3-fluoro-4-((1-oxotetrahydro-2H-1λ⁶-thiopyran-1-imino) phenyl)-7-methyl-7H-pyrrolo [2,3-d] pyrimidin-6-yl)-3-fluorophenyl) methacrylamide 7

Compound 7d (60 mg, 0.13 mmol), compound 1j (61 mg, 0.20 mmol), Pd(PPh₃)₄ (30 mg, 0.03 mmol), and potassium phosphate (82.8 mg, 0.39 mmol) were dissolved in 1,4-dioxane (4 mL) and water (1 mL), after replaced with nitrogen three times, the reaction was refluxed at 100° C. for 12 h. The reaction solution was cooled down to room temperature, filtered through diatomaceous earth, concentrated under reduced pressure to remove the solvent, extracted with DCM/H₂O three times, and the organic layers were combined, washed with saturated saline solution, dried over anhydrous sodium sulfate, concentrated under reduced pressure, purified by column chromatography (DCM/MEOH=40/1) to obtain compound 7 (57 mg), light yellow solid.

MS (ESI) m/z 551 [M+H]⁺.

¹H NMR (400 MHz, MeOD-d₄) δ 8.19 (s, 1H), 7.73 (dd, J=12.1, 2.1 Hz, 1H), 7.38 (dd, J=8.4, 2.1 Hz, 1H), 7.25-7.14 (m, 2H), 7.01-6.88 (m, 2H), 5.82 (s, 1H), 5.55 (q, J=1.6 Hz, 1H), 3.62 (s, 3H), 3.42-3.33 (m, 2H), 3.29-3.20 (m, 2H), 2.11-1.98 (m, 7H), 1.79-1.70 (m, 1H), 1.69-1.70 (m, 1H) 1.58 (m, 1H).

### Example 8 N-(4-(4-amino-7-methyl-5-(4-(1-oxo-1λ⁶-thietan-1-ylidene) amino) phenyl)-7H-pyrrolo [2,3-d] pyrimidin-6-yl)-3-fluorophenyl) methacrylamide

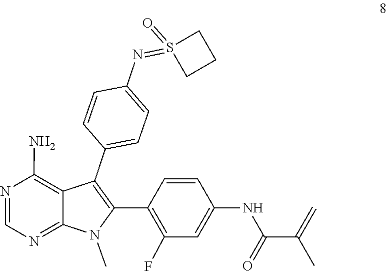 [SMILES: C=C(C)C(=O)Nc1ccc(-c2c(-c3ccc(N=S4(=O)CCC4)cc3)c3c(N)ncnc3n2C)c(F)c1]

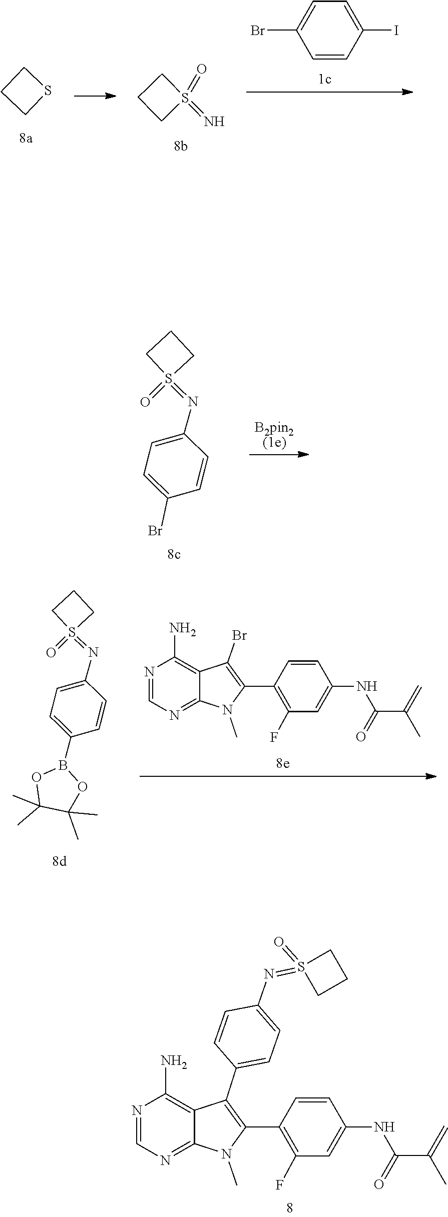

### Step 1 1-Amino-1λ⁶-thietane-1-oxide 8b

Thietane 8a (5 g, 67.5 mmol) and ammonium carbamate (7.85 g, 101 mmol) were added to a flask sequentially, and then MeOH (100 mL) and iodobenzene diethyl ester (43.5 g, 135 mmol) were added. The reaction solution was stirred open at room temperature for 30 min, and then concentrated under reduced pressure to remove the solvent to obtain a crude product. The crude product was purified by column chromatography (MeOH/DCM=1/20) to obtain compound 8b (3.6 g), colorless oil.

¹H NMR (400 MHz, CDCl₃) δ 4.12-3.98 (m, 4H), 3.39 (brs, 1H), 2.24-2.14 (m, 2H).

### Step 2 1-((4-bromophenyl) imino)-1λ⁶-thietane-1-oxide 8c

Compound 8b (210 mg, 2.0 mmol), cesium carbonate (0.99 g, 3.0 mmol), 1-bromo-4-iodobenzene 1c (0.688 g, 2.4 mmol), Pd₂(dba)₃ (55 mg, 0.06 mmol), and Xantphos (104 mg, 0.18 mmol) were added to 1,4-dioxane (6 mL), and the reaction solution was heated up to 100° C. to react for 16 h. The reaction solution was filtered through diatomaceous earth, and the filter cake was washed with ethyl acetate (100 mL) and saturated saline solution (50 mL). The organic phase was dried over anhydrous sodium sulfate and concentrated under reduced pressure to obtain a crude product. The crude product was purified by column chromatography (PE/EtOAc=4/1 to 3/2) to obtain compound 8c (470 mg), light yellow solid.

¹H NMR (400 MHz, CDCl₃) δ 7.33 (d, J=8.8 Hz, 2H), 6.90 (d, J=8.8 Hz, 2H), 4.18 (d, J=8.0 Hz, 4H), 2.40-2.28 (m, 2H).

### Step 3 1-(4-(4,4,5,5-tetramethyl-1,3,2-dioxaborolan-2-yl) phenyl) imino)-1λ⁶-thietane-1-oxide 8d

Compound 8c (260 mg, 1.0 mmol), bis(pinacolato)diboron 1e (305 mg, 1.2 mmol), Pd(dppf)Cl₂ (146 mg, 0.2 mmol), potassium acetate (294 mg, 3.0 mmol), and 1,4-dioxane (5 mL) were added into a sealed tube, after replaced with argon gas three times, sealed, and the reaction solution was heated up to 90° C. and stirred for 16 h. Then water (10 mL) was added, and extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 8d (256 mg), white solid.

¹H NMR (400 MHz, CDCl₃) δ 7.66 (d, J=8.4 Hz, 2H), 6.95 (d, J=8.4 Hz, 2H), 4.10 (t, J=8.0 Hz, 4H), 2.27-2.19 (m, 2H), 1.28 (s, 12H).

### Step 4 N-(4-(4-amino-7-methyl-5-(4-(1-oxo-1λ⁶-thiocyclobutan-1-ylidene) amino) phenyl)-7H-pyrrolo [2,3-d] pyrimidin-6-yl)-3-fluorophenyl) methacrylamide 8

Compound 8e (60 mg, 0.15 mmol), compound 8d (55 mg, 0.18 mmol), Pd(PPh₃)₄ (17 mg, 0.015 mmol), and K₃PO₄ (95 mg, 0.45 mmol) were added into a sealed tube, after replaced with argon gas three times, 1,4-dioxane (2 mL) and H₂O (0.2 mL) were added, sealed, the reaction solution was heated up to 100° C. and stirred for 16 h. then dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 8 (12 mg), white solid.

MS (ESI) m/z 505 [M+H]⁺.

¹H NMR (400 MHz, CDCl₃) δ 8.36 (s, 1H), 7.70 (dd, J=11.6, 1.9 Hz, 1H), 7.58 (s, 1H), 7.14-7.03 (m, 4H), 6.94 (d, J=8.4 Hz, 2H), 5.80 (s, 1H), 5.51 (d, J=1.6 Hz, 1H), 5.00 (s, 2H), 4.21 (dd, J=9.4, 7.3 Hz, 4H), 3.65 (s, 3H), 2.40-2.32 (m, 2H), 2.07 (s, 3H).

### Example 9 N-(4-(4-amino-5-(4-(diethyl (oxo) 1λ⁶-sulfanylidene)-3-fluorophenyl)-7-methyl 7H-pyrrolo [2,3-d] pyrimidin-6-yl)-phenyl) methacrylamide

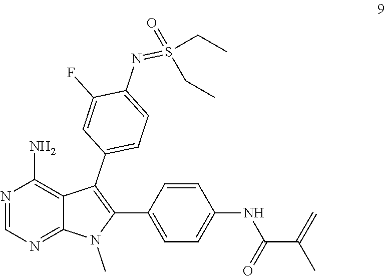 [SMILES: C=C(C)C(=O)Nc1ccc(-c2c(-c3ccc(N=S(=O)(CC)CC)c(F)c3)c3c(N)ncnc3n2C)cc1]

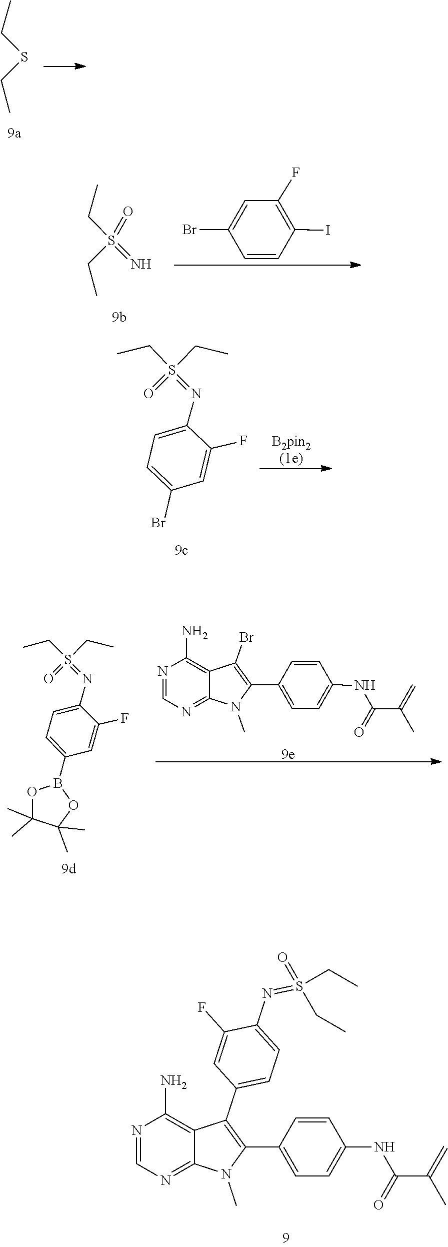

### Step 1 imine-1λ⁶-diethylsulfoxide 9b

Diethylthioether 9a (4.5 g, 50 mmol) and ammonium carbamate (5.85 g, 75 mmol) were added into a flask in sequence, then MeOH (100 mL) and iodobenzene diethyl ester (32.2 g, 100 mmol) were added. The reaction solution was stirred open at room temperature for 30 min and then concentrated under reduced pressure to remove the solvent to obtain a crude product. The crude product was purified by column chromatography (MeOH/DCM=1/20) to obtain compound 9b (3.6 g), colorless oil.

¹H NMR (400 MHz, CDCl₃) δ 3.12-2.93 (q, J=7.5 Hz, 4H), 2.66 (s, 1H), 1.38 (t, J 7.5 Hz, 6H).

### Step 2 1-((4-bromo-2-fluorophenyl) imino)-1λ⁶-diethylsulfoxide 9c

Compound 9b (400 mg, 3.3 mmol), cesium carbonate (1.6 g, 5 mmol), 1-bromo-3-fluoro-4-iodobenzene (1.2 g, 4 mmol), Pd₂(dba)₃ (92 mg, 0.1 mmol), and Xantphos (173 mg, 0.3 mmol) were added to 1,4-dioxane (12 mL), the reaction solution was heated up to 100° C. to react for 16 h. The reaction solution was filtered through diatomaceous earth, the filter cake was washed with ethyl acetate (100 mL), and saturated saline solution (50 mL) was used for washing. The organic phase was dried over anhydrous sodium sulfate and concentrated under reduced pressure to obtain a crude product. The crude product was purified by column chromatography (PE/EtOAc=4/1 to 3/2) to obtain compound 9c (0.8 g), brownish yellow oil.

¹H NMR (400 MHz, CDCl₃) δ 7.18 ((dd, J=10.0, 2.4 Hz, 1H), 7.15 (t, J=8.4 Hz, 1H), 7.09 (dd, J=8.4, 2.4 Hz, 1H), 3.27-3.12 (m, 4H), 1.41 (t, J=7.4 Hz, 6H).

### Step 3 1-(4-(4,4,5,5-methyl-1,3,2-dioxaborolan-2-yl)-3-fluorophenyl) imino)-1λ⁶-diethylsulfoxide 9d

Compound 9c (500 mg, 1.7 mmol), bis(pinacolato)diboron 1e (518 mg, 2.0 mmol), Pd(dppf)Cl₂ (249 mg, 0.34 mmol), potassium acetate (500 mg, 5.1 mmol), and 1,4-dioxane (10 mL) were added into a sealed tube, after replaced with argon gas three times, sealed, the reaction solution was heated up to 90° C. and stirred for 16 h. Water (10 mL) was added, and it was extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 9d (360 mg), white solid.

MS (ESI) m/z 342 [M+H]⁺.

¹H NMR (400 MHz, CDCl₃) δ 7.45 (dd, J=11.2, 1.6 Hz, 1H), 7.41 (dd, J=8.0, 1.6 Hz, 1H), 7.27 (t, J=8.0 Hz, 1H), 3.31-3.16 (m, 4H), 1.41 (t, J=7.4 Hz, 6H), 1.32 (s, 12H).

### Step 4 N-(4-(4-amino-5-(4-(diethyl (oxo) 1λ⁶-sulfanylidene)-3-fluorophenyl)-7-methyl 7H-pyrrolo [2,3-d] pyrimidin-6-yl)-phenyl) methacrylamide 9

Compound 9e (300 mg, 0.88 mmol), compound 9d (283 mg, 0.73 mmol), Pd(PPh₃)₄ (84 mg, 0.072 mmol), and K₃PO₄ (464 mg, 2.2 mmol) were added into a sealed tube, after replaced with argon gas three times, 1,4-dioxane (10 mL) and H₂O (1 mL) was added, sealed, the reaction solution was heated up to 100° C. and stirred for 16 h. Dichloromethane (20 mL) and saturated saline solution (10 mL) were added, and it was extracted, the organic phase was dried over anhydrous sodium sulfate, and concentrated under reduced pressure to obtain a crude product. The crude product was purified by column chromatography (MeOH/DCM=1/10) to obtain compound 9 (52 mg), white solid.

MS (ESI) m/z 521 [M+H]⁺.

¹H NMR (400 MHz, CDCl₃) δ 8.26 (s, 1H), 7.56-7.42 (m, 3H), 7.20-7.12 (m, 3H), 6.82 (d, J=10.5 Hz, 2H), 5.74 (s, 1H), 5.43 (s, 1H), 5.27 (brs, 2H), 3.63 (s, 3H), 3.20-3.09 (m, 4H), 2.01 (s, 3H), 1.37 (t, J=7.4 Hz, 6H).

### Example 10 N-(4-(4-amino-5-(4-(3,3-dimethoxy-1-oxo-12′-thiocyclobutan-1-ylidene) amino)-3-fluorophenyl)-7-methyl-7H-pyrrolo [2,3-d] pyrimidin-6-yl)-phenyl) methacrylamide

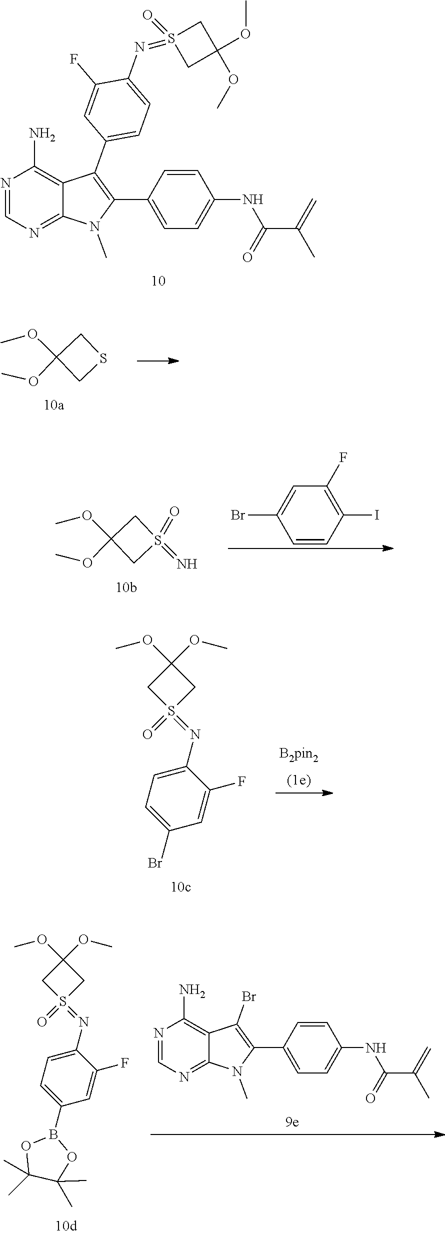

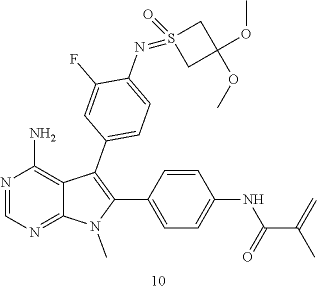 [SMILES: C=C(C)C(=O)Nc1ccc(-c2c(-c3ccc(N=S4(=O)CC(OC)(OC)C4)c(F)c3)c3c(N)ncnc3n2C)cc1]

### Step 1 1-imino-3,3-dimethoxy-1λ⁶-thietane-1-oxide 10b

3,3-dimethoxythietane 10a (2.68 g, 20 mmol) and ammonium carbamate (2.34 g, 30 mmol) were added to a flask sequentially, and then MeOH (80 mL) and iodobenzene diethyl ester (12.9 g, 40 mmol) were added. The reaction solution was stirred open at room temperature for 30 min and then concentrated under reduced pressure to remove the solvent to obtain a crude product. The crude product was purified by column chromatography (MeOH/DCM=1/20) to obtain compound 10b (0.9 g), colorless oil.

¹H NMR (400 MHz, CDCl₃) δ 4.15 (s, 4H), 3.25 (s, 6H).

### Step 2 1-((4-bromo-2-fluorophenyl) imino)-3,3-dimethoxy-1λ⁶-thietan-1-oxide 10c

Compound 10b (380 mg, 2.3 mmol), cesium carbonate (1.13 g, 3.45 mmol), 1-bromo-3-fluoro-4-iodobenzene (832 mg, 2.76 mmol), Pd₂(dba)₃ (105 mg, 0.12 mmol), and Xantphos (200 mg, 0.36 mmol) were added to 1,4-dioxane (10 mL), and the reaction solution was heated up to 100° C. to react for 16 h. The reaction solution was filtered through diatomaceous earth, and the filter cake was washed with ethyl acetate (100 mL) and saturated saline solution (50 mL). The organic phase was dried over anhydrous sodium sulfate and concentrated under reduced pressure to obtain a crude product. The crude product was purified by column chromatography (PE/EtOAc=4/1 to 3/2) to obtain compound 10c (0.4 g), light yellow solid.

¹H NMR (400 MHz, CDCl₃) δ 7.20 (dd, J=10.4, 2.4 Hz, 1H), 7.14 (dd, J=8.6, 2.4 Hz, 1H), 6.95 (t, J 8.6 Hz, 1H), 4.35 (d, J=13.2 Hz, 1H), 4.27 (d, J=13.2 Hz, 1H), 3.28 (s, 3H), 3.26 (s, 3H).

### Step 3 1-((2-fluoro-4-(4,4,5,5-tetramethyl-1,3,2-dioxaborolan-2-yl) phenyl) imino)-3,3-dimethoxy-1λ⁶-thietane-1-oxide 10d

Compound 1d (600 mg, 1.77 mmol), bis(pinacolato)diboron (541 mg, 2.13 mmol), Pd(dppf)Cl₂ (259 mg, 0.35 mmol), potassium acetate (520 mg, 5.31 mmol), and 1,4-dioxane (10 mL) were added into a sealed tube, it was replaced with argon gas three times, sealed, the reaction solution was heated up to 90° C. and stirred for 16 h. Water (10 mL) was added and it was extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 10d (580 mg), brownish yellow solid.

¹H NMR (400 MHz, CDCl₃) δ 7.50-7.45 (m, 1H), 7.45 (s, 1H), 7.05 (t, J=7.9 Hz, 1H), 4.41-4.25 (m, 4H), 3.28 (s, 3H), 3.25 (s, 3H), 1.32 (s, 12H).

### Step 4 N-(4-(4-amino-5-(4-(3,3-dimethoxy-1-oxo-1λ⁶-thiocyclobutan-1-ylidene) amino)-3-fluorophenyl)-7-methyl-7H-pyrrolo [2,3-d] pyrimidin-6-yl)-phenyl) methacrylamide 10

Compound 9e (185 mg, 0.48 mmol), compound 10d (154 mg, 0.4 mmol), Pd(PPh₃)₄ (46 mg, 0.04 mmol), and K₃PO₄ (254 mg, 1.2 mmol) were added into a sealed tube, after replaced with argon gas three times, 1,4-dioxane (5 mL) and H₂O (0.5 mL) were added, sealed, the reaction solution was heated up to 100° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 10 (19 mg), white solid.

MS (ESI) m/z 565 [M+H]⁺.

¹H NMR (400 MHz, CDCl₃) δ 8.34 (s, 1H), 7.65 (s, 1H), 7.57 (d, J=8.6 Hz, 2H), 7.20 (d, J=8.6 Hz, 2H), 7.00 (dd, J=9.2, 7.6 Hz, 1H), 6.95-6.88 (m, 2H), 5.79 (s, 1H), 5.47 (d, J=1.6 Hz, 1H), 5.07 (brs, 2H), 4.37 (d, J=13.6 Hz, 2H), 4.29 (d, J 13.6 Hz, 2H), 3.68 (s, 3H), 3.28 (s, 3H), 3.26 (s, 3H), 2.06 (s, 3H).

### Example 11 N-(4-(4-amino-5-(3-fluoro-4-(6-oxo-2-oxa-6λ⁶-thiaspiro [3.3]heptan-6-ylidene) amino) phenyl)-7-methyl-7H-pyrrolo [2,3-d] pyrimidin-6-yl)-phenyl) methacrylamide

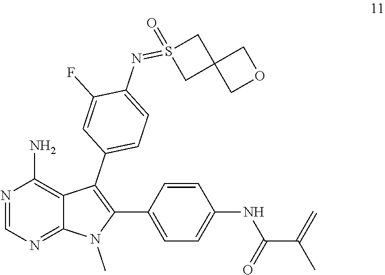 [SMILES: C=C(C)C(=O)Nc1ccc(-c2c(-c3ccc(N=S4(=O)CC5(COC5)C4)c(F)c3)c3c(N)ncnc3n2C)cc1]

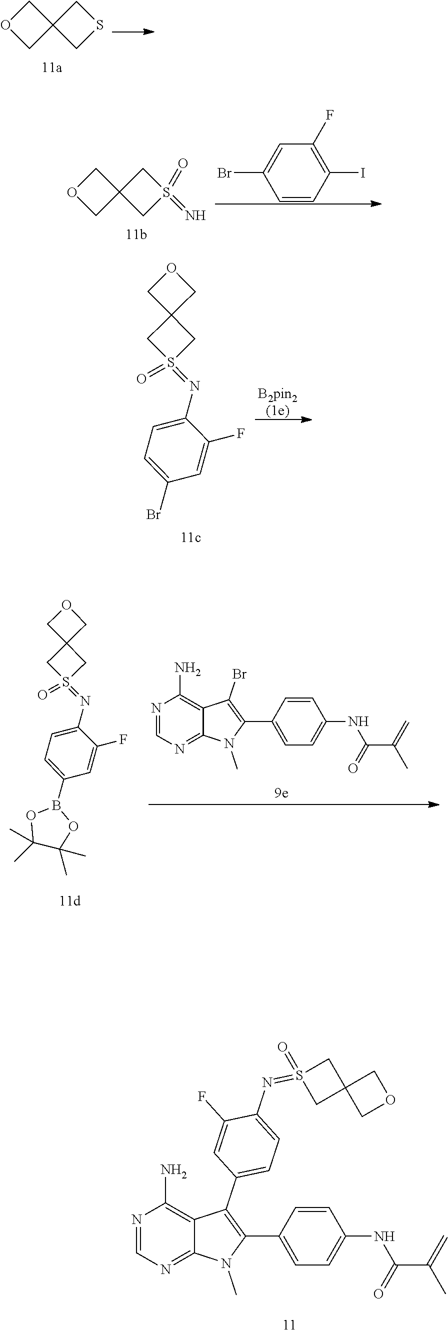

### Step 1 6-amino-2-oxo-6λ⁶-thiaspiro [3.3] heptane-6-oxide 11b

Compound 11a (1.74 g, 15 mmol) and ammonium carbamate (1.75 g, 22.5 mmol) were added to a flask sequentially, and then MeOH (100 mL) and iodobenzene diethyl ester (9.66 g, 30 mmol) were added. The reaction solution was stirred open at room temperature for 30 min and then concentrated under reduced pressure to remove the solvent to obtain a crude product. The crude product was purified by column chromatography (MeOH/DCM=1/20) to obtain compound 11b (0.7 g), colorless oil.

¹H NMR (400 MHz, CDCl₃) δ 4.85 (s, 2H), 4.81 (s, 2H), 4.28 (s, 4H), 2.04 (s, 1H).

### Step 2 6-((4-bromo-2-fluorophenyl) imino)-2-oxo-6λ⁶-thiaspiro [3.3] heptane-6-oxide 11c

Compound 11b (480 mg, 3.3 mmol), cesium carbonate (1.59 g, 4.9 mmol), 1-bromo-3-fluoro-4-iodobenzene (1.2 g, 3.9 mmol), Pd₂(dba)₃ (92 mg, 0.1 mmol), and Xantphos (174 mg, 0.3 mmol) were added to 1,4-dioxane (15 mL), and the reaction solution was heated up to 100° C. for 16 h. The reaction solution was filtered through diatomaceous earth, and the filter cake was washed with ethyl acetate (100 mL) and saturated saline solution (50 mL). The organic phase was dried over anhydrous sodium sulfate and concentrated under reduced pressure to obtain a crude product. The crude product was purified by column chromatography (PE/EtOAc=4/1 to 3/2) to obtain compound 11c (0.6 g), brownish yellow solid.

¹H NMR (400 MHz, CDCl₃) δ 7.20 (dd, J=10.4, 2.4 Hz, 1H), 7.13 (ddd, J=8.4, 2.4, 1.0 Hz, 1H), 6.93 (t, J=8.8 Hz, 1H), 4.84 (s, 2H), 4.82 (s, 2H), 4.45 (d, J=14.0 Hz, 2H), 4.39 (d, J=14.0 Hz, 2H).

### Step 3 6-((2-fluoro-4-(4,4,5,5-tetramethyl-1,3,2-dioxaborolan-2-yl) phenyl) imino)-2-oxo-6λ⁶-thiaspiro [3.3] heptane-6-oxide 11d

Compound 11c (260 mg, 0.81 mmol), bis(pinacolato)diboron (248 mg, 0.98 mmol), Pd(dppf)Cl₂ (119 mg, 0.16 mmol), potassium acetate (238 mg, 2.43 mmol), and 1,4-dioxane (5 mL) were added into a sealed tube, after replaced with argon gas three times, sealed, the reaction solution was heated up to 90° C. and stirred for 16 h. Water (10 mL) was added and extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 11d (230 mg), brownish yellow solid.

¹H NMR (400 MHz, CDCl₃) δ 7.47 (dd, J=5.2, 1.2 Hz, 1H), 7.45 (s, 1H), 7.04 (t, J=8.0 Hz, 1H), 4.86 (s, 2H), 4.85 (s, 2H), 4.50 (d, J=14.0 Hz, 2H), 4.42 (d, J=14.0 Hz, 2H), 1.32 (s, 12H).

### Step 4 N-(4-(4-amino-5-(3-fluoro-4-(6-oxo-2-oxa-6λ⁶-thiaspiro [3.3]heptan-6-ylidene) amino) phenyl)-7-methyl-7H-pyrrolo [2,3-d] pyrimidin-6-yl)-phenyl) methacrylamide 11

Compound 8e (150 mg, 0.4 mmol), compound 11d (143 mg, 0.37 mmol), Pd(PPh₃)₄ (43 mg, 0.037 mmol), and K₃PO₄ (235 mg, 1.1 mmol) were added into a sealed tube, after replaced with argon gas three times, 1,4-dioxane (4 mL) and H₂O (0.4 mL) were added, sealed, and the reaction solution was heated up to 100° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 11 (15 mg), white solid.

MS (ESI) m/z 547 [M+H]⁺.

¹H NMR (400 MHz, CDCl₃) δ 8.29 (s, 1H), 7.58-7.43 (m, 3H), 7.15 (d, J=8.6 Hz, 2H), 6.98-6.80 (m, 3H), 5.73 (s, 1H), 5.42 (d, J=1.6 Hz, 1H), 4.92 (s, 2H), 4.82 (s, 2H), 4.79 (s, 2H), 4.45 (d, J=14.1 Hz, 2H), 4.37 (d, J=14.1 Hz, 2H), 3.62 (s, 3H), 2.00 (s, 3H).

### Example 12 N-(3-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl)-4-fluorophenyl) methacrylamide

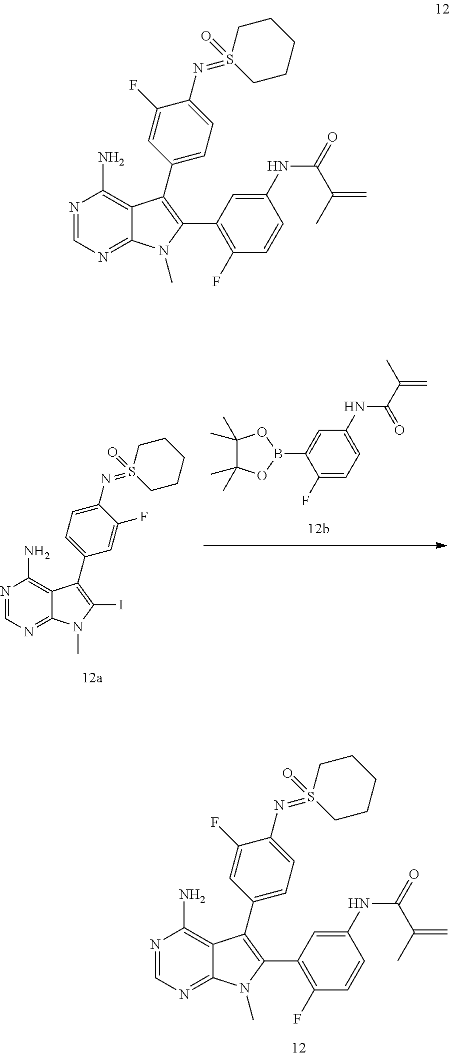

Compound 12a (150 mg, 0.3 mmol), compound 12b (105 mg, 0.36 mmol), Pd(PPh₃)₄ (35 mg, 0.03 mmol), and K₃PO₄ (192 mg, 0.9 mmol) were added into a sealed tube, after replaced with argon gas three times, 1,4-dioxane (3 mL) and H₂O (0.3 mL) was added, sealed, and the reaction solution was heated up to 90° C. and stirred for 16 h. Dichloromethane (40 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 12 (10 mg), white solid.

MS (ESI) m/z 551 [M+H]⁺.

¹H NMR (400 MHz, CDCl₃) δ 8.36 (s, 1H), 7.65 (ddd, J=8.8, 4.4, 2.8 Hz, 1H), 7.45 (s, 1H), 7.30 (dd, J=6.0, 2.8 Hz, 1H), 7.19 (t, J=8.8 Hz, 1H), 7.12 (t, J=8.8 Hz, 1H), 7.00-6.88 (m, 2H), 5.76 (s, 1H), 5.46 (d, J=1.6 Hz, 1H), 5.11 (brs, 2H), 3.65 (s, 3H), 3.36-3.28 (m, 2H), 3.17-3.10 (m, 2H), 2.18-2.10 (m, 4H), 2.03 (s, 3H), 1.66-1.58 (m, 2H).

### Example 13 N-(4-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl) phenyl) acrylamide

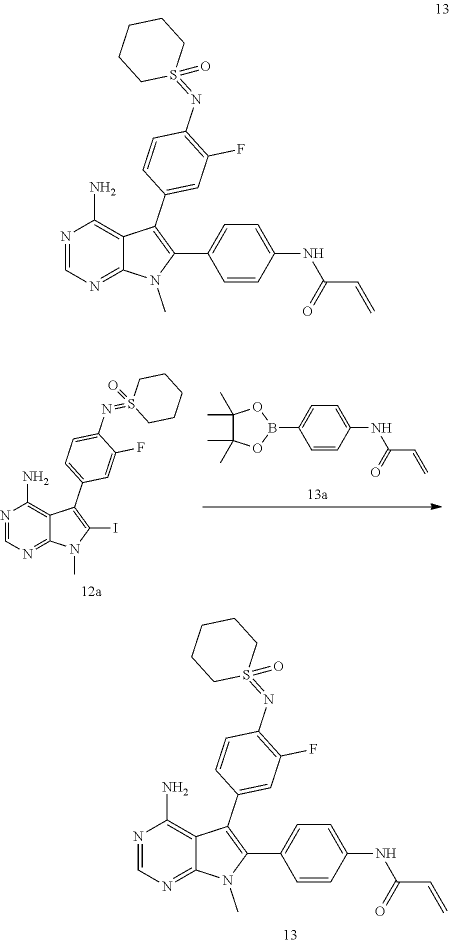

Compound 12a (150 mg, 0.3 mmol), compound 13a (107 mg, 0.39 mmol), Pd(PPh₃)₄ (69 mg, 0.06 mmol), and K₃PO₄ (192 mg, 0.9 mmol) were added into a sealed tube, after replaced with argon gas three times, 1,4-dioxane (6 mL) and H₂O (0.6 mL) were added, sealed, and the reaction solution was heated up to 90° C. and stirred for 16 h. Dichloromethane (40 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 13 (13 mg), white solid.

MS (ESI) m/z 519 [M+H]⁺.

¹H NMR (400 MHz, CDCl₃) δ 8.28 (s, 1H), 7.76 (s, 1H), 7.59 (d, J=8.4 Hz, 2H), 7.19 (d, J=8.4 Hz, 2H), 7.17 (m, 1H), 6.90-6.84 (m, 2H), 6.44 (dd, J=16.8, 1.2 Hz, 1H), 6.28 (dd, J 16.8, 10.0 Hz, 1H), 5.77 (dd, J=10.0, 1.2 Hz, 1H), 5.60 (brs, 2H), 3.68 (s, 3H), 3.35-3.29 (m, 2H), 3.18-3.11 (m, 2H), 2.13-2.06 (m, 4H), 1.79-1.70 (m, 1H), 1.66-1.58 (m, 1H).

### Example 14 N-(4-(4-amino-5-(3-fluoro-4-((1-oxotetrahydro-2H-1λ⁶-thiopyran-1-ylidene)amino)phenyl)-7-methyl-7H-pyrrolo[2,3-d] pyrimidin-6-yl)-3-methoxyphenyl) methacrylamide

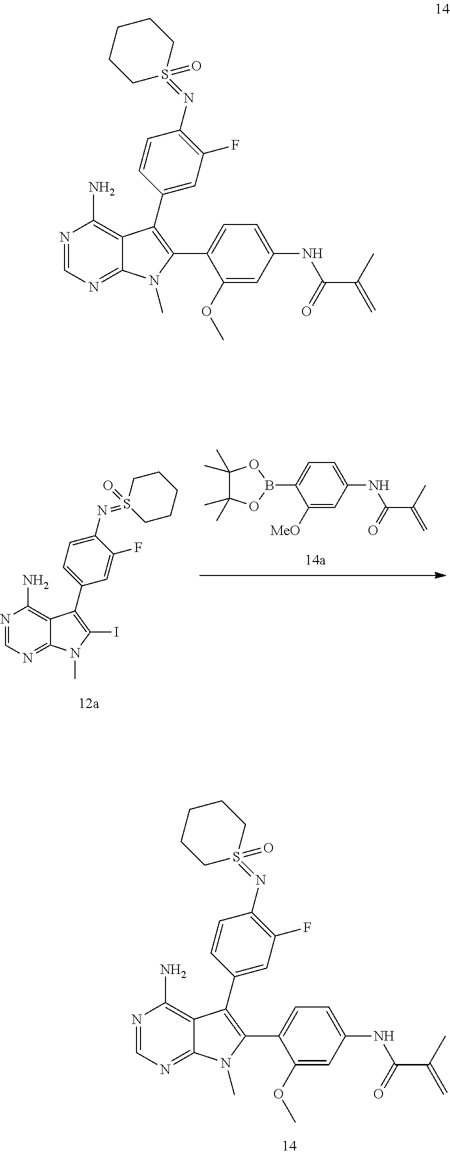

Compound 12a (50 mg, 0.1 mmol), compound 14a (38 mg, 0.12 mmol), Pd(PPh₃)₄ (23 mg, 0.02 mmol), and K₃PO₄ (64 mg, 0.3 mmol) were added into a sealed tube, after replaced with argon gas three times, 1,4-dioxane (2 mL) and H₂O (0.2 mL) were added, sealed, and the reaction solution was heated up to 90° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 14 (10 mg), white solid.

MS (ESI) m/z 563 [M+H]⁺.

¹H NMR (400 MHz, CDCl₃) δ 8.27 (s, 1H), 7.71 (d, J=2.0 Hz, 1H), 7.62 (s, 1H), 7.16 (t, J=8.0 Hz, 1H), 6.93 (d, J=8.0 Hz, 2H), 6.91-6.85 (m, 1H), 6.76 (dd, J=8.0, 2.0 Hz, 1H), 5.81 (s, 1H), 5.81 (brs, 2H), 5.50 (s, 1H), 3.79 (s, 3H), 3.58 (s, 3H), 3.30 (m, 2H), 3.14 (m, 2H), 2.14-2.06 (m, 4H), 2.07 (s, 3H), 1.78-1.69 (m, 1H), 1.66-1.56 (m, 1H).

### Example 15 (E)-N-(4-(4-amino-5-(3-fluoro-4-((1-oxotetrahydro-2H-12′-thiopyran-1-ylidene) amino) phenyl)-7-methyl-7H-pyrrolo [2,3-d] pyrimidin-6-yl)-phenyl)-2-butenamide

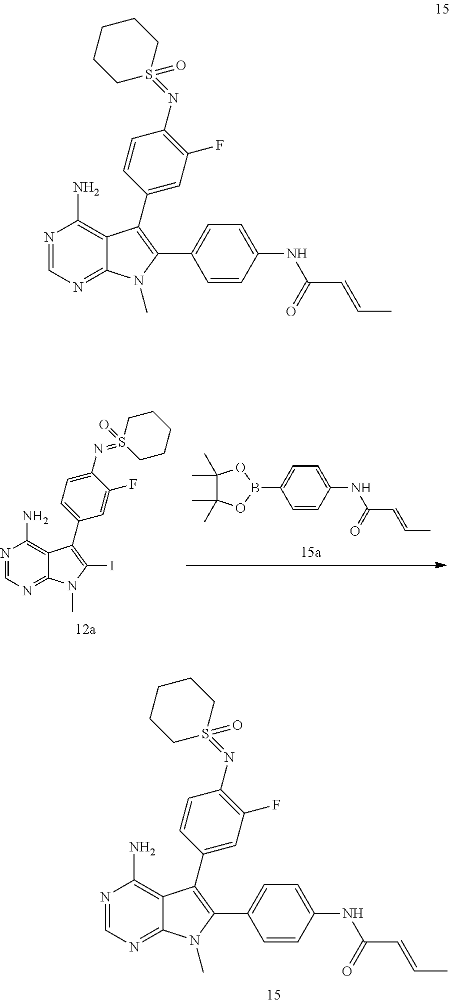

Compound 12a (150 mg, 0.3 mmol), compound 15a (103 mg, 0.36 mmol), Pd(PPh₃)₄ (35 mg, 0.03 mmol), and K₃PO₄ (192 mg, 0.9 mmol) were added into a sealed tube, after replaced with argon gas three times, 1,4-dioxane (5 mL) and H₂O (0.5 mL) were added, sealed, and the reaction solution was heated up to 90° C. and stirred for 16 h. Dichloromethane (40 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 15 (7 mg), white solid.

MS (ESI) m/z 533 [M+H]⁺.

¹H NMR (400 MHz, CDCl₃) δ 8.36 (s, 1H), 7.56 (d, J=8.5 Hz, 2H), 7.21-7.16 (m, 4H), 7.05-6.96 (m, 1H), 6.92-6.87 (m, 2H), 5.96 (dd, J=15.1, 1.8 Hz, 1H), 4.98 (s, 2H), 3.69 (s, 3H), 3.38-3.28 (m, 2H), 3.20-3.11 (m, 2H), 2.15-2.06 (m, 4H), 1.93 (dd, J=6.9, 1.7 Hz, 3H), 1.79-1.71 (m, 1H), 1.69-1.61 (m, 1H).

### Example 16 (E)-N-(4-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl)-phenyl)-4-(dimethylamino)-2-butenamide

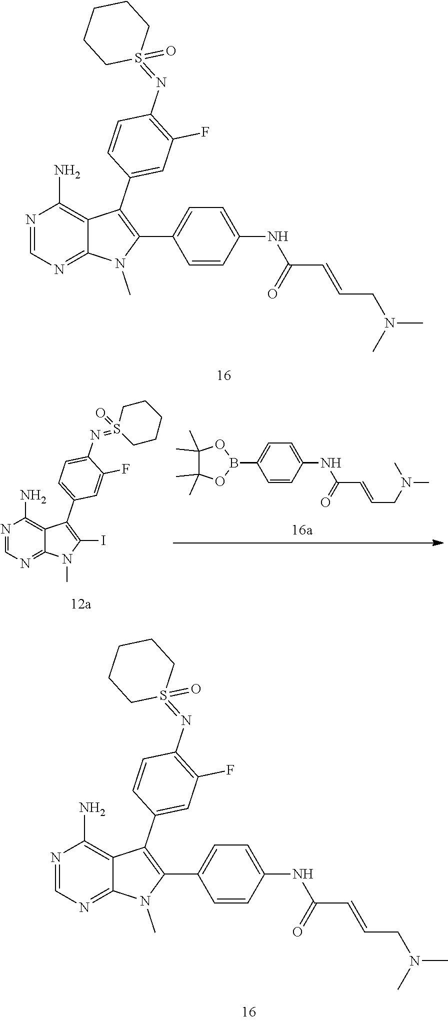

Compound 12a (100 mg, 0.2 mmol), compound 16a (77 mg, 0.24 mmol), Pd(PPh₃)₄ (23 mg, 0.02 mmol), and K₃PO₄ (127 mg, 0.6 mmol) were added into a sealed tube, after replaced with argon gas three times, 1,4-dioxane (3 mL) and H₂O (0.3 mL) were added, sealed, and the reaction solution was heated up to 90° C. and stirred for 16 h. Dichloromethane (40 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 16 (16 mg), white solid.

MS (ESI) m/z 574 [M−H]−.

¹H NMR (400 MHz, CDCl₃) δ 8.44 (s, 1H), 7.68-7.60 (m, 3H), 7.36-7.28 (m, 3H), 7.11-7.04 (m, 1H), 6.99 (d, J=9.5 Hz, 2H), 6.23 (d, J=15.6 Hz, 1H), 5.15 (s, 2H), 3.78 (s, 3H), 3.45-3.40 (m, 2H), 3.26-3.20 (m, 4H), 2.37 (s, 6H), 2.28-2.16 (m, 4H), 1.86-1.79 (m, 1H), 1.78-1.66 (m, 1H).

### Example 17 N-[4-(4-amino-7-methyl-5-(6-((1-oxotetrahydro-2H-1λ⁶-thiopyran-1-ylidene) amino) pyridin-3-yl) pyrrolo [2,3-d] pyrimidin-6-yl) phenyl) methacrylamide

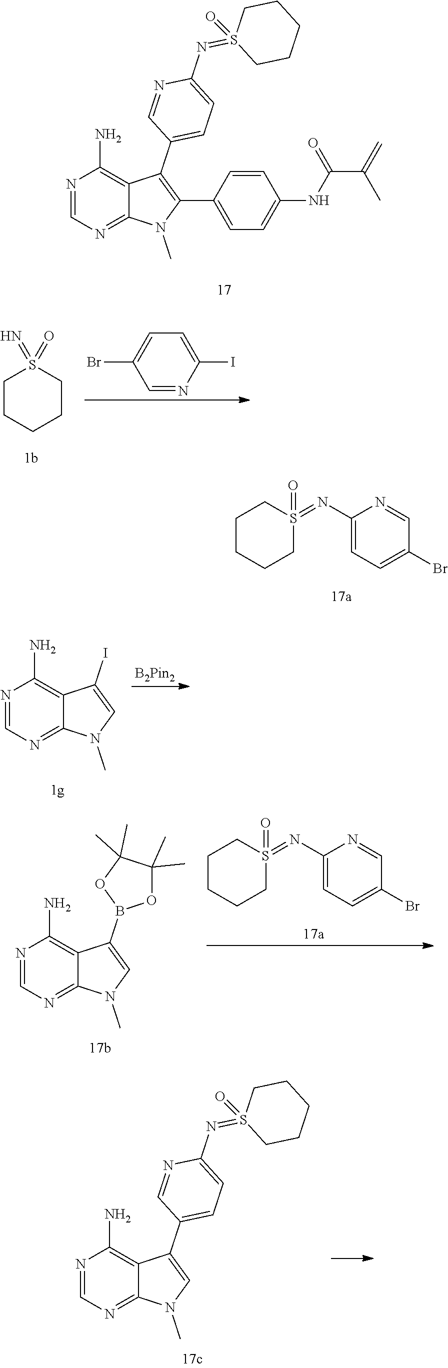

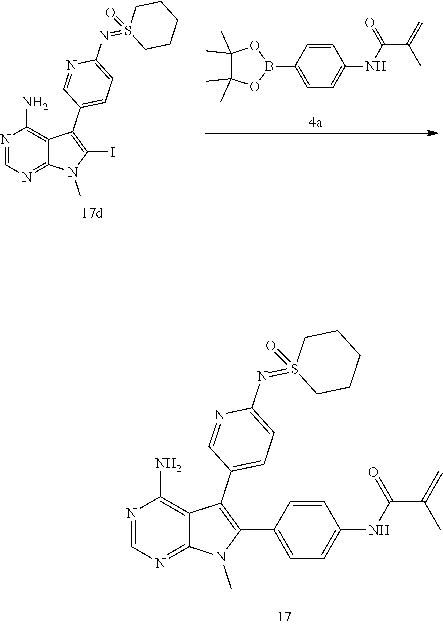 [SMILES: Cn1c(I)c(-c2ccc(N=S3(=O)CCCCC3)nc2)c2c(N)ncnc21 ; C=C(C)C(=O)Nc1ccc(B2OC(C)(C)C(C)(C)O2)cc1 ; C=C(C)C(=O)Nc1ccc(-c2c(-c3ccc(N=S4(=O)CCCCC4)nc3)c3c(N)ncnc3n2C)cc1]

### Step 1 1-[(5-bromopyridin-2-yl) imino]-1λ⁶-thiopyran-1-oxide 17a

At room temperature, compound 1b (1 g, 7.52 mmol), 5-bromo-2-iodopyridine (2.35 g, 8.27 mmol), tris (dibenzylideneacetone) dipalladium (173.99 mg, 0.19 mmol), 4,5-diphenylphosphine-9,9-dimethyloxanthracene (324.03 mg, 0.56 mmol), cesium carbonate (3.43 g, 10.53 mmol), 1,4-dioxane (20 ml), and a stirrer were added to a round bottom flask sequentially, followed by vacuum pumping and nitrogen gas purging three times. The reaction was heated up to 100° C. and stirred for 16 h. After the reaction was completed, the mixture was cooled down to room temperature and concentrated under reduced pressure to obtain a crude product. The crude product was purified by column chromatography (PE/EtOAc=1/1) to obtain compound 17a (700 mg), light yellow solid.

MS (ESI) m/z 289 [M+H]⁺.

¹H NMR (400 MHz, DMSO-d₆) δ 8.16 (d, J=4.0 Hz, 1H), 7.72 (dd, J=4.0, 4.0 Hz, 1H), 6.67 (d, J=8.0 Hz, 1H), 3.63-3.62 (m, 2H), 3.43-3.42 (m, 2H), 2.02-1.81 (m, 4H), 1.62-1.59 (m, 2H).

### Step 2 7-methyl-5-(4,4,5,5-tetramethyl-1,3,2-dioxaborolan-2-yl) pyrrolo [2,3-d]pyrimidin-4-amine

At room temperature, compound 1g (5 g, 18.25 mmol), bis(pinacolato)diboron (6.95 g, 27.37 mmol), potassium acetate (5.34 g, 54.75 mmol), of 1,1′-biphenylphosphine ferrocene palladium chloride (1.34 mg, 1.83 mmol), and dimethyl sulfoxide (25 mL) were added to a round bottom flask sequentially, followed by vacuum pumping and nitrogen gas purging three times. The reaction was heated up to 100° C. and stirred for 1 h. After the reaction was completed, the mixture was cooled down to room temperature, diluted with water, extracted with ethyl acetate (20 mL×3), washed with saturated saline (30 mL), and the organic phases were combined, dried over anhydrous sodium sulfate and concentrated under reduced pressure to obtain a crude product. The crude product was purified by column chromatography (DCM/MeOH=15/1) to obtain compound 7b (700 mg), reddish brown solid.

MS (ESI) m/z 275 [M+H]⁺.

### Step 3 1-((5-(4-amino-7-methyl-7H-pyrrolo [2,3-d] pyrimidin-5-yl) pyridin-2-yl) imino) hexahydro-1λ⁶-thiopyran-1-oxide 17c

At room temperature, compound 7b (500 mg, 1.82 mmol), compound 17a (471.74 mg, 1.64 mmol), 1,1′-bis(ditert-butylphosphine) ferrocene palladium dichloro (117.68 mg, 0.18 mmol), cesium fluoride (829.37 mg, 5.46 mmol), N,N-dimethylformamide (10 mL), and water (2.5 mL) were added to a round bottom flask in sequence, followed by vacuum pumping and nitrogen gas purging three times. The reaction was heated up to 100° C. and stirred for 1 h. After the reaction was completed, the mixture was cooled down to room temperature, diluted with water, extracted with ethyl acetate (15 ml×3), washed with saturated saline (20 ml), and the organic phases were combined, dried over anhydrous sodium sulfate and concentrated under reduced pressure to obtain a crude product. The crude product was purified by column chromatography (DCM/MeOH=10/1) to obtain compound 17c (400 mg), brown solid.

MS (ESI) m/z 357 [M+H]⁺.

¹H NMR (400 MHz, DMSO-d₆) δ 8.17-8.16 (m, 2H), 7.61 (dd, J=4.0, 4.0 Hz, 1H), 7.28 (s, 1H), 6.79 (d, J=8.0 Hz, 1H), 6.07 (s, 2H), 3.74 (s, 3H), 3.69-3.64 (m, 2H), 3.47-3.41 (m, 2H), 1.96-1.94 (m, 4H), 1.64-1.61 (m, 2H).

### Step 4 1-(5-(4-amino-6-iodo-7-methyl-7H-pyrrolo [2,3-d] pyrimidin-5-yl) pyridine-2-yl) imino) hexahydro-1λ⁶-thiopyran-1-oxide 17d

Compound 17c (300 mg, 0.84 mmol), dichloromethane (3 ml), and trifluoroacetic acid (287.33 mg, 2.52 mmol) were added to a round bottom flask at room temperature. After it was cooled down to 0° C., N-iodosuccinimide (283.47 mg, 1.26 mmol) was added in batches, and the reaction was warmed up to room temperature naturally and stirred for additional 2 h. After TLC monitored the completion of the reaction, the mixture was concentrated under reduced pressure to obtain a crude product. The crude product was purified by column chromatography (DCM/MeOH=10/1) to obtain compound 17d (65 mg), white solid.

MS (ESI) m/z 483 [M+H]⁺.

¹H NMR (400 MHz, DMSO-d₆) δ 8.10-8.09 (m, 2H), 7.55 (dd, J=4.0, 4.0 Hz, 1H), 6.81 (d, J=8.0 Hz, 1H), 5.95 (s, 2H), 3.74 (s, 3H), 3.70-3.66 (m, 1H), 3.51-3.45 (m, 2H), 3.17 (d, J=4.0 Hz, 1H), 1.97-1.96 (m, 4H), 1.66-1.62 (m, 2H).

### Step 5 N-[4-(4-amino-7-methyl-5-(6-(1-oxotetrahydro-2H-1λ⁶-thiopyran-1-ylidene) amino) pyridin-3-yl) pyrrolo [2,3-d] pyrimidin-6-yl) phenyl) methacrylamide 17

At room temperature, compound 17d (65 mg, 0.13 mmol), compound 4a (40.20 mg, 0.14 mmol), chlorine[(n-butyl di (1-adamantanyl) phosphine)-2-(2-aminobiphenyl)] palladium (II) (8.69 mg, 0.013 mmol), potassium phosphate (55.12 mg, 0.26 mmol), N,N-dimethylacetamide (2 mL)/water (0.5 mL) were added to a round bottom flask sequentially, followed by vacuum pumping and nitrogen gas purging three times. The reaction was heated up to 80° C. and stirred for 3 h. After the reaction was completed, the mixture was cooled down to room temperature and concentrated under reduced pressure to obtain a crude product. The crude product was purified by column chromatography (DCM/MeOH=10/1) to obtain compound 17 (11.2 mg), white solid.

MS (ESI) m/z 516 [M+H]⁺.

¹H NMR (400 MHz, DMSO-d₆) δ 9.93 (s, 1H), 8.19 (s, 1H), 7.90 (d, J=4.0 Hz, 1H), 7.73 (d, J=8.0 Hz, 2H), 7.40 (dd, J=4.0, 4.0 Hz, 1H), 7.28 (d, J=8.0 Hz, 2H), 6.67 (d, J=8.0 Hz, 1H), 5.88 (s, 2H), 5.82 (s, 1H), 5.53 (s, 1H), 3.62 (s, 1H), 3.60 (s, 3H), 3.34-3.41 (m, 1H), 3.18-3.17 (m, 2H), 1.96 (s, 3H), 1.95-1.93 (m, 4H), 1.61-1.58 (m, 2H).

### Example 18 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl) phenyl)-2-(methoxymethyl) acrylamide

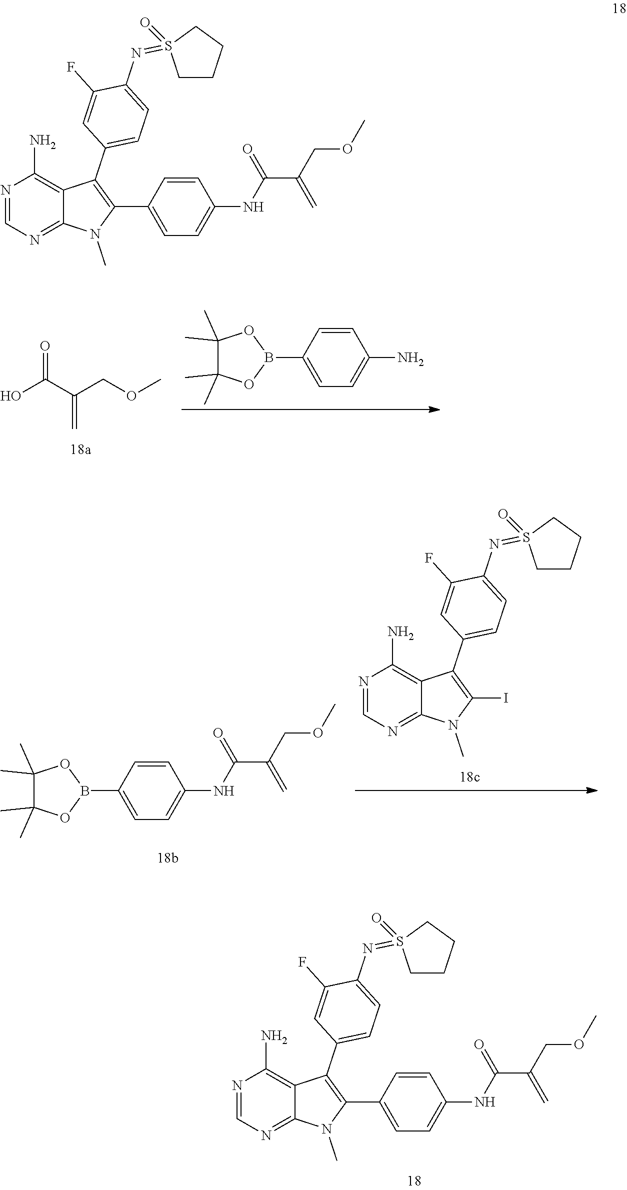

### Step 1 7-methyl-5-(4,4,5,5-tetramethyl-1,3,2-dioxaborolan-2-yl) pyrrolo [2,3-d]pyrimidin-4-amine 18b

Compound 18a (150 mg, 1.29 mmol) and 4-(4,4,5,5-tetramethyl-1,3,2-dioxaborolan-2-yl) aniline (197.23 mg, 0.90 mmol) were dissolved in N,N-dimethylformamide (3 mL), and then triethylamine (391.6 mg, 3.87 mmol) was added. It was cooled down to 0° C., a solution of 1-propylphosphonic anhydride in 50% ethyl acetate (820.90 mg, 1.93 mmol) was added slowly dropwise, and heated up to room temperature and stirred to react for 4 h. After the reaction was completed, the mixture was diluted with water, extracted with ethyl acetate (5 ml×3), washed with saturated saline solution (10 mL), and the organic phases were combined and dried over anhydrous sodium sulfate. The crude product was concentrated under reduced pressure and purified by column chromatography (DCM/MeOH=15/1) to obtain compound 18b (60 mg), light yellow solid.

MS (ESI) m/z 275 [M+H]⁺.

¹H NMR (400 MHz, DMSO-d₆) δ 10.00 (s, 1H), 7.71 (d, J=8.0 Hz, 2H), 7.62 (d, J=8.0 Hz, 2H), 5.97 (s, 1H), 5.70 (s, 1H), 4.16 (s, 2H), 3.30 (s, 3H), 1.29 (s, 12H).

### Step 2 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl) phenyl)-2-(methoxymethyl) acrylamide 18

At room temperature, compound 18b (60.00 mg, 0.19 mmol), compound 18c (82.50 mg, 0.17 mmol), chlorine [(n-butyl di (1-adamantanyl) phosphine)-2-(2-aminobiphenyl)]palladium (II) (12.70 mg, 0.019 mmol), potassium phosphate (80.56 mg, 0.38 mmol), N,N-dimethylacetamide (2 mL), and water (0.5 mL) were sequentially added to a round bottom flask, followed by vacuum pumping and nitrogen gas purging three times, then heat up to 80° C. and stirred for 3 h. After the reaction was completed, the mixture was cooled to room temperature and concentrated under reduced pressure to obtain a crude product. The crude product was purified by column chromatography (DCM/MeOH=15/1) to obtain compound 18 (12.3 mg), white solid.

MS (ESI) m/z 549 [M+H]⁺.

¹H NMR (400 MHz, DMSO-d₆) δ 10.09 (s, 1H), 8.24 (s, 1H), 7.77 (d, J=8.0 Hz, 2H), 7.35 (d, J=8.0 Hz, 2H), 7.10 (t, J=20.0 Hz, 1H), 6.99-6.95 (m, 2H), 6.02 (s, 1H), 5.76 (s, 1H), 4.22 (s, 2H), 3.65 (s, 3H), 3.42 (s, 2H), 3.37 (s, 3H), 2.29-2.06 (m, 4H), 1.31 (s, 1H).

### Example 19 (4-(4-amino-6-(4,4-dimethyl-2-(propyl-1-yn-1-yl)-4H benzo [d] [1,3]oxazin-6-yl)-7-methyl-7H-pyrrolo [2,3-d] pyrimidin-5-yl)-2-fluorophenyl) imino) dimethyl-λ⁶-sulfoxide

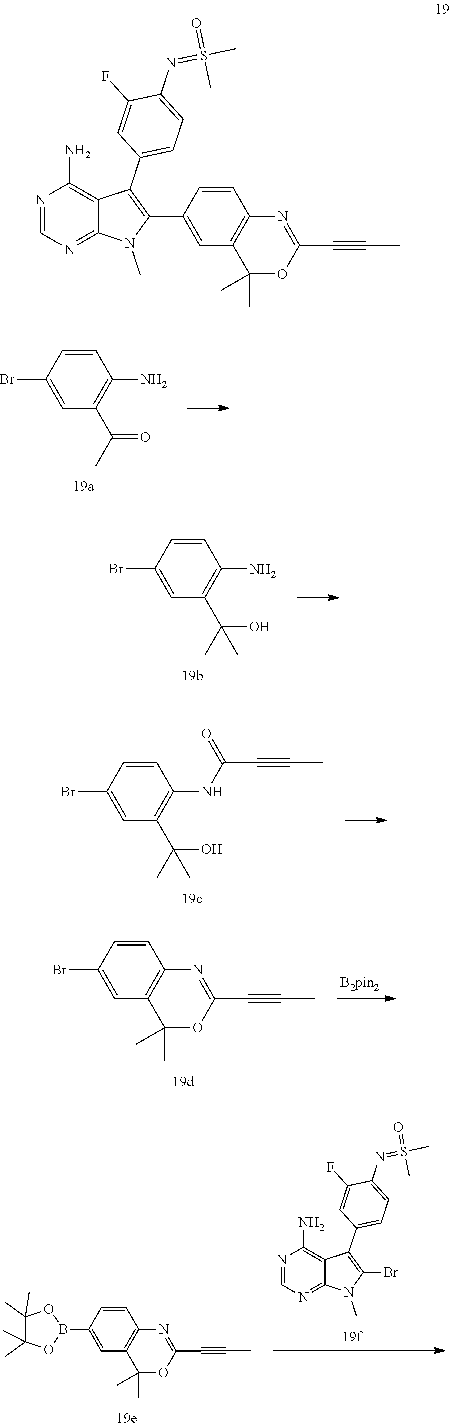

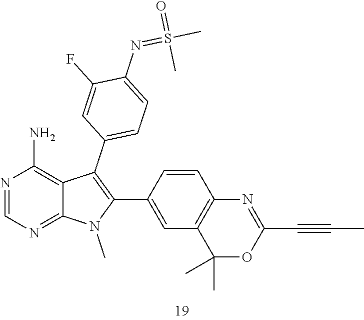 [SMILES: CC#CC1=Nc2ccc(-c3c(-c4ccc(N=S(C)(C)=O)c(F)c4)c4c(N)ncnc4n3C)cc2C(C)(C)O1]

### Step 1 2-(2-amino-5-bromophenyl) propan-2-ol 19b

Compound 19a (1 g, 4.67 mmol) was dissolved in tetrahydrofuran (5 mL), and it was cooled to around 0° C., a solution of methyl magnesium bromide in tetrahydrofuran (1 M, 7 mL) was added, and then the temperature was restored to room temperature to react for 2 h. The reaction solution was quenched by added saturated amine chloride solution (20 mL), extracted with ethyl acetate (20 mL×3), and the organic phases were combined, dried over anhydrous sodium sulfate and concentrated. The crude product was purified by column chromatography (PE/EtOAc=10/1) to obtain compound 19b (0.7 g), white solid.

MS (ESI) m/z 230 [M+H]⁺.

### Step 2 N-(4-bromo-2-(2-hydroxypropyl-2-yl) phenyl) but-2-amide 19c

Compound 19b (200 mg, 0.87 mmol) and 2-butyne acid (73 mg, 0.87 mmol) were dissolved in dry dichloromethane (5 mL), after cooled down to around 0° C., 1-ethyl-(3-dimethylaminopropyl) carbodiimide hydrochloride (183 mg, 0.95 mmol) was added, and then the temperature was restored to room temperature to react for 3 h. The reaction solution was concentrated, and the crude product was purified by column chromatography (PE/EtOAc=10/1) to obtain compound 19c (200 mg), yellow oil.

MS (ESI) m/z 296 [M+H]⁺.

¹H NMR (400 MHz, DMSO-d₆) δ 10.90 (s, 1H), 8.04 (d, J=9.2 Hz, 1H), 7.52-7.35 (m, 2H), 6.38 (s, 1H), 2.04 (s, 3H), 1.53 (s, 6H).

### Step 3 6-bromo-4,4-dimethyl-2-(propyl-1-acetyl-1-yl)-4H benzo [d] [1,3] oxazine 19d

Compound 19c (540 mg, 1.82 mmol) was dissolved in dry dichloromethane (5 mL), methanesulfonic acid (876 mg, 9.13 mmol) was added, and under nitrogen protection, it was heated up to 45° C. to react for 16 h, and then cooled down to room temperature, concentrated to dry solvent. Saturated sodium bicarbonate solution (20 mL) was added and extracted with ethyl acetate (20 mL×3), and the organic phases were combined, washed with saturated saline water (50 mL), and concentrated under reduced pressure to obtain a crude product. The crude product was purified by column chromatography (PE/EtOAc=50/1) to obtain compound 19d (240 mg), yellow oil.

MS (ESI) m/z 278 [M+H]⁺.

¹H NMR (400 MHz, DMSO-d₆) δ 7.52 (d, J=2.2 Hz, 1H), 7.49-7.45 (m, 1H), 7.01 (d, J=8.3 Hz, 1H), 2.05 (s, 3H), 1.58 (s, 6H).

### Step 4 4,4-dimethyl-2-(propyl-1-yn-1-yl)-6-(4,4,5,5-tetramethyl-1,3,2-dioxaborolan-2-yl)-4H benzo [d] [1,3] oxazine 19e

Compound 19d (270 mg, 0.97 mmol) was dissolved in dry 1,4-dioxane (10 mL), bis(pinacolato)diboron (370 mg, 1.46 mmol), 1,1′-biphenylphosphine ferrocene palladium chloride (71 mg, 0.097 mmol), and potassium acetate (285 mg, 2.91 mmol) were added sequentially, after replaced with nitrogen three times, heated up to 90° C. under nitrogen protection to react for 18 h. Then cooled down to room temperature, filtered, concentrated, and the crude product was purified by column chromatography (PE/EtOAc=50/1) to obtain compound 19e (140 mg), yellow oil.

MS (ESI) m/z 325 [M+H]⁺.

### Step 5 (4-(4-amino-6-(4,4-dimethyl-2-(propyl-1-yn-1-yl)-4H benzo [d] [1,3] oxazine-6-yl)-7-methyl-7H-pyrrolo [2,3-d] pyrimidin-5-yl)-2-fluorophenyl) imino) dimethyl-λ⁶-sulfoxide 19

Compound 19e (66 mg, 0.20 mmol), ((4-(4-amino-6-bromo-7-methyl-7H-pyrrolo [2,3-d]pyrimidin-5-yl)-2-fluorophenyl) imino) dimethyl-λ⁶-sulfone 19f (70 mg, 0.17 mmol) were dissolved in 1,4-dioxane (7 mL) and water (1.5 mL), tetrakis (triphenylphosphine) palladium (39.2 mg, 0.034 mmol), K₃PO₄ (108 mg, 0.51 mmol) were added, after replaced with nitrogen gas three times, heated up to 90° C. to react for 2 h. Then cooled down to room temperature, diluted with water (10 mL), extracted with dichloromethane (10 mL×3), and the organic phases were combined, dried with anhydrous sodium sulfate, concentrated, and the crude product was separated by high-performance liquid chromatography (preparation column: YMCC18, mobile phase A: 0.05% ammonia solution in H₂O, mobile phase B: acetonitrile, 30%~60% acetonitrile, 40 mL/min) to obtain compound 19 (13 mg), white solid.

MS (ESI) m/z 531 [M+H]⁺.

¹H NMR (400 MHz, DMSO-d₆) δ 8.20 (s, 1H), 7.29 (dd, J=8.0, 1.8 Hz, 1H), 7.12 (dd, J 5=15.3, 5.1 Hz, 2H), 7.04 (d, J=8.0 Hz, 1H), 6.96-6.86 (m, 2H), 6.00 (brs, 2H), 3.66 (s, 3H), 3.26 (s, 6H), 2.04 (s, 3H), 1.44 (s, 6H).

### Example 20 N-(4-(4-amino-5-(4-(dimethyl (oxo)-λ⁶-sulfanylidene) amino)-3-fluorophenyl)-7-methyl-7H-pyrrolo [2,3-d] pyrimidin-6-yl)-2-(2-hydroxypropan-2-yl) phenyl) but-2-amide

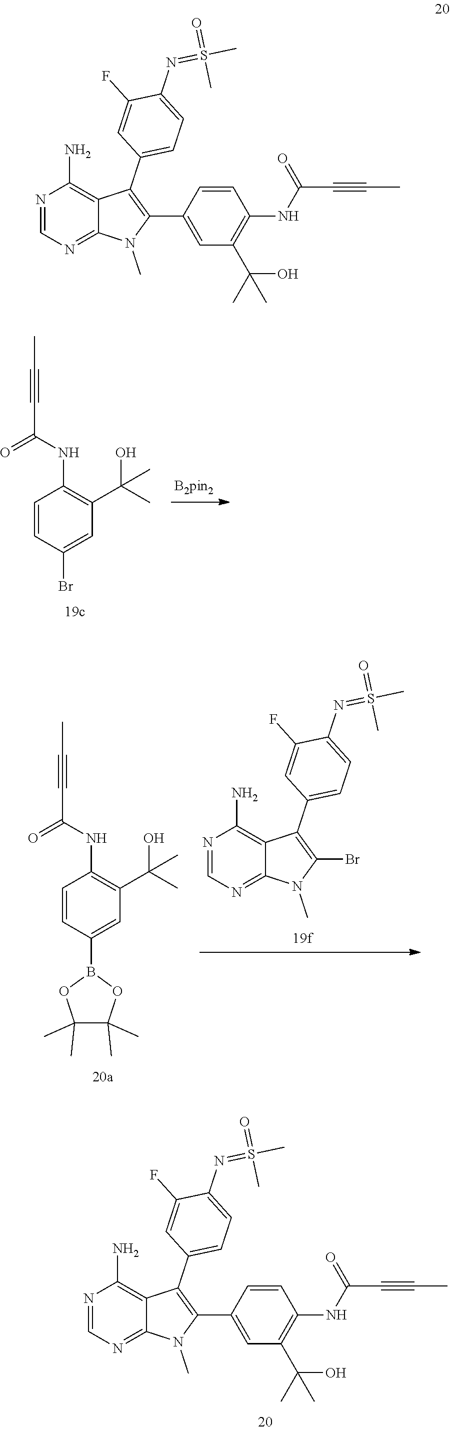

### Step 1 N-(2-(2-hydroxypropan-2-yl)-4-(4,4,5,5-tetramethyl-1,3,2-dioxaborolan-2-yl) phenyl) but-2-amide 20a

Compound 19c (270 mg, 0.97 mmol) was dissolved in dry 1,4-dioxane (10 mL), bis(pinacolato)diboron (370 mg, 1.46 mmol), Pd(dppf)Cl₂ (71 mg, 0.097 mmol), and potassium acetate (285 mg, 2.91 mmol) were added sequentially, after replaced with nitrogen three times, heated up to 90° C. under nitrogen protection, and reacted for 18 h. Then cooled to room temperature, filtered, concentrated, and the crude product was purified by column chromatography (PE/EtOAc=50/1) to obtain compound 20a (140 mg), yellow oil.

MS (ESI) m/z 343 [M+H]⁺.

### Step 2 N-(4-(4-amino-5-(4-(dimethyl (oxo)-λ⁶-sulfanylidene) amino)-3-fluorophenyl)-7-methyl-7H-pyrrolo [2,3-d] pyrimidin-6-yl)-2-(2-hydroxypropan-2-yl) phenyl) but-2-amide 20

Compound 20a (70 mg, 0.20 mmol), (4-(4-amino-6-bromo-7-methyl-7H-pyrrolo [2,3-d]pyrimidin-5-yl)-2-fluorophenyl) imino) dimethyl-λ⁶-sulfone 19f (66 mg, 0.16 mmol) were dissolved in 1,4-dioxane (7 mL) and water (1.5 mL), tetrakis (triphenylphosphine) palladium (39.2 mg, 0.034 mmol), K₃PO₄ (108 mg, 0.51 mmol) were added, after replaced with nitrogen gas three times, heated up to 90° C. and reacted for 2 h. Then cooled down to room temperature, diluted with water (10 mL), extracted with dichloromethane (10 mL×3), and the organic phases were combined, dried with anhydrous sodium sulfate, concentrated, and the crude product was separated by high-performance liquid chromatography (preparation column: YMCC18, mobile phase A: 0.05% ammonia solution in H₂O, mobile phase B: acetonitrile, 30%~60% acetonitrile, 40 mL/min) to obtain compound 20 (7.6 mg), white solid.

MS (ESI) m/z 549 [M+H]⁺.

¹H NMR (400 MHz, DMSO-d₆) δ 12.71 (s, 1H), 8.21 (s, 1H), 7.34 (d, J=8.2 Hz, 1H), 7.21-7.10 (m, 3H), 6.92 (dd, J=16.5, 11.0 Hz, 2H), 6.07 (brs, 2H), 5.02 (s, 1H), 3.66 (s, 3H), 3.26 (s, 6H), 2.00 (s, 3H), 1.49 (s, 6H).

### Example 21 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl) phenyl)-2-fluoroacrylamide

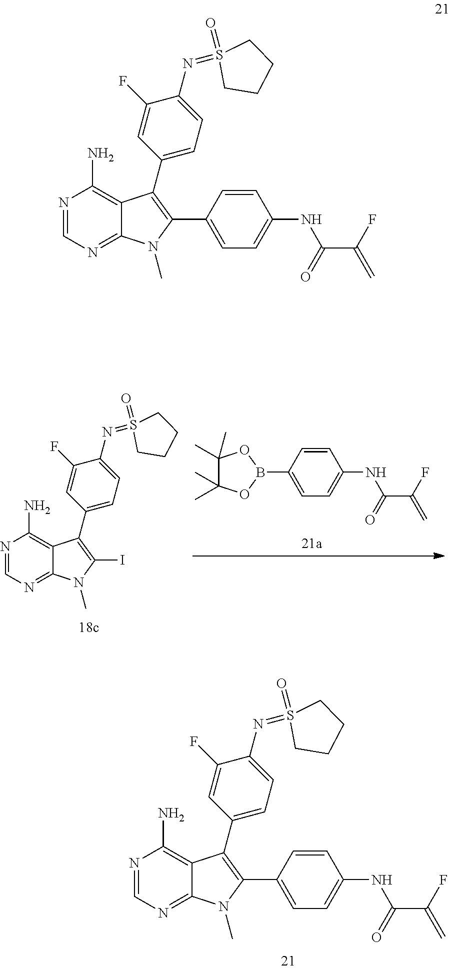

Compound 18c (100 mg, 0.20 mmol) and compound 21a (71 mg, 0.24 mmol) were dissolved in N,N-dimethylacetamide (4 mL) and water (1 mL), chlorine [(n-butyl di (1-adamantanyl) phosphine)-2-(2-aminobiphenyl)] palladium (II) (13.8 mg, 0.02 mmol) and K₃PO₄ (87.4 mg, 0.4 mmol) were added, after replaced with nitrogen gas three times, heated up to 80° C. and reacted for 2 h. Then cooled down to room temperature, diluted with water (20 mL), extracted with dichloromethane (20 mL×3), and the organic phases were combined, dried with anhydrous sodium sulfate, concentrated, and the crude product were separated by high-performance liquid chromatography (preparation column: YMCC18, mobile phase A: 0.05% ammonia solution in H₂O, mobile phase B: acetonitrile, 30%~60% acetonitrile, 40 mL/min) to obtain compound 21 (23.6 mg), white solid.

MS (ESI) m/z 523 [M+H]⁺.

¹H NMR (400 MHz, DMSO-d₆) δ 10.40 (s, 1H), 8.19 (s, 1H), 7.77 (d, J=8.0 Hz, 2H), 7.33 (d, J 8.1 Hz, 2H), 7.26-7.16 (m, 1H), 7.05 (t, J=8.6 Hz, 1H), 6.93 (d, J=8.5 Hz, 1H), 5.93 (brs, 2H), 5.80 (s, 0.5H), 5.68 (s, 0.5H), 5.46 (d, J=14.6 Hz, 1H), 3.60 (s, 3H), 3.29-3.12 (m, 4H), 2.27-2.08 (m, 4H).

### Example 22 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl)-3-methylphenyl) methacrylamide

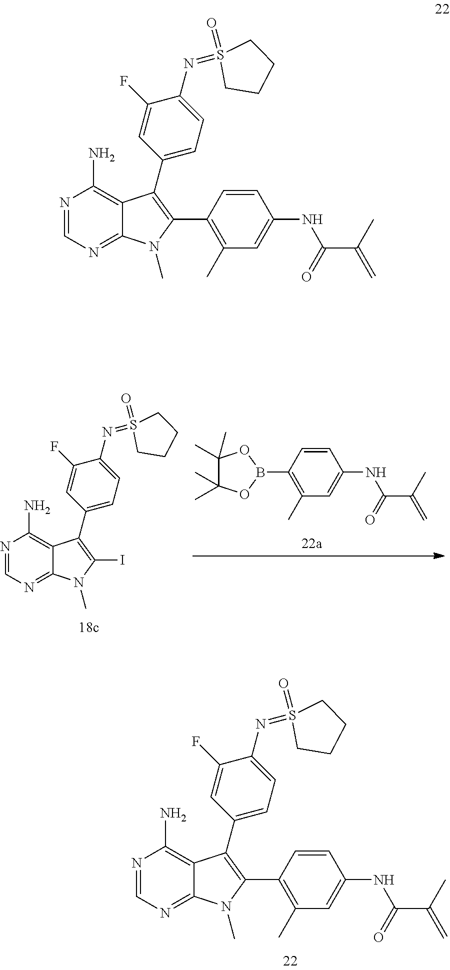

Compound 18c (100 mg, 0.20 mmol) and compound 22a (74 mg, 0.24 mmol) were dissolved in N,N-dimethylacetamide (4 mL) and water (1 mL), chlorine [(n-butyl di (1-adamantanyl) phosphine)-2-(2-aminobiphenyl)] palladium (II) (13.8 mg, 0.02 mmol) and K₃PO₄ (87.4 mg, 0.4 mmol) were added, after replaced with nitrogen three times, heated up to 80° C. to react for 2 h. Then cooled down to room temperature, diluted with water (20 mL), extracted with dichloromethane (20 mL×3), and the organic phases were combined, dried with anhydrous sodium sulfate, concentrated, and the crude product was separated by high-performance liquid chromatography (preparation column: YMCC18, mobile phase A: 0.05% ammonia solution in H₂O, mobile phase B: acetonitrile, 30%~60% acetonitrile, 40 mL/min) to obtain compound 22 (30.9 mg), white solid.

MS (ESI) m/z 533 [M+H]⁺.

¹H NMR (400 MHz, DMSO-d₆) δ 9.83 (s, 1H), 8.19 (s, 1H), 7.67-7.58 (m, 2H), 7.27 (m, 2H), 7.01 (m, 1H), 6.87 (m, 1H), 5.99 (brs, 2H), 5.82 (s, 1H), 5.53 (s, 1H), 3.41 (s, 3H), 3.28 (m, 4H), 2.27-2.08 (m, 4H), 1.96 (s, 3H), 1.93 (s, 3H).

### Example 23 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl)-3-fluorophenyl) methacrylamide

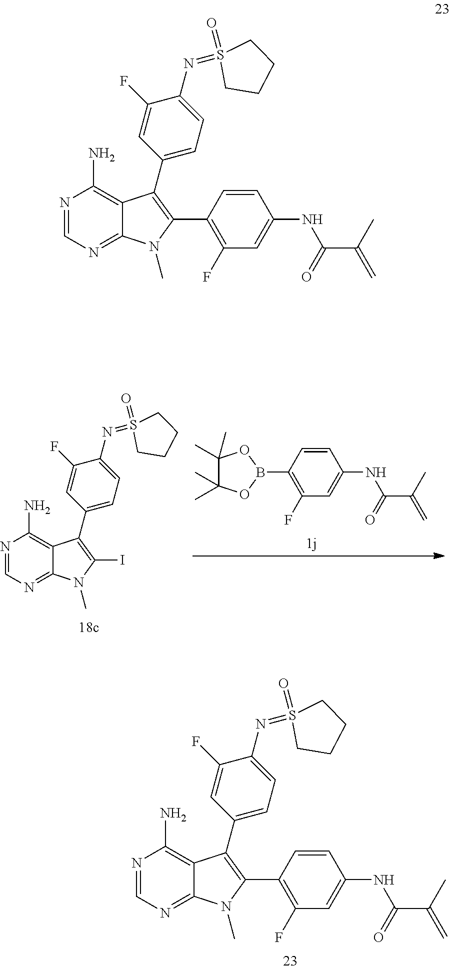

Compound 18c (100 mg, 0.20 mmol) and compound 1j (70 mg, 0.23 mmol) were dissolved in N,N-dimethylacetamide (4 mL) and water (1 mL), chlorine [(n-butyl di (1-adamantanyl) phosphine)-2-(2-aminobiphenyl)] palladium (II) (13.8 mg, 0.02 mmol), K₃PO₄ (87.4 mg, 0.4 mmol) were added, after replaced with nitrogen three times, heated up to 80° C. to react for 2 h. Then cooled to room temperature, diluted with water (20 mL), extracted with dichloromethane (20 mL×3), and the organic phases were combined, dried with anhydrous sodium sulfate, concentrated, and the crude product was separated by high-performance liquid chromatography (preparation column: YMCC18, mobile phase A: 0.05% ammonia solution in H₂O, mobile phase B: acetonitrile, 30%-60% acetonitrile, 40 mL/min) to obtain compound 23 (17.5 mg), white solid.

MS (ESI) m/z 537 [M+H]⁺.

¹H NMR (400 MHz, DMSO-d₆) δ 10.12 (s, 1H), 8.21 (s, 1H), 7.78 (d, J=12.4 Hz, 1H), 7.52 (d, J=8.0 Hz, 1H), 7.31 (t, J=8.0 Hz, 1H), 7.05 (t, J=8.4 Hz, 1H), 6.92 (d, J=10.7 Hz, 2H), 6.04 (brs, 2H), 5.84 (s, 1H), 5.59 (s, 1H), 3.54 (s, 3H), 3.31 (m, 4H), 2.18 (dd, J=33.7, 6.6 Hz, 4H), 1.97 (s, 3H).

### Example 24 N-(4-(4-amino-7-methyl-5-(4-(1-oxotetrahydro-1λ⁶-thiophene-1-ylidene) amino) phenyl)-7H-pyrrolo [2,3-d] pyrimidin-6-yl)-3-fluorophenyl) methacrylamide

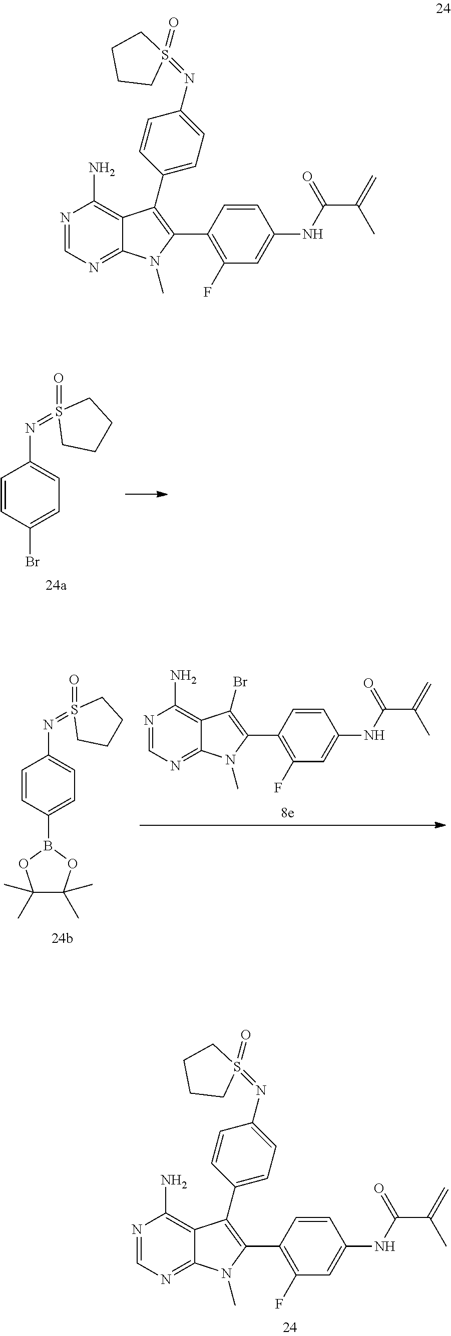

### Step 1 1-(4-(4,4,5,5-tetramethyl-1,3,2-dioxaborolan-2-yl) phenyl) imino) tetrahydro-1H-1λ⁶-thiophene 1-oxide 24b

To a 100 mL round bottom flask, compound 24h (1.02 g, 3.10 mmol), bis(pinacolato)diboron (0.973 g, 3.83 mmol), Pd(dppf)Cl₂ (0.461 mg, 0.63 mmol), potassium acetate (0.943 g, 9.61 mmol), 1,4-dioxane (20 mL) were added sequentially, then heated up to 100° C. to react for 4 h, after TLC monitored the completion of the reaction, it was cooled down to room temperature, filtered through diatomaceous earth, extracted with ethyl acetate (50 mL×3), and the organic phases were combined, washed with saturated saline (50 mL), and concentrated under reduced pressure to obtain a crude product. The crude product was purified by column chromatography (PE/EtOAc=1/1) to obtain compound 24b (0.80 g), yellow solid.

MS (ESI) m/z 322.2 [M+H]⁺.

¹H NMR (400 MHz, CDCl₃) δ 7.62 (d, J=8.3 Hz, 2H), 6.98 (d, J=8.3 Hz, 2H), 3.37-3.30 (m, 1H), 3.15-3.08 (m, 1H), 2.20 (dd, J=29.3, 6.9 Hz, 2H), 1.26 (s, 6H).

### Step 2 N-(4-(4-amino-7-methyl-5-(4-(1-oxotetrahydro-1λ⁶-thiophene-1-ylidene) amino) phenyl)-7H-pyrrolo [2,3-d] pyrimidin-6-yl)-3-fluorophenyl) methacrylamide 24

To a 25 mL round bottom flask, compound 4b (45 mg, 0.14 mmol), compound 8e (50 mg, 0.12 mmol), and Pd(dppf)Cl₂ (20 mg, 0.03 mmol), potassium phosphate (80 mg, 0.38 mmol), 1,4-dioxane (5 mL), and water (1 mL) were added in sequence, then heated up to 80° C. to react for 0.5 h, after TLC monitored the completion of the reaction, it was cooled down to room temperature, filtered through diatomaceous earth, extracted with ethyl acetate (20 mL×3), and the organic phases were combined, washed with saturated saline (20 mL), and concentrated under reduced pressure to obtain a crude product. The crude product was purified by column chromatography (DCM/MeOH=6/1) to obtain compound 24 (22 mg), white solid.

MS (ESI) m/z 519.41 [M+H]⁺.

¹H NMR (400 MHz, CDCl₃) δ 8.28 (s, 1H), 7.65-7.61 (m, 1H), 7.48 (s, 1H), 7.05 (d, J=7.7 Hz, 2H), 7.00 (t, J=4.1 Hz, 2H), 6.91 (d, J=8.5 Hz, 2H), 5.73 (s, 1H), 5.45 (d, J=1.3 Hz, 1H), 4.98 (s, 2H), 3.59 (d, J=0.9 Hz, 3H), 3.36 (dd, J=12.6, 6.0 Hz, 2H), 3.13 (dd, J=12.8, 6.6 Hz, 2H), 2.29-2.19 (m, 3H), 1.99 (d, J=10.1 Hz, 3H).

### Example 25 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl)-3-cyanophenyl) methacrylamide

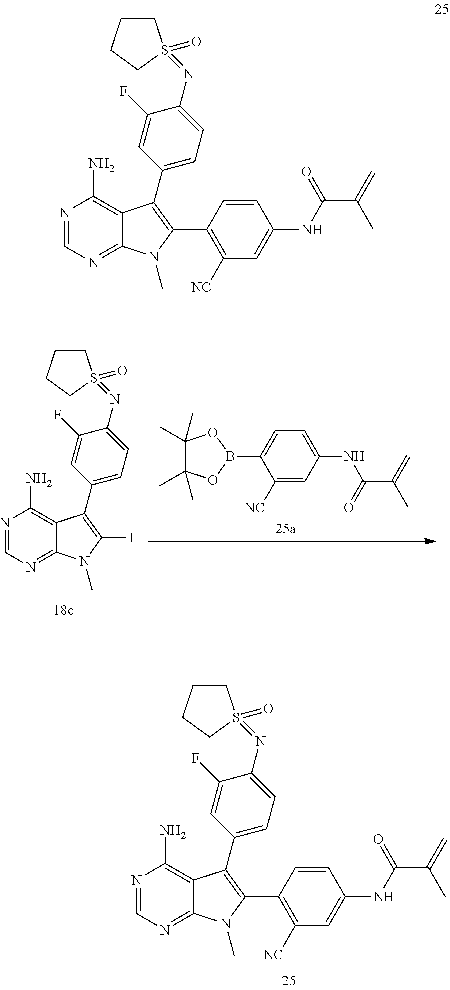

To a 25 mL round bottom flask, compound 18c (80 mg, 0.16 mmol), compound 25a (57 mg, 0.18 mmol), Pd(dppf)Cl₂ (12 mg, 0.016 mmol), potassium phosphate (102 mg, 0.48 mmol), 1,4-dioxane (10 mL), and water (1 mL) were added in sequence, then heated up to 80° C. to react for 1 h. After TLC monitored the completion of the reaction, it was cooled down to room temperature, filtered through diatomaceous earth, extracted with ethyl acetate (30 mL×3), and the organic phases were combined, washed with saturated saline (30 mL), and concentrated under reduced pressure to obtain a crude product. The crude product was purified by column chromatography (DCM/MeOH=6/1) to obtain compound 25 (4 mg), white solid.

MS (ESI) m/z 544.73 [M+H]⁺

¹H NMR (400 MHz, CDCl₃) δ 8.28 (s, 1H), 7.65-7.61 (m, 1H), 7.48 (s, 1H), 7.05 (d, J=7.7 Hz, 2H), 7.00 (t, J=4.1 Hz, 2H), 6.91 (d, J=8.5 Hz, 2H), 5.73 (s, 1H), 5.45 (d, J=1.3 Hz, 1H), 4.98 (s, 2H), 3.59 (d, J=0.9 Hz, 3H), 3.36 (dd, J=12.6, 6.0 Hz, 2H), 3.13 (dd, J=12.8, 6.6 Hz, 2H), 2.29-2.19 (m, 3H), 1.99 (d, J=10.1 Hz, 3H).

### Example 26 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl)-3-cyanophenyl) methacrylamide

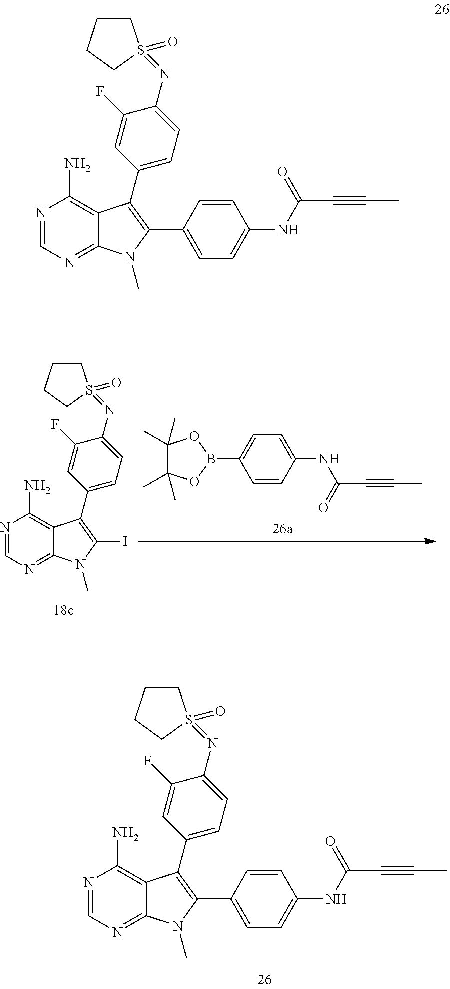

To a 25 mL round bottom flask, compound 18c (120 mg, 0.25 mmol), compound 26a (80 mg, 0.28 mmol), tetrakis (triphenylphosphine) palladium (29 mg, 0.025 mmol), anhydrous potassium phosphate (159 mg, 0.75 mmol), 1,4-dioxane (10 mL), and water (1 mL) were added in sequence, then heated up to 90° C. to react for 1 h, after the reaction was completed, it was cooled down to room temperature, filtered through diatomaceous earth, extracted with ethyl acetate (30 mL×3), the organic phases were combined, washed with saturated saline (30 mL), and concentrated under reduced pressure to obtain a crude product. The crude product was purified by column chromatography (DCM/MeOH=6/1) to obtain compound 26 (13.8 mg), white solid.

MS (ESI) m/z 517.97 [M+H]⁺

¹H NMR (400 MHz, CDCl₃) δ 8.28 (s, 1H), 7.43 (s, 2H), 7.34 (s, 1H), 7.14 (d, J=8.5 Hz, 2H), 7.04 (d, J=7.4 Hz, 1H), 6.84 (dd, J=8.7, 6.8 Hz, 2H), 4.95 (s, 2H), 3.62 (s, 1H), 3.36 (dd, J=12.9, 6.8 Hz, 2H), 3.21-3.14 (m, 2H), 2.25 (d, J=7.6 Hz, 4H), 1.96 (d, J=3.9 Hz, 3H).

### Example 27 N-(4-(4-amino-5-(3,5-difluoro-4-((1-oxotetrahydro-1λ⁶-thiophene-1-ylidene) amino) phenyl)-7-methyl-7H-pyrrolo [2,3-d] pyrimidin-6-yl) phenyl) methacrylamide

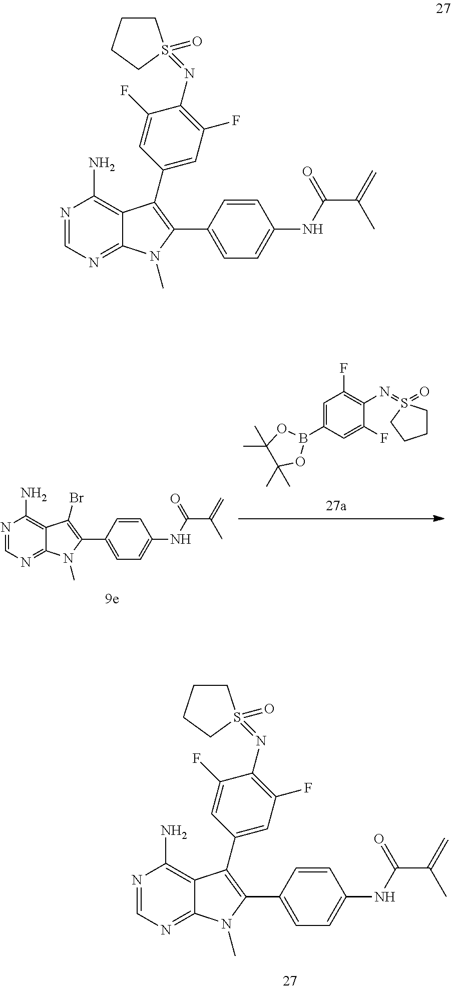

To a 25 mL round bottom flask, compound 9e (78 mg, 0.20 mmol), compound 27a (80 mg, 0.22 mmol), tetrakis (triphenylphosphine) palladium (25 mg, 0.02 mmol), anhydrous potassium phosphate (127 mg, 0.60 mmol), 1,4-dioxane (10 mL), and water (1 mL) were added in sequence, then heated up to 90° C. to react for 1 h. After TLC monitored the completion of the reaction, it was cooled down to room temperature, filtered through diatomaceous earth, extracted with ethyl acetate (30 mL×3), and the organic phases were combined, washed with saturated saline (30 mL), and concentrated under reduced pressure to obtain a crude product. The crude product was purified by column chromatography (DCM/MeOH=6/1) to obtain compound 27 (18 mg), white solid.

MS (ESI) m/z 537.63 [M+H]⁺.

¹H NMR (400 MHz, CDCl₃) δ 8.38 (s, 1H), 7.62 (d, J=8.5 Hz, 3H), 7.24 (d, J=8.5 Hz, 2H), 6.79 (d, J=8.7 Hz, 2H), 5.84 (s, 1H), 5.53 (s, 1H), 5.21 (s, 2H), 3.72 (s, 3H), 3.52-3.44 (m, 2H), 3.31 (dd, J=12.8, 7.1 Hz, 2H), 2.35 (d, J=2.1 Hz, 4H), 2.11 (s, 3H).

### Example 28 N-(4-(4-amino-5-(4-((dimethylamino) (oxo) (trifluoromethyl)-λ⁶-sulfanylidene) amino) phenyl)-7-methyl-7H-pyrrolo [2,3-d] pyrimidin-6-yl) phenyl) methacrylamide

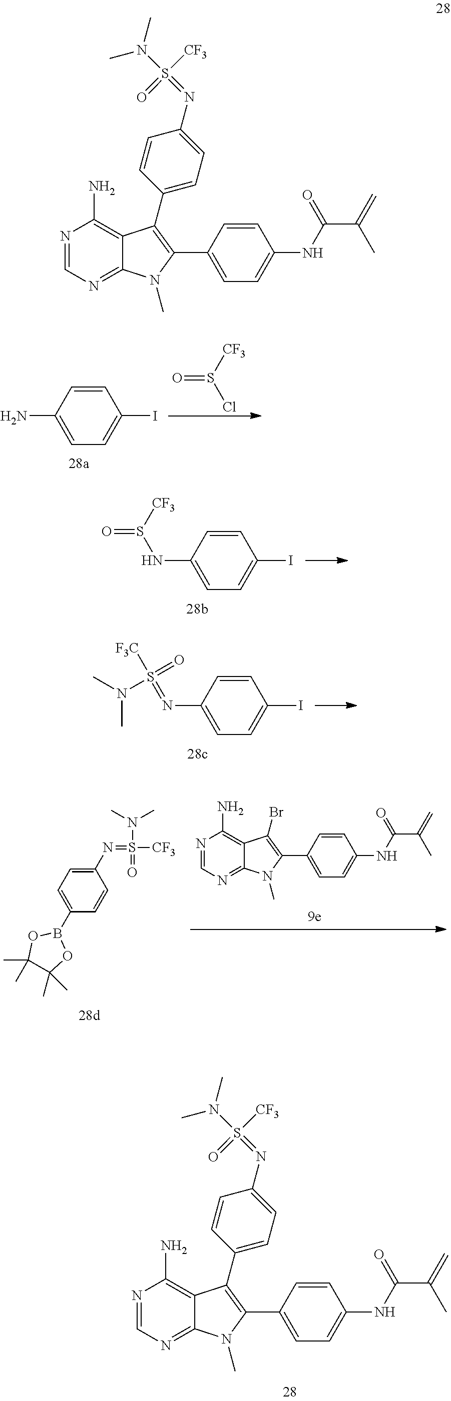

### Step 1 1,1,1-trifluoro-N-(4-iodophenyl) methylthioamide

Compound 28a (1.0 g, 4.6 mmol) was dissolved in DCM (10 mL), triethylamine (0.51 g, 5.0 mmol) was added, and it was cooled down to 0° C., trifluoromethanesulfonyl chloride (0.73 g, 4.8 mmol) was added dropwise, and warmed up to room temperature naturally to react for 1 h. The reaction solution was poured into water, extracted with ethyl acetate three times, dried with anhydrous sodium sulfate, and concentrated to obtain a crude product. The crude product was purified by column chromatography (PE/EtOAc=0-20%) to obtain compound 8b (1.25 g), yellow oily liquid.

MS (ESI) m/z 335 [M+H]⁺.

¹H NMR (400 MHz, CDCl₃) δ 7.69 (d, J=8.7 Hz, 2H), 6.97-6.81 (m, 2H), 6.46 (s, 1H).

### Step 2 1,1,1-trifluoro-N′-(4-iodophenyl)-N,N-dimethylmethanesulfonamide 28c

Compound 28a (1.4 g, 4.2 mmol) and N-chlorosuccinimide (0.62 g, 4.6 mmol) were dissolved in acetonitrile (30 mL), a solution of tetrabutylammonium fluoride in THE (4.6 mL, 4.6 mmol, 1M) was added at 0° C. to react at room temperature for 30 min, and the reaction solution was concentrated, then acetonitrile (20 mL) and a solution of dimethylamine in THE (8.35 mL, 16.7 mmol, 2M) were added, and reacted at room temperature for 2 h. The reaction solution was concentrated and dried, and the obtained crude product was purified by column chromatography (PE/EtOAc=0-20%) to obtain compound 28c (1.4 g), reddish brown solid.

MS (ESI) m/z 379 [M+H]⁺.

¹H NMR (400 MHz, CDCl₃) δ 7.57 (d, J=8.6 Hz, 2H), 6.87 (d, J=8.6 Hz, 2H), 3.08 (s, 6H).

### Step 3 1,1,1-trifluoro-N,N-dimethyl-N′-(4-(4,4,5,5-tetramethyl-1,3,2-dioxaborolan-2-yl) phenyl) methanesulfonamide 28d

28c (1.0 g, 2.6 mmol), bis(pinacolato)diboron (1.0 g, 4.0 mmol), Pd(dppf)Cl₂ (193 mg, 0.26 mmol), and potassium acetate (778 mg, 7.9 mmol) were placed in a bottle, 1,4-dioxane (15 mL) was added, after replaced with N₂ three times, the reaction was carried out at 100° C. for 18 h. The reaction solution was filtered and concentrated, and the obtained crude product was purified by column chromatography (PE/EtOAc=0-20%) to obtain compound 28d (0.65 g), white solid.

MS (ESI) m/z 379 [M+H]⁺.

### Step 4 N-(4-(4-amino-5-(4-((dimethylamino) (oxo) (trifluoromethyl)-6-sulfanylidene) amino) phenyl)-7-methyl-7H-pyrrolo [2,3-d] pyrimidin-6-yl) phenyl) methacrylamide 28

To a 25 mL round bottom flask, compound 9e (93 mg, 0.24 mmol), compound 28d (100 mg, 0.26 mmol), tetrakis (triphenylphosphine) palladium (30 mg, 0.024 mmol), anhydrous potassium phosphate (120 mg, 0.54 mmol), 1,4-dioxane (10 mL), and water (1 mL) were added in sequence, and heated up to 90° C. to react for 1 h. After TLC monitored the completion of the reaction, it was cooled down to room temperature, filtered through diatomaceous earth, extracted with ethyl acetate (30 mL×3), and the organic phases were combined, washed with saturated saline (30 mL), and concentrated under reduced pressure to obtain a crude product. The crude product was purified by column chromatography (DCM/MeOH=6/1) to obtain compound 28 (12.5 mg), white solid.

MS (ESI) m/z 558.53 [M+H]⁺.

¹H NMR (400 MHz, CDCl₃) δ 8.37 (s, 1H), 7.63-7.54 (m, 3H), 7.24 (d, J=8.5 Hz, 2H), 7.16 (d, J=8.3 Hz, 2H), 7.05 (d, J=8.3 Hz, 2H), 5.83 (s, 1H), 5.52 (s, 1H), 5.19 (s, 2H), 3.74 (s, 3H), 3.11 (s, 6H), 2.10 (s, 3H).

### Example 29 N-(4-(4-amino-7-methyl-5-(6-(1-oxotetrahydro-1λ⁶-thiophene-1-ylidene) amino) pyridin-3-yl)-7H-pyrrolo [2,3-d] pyrimidin-6-yl) phenyl) methacrylamide

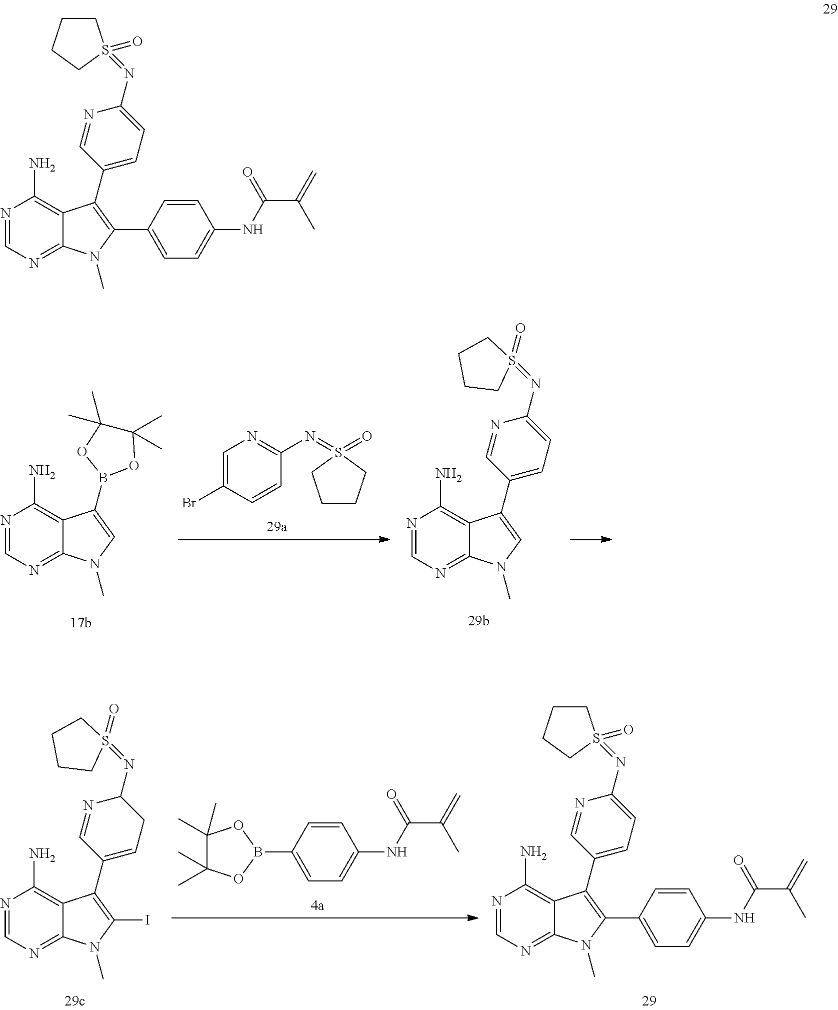

### Step 1 1-(5-(4-amino-7-methyl-7H-pyrrolo [2,3-d] pyrimidin-5-yl) pyridin-2-yl) imino) tetrahydro-1H-1λ⁶-thiophene-1-oxide

To a 100 mL round bottom flask, compound 7b (350 mg, 1.28 mmol), compound 9a (320 mg, 1.17 mmol), tetrakis (triphenylphosphine) palladium (135 mg, 0.012 mmol), anhydrous potassium phosphate (743 mg, 3.50 mmol), 1,4-dioxane (10 mL), and water (1 mL) were added in sequence, and heated up to 90° C. to react for 1 h. After TLC monitored the completion of the reaction, it was cooled down to room temperature, filtered through diatomaceous earth, extracted with ethyl acetate (50 mL×3), the organic phases were combined, washed with saturated saline (50 mL), and concentrated under reduced pressure to obtain a crude product. The crude product was purified by column chromatography (DCM/MeOH=6/1) to obtain compound 29b (110 mg), white solid.

MS (ESI) m/z 343 [M+H]⁺.

¹H NMR (400 MHz, CDCl₃) δ 8.38-8.33 (m, 2H), 7.65 (dd, J=8.3, 2.4 Hz, 1H), 6.95-6.89 (m, 2H), 5.15 (s, 2H), 3.87 (s, 3H), 3.70 (dd, J=13.3, 6.9 Hz, 2H), 3.44 (dd, J=13.3, 6.8 Hz, 2H), 2.35 (ddd, J=20.5, 12.3, 5.4 Hz, 4H).

### Step 2 1-(5-(4-amino-6-iodo-7-methyl-7H-pyrrolo [2,3-d] pyrimidin-5-yl) pyridin-2-yl) imino) tetrahydro-1H-1λ⁶-thiophene-1-oxide 29c

To a 100 mL round bottom flask, compound 29b (110 mg, 0.32 mmol), N-iodosuccinimide (87 mg, 0.38 mmol), trifluoroacetic acid (110 mg, 0.96 mmol), and dichloromethane (4 mL) were added in sequence, and reacted at room temperature for 2 h, after TLC monitored the completion of the reaction, it was cooled down to room temperature, filtered through diatomaceous earth, extracted with ethyl acetate (30 mL×3), the organic phases were combined, washed with saturated saline (30 mL), and concentrated under reduced pressure to obtain a crude product. The crude product was purified by column chromatography (DCM/MeOH=10/1) to obtain compound 29c (20 mg), white solid.

MS (ESI) m/z 471 [M+H]⁺.

### Step 3 N-(4-(4-amino-7-methyl-5-(6-(1-oxotetrahydro-1λ⁶-thiophene-1-ylidene) amino) pyridine-3-yl)-7H-pyrrolo [2,3-d] pyrimidin-6-yl) phenyl) methacrylamide 29

To a 25 mL round bottom flask, compound 29c (20 mg, 0.05 mmol), compound 4a (16 mg, 0.05 mmol), tetrakis (triphenylphosphine) palladium (6 mg, 0.005 mmol), anhydrous potassium phosphate (32 mg, 0.15 mmol), 1,4-dioxane (2 mL), and distilled water (0.5 mL) were added in sequence, and heated up to 90° C. to react for 1 h. After TLC monitored the completion of the reaction, it was cooled down to room temperature, filtered through diatomaceous earth, extracted with ethyl acetate (20 mL×3), the organic phases were combined, washed with saturated saline (20 mL), and concentrated under reduced pressure to obtain a crude product. The crude product was purified by column chromatography (DCM/MeOH=6/1) to obtain compound 29 (7 mg), white solid.

MS (ESI) m/z 587.10 [M+H]⁺.

¹H NMR (400 MHz, CDCl₃) δ 8.37 (s, 1H), 8.14 (d, 1.8 Hz, 1H), 7.68-7.53 (m, 3H), 7.41 (dd, J=8.4, 2.4 Hz, 1H), 7.26 (d, J=8.6 Hz, 2H), 6.78 (d, J=8.1 Hz, 1H), 5.83 (s, 1H), 5.52 (s, 1H), 5.33 (s, 2H), 3.74 (s, 3H), 3.65 (dd, J=13.5, 6.8 Hz, 2H), 3.40 (dd, J=13.2, 6.7 Hz, 2H), 2.43-2.25 (m, 4H), 2.10 (s, 3H).

### Example 30 N-(4-(4-amino-5-(3-fluoro-4-((4-methyl-1-oxo-1λ⁶-thiomorpholin-1-ylidene) amino) phenyl)-7-methyl-7H-pyrrolo [2,3-d] pyrimidin-6-yl) phenyl)

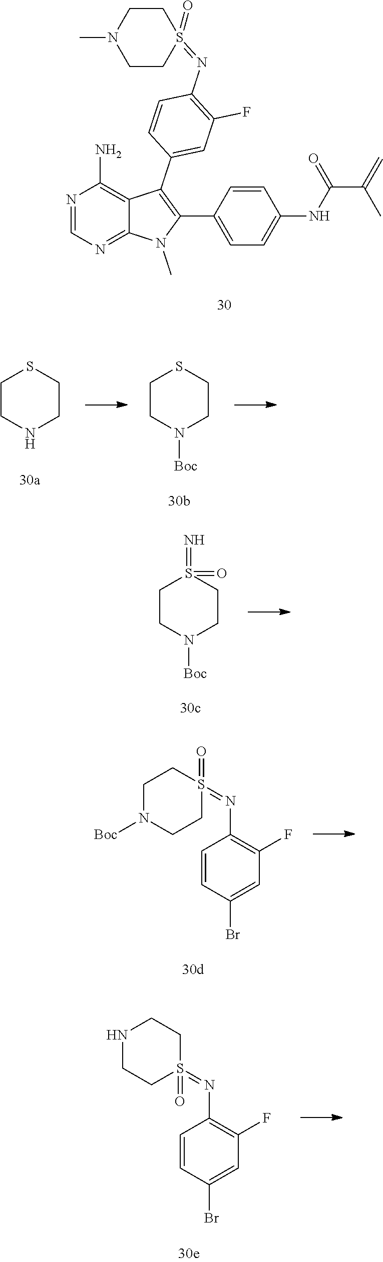 [SMILES: C=C(C)C(=O)Nc1ccc(-c2c(-c3ccc(N=S4(=O)CCN(C)CC4)c(F)c3)c3c(N)ncnc3n2C)cc1 ; C1CSCCN1 ; CC(C)(C)OC(=O)N1CCSCC1 ; CC(C)(C)OC(=O)N1CCS(=N)(=O)CC1 ; CC(C)(C)OC(=O)N1CCS(=O)(=Nc2ccc(Br)cc2F)CC1 ; O=S1(=Nc2ccc(Br)cc2F)CCNCC1]

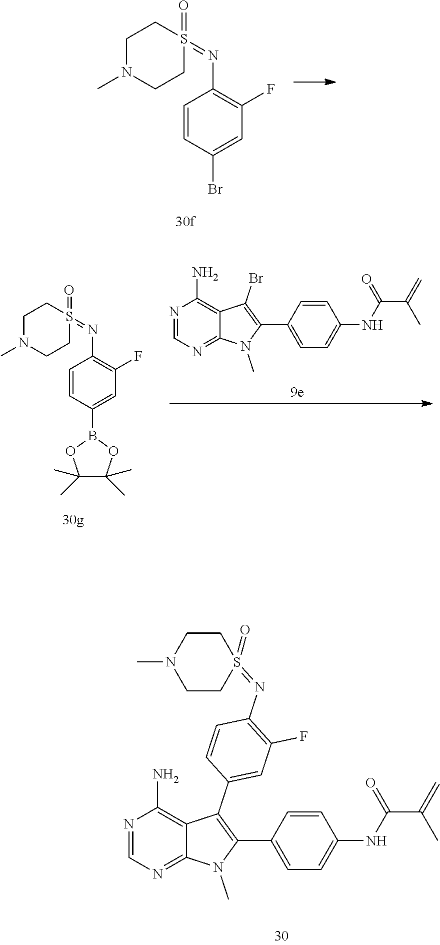

### Step 1 thiomorpholine-4-carboxylic acid tert-butyl ester 30b

To a 250 mL round bottom flask, compound 30a (3 g, 29.07 mmol), Boc anhydride (6 g, 29.07 mmol), DMAP (350 mg, 2.91 mmol), triethylamine (7 mL, 58.14 mmol), and anhydrous dichloromethane (30 mL) were added in sequence, and reacted at room temperature for 5 h. After TLC monitored the completion of the reaction, it was extracted with dichloromethane (50 mL×3), and the organic phases were combined, washed with saturated saline solution (50 mL), and concentrated under reduced pressure to obtain a crude product. The crude product was purified by column chromatography (PE/EA=8/1) to obtain compound 30b (3.5 mg), white solid.

### Step 2 1-imino-1λ⁶-thiomorpholine-4-carboxylic acid tert-butyl ester 1-oxide 30c

To a 100 mL round bottom flask, compound 30b (1.00 g, 4.92 mmol), ammonium carbamate (577 mg, 7.38 mmol), iodobenzene acetate (3.33 g, 10.3 mmol), and methanol (40 mL) were added in sequence, and reacted at room temperature for 1 h, after TLC monitored the completion of the reaction, it was concentrated under reduced pressure to obtain a crude product. The crude product was purified by column chromatography (PE/EA=1/2) to obtain compound 30c (930 mg), white solid.

MS (ESI) m/z 235 [M+H]⁺.

### Step 3 1-(4-bromo-2-fluorophenyl) imino)-1λ⁶-thiomorpholine-4-carboxylic acid tert-butyl ester 1-oxide 30d

To a 100 mL round bottom flask, compound 30c (900 mg, 3.84 mmol), 4-bromo-2-fluoro-1-iodobenzene (930 mg, 3.2 mmol), Pd₂(dba)₃ (74 mg, 0.08 mmol), Xantphos (140 mg, 0.24 mmol), and cesium carbonate (1.5 g, 4.60 mmol) were added in sequence, and reacted at 90° C. for 5 h, after TLC monitored the completion of the reaction, it was cooled down to room temperature, filtered through diatomaceous earth, extracted with ethyl acetate (30 mL×3), and the organic phases were combined, washed with saturated saline solution (30 mL), and concentrated under reduced pressure to obtain a crude product. The crude product was purified by column chromatography (PE/EA=1/1) to obtain compound 30d (800 mg), light yellow solid.

MS (ESI) m/z 407 [M+H]⁺.

### Step 4 1-((4-bromo-2-fluorophenyl) imino)-1λ⁶-thiomorpholine 1-oxide 30e

To a 100 mL round bottom flask, compound 30d (400 mg, 1.30 mmol), trifluoroacetic acid (2 mL), and anhydrous dichloromethane (8 mL) were added in sequence, and reacted at room temperature for 1 h, after TLC monitored the completion of the reaction, it was extracted with dichloromethane (30 mL×3), and the organic phases were combined, washed with saturated saline solution (30 mL), concentrated under reduced pressure to obtain a crude product, dried to obtain compound 30e (350 mg), light yellow solid, which was directly used in the next step.

MS (ESI) m/z 307 [M+H]⁺.

### Step 5 1-((4-bromo-2-fluorophenyl) imino)-4-methyl-1λ⁶-thiomorpholine 1-oxide 30f

To a 100 mL round bottom flask, compound 30e (350 mg, 1.15 mmol), polyformaldehyde (71 mg, 2.30 mmol), sodium cyanoborohydride (216 mg, 3.45 mmol) and methanol (10 mL) were added in sequence. After 6 h of reaction at room temperature, the reaction was quenched with sodium bicarbonate and extracted with ethyl acetate (30 mL×3), and the organic phases were combined and washed with saturated saline solution (30 mL). The crude product was concentrated under reduced pressure and purified by column chromatography (PE/EA=1/1) to obtain compound 30f (170 mg), white solid.

MS (ESI) m/z 321 [M+H]⁺.

¹H NMR (400 MHz, CDCl₃) δ 7.25-7.21 (m, 1H), 7.17-7.11 (m, 1H), 3.31 (tdd, J=13.3, 9.2, 4.8 Hz, 1H), 3.02-2.92 (m, 1H), 2.44 (s, 3H).

### Step 6 1-((2-Fluoro-4-(4,4,5,5-tetramethyl-1,3,2-dioxaborolan-2-yl) phenyl) imino)-4-methyl-1λ⁶-thiomorpholine 1-oxide 30g

To a 100 mL round bottom flask, compound 30f (170 mg, 0.53 mmol), bis(pinacolato)diboron (162 mg, 0.64 mmol), Pd(dppf)Cl₂ (77 mg, 0.11 mmol), potassium acetate (157 mg, 1.59 mmol) and 1,4-dioxane (10 mL) were added in sequence, and heated up to 90° C. to react for 3 h. After the reaction was completed, it was cooled down to room temperature, filtered through diatomaceous earth, extracted with ethyl acetate (30 mL×3), and the organic phases were combined, washed with saturated saline (30 mL), and concentrated under reduced pressure to obtain a crude product. The crude product was purified by column chromatography (PE/EA=1/1) to obtain compound 30g (120 mg), yellow oil.

MS (ESI) m/z 369 [M+H]⁺.

### Step 7 N-(4-(4-amino-5-(3-fluoro-4-((4-methyl-1-oxo-1λ⁶-thiomorpholine-1-ylidene) amino) phenyl)-7-methyl-7H-pyrrolo [2,3-d] pyrimidin-6-yl) phenyl) methacrylamide 30

To a 25 mL round bottom flask, compound 30g (126 mg, 0.34 mmol), compound 9e (120 mg, 0.31 mmol), tetrakis (triphenylphosphine) palladium (36 mg, 0.03 mmol), anhydrous potassium phosphate (477 mg, 2.25 mmol), 1,4-dioxane (10 mL), and water (1 mL) were added in sequence, and heated up to 90° C. to react for 1 h. After TLC monitored the completion of the reaction, it was cooled down to room temperature, filtered through diatomaceous earth, extracted with ethyl acetate (20 mL×3), and the organic phases were combined, washed with saturated saline (20 mL), and concentrated under reduced pressure to obtain a crude product. The crude product was purified by column chromatography (DCM/MeOH=6/1) to obtain compound 30 (38 mg), white solid.

MS (ESI) m/z 548.20 [M+H]⁺.

¹H NMR (400 MHz, CDCl₃) δ 8.37 (s, 1H), 7.62-7.55 (m, 3H), 7.23 (dd, J=14.9, 8.4 Hz, 3H), 6.94 (dd, J=8.3, 4.6 Hz, 2H), 5.83 (s, 1H), 5.52 (s, 1H), 5.20 (s, 2H), 5.20 (s, 4H), 3.73 (s, 3H), 2.45 (s, 4H), 2.10 (s, 3H).

### Example 31 N-(4-(4-amino-7-methyl-5-(4-(1-oxotetrahydro-1λ⁶-thiophene-1-ylidene) amino) phenyl)-7H-pyrrolo [2,3-d] pyrimidin-6-yl) phenyl) methacrylamide

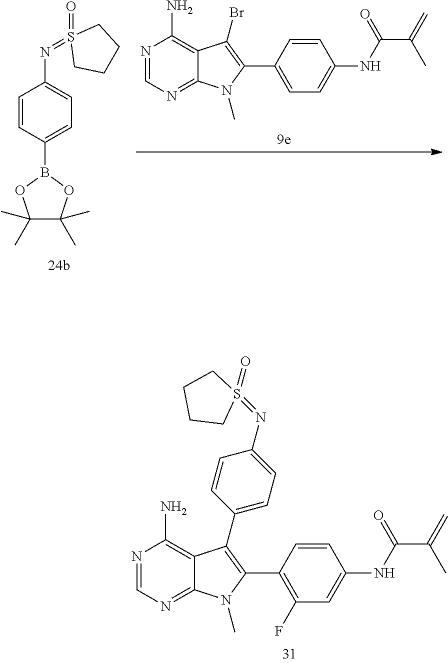

To a 25 mL round bottom flask, compound 9e (150 mg, 0.39 mmol), compound 4b (137 mg, 0.43 mmol), tetrakis (triphenylphosphine) palladium (50 mg, 0.039 mmol), anhydrous potassium phosphate (200 mg, 0.87 mmol), 1,4-dioxane (10 mL), and water (1 mL) were added in sequence, and heated up to 90° C. to react for 1 h. After TLC monitored the completion of the reaction, it was cooled down to room temperature, filtered through diatomaceous earth, extracted with ethyl acetate (30 mL×3), and the organic phases were combined, washed with saturated saline (30 mL), and concentrated under reduced pressure to obtain a crude product. The crude product was purified by column chromatography (DCM/MeOH=6/1) to obtain compound 31 (48.3 mg), white solid.

MS (ESI) m/z 501.66 [M+H]⁺.

¹H NMR (400 MHz, CDCl₃) δ 8.41-8.34 (m, 1H), 7.61-7.55 (m, 3H), 7.25 (d, J=8.6 Hz, 2H), 7.14 (d, J=8.3 Hz, 2H), 7.01 (d, J=8.3 Hz, 2H), 5.82 (s, 1H), 5.51 (s, 1H), 5.12 (s, 2H), 3.76-3.71 (m, 3H), 3.46 (dd, J=12.5, 6.3 Hz, 2H), 3.25-3.19 (m, 2H), 2.39-2.27 (m, 4H), 2.08 (d, J=10.1 Hz, 3H).

### Example 32 N-(4-(4-amino-5-(4-((dimethylamino) (oxo) (trifluoromethyl)-λ⁶-sulfanylidene)amino)phenyl)-7-methyl-7H-pyrrolo[2,3-d] pyrimidin-6-yl)-3-fluorophenyl) methacrylamide

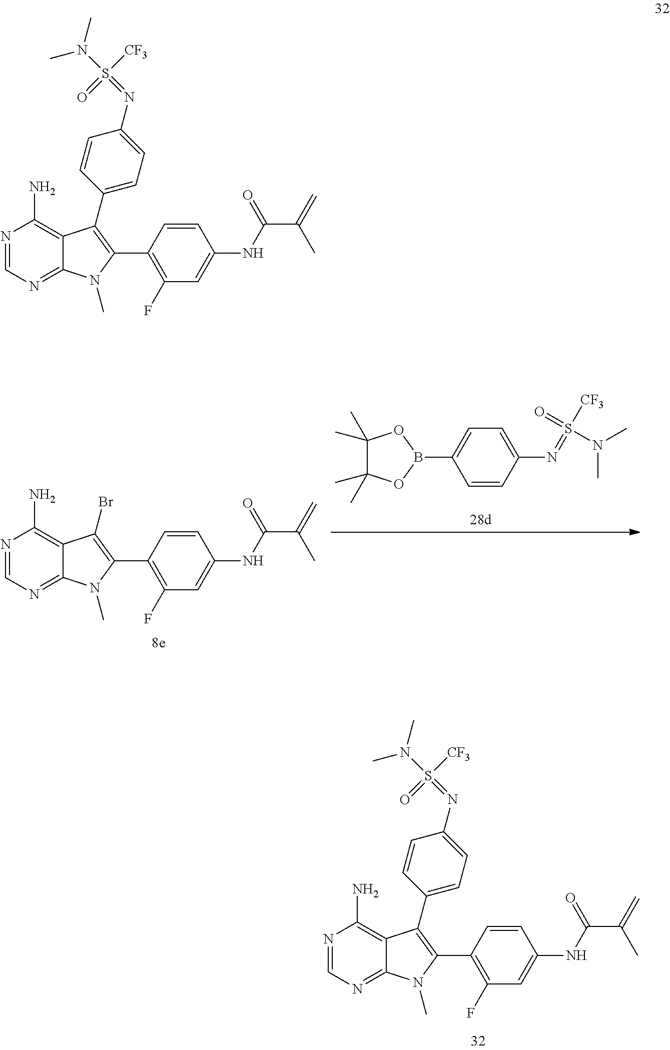

To a 25 mL round bottom flask, compound 8e (100 mg, 0.25 mmol), compound 28d (104 mg, 0.275 mmol), tetrakis (triphenylphosphine) palladium (29 mg, 0.025 mmol), anhydrous potassium phosphate (118 mg, 0.56 mmol), 1,4-dioxane (10 mL), and water (1 mL) were added in sequence, and heated up to 90° C. to react for 1 h. After TLC monitored the completion of the reaction, it was cooled down to room temperature, filtered through diatomaceous earth, extracted with ethyl acetate (30 mL×3), the organic phases were combined, washed with saturated saline (30 mL), and concentrated under reduced pressure to obtain a crude product. The crude product was purified by column chromatography (DCM/MeOH=6/1) to obtain compound 32 (6 mg), white solid.

MS (ESI) m/z 508.76 [M+H]⁺.

¹H NMR (400 MHz, CDCl₃) δ 8.40 (s, 1H), 7.79 (dd, J=25.0, 9.0 Hz, 2H), 7.19 (s, 2H), 7.07 (s, 4H), 5.84 (s, 1H), 5.56 (s, 1H), 5.15 (s, 2H), 3.70 (s, 3H), 3.12 (s, 7H), 2.11 (s, 4H).

### Example 33 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl) phenyl) methacrylamide

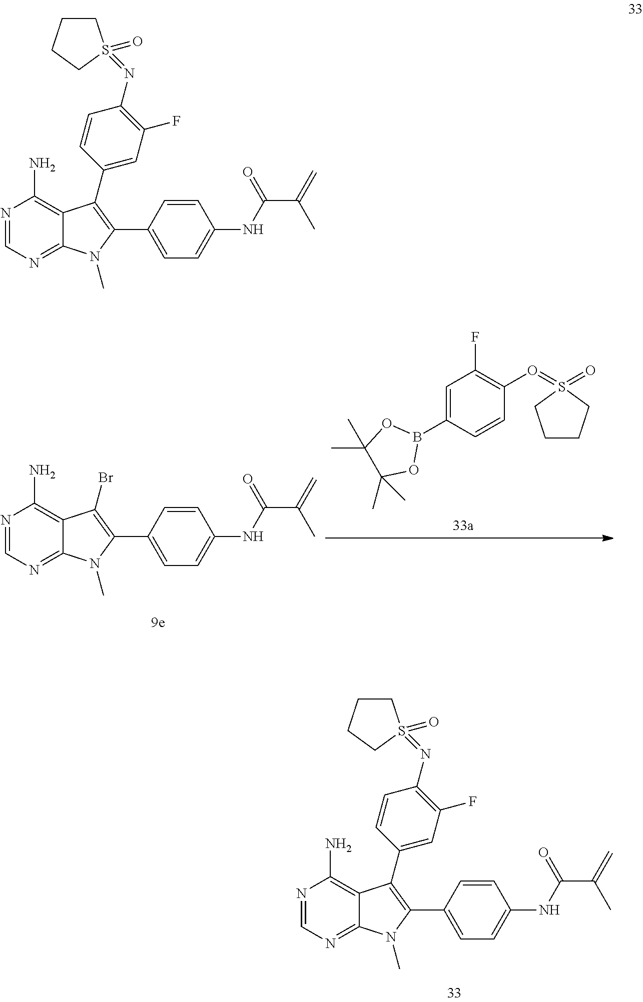

Compound 33 was synthesized with reference to Example 32.

MS (ESI) m/z 519.70 [M+H]⁺.

¹H NMR (400 MHz, CDCl₃) δ 8.28 (s, 1H), 7.51 (s, 1H), 7.49 (d, J=3.2 Hz, 2H), 7.16 (s, 1H), 7.14 (s, 1H), 7.04 (s, 1H), 6.87-6.82 (m, 2H), 5.74 (s, 1H), 5.43 (d, J=1.0 Hz, 1H), 5.08 (s, 2H), 3.63 (s, 3H), 3.41-3.34 (m, 2H), 3.17 (dd, J=13.0, 6.9 Hz, 2H), 2.25 (d, J=7.5 Hz, 4H), 2.01 (s, 3H).

### Example 34 N-(4-(4-amino-7-methyl-5-(3-methyl-4-((1-oxotetrahydro-2H-1λ⁶-thiopyran-1-ylidene) amino) phenyl)-7H-pyrrolo [2,3-d] pyrimidin-6-yl) phenyl) methacrylamide

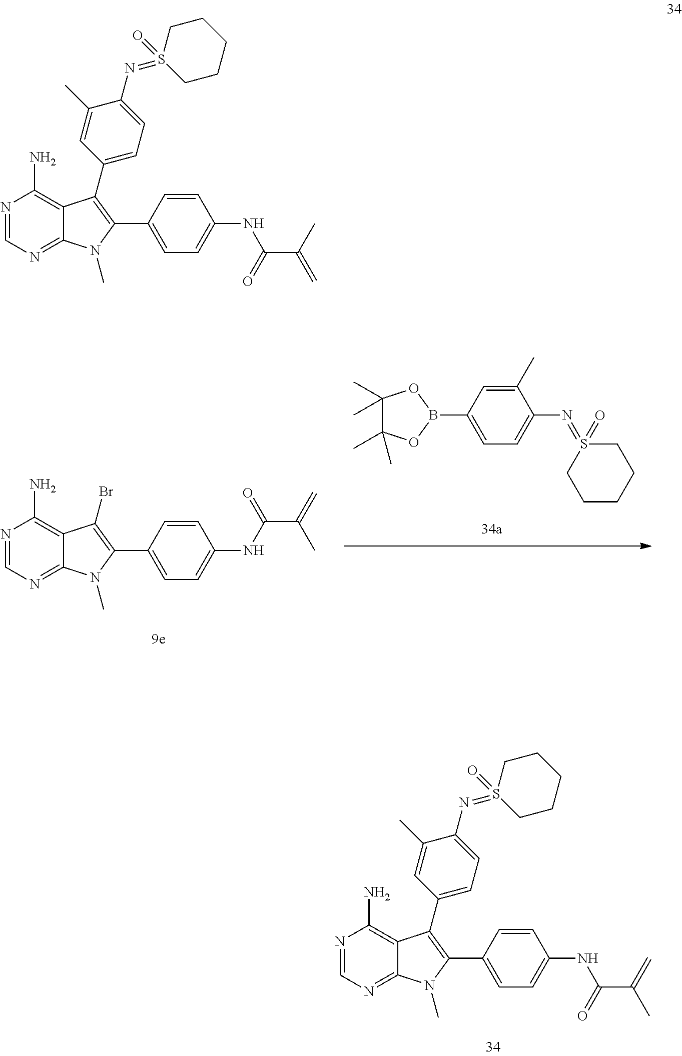 [SMILES: C=C(C)C(=O)Nc1ccc(-c2c(-c3ccc(N=S4(=O)CCCCC4)c(C)c3)c3c(N)ncnc3n2C)cc1 ; C=C(C)C(=O)Nc1ccc(-c2c(Br)c3c(N)ncnc3n2C)cc1 ; Cc1cc(B2OC(C)(C)C(C)(C)O2)ccc1N=S1(=O)CCCCC1 ; C=C(C)C(=O)Nc1ccc(-c2c(-c3ccc(N=S4(=O)CCCCC4)c(C)c3)c3c(N)ncnc3n2C)cc1]

Compound 34 was synthesized with reference to Example 32.

MS (ESI) m/z 529.08 [M+H]⁺.

¹H NMR (400 MHz, CDCl₃) δ 8.35 (s, 1H), 7.57 (d, J=8.6 Hz, 3H), 7.26 (d, J=8.5 Hz, 2H), 7.14 (d, J=8.1 Hz, 1H), 7.07 (s, 1H), 6.91 (d, J=8.0 Hz, 1H), 5.83 (s, 1H), 5.52 (s, 1H), 5.37 (s, 2H), 3.73 (s, 3H), 3.41 (d, J=13.1 Hz, 2H), 3.13 (dd, J=17.1, 10.5 Hz, 2H), 2.27 (d, J=7.5 Hz, 3H), 2.11 (d, J=5.5 Hz, 7H), 1.79 (d, J=5.2 Hz, 3H).

### Example 35 N-(4-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl) phenyl) but-2-ynamide

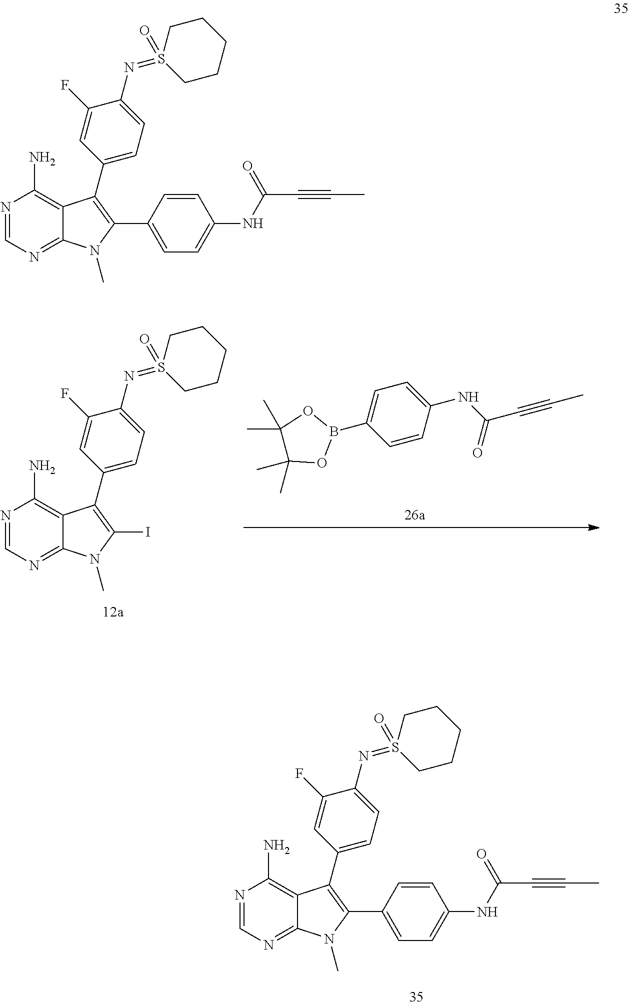

Compound 12a (0.10 g, 0.20 mmol) was dissolved in 1,4-dioxane (6 mL) and H₂O (2 mL), compound 26a (68.0 mg, 0.24 mmol), potassium phosphate (0.13 g, 0.60 mmol), and tetrakis (triphenylphosphine) palladium (23.0 mg, 0.02 mmol) were added, and reacted at 90° C. for 14 h under N₂ protection. After the reaction was completed, it was extracted with ethyl acetate (50 mL×3), and the organic phases were combined and concentrated under reduced pressure to obtain a crude product. The crude product was purified by preparative TLC plate (EA/THF=3/1) to obtain compound 35 (10.5 mg), white solid.

MS (ESI) m/z 531.20 [M+H]⁺.

¹H NMR (400 MHz, CDCl₃) δ 8.37 (s, 1H), 7.57-7.43 (m, 3H), 7.26-7.20 (m, 2H), 6.92 (dd, J=12.6, 5.0 Hz, 2H), 5.23 (s, 2H), 3.72 (s, 3H), 3.36 (d, J=14.3 Hz, 2H), 3.23-3.12 (m, 2H), 2.26-2.09 (m, 4H), 2.01 (s, 3H), 1.73 (m, 2H).

### Example 36 N-(4-(4-amino-5-(3-fluoro-5-((1-oxotetrahydro-2H-1λ⁶-thiopyran-1-ylidene) amino) pyridin-2-yl)-7-methyl-7H-pyrrole [2,3-d] pyrimidin-6-yl) phenyl) methacrylamide

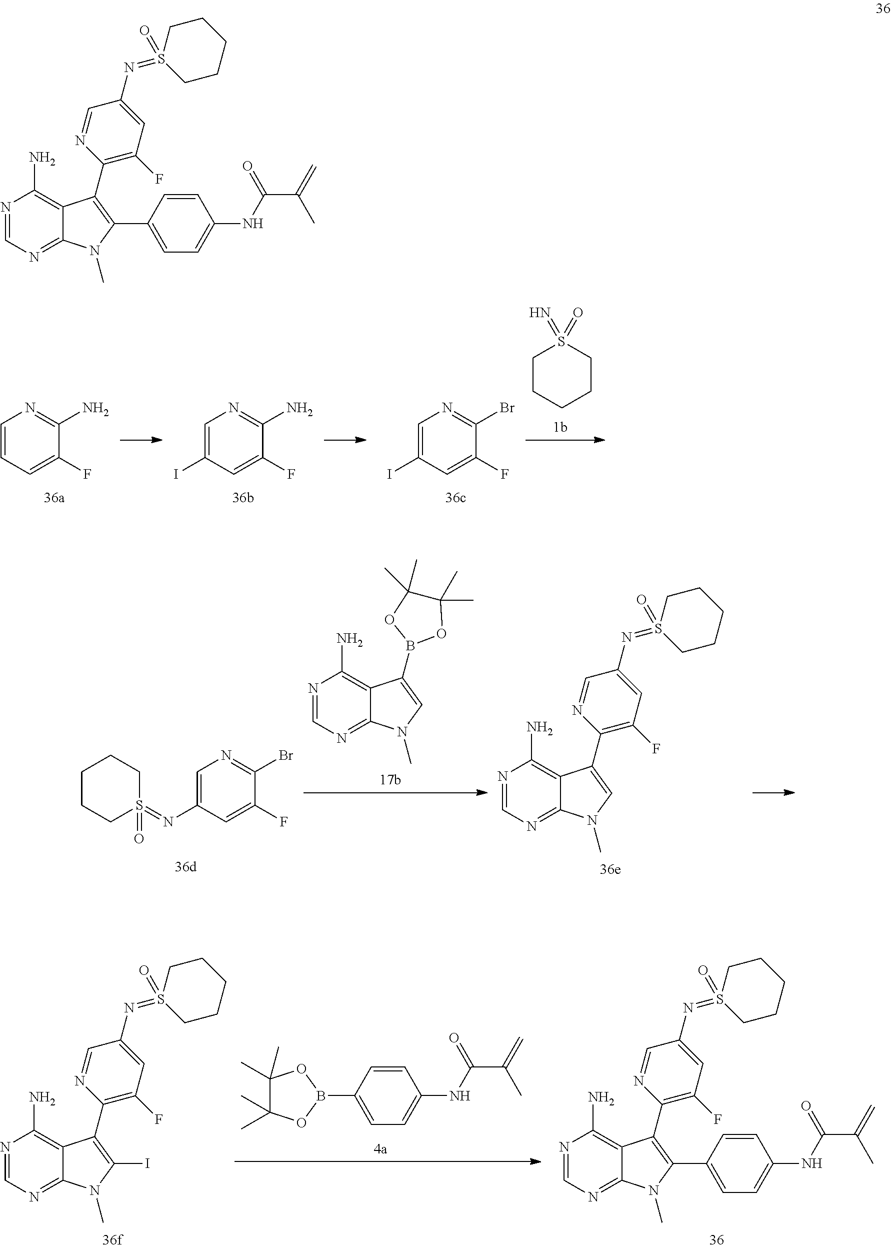

### Step 1 3-fluoro-5-iodo-pyridine-2-amine

Compound 36a (3.00 g, 26.8 mmol) was dissolved in DMF (20 mL), N-iodosuccinimide (6.63 g, 29.5 mmol) was added at room temperature, and the reaction was carried out at room temperature for 14 h. After the reaction was completed, it was extracted with ethyl acetate (50 mL×3), and the organic phases were combined and concentrated under reduced pressure to obtain a crude product. The crude product was purified by column chromatography (PE/EtOAc=1/1) to obtain compound 36b (4.5 g), white solid.

MS (ESI) m/z 239.10 [M+H]⁺.

### Step 2 3-fluoro-5-iodo-pyridine-2-amine 36c

Compound 6b (2.00 g, 8.40 mmol) was dissolved in acetonitrile (20 mL), copper bromide (2.25 g, 10.1 mmol) was added at room temperature, and isoamyl nitrite (1.47 g, 12.6 mmol) was added slowly at 50° C. to react at that temperature for 3 h. After the reaction was completed, it was extracted with ethyl acetate (50 mL×3), and the organic phases were combined and concentrated under reduced pressure to obtain a crude product. The crude product was purified by column chromatography (PE/EtOAc=5/1) to obtain compound 36c (1.5 g), white solid.

MS (ESI) m/z 302.20 [M+H]⁺.

### Step 3 1-((6-bromo-5-fluoropyridin-3-yl) imino) hexahydro-1λ⁶-thiopyran-1-oxide 36d

Compound 36c (0.50 g, 1.66 mmol) was dissolved in 1,4-dioxane (10 mL), compound 1b (0.22 g, 1.66 mmol), Cs₂CO₃ (1.62 g, 4.98 mmol), Xantphos (0.19 g, 0.332 mmol), and Pd₂(dba)₃ (0.15 g, 0.166 mmol) were added at room temperature, and it was protected with N₂ and reacted at 100° C. for 14 h. After TLC monitored the completion of the reaction, it was extracted with ethyl acetate (50 mL×3), and the organic phases were combined and concentrated under reduced pressure to obtain a crude product. The crude product was purified by column chromatography (PE/EtOAc=1/1) to obtain compound 36d (0.25 g), white solid.

MS (ESI) m/z 307.20 [M+H]⁺.

### Step 4 1-(6-(4-amino-7-methyl-7H-pyrrolo[2,3-d] pyrimidin-5-yl)-5-fluoropyridine-3-yl) imino) hexahydro-1λ⁶-thiopyran-1-oxide 36e

Compound 17b (0.20 g, 0.73 mmol) was dissolved in 1,4-dioxane (6 mL) and H₂O (2 mL), compound 36d (0.25 g, 0.80 mmol), potassium phosphate (0.46 g, 2.20 mmol), and tetrakis (triphenylphosphine) palladium (84.0 mg, 0.073 mmol) were added, and it was protected with N₂ and reacted at 90° C. for 14 h. After the reaction was completed, it was extracted with ethyl acetate (50 mL×3), and the organic phases were combined and concentrated under reduced pressure to obtain a crude product. The crude product was purified by column chromatography (DCM/MeOH=10/1) to obtain compound 36e (180 mg), white solid.

MS (ESI) m/z 375.10 [M+H]⁺.

### Step 5 1-(6-(4-amino-6-iodo-7-methyl-7H-pyrrolo[2,3-d] pyrimidin-5-yl)-5-fluoropyridine-3-yl) imino) hexahydro-1λ⁶-thiopyran-1-oxide 36f

Compound 36e (0.15 g, 0.40 mmol) was dissolved in dichloromethane (10 mL), and it was cooled down to 0° C., and at this temperature, trifluoroacetic acid (0.14 g, 1.2 mmol) and N-iodobutylimide (110 mg, 0.48 mmol) were added to react at 0° C. for 1 h. After the reaction was completed, it was extracted with ethyl acetate (50 mL×3), and the organic phases were combined and concentrated under reduced pressure to obtain crude product compound 36f (180 mg), white solid.

MS (ESI) m/z 501.11 [M+H]⁺.

### Step 6 N-(4-(4-amino-5-(3-fluoro-5-((1-oxotetrahydro-2H-1λ⁶-thiopyran-1-ylidene) amino) pyridine-2-yl)-7-methyl-7H-pyrrole [2,3-d] pyrimidin-6-yl) phenyl) methacrylamide 36

Compound 36f (0.18 g, 0.36 mmol) was dissolved in dioxane (6 mL) and H₂O (2 mL), compound 4a (124 mg, 0.43 mmol), potassium phosphate (0.23 g, 1.10 mmol), and tetrakis (triphenylphosphine) palladium (41.0 mg, 0.036 mmol) were added, and reacted at 90° C. for 14 h under N₂ protection. After the reaction was completed, it was extracted with ethyl acetate (50 mL×3), and the organic phases were combined and concentrated under reduced pressure to obtain a crude product. The crude product was purified by preparative TLC plate (EtOAc/THF=3/1) to obtain compound 36 (78.3 mg), white solid.

MS (ESI) m/z 534.21 [M+H]⁺.

¹H NMR (400 MHz, CDCl₃) δ 8.38 (s, 1H), 8.33-8.29 (m, 1H), 7.70-7.55 (m, 4H), 7.30 (s, 1H), 7.00 (dt, J=11.5, 4.0 Hz, 1H), 6.30 (s, 2H), 5.84 (s, 1H), 5.52 (d, J=1.1 Hz, 1H), 3.74 (d, J=3.4 Hz, 4H), 3.40-3.33 (m, 3H), 3.24-3.16 (m, 3H), 2.19-2.09 (m, 9H), 1.83-1.76 (m, 3H).

### Example 37 N-(4-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl)-2-fluorophenyl) methacrylamide

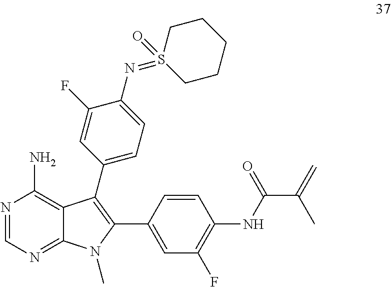 [SMILES: C=C(C)C(=O)Nc1ccc(-c2c(-c3ccc(N=S4(=O)CCCCC4)c(F)c3)c3c(N)ncnc3n2C)cc1F]

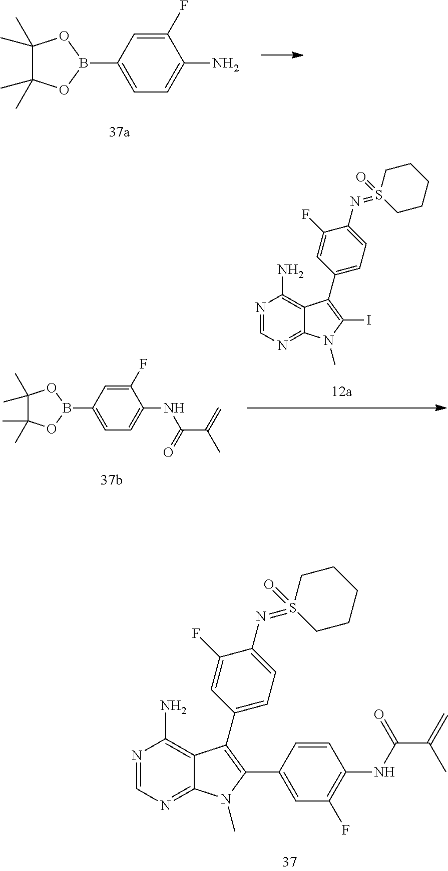 [SMILES: CC1(C)OB(c2ccc(N)c(F)c2)OC1(C)C ; C=C(C)C(=O)Nc1ccc(B2OC(C)(C)C(C)(C)O2)cc1F ; Cn1c(I)c(-c2ccc(N=S3(=O)CCCCC3)c(F)c2)c2c(N)ncnc21 ; C=C(C)C(=O)Nc1ccc(-c2c(-c3ccc(N=S4(=O)CCCCC4)c(F)c3)c3c(N)ncnc3n2C)cc1F]

### Step 1 N-(2-fluoro-4-(4,4,5,5-tetramethyl-1,3,2-dioxaborolan-2-yl) phenyl) methacrylamide 37b

Compound 37a (1.00 g, 4.22 mmol) was dissolved in dichloromethane (20 mL), triethylamine (0.85 g, 8.44 mmol) was added at room temperature, cooled down to 0° C., methacryloyl chloride (0.46 g, 4.33 mmol) was added to react at room temperature for 1 h. After the reaction was completed, it was extracted with ethyl acetate (50 mL×3), and the organic phases were combined and concentrated under reduced pressure to obtain a crude product. The crude product was purified by column chromatography (PE/EtOAc=5/1) to obtain compound 37b (0.95 g), colorless oily liquid.

MS (ESI) m/z 306.20 [M+H]⁺.

¹H NMR (400 MHz, CDCl₃) δ 8.62-8.23 (m, 1H), 8.04-7.75 (m, 1H), 7.52 (ddd, J 25.4, 13.8, 11.0 Hz, 2H), 6.02-5.58 (m, 1H), 5.59 (s, 1H), 2.07 (s, 3H), 1.33 (s, 12H).

### Step 2 N-(4-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl)-2-fluorophenyl) methacrylamide 37

Compound 12a (0.10 g, 0.20 mmol) was dissolved in 1,4-dioxane (6 mL) and H₂O (2 mL), compound 37b (73.0 mg, 0.24 mmol), potassium phosphate (0.13 g, 0.60 mmol), and tetrakis (triphenylphosphine) palladium (23.0 mg, 0.02 mmol) were added, and reacted at 90° C. for 14 h under N₂ protection. After the reaction was completed, it was extracted with ethyl acetate (50 mL×3), and the organic phases were combined and concentrated under reduced pressure to obtain a crude product. The crude product was purified by preparative TLC plate (EtOAc/THF=3/1) to obtain compound 37 (36.3 mg)

MS (ESI) m/z 551.10 [M+H]⁺.

¹H NMR (400 MHz, CDCl₃) δ 8.46 (t, J=8.4 Hz, 1H), 8.38 (s, 1H), 7.82 (s, 1H), 7.25 (t, J=8.5 Hz, 1H), 7.11 (d, J=8.9 Hz, 1H), 6.99 (dd, J=11.6, 1.9 Hz, 1H), 6.94 (dd, J=7.1, 5.0 Hz, 2H), 5.89 (s, 1H), 5.56 (d, J=1.3 Hz, 1H), 5.27 (s, 2H), 3.77 (d, J=20.7 Hz, 3H), 3.37 (d, J=14.5 Hz, 2H), 3.19 (ddd, J=13.3, 8.9, 4.2 Hz, 2H), 2.23-2.07 (m, 6H), 1.84-1.73 (m, 3H).

### Example 38 N-(5-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl)-2-fluorophenyl) methacrylamide KTH021115

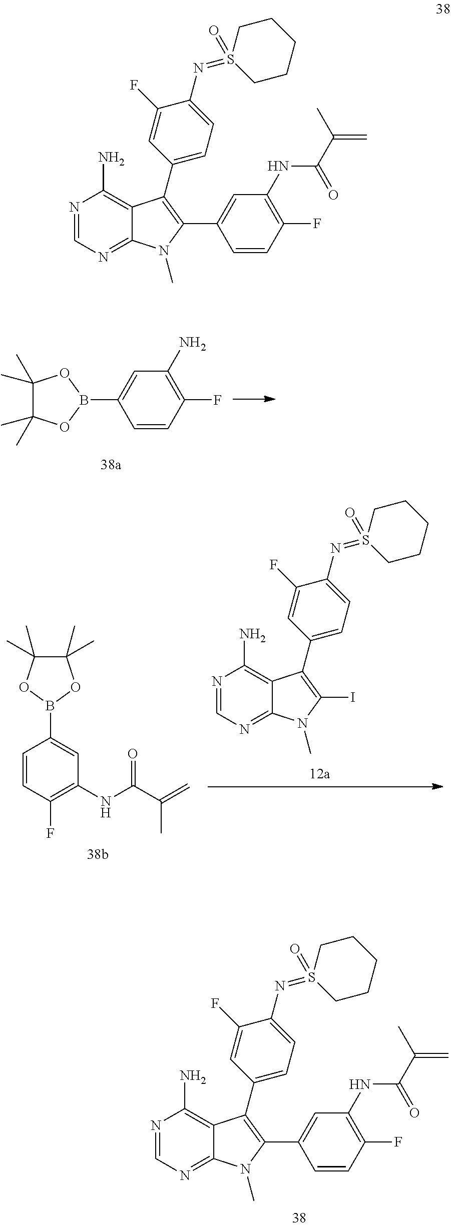

### Step 1 N-(2-fluoro-5-(4,4,5,5-tetramethyl-1,3,2-dioxaborolan-2-yl) phenyl) methacrylamide

Compound 38a (1.00 g, 4.22 mmol) was dissolved in dichloromethane (20 mL), triethylamine (0.85 g, 8.44 mmol) was added at room temperature, cooled down to 0° C., methacryloyl chloride (0.46 g, 4.33 mmol) was added to react at room temperature for 1 h. After the reaction was completed, it was extracted with ethyl acetate (50 mL×3), and the organic phases were combined and concentrated under reduced pressure to obtain a crude product. The crude product was purified by column chromatography (PE/EtOAc=5/1) to obtain compound 38b (1.1 g), colorless oily liquid.

MS (ESI) m/z 306.20 [M+H]⁺.

¹H NMR (400 MHz, CDCl₃) δ 8.74 (dd, J=8.5, 1.3 Hz, 1H), 7.72 (s, 1H), 7.61-7.49 (m, 1H), 7.16-7.06 (m, 1H), 5.84 (d, J=19.4 Hz, 1H), 5.57-5.47 (m, 1H), 2.10 (s, 3H), 1.34 (s, 14H).

### Step 2 N-(5-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl)-2-fluorophenyl) methacrylamide 38

Compound 12a (0.10 g, 0.20 mmol) was dissolved in 1,4-dioxane (6 mL) and H₂O (2 mL), compound 38b (73.0 mg, 0.24 mmol), potassium phosphate (0.13 g, 0.60 mmol), and tetrakis (triphenylphosphine) palladium (23.0 mg, 0.02 mmol) were added, and reacted at 90° C. for 14 h under N2 protection. After the reaction was completed, it was extracted with ethyl acetate (50 mL×3), and the organic phases were combined and concentrated under reduced pressure to obtain a crude product. The crude product was purified by preparative TLC plate (EA/THF=3/1) to obtain compound 38 (37.3 mg).

MS (ESI) m/z 551.10 [M+H]⁺.

¹H NMR (400 MHz, CDCl₃) δ 8.45 (dd, J=7.5, 2.1 Hz, 1H), 8.37 (d, J=5.4 Hz, 1H), 7.80 (s, 1H), 7.23 (t, J=8.6 Hz, 1H), 7.07 (dd, J=10.7, 8.5 Hz, 1H), 6.98-6.92 (m, 2H), 6.91-6.86 (m, 1H), 5.88 (s, 1H), 5.56 (t, J=4.8 Hz, 1H), 5.14 (s, 2H), 3.76 (s, 3H), 3.41-3.31 (m, 2H), 3.17 (ddd, J=13.5, 9.0, 4.3 Hz, 2H), 2.20-2.06 (m, 7H), 1.77 (d, J=15.1 Hz, 2H).

### Example 39 N-(3-(4-amino-5-(4-((dimethylamino) (oxo) (trifluoromethyl)-λ⁶-sulfanylidene) amino) phenyl)-7-methyl-7H-pyrrole [2,3-d] pyrimidin-6-yl)-4-fluorophenyl) methacrylamide

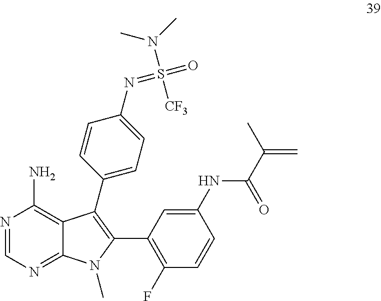 [SMILES: C=C(C)C(=O)Nc1ccc(F)c(-c2c(-c3ccc(N=S(=O)(N(C)C)C(F)(F)F)cc3)c3c(N)ncnc3n2C)c1]

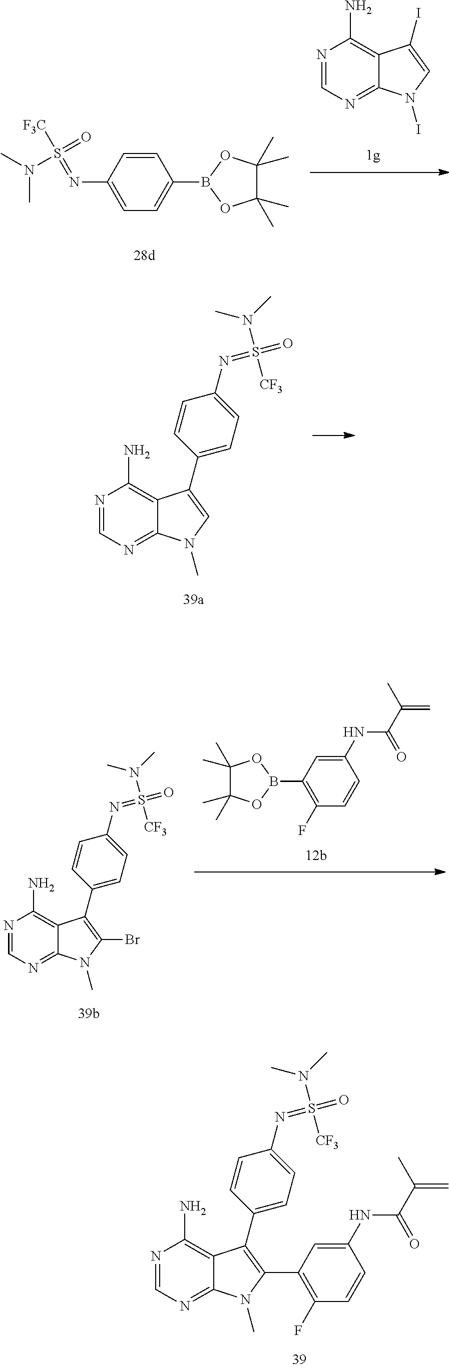

### Step 1 N′-(4-(4-amino-7-methyl-7H-pyrrole [2,3-d] pyrimidin-5-yl) phenyl)-1,1,1-trifluoro-N,N-dimethylmethane sulfonamide 39a

Compound 28 (500 mg, 1.3 mmol), compound 1g (302 mg, 1.1 mmol), Pd(dppf)Cl₂ (81 mg, 0.11 mmol), and potassium phosphate (584 mg, 2.8 mmol) were placed in a bottle, and 1,4-dioxane/H₂O (10 mL/1 mL) was added. After replaced with N₂ three times, the reaction was carried out at 85° C. for 16 h. The reaction solution was filtered and concentrated, and the obtained crude product was subjected to column chromatography (DCM/MeOH=0-10%) to obtain compound 39a (200 mg), brown solid.

MS (ESI) m/z 399 [M+H]⁺.

### Step 2 N′-(4-(4-amino-6-bromo-7-methyl-7H-pyrrole[2,3-d] pyrimidin-5-yl) phenyl)-1,1,1-trifluoro-N,N-dimethylmethane sulfonamide 39b

Compound 39a (100 mg, 0.25 mmol) was dissolved in DMF (3 mL), after it was cooled down to 0° C., N-bromosuccinimide (49 mg, 0.28 mmol) was added to react at 0° C. for 1 h. The reaction solution was diluted with DCM, washed with water three times, and the organic phase was dried over anhydrous sodium sulfate and concentrated, the obtained crude product was purified by column chromatography (DCM/MeOH=0-10%) to obtain compound 39b (100 mg), red solid.

MS (ESI) m/z 476 [M+H]⁺.

### Step 3 N-(3-(4-amino-5-(4-((dimethylamino) (oxo) (trifluoromethyl)-λ⁶-sulfanylidene) amino) phenyl)-7-methyl-7H-pyrrole [2,3-d] pyrimidin-6-yl)-4-fluorophenyl) methacrylamide 39

Compound 39b (100 mg, 0.21 mmol), compound 12b (83 mg, 0.27 mmol), Pd(PPh₃)₄ (49 mg, 0.042 mmol), and potassium phosphate (133 mg, 0.63 mmol) were placed in a bottle, and 1,4-dioxane/H₂O (4 mL/0.8 mL) was added. After replaced with N₂ three times, the reaction was carried out at 100° C. for 16 h. The reaction solution was filtered and concentrated, and the obtained crude product was separated by column chromatography (DCM/MeOH=10/1) to obtain compound 39 (28 mg), light yellow solid.

MS (ESI) m/z 576 [M+H]⁺.

¹H NMR (400 MHz, DMSO-d₆) δ 9.93 (s, 1H), 8.27 (s, 1H), 7.87-7.80 (m, 1H), 7.71 (dd, J=6.6, 2.6 Hz, 1H), 7.32 (t, J=9.2 Hz, 1H), 7.21 (d, J=8.3 Hz, 2H), 7.05 (d, J=8.4 Hz, 2H), 5.82 (s, 1H), 5.57 (s, 1H), 3.60 (s, 3H), 3.08 (s, 6H), 1.98 (s, 3H), 1.30 (s, 2H).

### Example 40 N-(4-(4-amino-5-(3-chloro-4-((1-oxotetrahydro-2H-1λ⁶-thiopyran-1-ylidene) amino) phenyl)-7-methyl-7H-pyrrolo [2,3-d] pyrimidin-6-yl) phenyl) methacrylamide

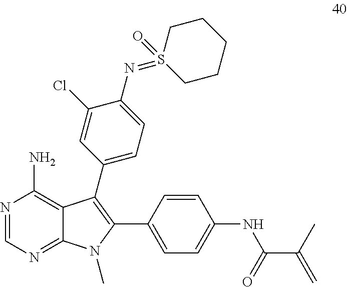 [SMILES: C=C(C)C(=O)Nc1ccc(-c2c(-c3ccc(N=S4(=O)CCCCC4)c(Cl)c3)c3c(N)ncnc3n2C)cc1]

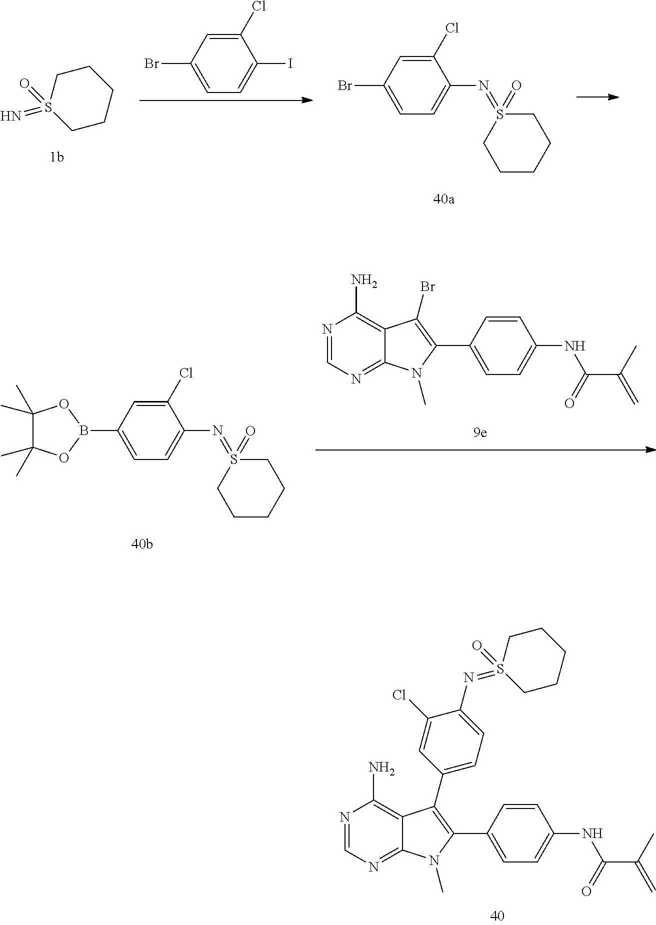 [SMILES: N=S1(=O)CCCCC1 ; Clc1cc(Br)ccc1I ; O=S1(=Nc2ccc(Br)cc2Cl)CCCCC1 ; CC1(C)OB(c2ccc(N=S3(=O)CCCCC3)c(Cl)c2)OC1(C)C ; C=C(C)C(=O)Nc1ccc(-c2c(Br)c3c(N)ncnc3n2C)cc1 ; C=C(C)C(=O)Nc1ccc(-c2c(-c3ccc(N=S4(=O)CCCCC4)c(Cl)c3)c3c(N)ncnc3n2C)cc1]

### Step 1 1-((4-bromo-2-chlorophenyl) imino) hexahydro-1λ⁶-thiopyran-1-oxide

Compound 1b (0.25 g, 1.9 mmol), cesium carbonate (0.72 g, 2.2 mmol), 4-bromo-2-chloro-1-iodobenzene (0.5 g, 1.58 mmol), Pd₂(dba)₃ (36 mg, 0.0395 mmol), and Xantphos (68 mg, 0.12 mmol) were added to 1,4-dioxane (8 mL), after replaced with N₂ three times, the reaction solution was heated up to 100° C. for 5 h. The reaction solution was filtered through diatomaceous earth, and the filter cake was washed with ethyl acetate (30 mL) and saturated saline solution (50 mL). The organic phase was dried over anhydrous sodium sulfate and concentrated under reduced pressure to obtain a crude product. The crude product was purified by column chromatography (PE/EtOAc=4/1 to 3/2) to obtain compound 40a (0.43 g), light yellow solid.

MS (ESI) m/z 321.90[M+H]⁺.

### Step 2 1-((2-chloro-4-(4,4,5,5-tetramethyl-1,3,2-dioxaborolan-2-yl) phenyl) imino) hexahydro-1λ⁶-thiophene 1-oxide 40a

Compound 40a (0.43 g, 1.33 mmol), bis(pinacolato)diboron (0.41 g, 1.6 mmol), Pd(dppf)Cl₂ (200 mg, 0.266 mmol), potassium acetate (390 mg, 3.99 mmol), and 1,4-dioxane (8 mL) were added into a sealed tube, after replaced with N₂ three times, sealed, the reaction solution was heated up to 100° C. and stirred for 16 h. Then water (10 mL) was added and extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=5/1) to obtain compound 40b (450 mg), yellow oily liquid.

MS (ESI) m/z 369[M+H]⁺.

### Step 3 N-(4-(4-amino-5-(3-chloro-4-(1-oxotetrahydro-2H-1λ⁶-thiopyran-1-ylidene) amino) phenyl)-7-methyl-7H-pyrrolo [2,3-d] pyrimidin-6-yl) phenyl) methacrylamide 40

Compound 40b (115 mg, 0.31 mmol), compound 9e (100 mg, 0.26 mmol), Pd(PPh₃)₄ (60 mg, 0.052 mmol), and K₃PO₄ (165 mg, 0.78 mmol) were added into a sealed tube, after replaced with N₂ three times, 1,4-dioxane (5 mL) and H₂O (0.5 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/20) to obtain compound 40 (100 mg), white solid.

MS (ESI) m/z 549.09 [M+H]⁺.

¹H NMR (400 MHz, CDCl₃) δ 8.35 (s, 1H), 7.61-7.47 (m, 3H), 7.26-7.16 (m, 3H), 6.98-6.90 (m, 1H), 5.81 (s, 1H), 5.50 (d, J=1.7 Hz, 1H), 5.40-5.00 (m, 3H), 3.70 (s, 3H), 3.40 (d, J=13.6 Hz, 2H), 3.18-3.03 (m, 2H), 2.26-2.12 (m, 3H), 2.05 (s, 3H), 2.04-2.00 (m, 1H), 1.89-1.75 (m, 2H).

### Example 41 N-(4-(4-amino-5-(3-fluoro-4-(4-oxo-1,4λ⁶-thiohexane-4-ylidene) amino) phenyl)-7-methyl-7H-pyrrolo [2,3-d] pyrimidin-6-yl) phenyl) methacrylamide

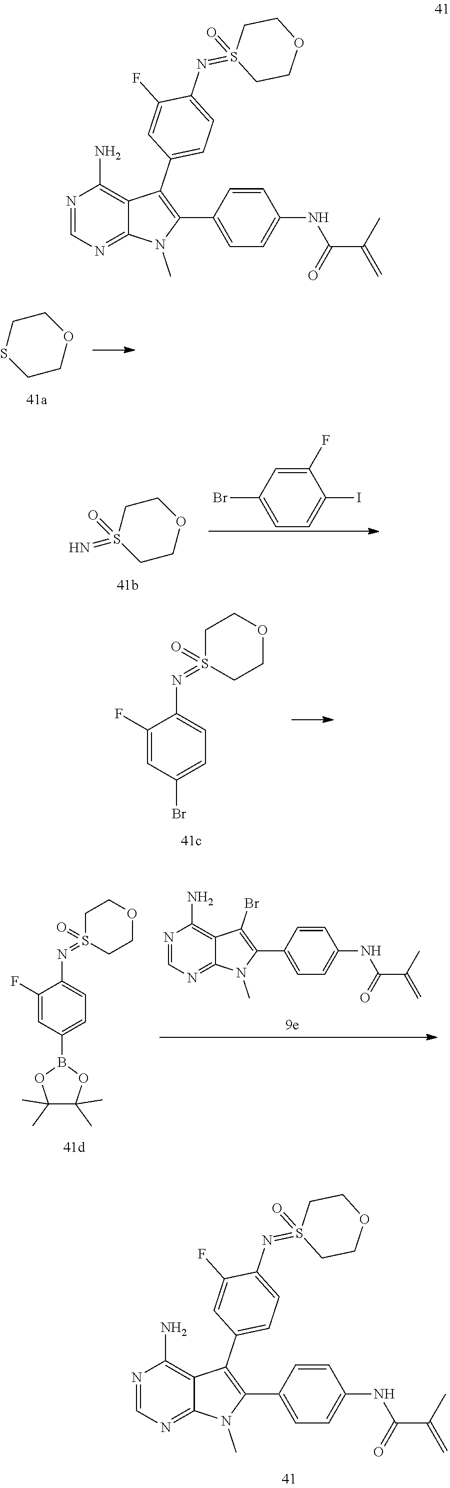

### Step 1 4-imino-1,4λ⁶-oxythiazole 4-oxide 41b

1,4-thiazole 41a (2.0 g, 19.2 mmol) and ammonium carbamate (2.24 g, 28.8 mmol) were added into a flask in sequence, then methanol (40 mL) and iodobenzene diethyl ester (13.9 g, 43.2 mmol) were added. The reaction solution was stirred open at room temperature for 30 min, and then concentrated under reduced pressure to remove the solvent to obtain a crude product. The crude product was purified by column chromatography (MeOH/DCM=1/20) to obtain compound 41b (1.9 g), white solid.

MS (ESI) m/z 136.04 [M+H]⁺.

### Step 2 4-((4-bromo-2-fluorophenyl) imino)-1,4λ⁶-thiohexane-4-oxide 41c

Compound 41b (700 mg, 5.17 mmol), cesium carbonate (1.9 g, 6.03 mmol), 4-bromo-2-fluoro-1-iodobenzene (1.28 g, 4.31 mmol), Pd₂(dba)₃ (99 mg, 0.108 mmol), and Xantphos (190 mg, 0.32 mmol) were added to 1,4-dioxane (15 mL), after replaced with N₂ three times, the reaction solution was heated up to 100° C. for 16 h. The reaction solution was filtered through diatomaceous earth, and the filter cake was washed with ethyl acetate (100 mL) and saturated saline solution (50 mL). The organic phase was dried over anhydrous sodium sulfate and concentrated under reduced pressure to obtain a crude product. The crude product was purified by column chromatography (PE/EtOAc=10/1 to 3/1) to obtain compound 41c (1.1 g), light yellow solid.

MS (ESI) m/z 307.97 [M+H]⁺.

### Step 3 4-((2-fluoro-4-(4,4,5,5-tetramethyl-1,3,2-dioxaborolan-2-yl) phenyl) imino)-1,4-λ⁶-thiooxane-4-oxide 41d

Compound 41c (300 mg, 0.97 mmol), bis(pinacolato)diboron (300 mg, 1.17 mmol), Pd(dppf)Cl₂ (140 mg, 0.194 mmol), potassium acetate (300 mg, 2.91 mmol), and 1,4-dioxane (6 mL) were added into a sealed tube, after replaced with N₂ three times, sealed, and the reaction solution was heated up to 100° C. and stirred for 16 h. Then water (10 mL) was added and extract with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=5/1) to obtain compound 41d (240 mg), yellow oily liquid.

MS (ESI) m/z 356 [M+H]⁺.

### Step 4 N-(4-(4-amino-5-(3-fluoro-4-(4-oxo-1,4λ⁶-thiohexane-4-ylidene) amino) phenyl)-7-methyl-7H-pyrrolo [2,3-d] pyrimidin-6-yl) phenyl) methacrylamide 41

Compound 41d (165 mg, 0.47 mmol), compound 9e (130 mg, 0.36 mmol), Pd(PPh₃)₄ (83 mg, 0.072 mmol), and K₃PO₄ (230 mg, 1.08 mmol) were added into a sealed tube, after replaced with N₂ three times, 1,4-dioxane (5 mL) and H₂O (0.5 mL) were added, sealed, and the reaction solution was heated up to 100° C. and stirred for 16 h. Then dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/30) to obtain compound 41 (90 mg), white solid.

MS (ESI) m/z 535.08[M+H]⁺.

¹H NMR (400 MHz, CDCl₃) δ 8.35 (s, 1H), 7.61-7.48 (m, 3H), 7.26-7.15 (m, 3H), 6.96-6.86 (m, 2H), 5.81 (s, 1H), 5.54-5.45 (s, 1H), 4.24-4.12 (m, 4H), 3.69 (s, 3H), 3.43-3.23 (m, 4H), 2.22 (s, 3H), 2.02-1.99 (m, 2H).

### Example 42 N-(4-(4-amino-5-(4-(dimethyl (oxo)-λ⁶-sulfanylidene) amino)-3-fluorophenyl)-7-methyl-7H-pyrrolo [2,3-d] pyrimidin-6-yl)-3-fluorophenyl) methacrylamide

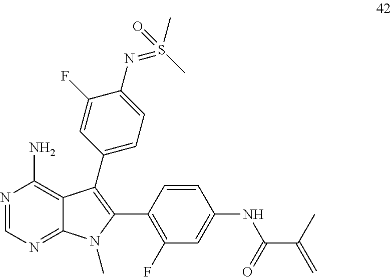 [SMILES: C=C(C)C(=O)Nc1ccc(-c2c(-c3ccc(N=S(C)(C)=O)c(F)c3)c3c(N)ncnc3n2C)c(F)c1]

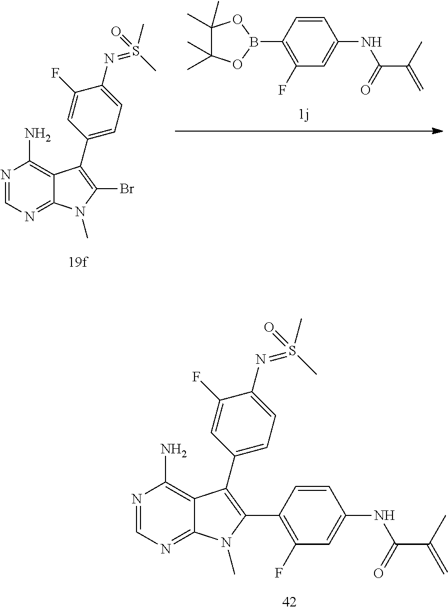

Compound 19f (100 mg, 0.24 mmol), compound 1j (89 mg, 0.29 mmol), Pd(PPh₃)₄ (56 mg, 0.049 mmol), and K₃PO₄ (156 mg, 0.73 mmol) were added into a sealed tube, after replaced with N₂ three times, 1,4-dioxane (5 mL) and H₂O (0.5 mL) were added, sealed, and the reaction solution was heated up to 100° C. and stirred for 16 h. Then dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/30) to obtain compound 42 (40 mg), white solid.

MS (ESI) m/z 511.75 [M+H]⁺.

¹H NMR (400 MHz, MeOD-d4) δ 8.22 (s, 1H), 7.76 (dd, J=12.1, 2.1 Hz, 1H), 7.41 (dd, J=8.4, 2.1 Hz, 1H), 7.21 (td, J=8.5, 4.7 Hz, 2H), 6.98 (d, J=11.1 Hz, 2H), 5.84 (s, 1H), 5.57 (d, J=1.6 Hz, 1H), 4.60 (s, 1H), 3.64 (d, J=0.9 Hz, 3H), 3.26 (s, 6H), 2.05 (s, 3H).

### Example 43 N-(4-(4-amino-5-(2-fluoro-4-((1-oxotetrahydro-2H-1λ⁶-thiopyran-1-ylidene) amino) phenyl)-7-methyl-7H-pyrrolo [2,3-d] pyrimidin-6-yl) phenyl) methacrylamide

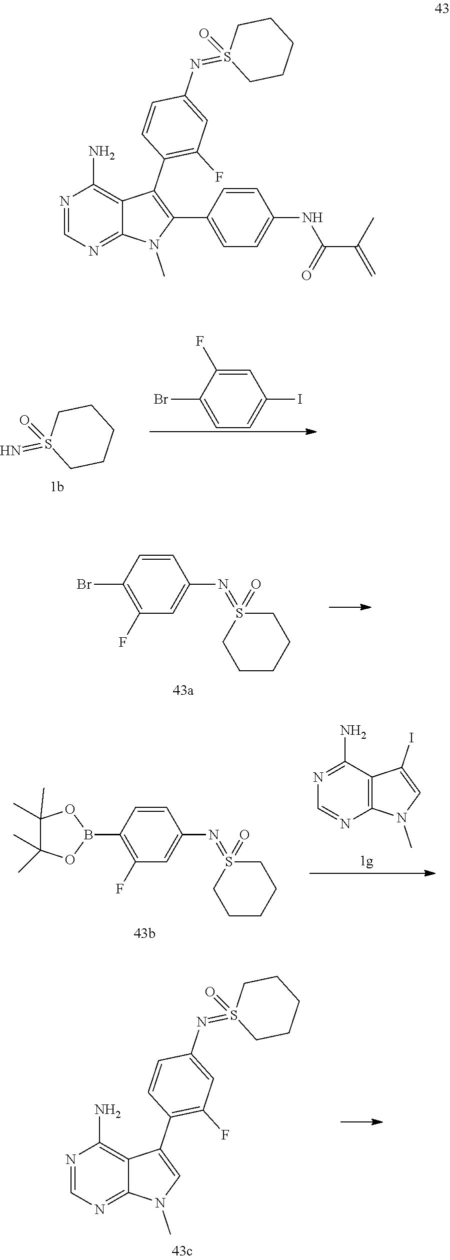

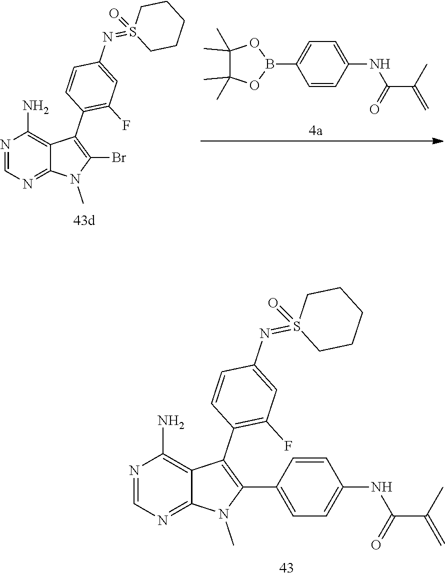

### Step 1 1-((4-bromo-3-fluorophenyl) imino) hexahydro-1λ⁶-thiopyran-1-oxide 43a

Compound 1b (270 mg, 2.02 mmol), carbofuran (820 mg, 2.52 mmol), 1-bromo-2-fluoro-4-iodobenzene (500 mg, 1.68 mmol), Pd₂(dba)₃ (39 mg, 0.042 mmol), and Xantphos (73 mg, 0.126 mmol) were added to 1,4-dioxane (10 mL), after replaced with N₂ three times, the reaction solution was heated up to 100° C. for 13 h. The reaction solution was filtered through diatomaceous earth, the filter cake was washed with ethyl acetate (50 mL), and saturated saline solution (50 mL) was used for washing. The organic phase was dried over anhydrous sodium sulfate and concentrated under reduced pressure to obtain a crude product. The crude product was purified by column chromatography (PE/EtOAc=4/1 to 1/1) to obtain compound 43a (560 mg).

MS (ESI) m/z 306.2 [M+H]⁺.

### Step 2 1-((3-Fluoro-4-(4,4,5,5-tetramethyl-1,3,2-dioxaborolan-2-yl) phenyl) imino) hexahydro-1λ⁶-thiophene 1-oxide 43b

Compound 43a (0.56 g, 1.73 mmol), bis(pinacolato)diboron (0.53 g, 2.1 mmol), Pd(dppf)Cl₂ (0.25 mg, 0.34 mmol), and potassium acetate (0.51 mg, 5.2 mmol) were placed in a bottle, 1,4-dioxane (5 mL) was added, after replaced with N₂ three times, the reaction was carried out at 100° C. for 18 h. The reaction solution was filtered and concentrated, and the obtained crude product was purified by column chromatography (PE/EtOAc=0-20%) to obtain compound 43b (427 mg), yellow oily liquid.

### Step 3 1-(4-(4-amino-7-methyl-7H-pyrrolo [2,3-d] pyrimidin-5-yl)-3-fluorophenyl) imino) hexahydro-1λ⁶-thiopyran-1-oxide 43c

Compound 43b (100 mg, 0.28 mmol), compound 1g (65 mg, 0.24 mmol), Pd(PPh₃)₄ (54 mg, 0.056 mmol), and K₃PO₄ (150 mg, 0.84 mmol) were added into a sealed tube, after replaced with argon gas three times, 1,4-dioxane (4 mL) and H₂O (0.5 mL) were added, sealed, and the reaction solution was heated up to 100° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/30) to obtain compound 43c (80 mg), yellow solid.

MS (ESI) m/z 373.4 [M+H]⁺.

### Step 4 (4-(4-amino-6-bromo-7-methyl-7H-pyrrolo [2,3-d] pyrimidin-5-yl)-2-fluorophenyl) imino) dimethyl-λ⁶-sulfonylone 43d

Compound 43c (55 mg, 0.15 mmol) was dissolved in DMF (3 mL), and a solution of N-bromosuccinimide (29 mg, 0.16 mmol) in DMF (0.5 mL) was added dropwise at 0° C., the reaction solution was stirred at 0° C. for 1 h. Then DCM (30 mL) was added, and washed with saturated saline solution (10 mL×3), and the organic phase was dried over anhydrous sodium sulfate, and concentrated under reduced pressure to obtain a crude product. The crude product was purified by column chromatography (MeOH/DCM=1/50) to obtain compound 43d (54 mg), yellow solid.

MS (ESI) m/z 412.3 [M+H]⁺.

### Step 5 N-(4-(4-amino-5-(2-fluoro-4-(1-oxotetrahydro-2H-1λ⁶-thiopyran-1-ylidene) amino) phenyl)-7-methyl-7H-pyrrolo [2,3-d] pyrimidin-6-yl) phenyl) methacrylamide 43

Compound 43d (57 mg, 0.15 mmol), compound 4a (50 mg, 0.18 mmol), Pd(PPh₃)₄ (30 mg, 0.03 mmol), and K₃PO₄ (100 mg, 0.45 mmol) were added into a sealed tube, after replaced with argon gas three times, 1,4-dioxane (3 mL) and H₂O (0.4 mL) were added, sealed, and the reaction solution was heated up to 100° C. and stirred for 16 h. Then dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/30) to obtain compound 43 (12 mg), white solid.

MS (ESI) m/z 533.54 [M+H]⁺.

¹H NMR (400 MHz, MeOD-d4) δ 8.18 (s, 1H), 7.70-7.62 (m, 2H), 7.32-7.26 (m, 2H), 7.21 (t, J=8.6 Hz, 1H), 7.00-6.91 (m, 2H), 5.81 (s, 1H), 5.53 (d, J=1.8 Hz, 1H), 4.58 (s, 1H), 3.68 (s, 3H), 3.43-3.34 (m, 2H), 3.30-3.19 (m, 2H), 2.13-1.99 (m, 5H), 1.82-1.52 (m, 4H).

### Example 44 N-(4-(4-amino-5-(4-(dimethyl (oxo)-λ⁶-sulfanylidene) amino)-3-fluorophenyl)-7-methyl-7H-pyrrolo [2,3-d] pyrimidin-6-yl) phenyl) methacrylamide

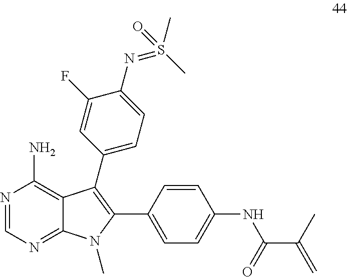 [SMILES: C=C(C)C(=O)Nc1ccc(-c2c(-c3ccc(N=S(C)(C)=O)c(F)c3)c3c(N)ncnc3n2C)cc1]

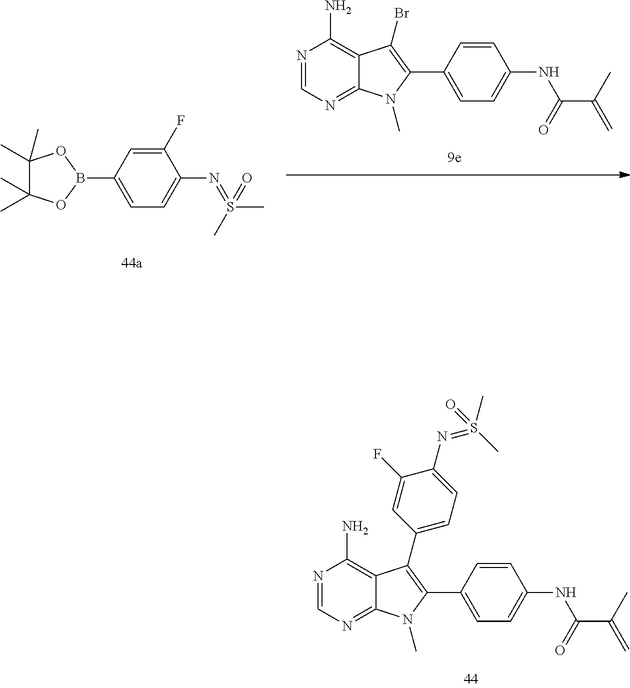

Compound 9e (210 mg, 0.53 mmol), compound 44a (200 mg, 0.64 mmol), Pd(PPh₃)₄ (120 mg, 0.11 mmol), and K₃PO₄ (340 mg, 1.59 mmol) were added into a sealed tube, after replaced with argon gas three times, 1,4-dioxane (5 mL) and H₂O (0.5 mL) were added, sealed, and the reaction solution was heated up to 100° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/30) to obtain compound 44 (100 mg), white solid.

MS (ESI) m/z 493.26 [M+H]⁺.

¹H NMR (400 MHz, MeOD-d4) δ 8.17 (s, 1H), 7.71-7.60 (m, 2H), 7.28 (d, J 8.6 Hz, 2H), 7.19 (t, J=8.4 Hz, 1H), 7.03-6.89 (m, 2H), 5.80 (s, 1H), 5.52 (s, 1H), 4.57 (s, 1H), 3.67 (s, 3H), 3.24 (s, 6H), 2.02 (s, 3H).

### Example 45N-(4-(4-amino-5-(4-(dimethyl (oxo)-λ⁶-sulfanylidene) amino) phenyl)-7-methyl-7H-pyrrolo [2,3-d] pyrimidin-6-yl) phenyl) methacrylamide

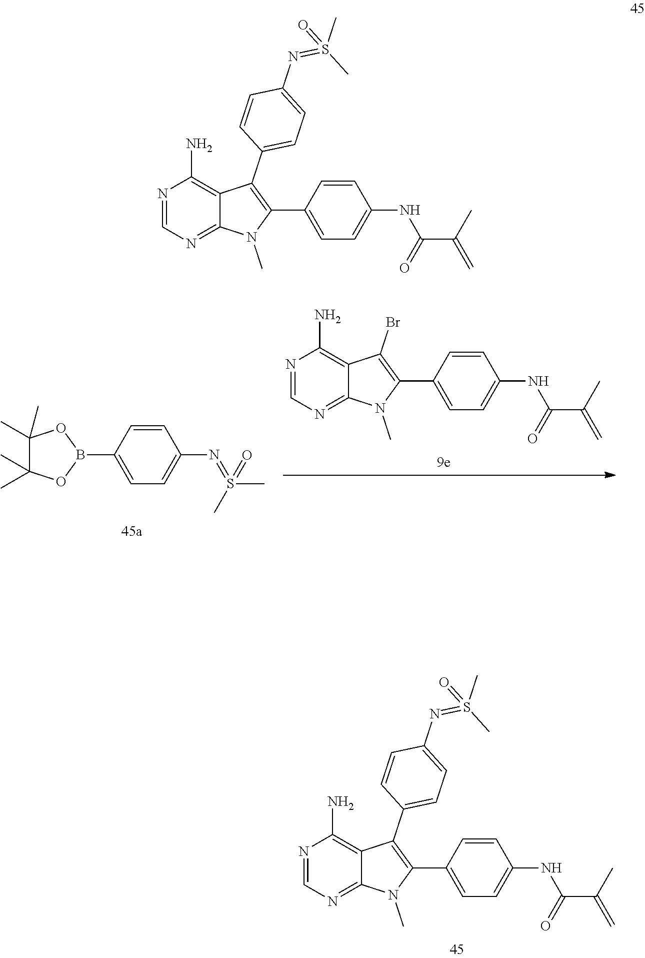

Compound 9e (120 mg, 0.31 mmol), compound 45a (110 mg, 0.37 mmol), Pd(PPh₃)₄ (72 mg 0.062 mmol), and K₃PO₄ (198 mg, 0.93 mmol) were added into a sealed tube, after replaced with argon gas three times, 1,4-dioxane (5 mL) and H₂O (0.5 mL) were added, seal, and the reaction solution was heated up to 100° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/30) to obtain compound 45 (80 mg), white solid.

MS (ESI) m/z 475.61 [M+H]⁺.

¹H NMR (400 MHz, MeOD-d4) δ 8.17 (s, 1H), 7.68-7.60 (m, 2H), 7.31-7.23 (m, 2H), 7.19-7.10 (m, 2H), 7.07-6.99 (m, 2H), 5.80 (s, 1H), 5.57-5.49 (m, 1H), 4.56 (s, 1H), 3.68 (s, 3H), 3.22 (s, 6H), 2.02 (t, J=1.2 Hz, 3H).

### Example 46 N-(4-(4-amino-5-(3-fluoro-4-((1-oxotetrahydro 2H-1λ⁶-thiopyran-1-ylidene) amino) phenyl)-7-methyl-7H-pyrrolo [2,3-d] pyrimidin-6-yl) phenyl)-N-methylmethacrylamide

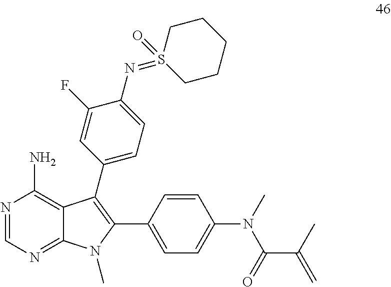 [SMILES: C=C(C)C(=O)N(C)c1ccc(-c2c(-c3ccc(N=S4(=O)CCCCC4)c(F)c3)c3c(N)ncnc3n2C)cc1]

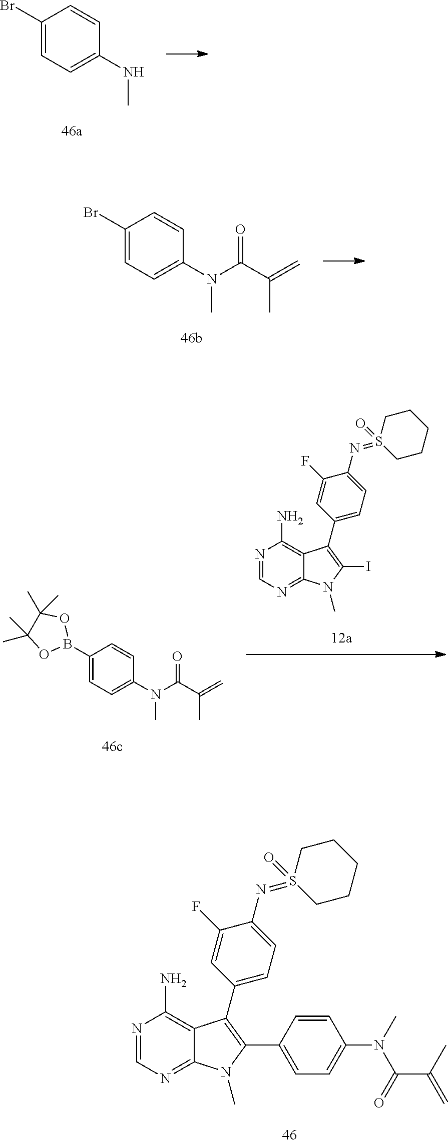

### Step 1 N-(4-bromophenyl)-N-methylmethacrylamide 46b

Compound 46a (500 mg, 2.69 mmol) and triethylamine (0.75 mL, 5.38 mmol) were dissolved in DCM (50 ml) at 0° C., 2-methylacryloyl chloride (290 mg, 2.82 mmol) was added dropwise to the reaction solution, and the reaction was stirred for 1 h. The reaction solution was then diluted with water (100 ml) and extracted with DCM (100 ml) three times. The organic phase was washed with saturated saline solution, dried over anhydrous Na₂SO₄, and concentrated under reduced pressure, the obtained crude product was purified by silica gel column chromatography (EA/PE=1/4) to obtain compound 46b (400 mg), yellow solid.

MS (ESI) m/z 254.1 [M+H]⁺.

### Step 2 N-methyl-N-(4-(4,4,5,5-tetramethyl-1,3,2-dioxaborolan-2-yl) phenyl) methacrylamide 46c

Compound 46b (300 mg, 1.18 mmol), bis(pinacolato)diboron (360 mg, 1.42 mmol), Pd(dppf)Cl₂ (172 mg, 0.236 mmol), and potassium acetate (347 mg, 3.54 mmol) were placed in a bottle, and dioxane (8 mL) was added, after replaced with N₂ three times, the reaction was carried out at 100° C. for 18 h. The reaction solution was filtered and concentrated, and the obtained crude product was purified by column chromatography (PE/EtOAc=0-20%) to obtain compound 46c (260 mg), yellow oily liquid.

MS (ESI) m/z 302 [M+H]⁺.

### Step 3 N-(4-(4-amino-5-(3-fluoro-4-((1-oxotetrahydro2H-1λ⁶-thiopyran-1-ylidene) amino) phenyl)-7-methyl-7H-pyrrolo [2,3-d] pyrimidin-6-yl) phenyl)-N-methylmethacrylamide 46

Compound 12a (110 mg, 0.22 mmol), compound 46c (80 mg, 0.27 mmol), Pd(PPh₃)₄ (51 mg, 0.044 mmol), and K₃PO₄ (140 mg, 0.66 mmol) were added into a sealed tube, after replaced with argon gas three times, 1,4-dioxane (5 mL) and H₂O (0.5 mL) were added, sealed, and the reaction solution was heated up to 100° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/30) to obtain compound 46 (75 mg), white solid.

MS (ESI) m/z 547.56 [M+H]⁺.

¹H NMR (400 MHz, CDCl₃) δ 8.36 (s, 1H), 7.22 (dd, J=8.5, 2.6 Hz, 3H), 7.13 (d, J=8.4 Hz, 2H), 6.92 (dd, J=8.1, 2.1 Hz, 1H), 6.82 (dd, J=11.5, 2.0 Hz, 1H), 5.08 (d, J=10.0 Hz, 3H), 5.01 (s, 1H), 3.71 (s, 3H), 3.38 (s, 5H), 3.15 (ddd, J=13.6, 8.7, 4.2 Hz, 2H), 2.12 (td, J=9.3, 8.5, 5.1 Hz, 4H), 1.76 (s, 3H), 1.25 (s, 2H).

### Example 47 N-(4-(4-amino-5-(3-fluoro-4-((oxo (pyrrolidine-1-yl) (trifluoromethyl) λ⁶-sulfanylidene) amino) phenyl)-7-methyl-7H-pyrrolo [2,3-d] pyrimidin-6-yl) phenyl) methacrylamide

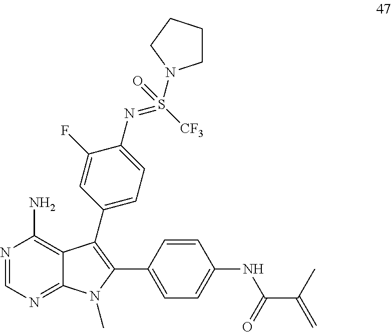 [SMILES: C=C(C)C(=O)Nc1ccc(-c2c(-c3ccc(N=S(=O)(N4CCCC4)C(F)(F)F)c(F)c3)c3c(N)ncnc3n2C)cc1]

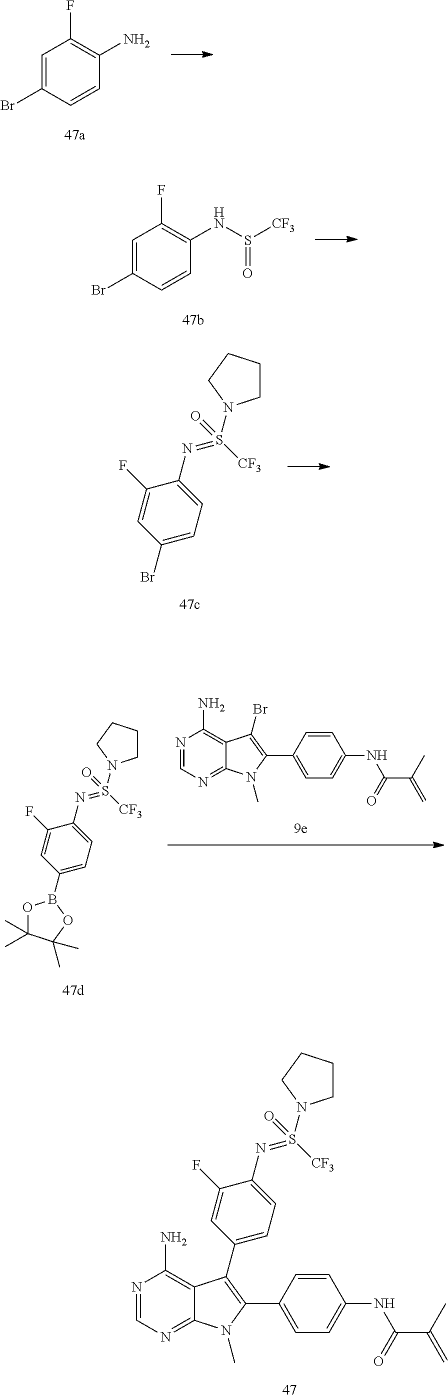

### Step 1 N-(4-bromo-2-fluorophenyl)-1,1,1-trifluoromethanesulfonamide 47b

Trifluoromethanesulfonyl chloride (2.4 g, 15.79 mmol) was dissolved in dichloromethane (30 mL), cooled down to 0° C., a solution of compound 47a (3.0 g, 15.79 mmol) in dichloromethane (10 mL) was added dropwise, then DIPEA (2.04 g, 15.79 mmol) was added, and heated up to room temperature to react for 2 h. The reaction solution was poured into water and extracted with DCM three times, dried over anhydrous sodium sulfate, and concentrated to obtain a crude product. The crude product was purified by column chromatography (PE/EtOAc=0-3%) to obtain compound 47b (2.8 g), white solid.

MS (ESI) m/z 306.56 [M+H]⁺.

### Step 2 1-(N-(4-bromo-2-fluorophenyl)-S-(trifluoromethyl) sulfonylimino) pyrrolidine 47c

Compound 47b (300 mg, 0.99 mmol) and N-chlorosuccinimide (145 mg, 1.1 mmol) were dissolved in acetonitrile (8 mL), tetrabutylammonium fluoride (1.1 mL, 1.1 mmol, 1M) was added at 0° C., and the reaction was carried out at room temperature for 30 min, the reaction solution was concentrated, and then acetonitrile (8 mL) and tetrahydropyridine (280 mg, 4.0 mmol) were added, and reacted at room temperature for 2 h. The reaction solution was concentrated and dried, and the crude product was purified by column chromatography (PE/EtOAc=0-20%) to obtain compound 47c (124 mg), yellow oily liquid MS (ESI) m/z 374.2 [M+H]⁺.

### Step 3 1-(N-(2-fluoro-4-(4,4,5,5-tetramethyl-1,3,2-dioxaborolan-2-yl) phenyl)-S-(trifluoromethyl) sulfonylimide) pyrrolidine 47d

Compound 47c (124 mg, 0.33 mmol), bis(pinacolato)diboron (100 mg, 0.4 mmol), Pd(dppf)Cl₂ (48 mg, 0.066 mmol), and potassium acetate (98 mg, 0.99 mmol) were placed in a bottle, 1,4-dioxane (3 mL) was added, after replaced with N₂ three times, the reaction was carried out at 100° C. for 18 h. The reaction solution was filtered and concentrated, and the crude product was purified by column chromatography (PE/EtOAc=0-20%) to obtain compound 47d (70 mg).

MS (ESI) m/z 423 [M+H]⁺.

### Step 4 N-(4-(4-amino-5-(3-fluoro-4-((oxo (pyrrolidine-1-yl) (trifluoromethyl)-λ⁶ sulfanylidene) amino) phenyl)-7-methyl-7H-pyrrolo [2,3-d] pyrimidin-6-yl) phenyl) methacrylamide 47

Compound 9e (25 mg, 0.065 mmol), compound 47d (30 mg, 0.07 mmol), Pd(PPh₃)₄ (15 mg, 0.013 mmol), and K₃PO₄ (42 mg, 0.2 mmol) were added into a sealed tube, after replaced with argon gas three times, 1,4-dioxane (5 mL) and H₂O (0.5 mL) were added, sealed, and the reaction solution was heated up to 100° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/30) to obtain compound 47 (10 mg), white solid.

MS (ESI) m/z 602.3 [M+H]⁺.

¹H NMR (400 MHz, MeOD-d4) δ 8.19 (s, 1H), 7.71-7.59 (m, 2H), 7.31-7.21 (m, 2H), 7.15 (t, J=8.4 Hz, 1H), 7.03-6.94 (m, 2H), 5.81 (s, 1H), 5.52 (d, J=1.8 Hz, 1H), 3.68 (s, 3H), 3.55 (d, J=7.5 Hz, 2H), 3.43 (d, J=8.6 Hz, 2H), 2.05-1.91 (m, 6H), 1.31 (d, J=17.8 Hz, 2H).

### Example 48 N-(4-(4-amino-5-(4-(((dimethylamino) (oxo) (trifluoromethyl)-λ⁶-sulfanylidene) amino)-3-fluorophenyl)-7-methyl-7H-pyrrolo [2,3)-d]pyrimidin-6-yl) phenyl) methacrylamide

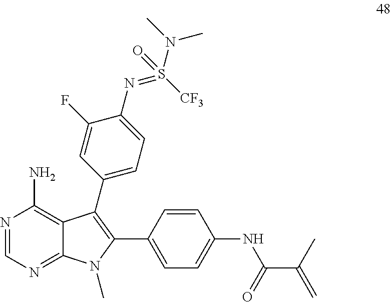 [SMILES: C=C(C)C(=O)Nc1ccc(-c2c(-c3ccc(N=S(=O)(N(C)C)C(F)(F)F)c(F)c3)c3c(N)ncnc3n2C)cc1]

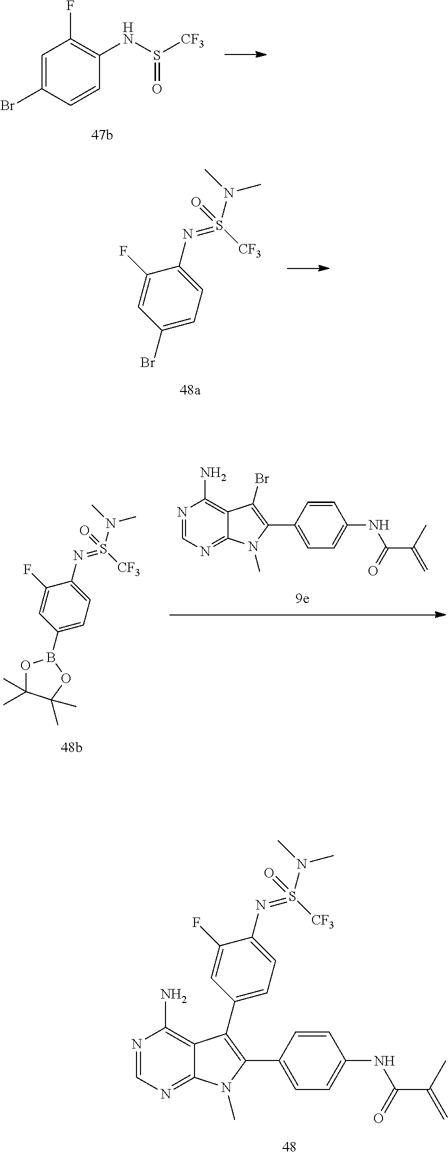

### Step 1 N′-(4-bromo-2-fluorophenyl)-1,1,1-trifluoro-N,N-dimethylmethanesulfonimide 48a

Compound 47b (500 mg, 1.6 mmol) and N-chlorosuccinimide (235 mg, 1.76 mmol) were dissolved in acetonitrile (8 mL), tetrabutylammonium fluoride (1.76 mL, 1.76 mmol, 1M) was added at 0° C., and the reaction was carried out at room temperature for 30 min, the reaction solution was concentrated, and then acetonitrile (8 mL) and dimethylamine (3.5 mL, 7.0 mmol, 2M in THF) were added, and reacted at room temperature for 2 h. The reaction solution was concentrated and dried, and the crude product was purified by Flash (PE/EtOAc=0-20%) to obtain compound 48a (400 mg), yellow oily liquid MS (ESI) m/z 349.2 [M+H]⁺.

### Step 2 1,1,1-trifluoro-N′-(2-fluoro-4-(4,4,5,5-tetramethyl-1,3,2-dioxaborolan-2-yl) phenyl)-N,N-dimethylsulfonamide 48b

Compound 48a (100 mg, 0.29 mmol), bis(pinacolato)diboron (87 mg, 0.34 mmol), Pd(dppf)Cl₂ (42 mg, 0.058 mmol), and potassium acetate (85 mg, 0.87 mmol) were placed in a bottle, 1,4-dioxane (5 mL) was added, after replaced with N₂ three times, the reaction was carried out at 100° C. for 18 h. The reaction solution was filtered and concentrated, and the crude product was purified by column chromatography (PE/EtOAc=0-20%) to obtain compound 48b (90 mg), yellow oily liquid.

MS (ESI) m/z 397 [M+H]⁺.

### Step 3 N-(4-(4-amino-5-(4-((dimethylamino) (oxo) (trifluoromethyl)-λ⁶-sulfanylidene) amino)-3-fluorophenyl)-7-methyl-7H-pyrrolo [2,3)-d]pyrimidin-6-yl) phenyl) methacrylamide 48

Compound 9e (65 mg, 0.17 mmol), compound 48b (80 mg, 0.20 mmol), Pd(PPh₃)₄ (38 mg, 0.034 mmol), and K₃PO₄ (110 mg, 0.51 mmol) were added into a sealed tube, after replaced with argon gas three times, 1,4-dioxane (5 mL) and H₂O (0.5 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/30) to obtain compound 48 (30 mg), white solid.

MS (ESI) m/z 576.54 [M+H]⁺.

¹H NMR (400 MHz, MeOD-d4) δ 8.09 (s, 1H), 7.60-7.55 (m, 2H), 7.18 (d, J=8.7 Hz, 2H), 7.05 (t, J=8.4 Hz, 1H), 6.95-6.83 (m, 2H), 5.71 (s, 1H), 5.42 (d, J=1.8 Hz, 1H), 4.45 (s, 1H), 3.58 (s, 2H), 2.97 (s, 5H), 1.93 (d, J=1.3 Hz, 2H), 1.19 (s, 3H).

### Example 49 N-(4-(4-amino-5-(4-(cyclopentyl (methyl) (oxo)-λ⁶-sulfanylidene) amino) phenyl)-7-methyl-7H-pyrrole [2,3-d] pyrimidin-6-yl)-3-fluorophenyl) methacrylamide

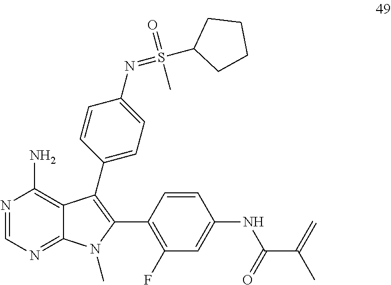 [SMILES: C=C(C)C(=O)Nc1ccc(-c2c(-c3ccc(N=S(C)(=O)C4CCCC4)cc3)c3c(N)ncnc3n2C)c(F)c1]

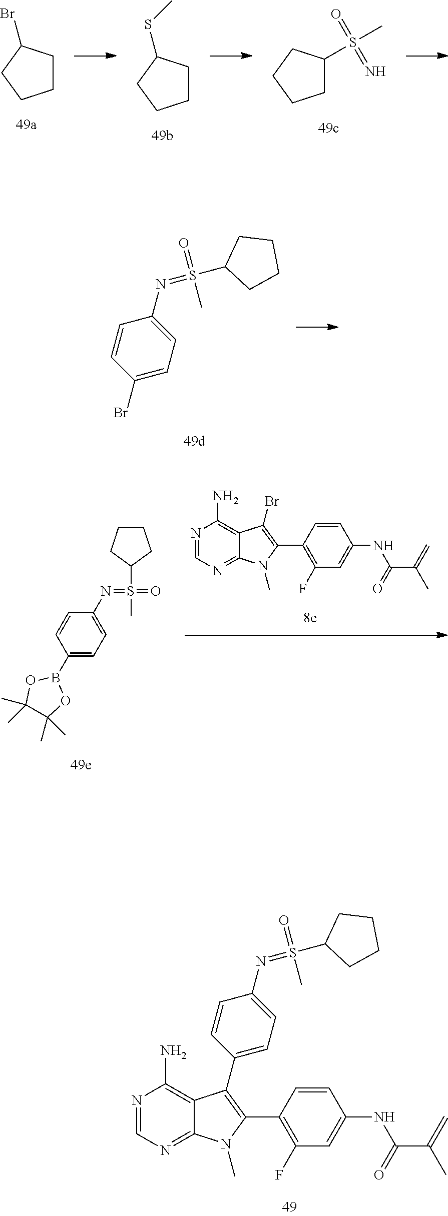

### Step 1 cyclopentyl-1-thioethane 49b

Compound 49a (1.4 g, 9.4 mmol) was dissolved in ethanol (20 mL), sodium methylthionate aqueous solution (4.9 g, 14.0 mmol) was added at room temperature sequentially to react at room temperature for 16 h. The reaction solution was cooled down to room temperature and extracted with ethyl acetate (50 mL). The organic phase was concentrated under reduced pressure to obtain crude product 49b, which was directly used in the next step.

### Step 2 cyclopentylmethylsulfoximine 49c

Compound 49b (0.9 g, 9.4 mmol) was dissolved in 20 mL of ethanol, ammonium formate (1.46 g, 15.0 mmol) and iodobenzene diethyl ester (7.5 g, 24 mmol) were added in an ice bath sequentially, the reaction was carried out at room temperature for 3 h, after TLC monitored the completion of the reaction, it was cooled down to room temperature, and the crude product was purified by column chromatography (DCM/MeOH=10/1) to obtain compound 49c (100 mg), brown oily substance.

MS (ESI) m/z 148 [M+H]⁺.

¹H NMR (400 MHz, CDCl₃) δ 3.52 (t, J=8.13 Hz, 1H), 2.85-2.99 (m, 3H), 1.94-2.15 (m, 4H), 1.73-1.87 (m, 2H), 1.59-1.73 (m, 2H).

### Step 3 4-bromophenyl-cyclopentylmethylsulfoximine 49d

To a 50 mL three necked bottle, 4-bromoiodobenzene (210 mg, 0.75 mmol) was dissolved in degassed 1,4-dioxane (3 mL), and then compound 49c (100 mg, 0.68 mmol), Pd₂(dba)₃ (60 mg, 0.06 mmol), Xantphos (68 mg, 0.12 mmol), and cesium carbonate (442 mg, 1.36 mmol) were added, the reaction was heated up to 90° C. and stirred for 4 h. After the reaction was completed, it was cooled down to room temperature, ethyl acetate (20 mL) and saturated sodium bicarbonate solution (10 mL) were added and extracted with ethyl acetate (20 mL×3), and the organic phases were combined, washed with saturated saline solution (50 mL), and concentrated under reduced pressure to obtain a crude product, and the crude product was purified by column chromatography (PE/EtOAc=3/1) to obtain compound 49d (150 mg). MS (ESI) m/z 302 [M+H]⁺.

### Step 4 cyclopentyl (methyl) (4-(4,4,4,5,5-tetramethyl-1,3,2-dioxaborolan-2-yl) phenyl) imino)-λ⁶-sulfone 49e

In a 50 mL three necked bottle, compound 49d (150 mg, 0.5 mmol) was dissolved in degassed 1,4-dioxane (5 mL), and then bis(pinacolato)diboron (152 mg, 0.6 mmol), KOAc (150 mg, 1.5 mmol), and Pd(dppf)Cl₂ (50 mg, 0.05 mmol) were added, the reaction was heated up to 90° C. and stirred for 3 h, after the reaction was completed, it was cooled down to room temperature, ethyl acetate (20 mL) and saturated sodium chloride solution (10 mL) were added and extracted with ethyl acetate (20 mL×3), and the organic phases were combined, washed with saturated saline solution (50 mL), and concentrated under reduced pressure to obtain a crude product, and the crude product was purified by column chromatography (PE/EtOAc=2/1) to obtain compound 49e (50 mg).

MS (ESI) m/z 350 [M+H]⁺.

### Step 5 N-(4-(4-amino-5-(4-(cyclopentyl (methyl) (oxo)-λ⁶-sulfanylidene) amino) phenyl)-7-methyl-7H-pyrrole [2,3-d] pyrimidin-6-yl)-3-fluorophenyl) methacrylamide 49

In a 50 mL three necked bottle, compound 49e (50 mg, 0.14 mmol) was dissolved in degassed DMF (2 mL) and water (0.2 mL), and then, compound 8e (111 mg, 0.27 mmol), K₃PO₄ (176 mg, 0.83 mmol), and Pd(dppf)Cl₂ (30 mg, 0.03 mmol) were added, the reaction was heated up to 90° C. and stirred for 3 h, after the reaction was completed, it was cooled down to room temperature, ethyl acetate (20 mL) and saturated sodium chloride solution (10 mL) were added and extracted with ethyl acetate (20 mL×3), and the organic phases were combined, washed with saturated saline solution (50 mL), and concentrated under reduced pressure to obtain a crude product, and the crude product was purified by column chromatography (DCM/MeOH=10/1) to obtain compound 49 (7.7 mg), white solid.

MS (ESI) m/z 533 [M+H]⁺.

¹H NMR (400 MHz, CDCl₃) δ 8.32 (s, 1H), 7.66-7.76 (m, 2H), 7.59 (s, 1H), 6.97-7.13 (m, 5H), 5.81 (s, 1H), 5.53 (s, 1H), 5.31 (s, 2H), 3.64-3.69 (m, 3H), 2.99 (s, 3H), 2.18-2.28 (m, 3H), 2.14 (br. s., 3H), 2.06-2.12 (m, 3H), 2.01 (br. s., 3H).

### Example 50 N-(4-(4-amino-5-(4-((cyclobutyl (methyl) (oxo)-λ⁶-sulfanylidene) amino) phenyl)-7-methyl-7H-pyrrole [2,3-d] pyrimidin-6-yl)-3-fluorophenyl) methacrylamide

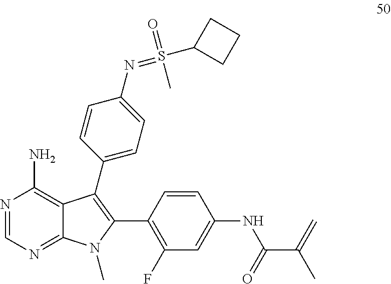 [SMILES: C=C(C)C(=O)Nc1ccc(-c2c(-c3ccc(N=S(C)(=O)C4CCC4)cc3)c3c(N)ncnc3n2C)c(F)c1]

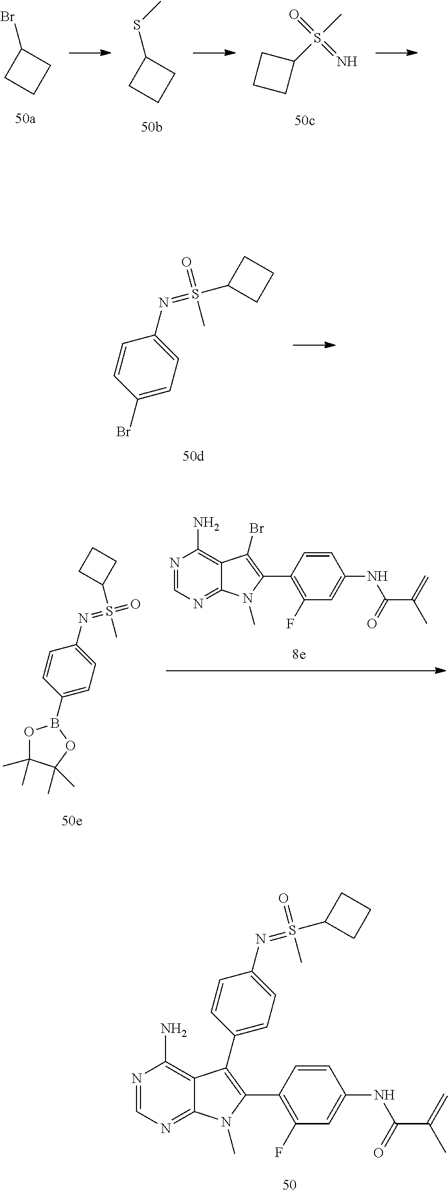

### Step 1 cyclobutyl methyl sulfide 50b

Compound 50a (1.26 g, 9.4 mmol) was dissolved in ethanol (20 mL), sodium methylthionate aqueous solution (4.9 g, 14.0 mmol) was added at room temperature sequentially, and then the reaction was carried out at room temperature for 16 h. The reaction solution was cooled down to room temperature and extracted with 50 mL of ethyl acetate. The extracted mixture was concentrated under reduced pressure to obtain crude product 50b, which was directly used in the next step.

### Step 2 cyclobutylmethylsulfonimide 50c

Compound 50b (0.9 g, 9.4 mmol) was dissolved in 20 mL of ethanol, ammonium formate (1.46 g, 15.0 mmol) was added sequentially, in ice bath, iodobenzene diacetate (7.5 g, 24 mmol) was added, and the reaction was carried out at room temperature for 3 h, after TLC monitored the completion of the reaction, it was cooled down to room temperature, concentrated under reduced pressure, and the crude product was purified by column chromatography (DCM/MeOH=10/1) to obtain compound 50c (75 mg), brown oily substance.

MS (ESI) m/z 134 [M+H]⁺.

¹H NMR (400 MHz, CDCl₃) δ 3.90 (t, J=8.33 Hz, 1H), 2.86 (s, 3H), 2.45-2.63 (m, 2H), 2.32 (dtd, J=12.22, 8.23, 8.23, 4.23 Hz, 2H), 1.95-2.13 (m, 3H).

### Step 3 4-bromophenyl-cyclobutylmethylsulfonimide 50d

To a 50 mL three necked bottle, 4-bromoiodobenzene (100 mg, 0.33 mmol) was dissolved in degassed 1,4-dioxane (3 mL), and then compound 50c (40 mg, 0.3 mmol), Pd₂(dba)₃ (30 mg, 0.03 mmol), Xantphos (34 mg, 0.06 mmol), cesium carbonate (295 mg, 0.9 mmol) were added, heated up to 90° C. and stirred for 4 h. After the reaction was completed, it was cooled down to room temperature, ethyl acetate (20 mL) and saturated sodium bicarbonate solution (10 mL) were added and extracted with ethyl acetate (20 mL×3), and the organic phases were combined, washed with saturated saline solution (50 mL), and concentrated under reduced pressure to obtain a crude product, and the crude product was purified by column chromatography (PE/EtOAc=3/1) to obtain compound 50d (75 mg).

MS (ESI) m/z 288 [M+H]⁺.

### Step 4 cyclobutyl (methyl) (4-(4,4,4,5,5-tetramethyl-1,3,2-dioxaborolan-2-yl) phenyl) imino)-λ⁶-thianone 50e

To a 50 mL three necked bottle, compound 50d (75 mg, 0.26 mmol) was dissolved in degassed 1,4-dioxane (5 mL), and then bis(pinacolato)diboron ester (75 mg, 0.3 mmol), KOAc (75 mg, 0.6 mmol), and Pd(dppf)Cl₂ (22 mg, 0.03 mmol) were added, the reaction was heated up to 90° C. and stirred for 3 h, after the reaction was completed, it was cooled down to room temperature, ethyl acetate (20 mL) and saturated sodium chloride solution (10 mL) were added and extracted with ethyl acetate (20 mL×3), and the organic phases were combined, washed with saturated saline solution (50 mL), and concentrated under reduced pressure to obtain a crude product, and the crude product was purified by column chromatography (PE/EtOAc=2/1) to obtain compound 50e (93 mg).

MS (ESI) m/z 335 [M+H]⁺.

### Step 5 N-(4-(4-amino-5-(4-((cyclobutyl (methyl) (oxo)-λ⁶-sulfanylidene) amino) phenyl)-7-methyl-7H-pyrrole [2,3-d] pyrimidin-6-yl)-3-fluorophenyl) methacrylamide 50

In a 50 mL three necked bottle, compound 50e (93 mg, 0.27 mmol) was dissolved in degassed DMF (3 mL) and water (0.3 mL), and then compound 8e (111 mg, 0.27 mmol), K₃PO₄ (176 mg, 0.83 mmol), and Pd(dppf)Cl₂ (30 mg, 0.03 mmol) were added, the reaction was heated up to 90° C. and stirred for 3 h, after the reaction was completed, it was cooled down to room temperature, ethyl acetate (20 mL) and saturated sodium chloride solution (10 mL) were added and extracted with ethyl acetate (20 mL×3), and the organic phases were combined, washed with saturated saline solution (50 mL), and concentrated under reduced pressure to obtain a crude product, and the crude product was purified by column chromatography (DCM/MeOH=10/1) to obtain compound 50 (4.5 mg), white solid.

MS (ESI) m/z 533 [M+H]⁺.

¹H NMR (400 MHz, CDCl₃) δ 8.38 (s, 1H), 7.72 (d, J=12.09 Hz, 1H), 7.59 (s, 1H), 6.98-7.20 (m, 5H), 5.83 (s, 1H), 5.54 (s, 1H), 5.06 (br. s., 2H), 4.00 (s, 1H), 3.68 (s, 3H), 2.94 (s, 3H), 2.68 (br. s., 2H), 2.35 (d, J=8.60 Hz, 2H), 1.96-2.15 (m, 5H).

### Example 51 N-(4-(4-amino-5-(4-((azetidin-1-yl (oxo) (trifluoromethyl)-λ⁶-sulfanylidene) amino) phenyl)-7-methyl-7H-pyrrolo [2,3-d] pyrimidin-6-yl) phenyl) methacrylamide

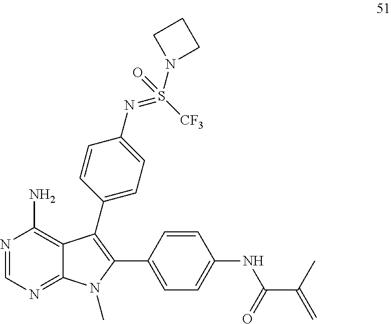 [SMILES: C=C(C)C(=O)Nc1ccc(-c2c(-c3ccc(N=S(=O)(N4CCC4)C(F)(F)F)cc3)c3c(N)ncnc3n2C)cc1]

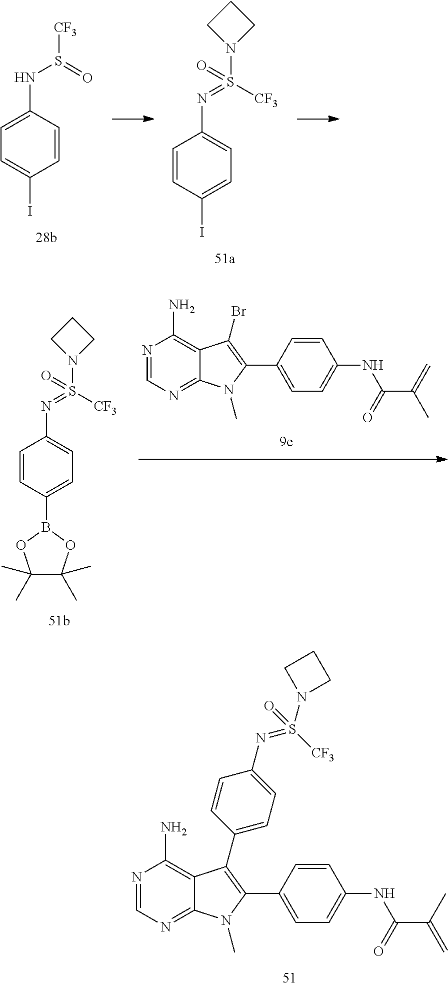

### Step 1 1-(N-(4-iodobenzene)-S-(trifluoromethyl) sulfonylimide) azetidine 51a

Compound 28b (500 mg, 1.49 mmol) and N-chlorosuccinimide (292 mg, 1.64 mmol) were dissolved in dry acetonitrile (8 mL), a solution of tetrabutylammonium fluoride in tetrahydrofuran (1.64 mL, 1.64 mmol, 1M) was added at 0° C., the reaction was carried out at room temperature for 30 min, the reaction solution was concentrated, and then dry acetonitrile (8 mL) and heterocyclic cyclohexane (340 mg, 6.0 mmol) were added, and reacted at room temperature for 2 h. The reaction solution was concentrated and dried, and the obtained crude product was purified by Flash (PE/EtOAc=0-20%) to obtain compound 51a (230 mg), yellow oily liquid.

MS (ESI) m/z 390 [M+H]⁺.

### Step 2 1-(N-(4-(4,4,5,5-tetramethyl-1,3,2-dioxaborolan-2-yl) phenyl)-S-(trifluoromethyl) sulfonylimide) azetidine 51b

Compound 51a (230 mg, 0.59 mmol), bis(pinacolato)diboron (180 mg, 0.71 mmol), Pd(dppf)Cl₂ (87 mg, 0.12 mmol), and potassium acetate (154 mg, 1.77 mmol) were placed in a bottle, 1,4-dioxane (6 mL) was added, after replaced with N₂ three times, the reaction was carried out at 100° C. for 18 h. The reaction solution was filtered and concentrated, and the crude product was purified by column chromatography (PE/EtOAc=0-20%) to obtain compound 51b (180 mg), yellow solid.

MS (ESI) m/z 391 [M+H]⁺.

### Step 3 N-(4-(4-amino-5-(4-((azetidine-1-yl-(oxo) (trifluoromethyl)-λ⁶-sulfanylidene) amino) phenyl)-7-methyl-7H-pyrrolo [2,3-d] pyrimidin-6-yl) phenyl) methacrylamide 51

Compound 9e (42 mg, 0.11 mmol), compound 51b (50 mg, 0.13 mmol), Pd(PPh₃)₄ (25 mg, 0.021 mmol), and K₃PO₄ (68 mg, 0.32 mmol) were added into a sealed tube, and it was replace with argon gas three times, 1,4-dioxane (5 mL) and H₂O (0.5 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/30) to obtain compound 51 (11 mg), white solid.

MS (ESI) m/z 570.05 [M+H]⁺.

¹H NMR (400 MHz, MeOD-d4) δ 8.19 (s, 1H), 7.72-7.59 (m, 2H), 7.39 (d, J 8.5 Hz, 2H), 7.33 (d, J=8.5 Hz, 2H), 7.30-7.21 (m, 2H), 5.80 (s, 1H), 5.52 (d, J=1.8 Hz, 1H), 4.58 (s, 1H), 4.28-4.19 (m, 1H), 3.86-3.72 (m, 2H), 3.68 (s, 4H), 2.30-2.18 (m, 1H), 2.07-1.93 (m, 4H).

### Example 52 N-(4-(4-amino-5-(4-(((dimethylamino) (oxo) (trifluoromethyl)-λ⁶-sulfanylidene) amino)-2-fluorophenyl)-7-methyl-7H-pyrrolo [2,3-d] pyrimidin-6-yl) phenyl) methacrylamide

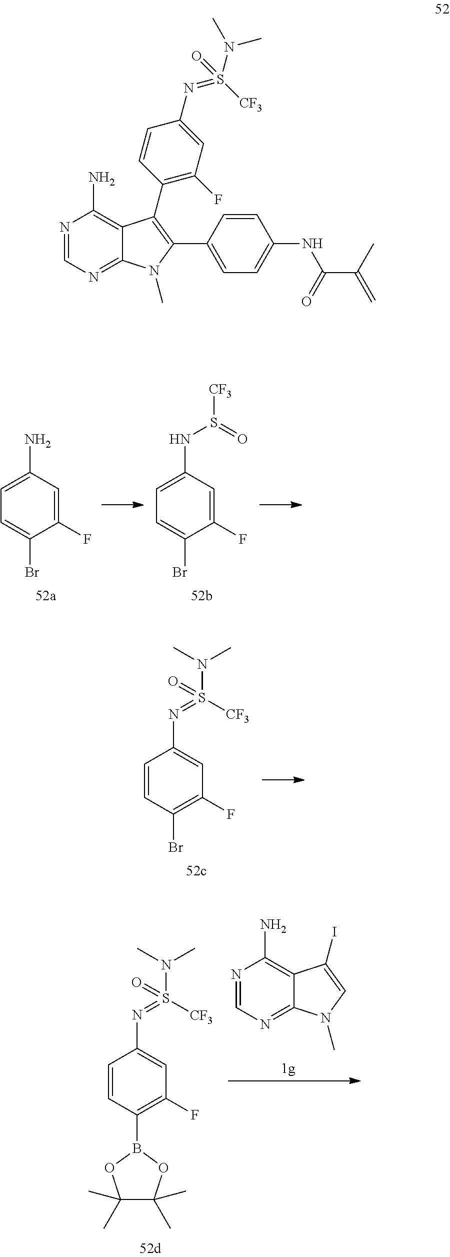

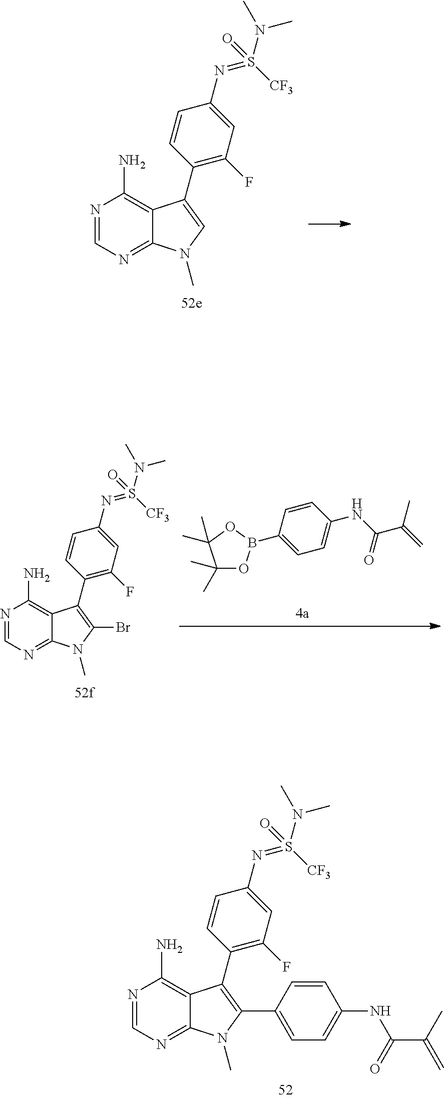

### Step 1 N-(4-bromo-3-fluorophenyl)-1,1,1-trifluoromethylsulfonimide 52b

Trifluoromethanesulfonyl chloride (2.41 g, 15.79 mmol) was dissolved in dichloromethane (15 mL), cooled down to 0° C., a solution of compound 52a (3.0 g, 15.79 mmol) in dichloromethane (10 mL) was added dropwise, and N,N-diisopropylethylamine (2.04 g, 15.79 mmol) was added, then heated up to room temperature and reacted for 2 h. The reaction solution was poured into water and extracted with dichloromethane three times, dried over anhydrous sodium sulfate, and concentrated to obtain a crude product, and the crude product was purified by column chromatography (PE/EtOAc=0-3%) to obtain compound 52b (2.6 g), brown solid.

MS (ESI) m/z 306.56 [M+H]⁺.

### Step 2 N′-(4-bromo-3-fluorophenyl)-1,1,1-trifluoro-N,N-dimethylmethanesulfonimide 52c

Compound 52b (600 mg, 1.98 mmol) and N-chlorosuccinimide (318 mg, 2.18 mmol) were dissolved in dry acetonitrile (10 mL), at 0° C., a solution of tetrabutylammonium fluoride in tetrahydrofuran (2.2 mL, 2.2 mmol, 1M) was added, and the reaction was carried out at room temperature for 30 min. The reaction solution was concentrated, and then dry acetonitrile (10 mL) and dimethylamine tetrahydrofuran solution (4.0 mL, 8.0 mmol, 2M in THF) were added, and reacted at room temperature for 2 h. The reaction solution was concentrated, dried, and purified by column chromatography (PE/EtOAc=0-20%) to obtain compound 52c (480 mg), yellow oily liquid MS (ESI) m/z 349.2 [M+H]⁺.

### Step 3 1,1,1-trifluoro-N′-(3-fluoro-4-(4,4,5,5-tetramethyl-1,3,2-dioxaborolan-2-yl) phenyl)-N,N-dimethylsulfonylimide 52d

Compound 52c (300 mg, 0.87 mmol), bis(pinacolato)diboron (261 mg, 1.02 mmol), Pd(dppf)Cl₂ (126 mg, 0.174 mmol), and potassium acetate (255 mg, 2.61 mmol) were placed into a bottle, 1,4-dioxane (mL) was added, after replaced with N₂ three times, the reaction was carried out at 100° C. for 18 h. The reaction solution was filtered and concentrated, and the crude product was purified by column chromatography (PE/EtOAc=0-20%) to obtain compound 52d (280 mg), yellow oily liquid.

MS (ESI) m/z 397 [M+H]⁺.

### Step 4 N′-(4-(4-amino-7-methyl-7H-pyrrolo[2,3-d] pyrimidin-5-yl)-3-fluorophenyl)-1,1,1-trifluoro-N,N-dimethylmethanesulfonimide 52e

Compound 1g (115 mg, 0.42 mmol), compound 52d (200 mg, 0.5 mmol), Pd(PPh₃)₄ (97 mg, 0.084 mmol), and K₃PO₄ (267 mg, 1.26 mmol) were added into a sealed tube, after replaced with N₂ three times, 1,4-dioxane (4 mL) and H₂O (0.5 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/30) to obtain compound 52e (85 mg), yellow solid.

MS (ESI) m/z 417 [M+H]⁺.

### Step 5 N′-(4-(4-amino-6-bromo-7-methyl-7H-pyrrolo [2,3-d] pyrimidin-5-yl)-3-fluorophenyl)-1,1,1-trifluoro-N,N-dimethylmethanesulfonimide 52f

Compound 52e (85 mg, 0.21 mmol) was dissolved in DMF (3 mL), and a solution of N-bromosuccinimide (41 mg, 0.23 mmol) in DMF (0.5 mL) was added dropwise at 0° C., and the reaction solution was stirred at 0° C. for 1 h. Then DCM (20 mL) was added, and washed with saturated saline solution (10 mL×3), and the organic phase was dried over anhydrous sodium sulfate, and concentrated under reduced pressure to obtain a crude product. The crude product was purified by column chromatography (MeOH/DCM=1/40) to obtain compound 52f (51 mg), yellow solid.

MS (ESI) m/z 495 [M+H]⁺.

### Step 6 N-(4-(4-amino-5-(4-(((dimethylamino) (oxo) (trifluoromethyl)-λ⁶-sulfanylidene) amino)-2-fluorophenyl)-7-methyl-7H-pyrrolo [2,3-d] pyrimidin-6-yl) phenyl) methacrylamide 52

Compound 52f (51 mg, 0.103 mmol), compound 4a (61.2 mg, 0.124 mmol), Pd(PPh₃)₄ (23 mg, 0.021 mmol), and K₃PO₄ (66 mg, 0.31 mmol) was added into a sealed tube, after replaced with N₂ three times, 1,4-dioxane (5 mL) and H₂O (0.5 mL) were added, sealed, and the reaction solution was heated up to 100° C. and stirred for 16 h. Dichloromethane (30 mL) and saturated saline solution (20 mL) were added to extract, and the organic phase was dried over anhydrous sodium sulfate, and concentrated under reduced pressure to obtain a crude product. The crude product was purified by column chromatography (MeOH/DCM=1/30) to obtain compound 52 (18 mg), white solid.

MS (ESI) m/z 576.44 [M+H]⁺.

¹H NMR (400 MHz, MeOD-d4) δ 8.18 (s, 1H), 7.64 (d, J=8.2 Hz, 2H), 7.28-7.19 (m, 3H), 6.87-6.79 (m, 2H), 5.80 (s, 1H), 5.52 (s, 1H), 4.58 (s, 2H), 3.71 (s, 3H), 3.08 (s, 5H), 2.02 (s, 3H).

### Example 53 N-(4-(4-amino-5-(4-(((ethyl (methyl) amino) (oxo) (trifluoromethyl)-λ⁶-sulfanylidene) amino)-3-fluorophenyl)-7-methyl-7H-pyrrolo [2,3-d] pyrimidin-6-yl) phenyl) methacrylamide

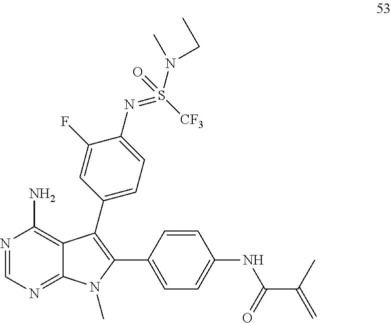 [SMILES: C=C(C)C(=O)Nc1ccc(-c2c(-c3ccc(N=S(=O)(N(C)CC)C(F)(F)F)c(F)c3)c3c(N)ncnc3n2C)cc1]

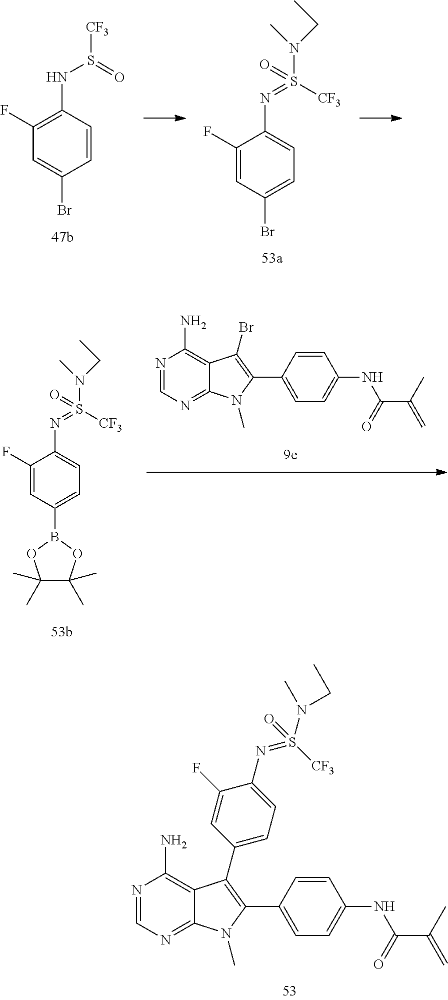 [SMILES: O=S(Nc1ccc(Br)cc1F)C(F)(F)F ; CCN(C)S(=O)(=Nc1ccc(Br)cc1F)C(F)(F)F ; CCN(C)S(=O)(=Nc1ccc(B2OC(C)(C)C(C)(C)O2)cc1F)C(F)(F)F ; C=C(C)C(=O)Nc1ccc(-c2c(Br)c3c(N)ncnc3n2C)cc1 ; C=C(C)C(=O)Nc1ccc(-c2c(-c3ccc(N=S(=O)(N(C)CC)C(F)(F)F)c(F)c3)c3c(N)ncnc3n2C)cc1]

### Step 1 N′-(4-bromo-2-fluorophenyl)-N-ethyl-1,1,1-trifluoro-N-methylsulfonylimide 53a

Compound 47b (500 mg, 1.49 mmol) and N-chlorosuccinimide (292 mg, 1.64 mmol) were added in acetonitrile (8 mL), a solution of tetrabutylammonium fluoride in THF (1.64 mL, 1.64 mmol, 1M) was added at 0° C., and the reaction was carried out at room temperature for 30 min. The reaction solution was concentrated, and then acetonitrile (8 mL) and N-methylethylamine (360 mg, 6.0 mmol) were added, and reacted at room temperature for 2 h. The reaction solution was concentrated and dried, and the obtained crude product was purified by column chromatography (PE/EtOAc=0-20%) to obtain compound 53a (420 mg).

MS (ESI) m/z 363.2 [M+H]⁺

### Step 2 N-ethyl-1,1,1-trifluoro-N′-(2-fluoro-4-(4,4,5,5-tetramethyl-1,3,2-dioxaborolan-2-yl) phenyl)-N-methylmethane sulfonamide 53b

Compound 53a (420 mg, 1.16 mmol), bis(pinacolato)diboron (352 mg, 1.39 mmol), Pd(dppf)Cl₂ (170 mg, 0.23 mmol), and potassium acetate (340 mg, 3.48 mmol) were placed in a bottle, and 1,4-dioxane (8 mL) was added, after replaced with N₂ three times The reaction was carried out at 100° C. for 18 h. The reaction solution was filtered and concentrated, and the crude product was purified by column chromatography (PE/EtOAc=0-20%) to obtain compound 53b (360 mg).

MS (ESI) m/z 411 [M+H]⁺

### Step 3 N-(4-(4-amino-5-(4-(((ethyl (methyl) amino) (oxo) (trifluoromethyl)-λ⁶-sulfanylidene) amino)-3-fluorophenyl)-7-methyl-7H-pyrrolo [2,3-d] pyrimidin-6-yl) phenyl) methacrylamide 53

Compound 9e (117 mg, 0.305 mmol), compound 53b (150 mg, 0.366 mmol), Pd(PPh₃)₄ (70 mg, 0.061 mmol), and K₃PO₄ (200 mg, 0.915 mmol) were added into a sealed tube, after replaced with N₂ three times, 1,4-dioxane (5 mL) and H₂O (0.5 mL) were added, sealed, and the reaction solution was heated up to 100° C. and stirred for 16 h. Dichloromethane (30 mL) and saturated saline solution (20 mL) were added to extract, and the organic phase was dried over anhydrous sodium sulfate, and concentrated under reduced pressure to obtain a crude product. The crude product was purified by column chromatography (MeOH/DCM=1/30) to obtain compound 53 (80 mg), white solid.

MS (ESI) m/z 589.90 [M+H]⁺.

¹H NMR (400 MHz, MeOD-d4) δ 8.32 (s, 1H), 7.78-7.52 (m, 2H), 7.26 (dd, J 8.4, 3.5 Hz, 2H), 7.21-7.10 (m, 1H), 7.05-6.90 (m, 2H), 5.81 (s, 1H), 5.52 (s, 1H), 4.65 (s, 3H), 4.56 (s, 1H), 3.68 (d, J=3.3 Hz, 3H), 3.47 (p, J=7.3 Hz, 2H), 3.03 (s, 3H), 1.11 (td, J=7.1, 1.9 Hz, 3H).

### Example 54 N-(4-(4-amino-5-(4-(((ethyl (methyl) amino) (oxo) (trifluoromethyl)-λ⁶-sulfanylidene) amino) phenyl)-7-methyl-7H-pyrrolo [2,3-d] pyrimidin-6-yl) phenyl) methacrylamide

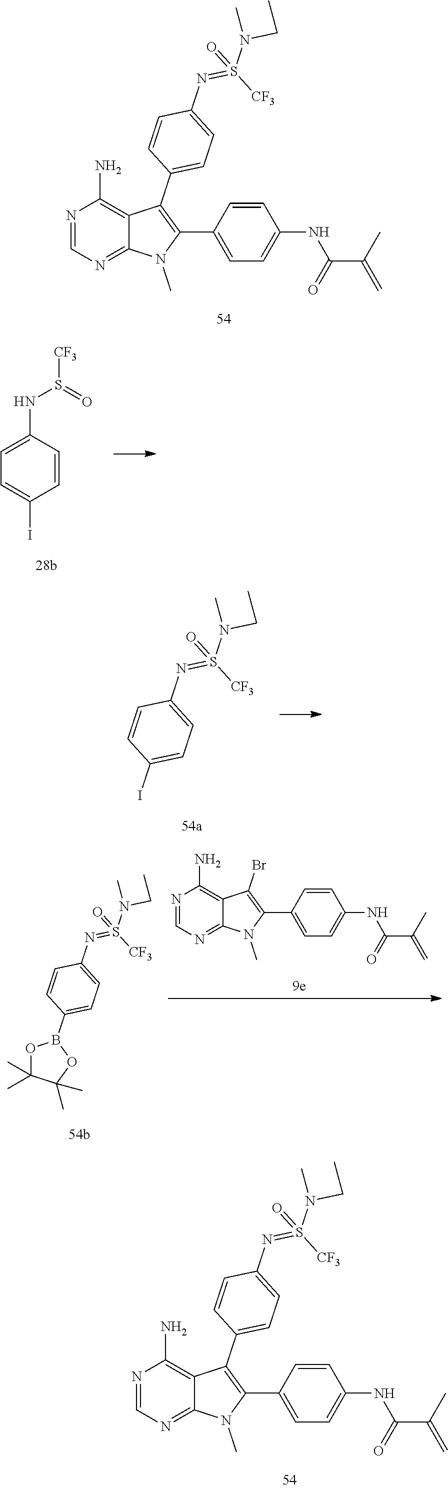

### Step 1 N-ethyl-1,1,1-trifluoro-N′-(4-iodophenyl)-N-methylmethylsulfonyl imide 54a

Compound 28b (500 mg, 1.49 mmol) and N-chlorosuccinimide (292 mg, 1.64 mmol) were added in acetonitrile (8 mL), a solution of tetrabutylammonium fluoride in THF (1.64 mL, 1.64 mmol, 1M) was added at 0° C., and the reaction was carried out at room temperature for 30 min. The reaction solution was concentrated, then acetonitrile (8 mL) and N-methylethylamine (360 mg, 5.96 mmol) were added, and reacted at room temperature for 2 h. The reaction solution was concentrated and dried, and the crude product was purified by column chromatography (PE/EtOAc=0-20%) to obtain compound 54a (420 mg).

MS (ESI) m/z 391.97 [M+H]⁺

### Step 2 N-ethyl-1,1,1-trifluoro-N-methyl-N′-(4-(4,4,5,5-tetramethyl-1,3,2-dioxaborolan-2-yl) phenyl) methane sulfonamide 54b

Compound 54a (220 mg, 0.56 mmol), bis(pinacolato)diboron (171 mg, 0.67 mmol), Pd(dppf)Cl₂ (83 mg, 0.11 mmol), and potassium acetate (165 mg, 1.68 mmol) were placed in a bottle, and dioxane (6 mL) was added. After replaced with N₂ three times, the reaction was carried out at 100° C. for 18 h. The reaction solution was filtered and concentrated, and the crude product was purified by column chromatography (PE/EtOAc=0-20%) to obtain compound 54b (180 mg).

MS (ESI) m/z 393 [M+H]⁺.

### Step 3 N-(4-(4-amino-5-(4-(((ethyl (methyl) amino) (oxo) (trifluoromethyl)-λ⁶-sulfanylidene) amino) phenyl)-7-methyl-7H-pyrrolo [2,3-d] pyrimidin-6-yl) phenyl) methacrylamide 54

Compound 9e (24.6 mg, 0.064 mmol), compound 54b (30 mg, 0.077 mmol), Pd(PPh₃)₄ (14.7 mg, 0.013 mmol), and K₃PO₄ (41 mg, 0.192 mmol) were added into a sealed tube, after replaced with N₂ three times, 1,4-dioxane (3 mL) and H₂O (0.4 mL) were added, sealed, and the reaction solution was heated up to 100° C. and stirred for 16 h. Then dichloromethane (20 mL) and saturated saline solution (10 mL) were added to extracted, and the organic phase was dried over anhydrous sodium sulfate, and concentrated under reduced pressure to obtain a crude product. The crude product was purified by column chromatography (MeOH/DCM=1/30) to 5 obtain compound 54 (12 mg), white solid.

MS (ESI) m/z 572.68 [M+H]⁺.

¹H NMR (400 MHz, MeOD-d4) δ 8.18 (s, 1H), 7.68-7.60 (m, 2H), 7.30-7.22 (m, 2H), 7.22-7.14 (m, 2H), 7.08-7.00 (m, 2H), 5.80 (s, 1H), 5.52 (d, J=1.9 Hz, 1H), 4.57 (s, 2H), 3.69 (s, 3H), 3.47-3.41 (m, 2H), 3.01 (s, 3H), 2.02 (d, J=1.2 Hz, 3H), 1.10 (t, J=7.1 Hz, 3H).

### Example 55 N-(4-(4-amino-5-(4-((azetidine 1-yl (oxo) (trifluoromethyl)-λ⁶-sulfanylidene) amino)-3-fluorophenyl)-7-methyl-7H-pyrrolo [2,3-d] pyrimidin-6-yl) phenyl) methacrylamide

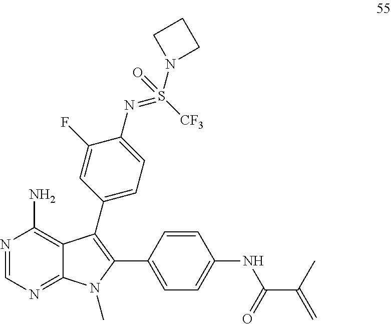 [SMILES: C=C(C)C(=O)Nc1ccc(-c2c(-c3ccc(N=S(=O)(N4CCC4)C(F)(F)F)c(F)c3)c3c(N)ncnc3n2C)cc1]

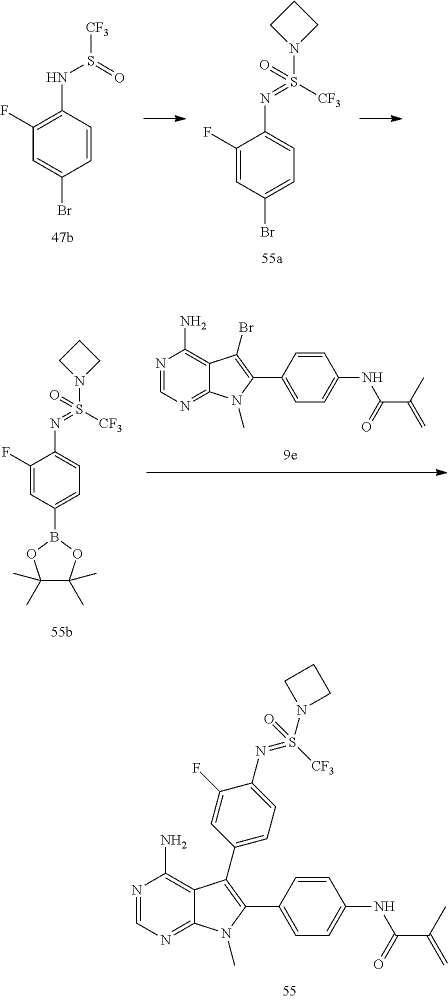 [SMILES: O=S(Nc1ccc(Br)cc1F)C(F)(F)F ; O=S(=Nc1ccc(Br)cc1F)(N1CCC1)C(F)(F)F ; CC1(C)OB(c2ccc(N=S(=O)(N3CCC3)C(F)(F)F)c(F)c2)OC1(C)C ; C=C(C)C(=O)Nc1ccc(-c2c(Br)c3c(N)ncnc3n2C)cc1 ; C=C(C)C(=O)Nc1ccc(-c2c(-c3ccc(N=S(=O)(N4CCC4)C(F)(F)F)c(F)c3)c3c(N)ncnc3n2C)cc1]

### Step 1 1-(N-(4-bromo-2-fluorophenyl)-S-(trifluoromethyl) sulfonylimino) azetidine 55a

Compound 47b (200 mg, 0.66 mmol) and N-chlorosuccinimide (106 mg, 0.73 mmol) were added in dry acetonitrile (6 mL), a solution of tetrabutylammonium fluoride in tetrahydrofuran (0.73 mL, 0.73 mmol, 1M) was added at 0° C., and the reaction was carried out at room temperature for 30 min. The reaction solution was concentrated, then dry acetonitrile (8 mL) and azetidine (128 mg, 2.6 mmol) were added, and reacted at room temperature for 2 h. The reaction solution was concentrated and dried, and the crude product was purified by column chromatography (PE/EtOAc=0-20%) to obtain compound 55a (130 mg), yellow oily liquid.

MS (ESI) m/z 361.2 [M+H]⁺.

### Step 2 1-(N-(2-bromo-4-(4,4,5,5-tetramethyl-1,3,2-dioxaborolan-2-yl) phenyl)-S-(trifluoromethyl) sulfonylimide) azetidine 55b

Compound 55a (130 mg, 0.36 mmol), bis(pinacolato)diboron (110 mg, 0.43 mmol), Pd(dppf)Cl₂ (53 mg, 0.072 mmol), and potassium acetate (110 mg, 1.1 mmol) were placed in a bottle, 1,4-dioxane (6 mL) was added, after replaced with N₂ three times, the reaction was carried out at 100° C. for 18 h. The reaction solution was filtered and concentrated, and the crude product was purified by column chromatography (PE/EtOAc=0-20%) to obtain compound 55b (100 mg).

MS (ESI) m/z 408 [M+H]⁺.

### Step 3 N-(4-(4-amino-5-(4-((azetidine 1-yl (oxo) (trifluoromethyl)-λ⁶-sulfanylidene) amino)-3-fluorophenyl)-7-methyl-7H-pyrrolo [2,3-d] pyrimidin-6-yl) phenyl) methacrylamide 55

Compound 9e (75 mg, 0.19 mmol), compound 55b (94 mg, 0.23 mmol), Pd(dppf)Cl₂ (28 mg, 0.0384 mmol), and K₃PO₄ (120 mg, 0.58 mmol) were added into a sealed tube, after replaced with N₂ three times, 1,4-dioxane (3 mL) and H₂O (0.4 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/30) to obtain compound 55 (20 mg), white solid.

MS (ESI) m/z 588.64 [M+H]⁺.

¹H NMR (400 MHz, MeOD-d₄) δ 8.19 (s, 1H), 7.67 (s, 2H), 7.28 (d, J=7.9 Hz, 3H), 7.16 (s, 1H), 7.00 (s, 2H), 5.81 (s, 1H), 5.53 (s, 1H), 4.24 (s, 2H), 4.18 (s, 2H), 3.68 (s, 3H), 2.37 (s, 2H), 2.03 (s, 3H), 1.30 (s, 1H).

### Example 56 N-(4-(4-amino-5-(3-fluoro-4-(1-oxo-1λ⁶-thiocyclobutan-1-ylidene) amino) phenyl)-7-methyl-7H-pyrrolo [2,3-d] pyrimidin-6-yl)-phenyl) methacrylamide

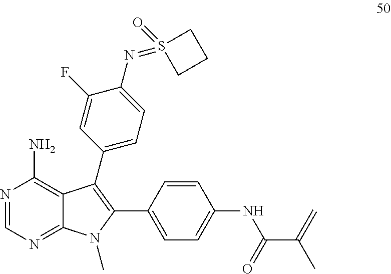 [SMILES: C=C(C)C(=O)Nc1ccc(-c2c(-c3ccc(N=S4(=O)CCC4)c(F)c3)c3c(N)ncnc3n2C)cc1]

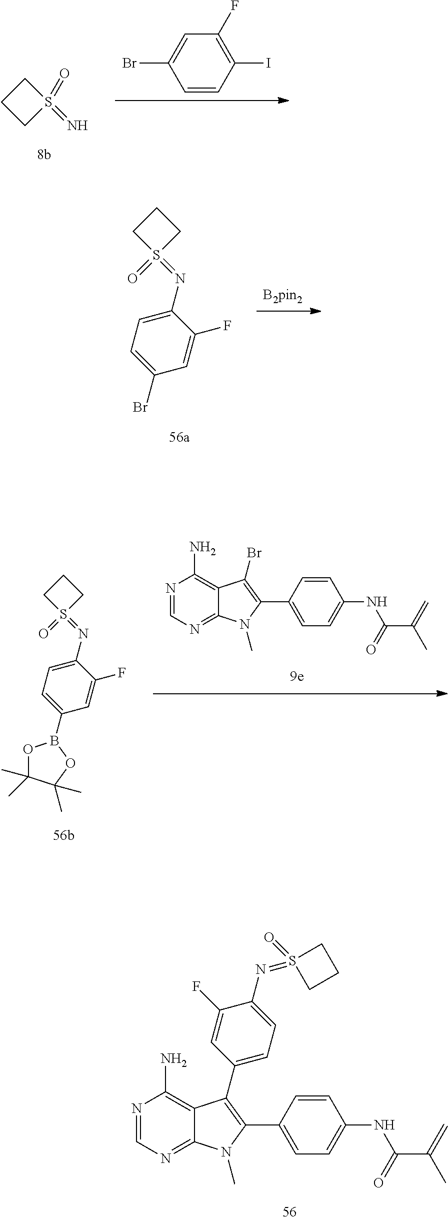 [SMILES: N=S1(=O)CCC1 ; Fc1cc(Br)ccc1I ; O=S1(=Nc2ccc(Br)cc2F)CCC1 ; CC1(C)OB(c2ccc(N=S3(=O)CCC3)c(F)c2)OC1(C)C ; C=C(C)C(=O)Nc1ccc(-c2c(Br)c3c(N)ncnc3n2C)cc1 ; C=C(C)C(=O)Nc1ccc(-c2c(-c3ccc(N=S4(=O)CCC4)c(F)c3)c3c(N)ncnc3n2C)cc1]

### Step 1 1-((4-bromo-2-fluorophenyl) imino)-1λ⁶-thietane-1-oxide 56a

Compound 8b (720 mg, 6.8 mmol), cesium carbonate (3.3 g, 10.2 mmol), 1-bromo-3-5 fluoro-4-iodobenzene (2.47 g, 8.2 mmol), Pd₂(dba)₃ (187 mg, 0.2 mmol), and Xantphos (354 mg, 0.6 mmol) were added to 1,4-dioxane (15 mL), and the reaction solution was heated up to 100° C. for 16 h. The reaction solution was filtered through diatomaceous earth, and the filter cake was washed with ethyl acetate (100 mL) and saturated saline solution (50 mL). The organic phase was dried over anhydrous sodium sulfate and concentrated under reduced pressure to obtain a crude product. The crude product was purified by column chromatography (PE/EtOAc=4/1 to 3/2) to obtain compound 56a (1.1 g), light yellow solid.

¹H NMR (400 MHz, CDCl₃) δ 7.20 (dd, J=10.0, 2.4 Hz, 1H), 7.13 (dd, J=8.4, 2.4 Hz, 1H), 6.96 (t, J=8.4 Hz, 1H), 4.31-4.19 (m, 4H), 2.32 (p, J=8.4 Hz, 2H).

### Step 2 1-((2-fluoro-4-(4,4,5,5-tetramethyl-1,3,2-dioxaborolan-2-yl) phenyl) imino)-1λ⁶-thietane-1-oxide 56b

Compound 56a (556 mg, 2.0 mmol), bis(pinacolato)diboron (660 mg, 2.6 mmol), Pd(dppf)Cl₂ (293 mg, 0.4 mmol), potassium acetate (588 mg, 6.0 mmol), and 1,4-dioxane (10 mL) were added into a sealed tube, after replaced with argon gas three times, sealed, and the reaction solution was heated up to 90° C. and stirred for 16 h. Water (10 mL) was added and extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 56b (356 mg), white solid.

¹H NMR (400 MHz, CDCl₃) δ 7.60-7.34 (m, 2H), 7.06 (t, J=8.1 Hz, 1H), 4.38-4.18 (m, 4H), 2.39-2.27 (m, 2H), 1.32 (s, 12H).

### Step 3 N-(4-(4-amino-5-(3-fluoro-4-(1-oxo-1λ⁶-thiocyclobutan-1-ylidene) amino) phenyl)-7-methyl-7H-pyrrolo [2,3-d] pyrimidin-6-yl)-phenyl) methacrylamide 56

Compound 56b (65 mg, 0.2 mmol), compound 9e (64 mg, 0.17 mmol), Pd(PPh₃)₄ (23 mg, 0.02 mmol), and K₃PO₄ (106 mg, 0.5 mmol) were added into a sealed tube, after replaced with argon gas three times, 1,4-dioxane (5 mL) and H₂O (0.5 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/10) to obtain compound 56 (12 mg), white solid.

MS (ESI) m/z 505 [M+H]⁺.

¹H NMR (400 MHz, CDCl₃) δ 8.33 (s, 1H), 7.57 (d, J=8.4 Hz, 3H), 7.21 (d, J=8.4 Hz, 2H), 7.02 (dd, J=9.2, 7.8 Hz, 1H), 6.97-6.86 (m, 2H), 5.80 (s, 1H), 5.49 (s, 1H), 5.18 (brs, 2H), 4.35-4.22 (m, 4H), 3.69 (s, 3H), 2.35 (q, J=8.4 Hz, 2H), 2.07 (s, 3H).

### Example 57 N-(4-(4-amino-5-(4-((3,3-dimethyl-1-oxo-1λ⁶-thiocyclobutyl-1-ylidene) amino)-3-fluorophenyl)-7-methyl-7H-pyrrolo [2,3-d] pyrimidin-6-yl) phenyl) methacrylamide

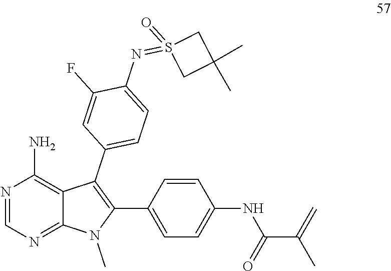 [SMILES: C=C(C)C(=O)Nc1ccc(-c2c(-c3ccc(N=S4(=O)CC(C)(C)C4)c(F)c3)c3c(N)ncnc3n2C)cc1]

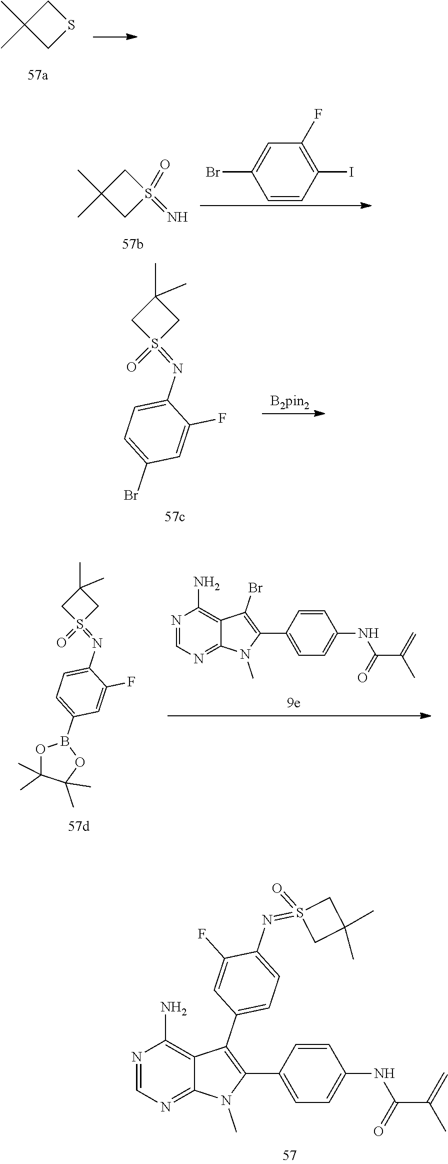

### Step 1 1-amino-3,3-dimethyl-1λ⁶-thietane-1-oxide 57b

3,3-dimethylthietane 57a (1 g, 10 mmol) and ammonium carbamate (1.17 g, 15 mmol) were added into a flask in sequence, then MeOH (100 mL) and iodobenzene diethyl ester (6.44 g, 20 mmol) were added, the reaction solution was stirred open at room temperature for 30 min, and then it was concentrated under reduced pressure to remove the solvent to obtain a crude product. The crude product was purified by column chromatography (MeOH/DCM=1/20) to obtain compound 57b (1.1 g), colorless oil.

¹H NMR (400 MHz, CDCl₃) δ 3.86 (s, 4H), 2.07 (s, 2H), 1.49 (d, J=7.3 Hz, 6H).

### Step 2 1-((4-bromo-2-fluorophenyl)imino)-3,3-dimethyl-1λ⁶-thietan-1-oxide 57c

Compound 57b (266 mg, 2 mmol), cesium carbonate (978 mg, 3 mmol), 1-bromo-3-fluoro-4-iodobenzene (722 mg, 2.4 mmol), Pd₂(dba)₃ (55 mg, 0.06 mmol), and Xantphos (104 mg, 0.18 mmol) were added to 1,4-dioxane (5 mL), and the reaction solution was heated up to 100° C. for 16 h. The reaction solution was filtered through diatomaceous earth, and the filter cake was washed with ethyl acetate (100 mL) and saturated saline solution (50 mL). The organic phase was dried over anhydrous sodium sulfate and concentrated under reduced pressure to obtain a crude product. The crude product was purified by column chromatography (PE/EtOAc=4/1 to 3/2) to obtain compound 57c (0.2 g), light yellow solid.

¹H NMR (400 MHz, CDCl₃) δ 7.20 (dd, J=10.4, 2.4 Hz, 1H), 7.13 (dd, J=8.4, 2.4 Hz, 1H), 6.97 (t, J=8.4 Hz, 1H), 4.04 (d, J=12.8 Hz, 2H), 3.99 (d, J=12.8 Hz, 2H), 1.54 (s, 3H), 1.52 (s, 3H).

### Step 3 1-((2-fluoro-4-(4,4,5,5-tetramethyl-1,3,2-dioxaborolan-2-yl) phenyl) imino)-3,3-dimethyl-1λ⁶-thietane-1-oxide 57d

Compound 57c (200 mg, 0.65 mmol), bis(pinacolato)diboron (216 mg, 0.85 mmol), Pd(dppf)Cl₂ (95 mg, 0.13 mmol), potassium acetate (191 mg, 1.95 mmol), and 1,4-dioxane (4 mL) were added into a sealed tube, after replaced with argon gas three times, sealed, and the reaction solution was heated up to 90° C. and stirred for 16 h. Water (10 mL) was added and extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 57d (190 mg), white solid.

¹H NMR (400 MHz, CDCl₃) δ 7.61-7.34 (m, 2H), 7.06 (t, J=8.1 Hz, 1H), 4.10-3.95 (m, 4H), 1.53 (s, 3H), 1.50 (s, 3H), 1.32 (s, 12H).

### Step 4 N-(4-(4-amino-5-(4-(3,3-dimethyl-1-oxo-1λ⁶-thiocyclobutyl-1-ylidene) amino)-3-fluorophenyl)-7-methyl-7H-pyrrolo [2,3-d] pyrimidin-6-yl) phenyl) methacrylamide 57

Compound 57d (190 mg, 0.53 mmol), compound 9e (164 mg, 0.42 mmol), Pd(PPh₃)₄ (59 mg, 0.05 mmol), and K₃PO₄ (267 mg, 1.26 mmol) were added into a sealed tube, after replaced with argon gas three times, 1,4-dioxane (5 mL) and H₂O (0.5 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/10) to obtain compound 57 (14 mg), white solid.

MS (ESI) m/z 533 [M+H]⁺.

¹H NMR (400 MHz, CDCl₃) δ 8.34 (s, 1H), 7.58 (d, J=8.8 Hz, 2H), 7.56 (m, 1H), 7.22 (d, J=8.8 Hz, 2H), 7.02 (t, J=8.4 Hz, 1H), 6.92 (dd, J=6.8, 2.0 Hz, 1H), 6.90 (dd, J=12.0, 2.0 Hz, 1H), 5.80 (s, 1H), 5.49 (s, 1H), 5.25 (brs, 2H), 4.09 (d, J=12.0 Hz, 2H), 4.01 (d, J=12.0 Hz, 2H), 3.70 (s, 3H), 2.07 (s, 3H), 1.54 (s, 3H), 1.53 (s, 3H).

### Example 58 N-(4-(4-amino-5-(4-(diethyl (oxo)-λ⁶-sulfanylidene) amino) phenyl)-7-methyl-7H-pyrrolo [2,3-d] pyrimidin-6-yl)-3-fluorophenyl) methacrylamide

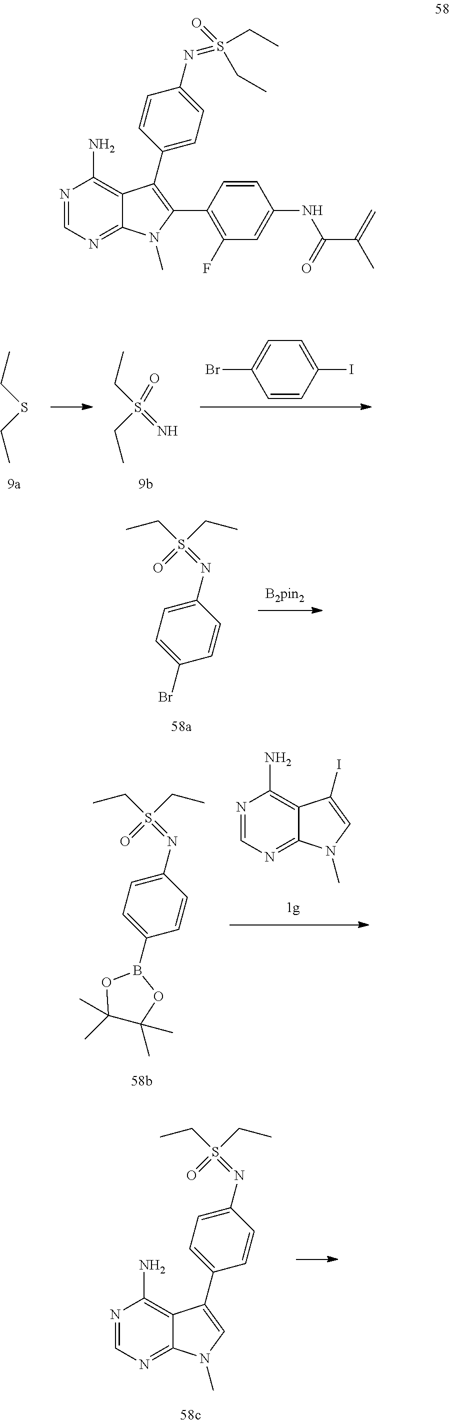

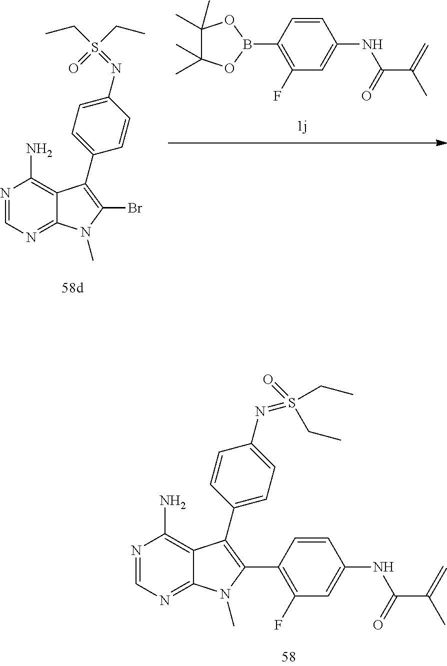

### Step 1 imine-1λ⁶-diethylsulfoxide 9b

Diethylthioether 9a (4.5 g, 50 mmol) and ammonium carbamate (5.85 g, 75 mmol) were added into a flask in sequence, then MeOH (100 mL) and iodobenzene diethyl ester (32.2 g, 100 mmol) were added. The reaction solution was stirred open at room temperature for 30 min, and then it was concentrated under reduced pressure to remove the solvent to obtain a crude product. The crude product was purified by column chromatography (MeOH/DCM=1/20) to obtain compound 9b (3.6 g), colorless oil.

¹H NMR (400 MHz, CDCl₃) δ 3.12-2.93 (q, J=7.5 Hz, 4H), 2.66 (s, 1H), 1.38 (t, J 7.5 Hz, 6H).

### Step 2 ((4-bromo-phenyl) imino)-1λ⁶-diethylsulfoxide 58a

Compound 9b (182 mg, 1.5 mmol), cesium carbonate (0.74 g, 2.25 mmol), 1-bromo-4-iodobenzene (0.5 g, 1.8 mmol), Pd₂(dba)₃ (46 mg, 0.05 mmol), and Xantphos (83 mg, 0.15 mmol) were added to 1,4-dioxane (5 mL), and the reaction solution was heated up to 100° C. for 16 h. The reaction solution was filtered through diatomaceous earth, the filter cake was washed with ethyl acetate (100 mL), and saturated saline solution (50 mL) was used for washing. The organic phase was dried over anhydrous sodium sulfate and concentrated under reduced pressure to obtain a crude product. The crude product was purified by column chromatography (PE/EtOAc=4/1 to 3/2) to obtain compound 58a (0.38 g), brownish yellow oil.

¹H NMR (400 MHz, CDCl₃) δ 7.30-7.28 (m, 2H), 7.00-6.96 (m, 2H), 3.22-3.13 (m, 4H), 1.39 (t, J=7.5 Hz, 6H).

### Step 3 1-(4-(4,4,5,5-methyl-1,3,2-dioxaborolan-2-yl)-phenyl) imino)-1λ⁶-diethylsulfoxide 58b

Compound 58a (380 mg, 1.37 mmol), bis(pinacolato)diboron (420 mg, 1.65 mmol), Pd(dppf)Cl₂ (200 mg, 0.274 mmol), potassium acetate (403 mg, 4.11 mmol), and 1,4-dioxane (10 mL) were added into a sealed tube, after replaced with argon gas three times, sealed, and the reaction solution was heated up to 90° C. and stirred for 16 h. Water (10 mL) was added and extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 58b (320 mg), white solid.

### Step 4 (4-(4-amino-7-methyl-7H-pyrrolo [2,3-d] pyrimidin-6-yl)-phenyl) imino)-1λ⁶-diethylsulfoxide 58c

Compound 58b (265 mg, 0.82 mmol), compound 1g (206 mg, 0.75 mmol), Pd(PPh₃)₄ (87 mg, 0.075 mmol), and K₃PO₄ (477 mg, 2.25 mmol) were added into a sealed tube, after replaced with argon gas three times, 1,4-dioxane (8 mL) and H₂O (0.8 mL) were added, sealed, and the reaction solution was heated up to 100° C. and stirred for 16 h. Dichloromethane (20 mL) and saturated saline solution (10 mL) were added to extract, the organic phase was dried over anhydrous sodium sulfate, and concentrated under reduced pressure to obtain a crude product. The crude product was purified by column chromatography (MeOH/DCM=1/10) to obtain compound 58c (152 mg), light yellow solid.

¹H NMR (400 MHz, CDCl₃) δ 8.33 (s, 1H), 7.31 (d, J=8.3 Hz, 2H), 7.18 (d, J=8.3 Hz, 2H), 6.88 (s, 1H), 5.11 (s, 2H), 3.83 (s, 3H), 3.24 (qd, J=7.5, 4.2 Hz, 4H), 1.44 (t, J=7.5 Hz, 6H).

### Step 5 (4-(4-amino-6-bromo-7-methyl-7H-pyrrolo [2,3-d] pyrimidin-5-yl)-phenyl) imino)-1λ⁶-diethylsulfoxide 58d

Compound 58c (70 mg, 0.2 mmol) was dissolved in anhydrous DMF (2 mL), a solution of N-bromosuccinimide (40 mg, 0.22 mmol) in DMF (2 mL) was added slowly dropwise at 0° C., and the reaction was carried out for 1 h. After the reaction was completed, the reaction was quenched with added sodium thiosulfate aqueous solution, extracted with DCM/H₂O twice, and the organic layers were combined, and washed once with saturated salt water, and the organic phase was dried over anhydrous sodium sulfate, concentrated under reduced pressure, and subjected to column chromatography (DCM/MEOH=20/1) to obtain compound 58d (39 mg), light yellow solid.

¹H NMR (400 MHz, CDCl₃) δ 8.26 (s, 1H), 7.30 (d, J=8.4 Hz, 2H), 7.20 (d, J=8.4 Hz, 2H), 5.25 (s, 2H), 3.82 (s, 3H), 3.30-3.22 (m, 4H), 1.45 (t, J 7.4 Hz, 6H).

### Step 6 N-(4-(4-amino-5-(4-(diethyl (oxo)-λ⁶-sulfanylidene) amino) phenyl)-7-methyl-7H-pyrrolo [2,3-d] pyrimidin-6-yl)-3-fluorophenyl) methacrylamide 58

Compound 58d (40 mg, 0.1 mmol), compound 1j (39 mg, 0.12 mmol), Pd(PPh₃)₄ (12 mg, 0.01 mmol), and K₃PO₄ (64 mg, 0.3 mmol) were added into a sealed tube, after replaced with argon gas three times, 1,4-dioxane (2 mL) and H₂O (0.2 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/10) to obtain compound 58 (22 mg), white solid.

MS (ESI) m/z 521 [M+H]⁺.

¹H NMR (400 MHz, CDCl₃) δ 8.35 (s, 1H), 7.69 (dd, J=11.6, 1.9 Hz, 1H), 7.61 (s, 1H), 7.10-7.00 (m, 6H), 5.80 (s, 1H), 5.51 (d, J=1.5 Hz, 1H), 5.04 (s, 2H), 3.65 (s, 3H), 3.28-3.12 (m, 4H), 2.07 (s, 3H), 1.41 (t, J=7.4 Hz, 6H).

### Example 59 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl)-3-(difluoromethyl) phenyl) methacrylamide

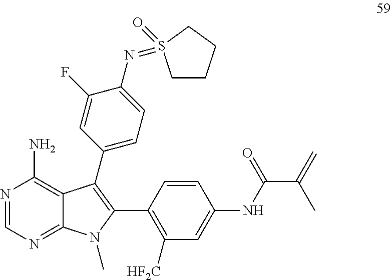

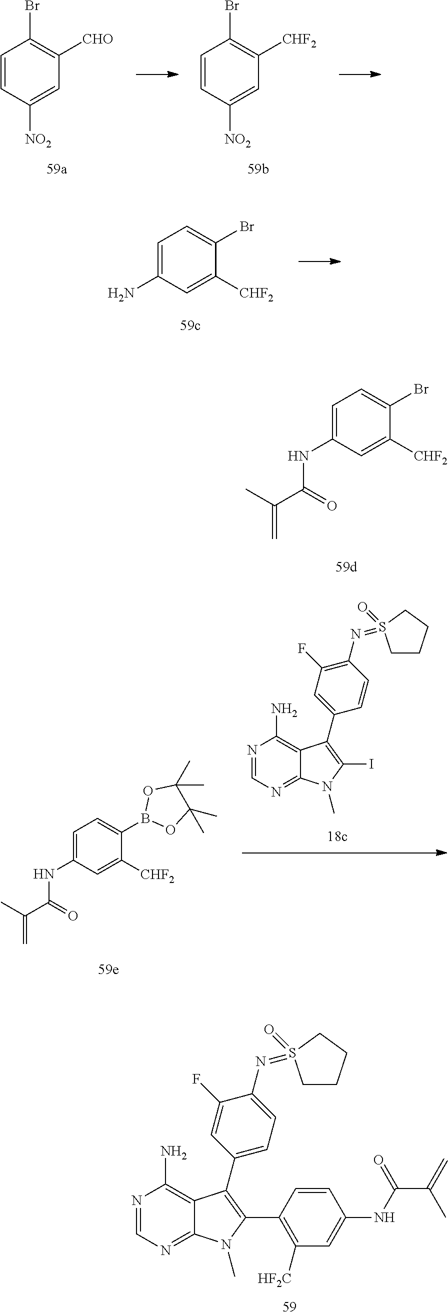

### Step 1 1-bromo-2-difluoromethyl-4-nitrobenzene 59b

To a 100 mL round bottom flask, compound 59a (2 g, 8.69 mmol), bis(2-methoxyethyl) aminotrifluoride (2.88 g, 13.02 mmol), anhydrous dichloromethane (20 mL) were added sequentially, and the reaction was carried out at room temperature for 4 h, after TLC monitored the completion of the reaction, the reaction was quenched with saturated ammonium chloride solution, extracted with dichloromethane (30 mL×3), and the organic phases were combined, washed with saturated saline solution (30 mL), and concentrated under reduced pressure to obtain a crude product. The crude product was purified by column chromatography (PE/EA=100/0) to obtain compound 59b (2.75 g), white solid.

### Step 2 4-bromo-3-difluoromethylaniline 59c

To a 100 mL round bottom flask, compound 59b (2.7 g, 10.71 mmol), reduced iron powder (3.0 g, 53.57 mmol), ammonium chloride (2.8 g, 52.35 mmol), ethanol (20 mL), and water (10 mL) were added in sequence, and the reaction was carried out at 80° C. for 4 h, after TLC monitored the completion of the reaction, it was cooled down to room temperature, filtered through diatomaceous earth, extracted with dichloromethane (30 mL×3), and the organic phases were combined, washed with saturated saline (30 mL), and concentrated under reduced pressure to obtain a crude product. The crude product was purified by column chromatography (PE/EA=3/1) to obtain compound 59c (1.02 g), white solid.

### Step 3 4-bromo-3-difluoromethylphenylmethacrylamide 59d

To a 100 mL round bottom flask, compound 59c (1.02 g, 4.59 mmol), methyl acryloyl chloride (0.458 g, 5.05 mmol), trifluoroacetic acid (0.929 g, 9.19 mmol), and dichloromethane (20 mL) were added in sequence, the reaction was carried out in an ice bath for 1 h, after TLC monitored the completion of the reaction, the reaction was quenched with saturated sodium bicarbonate solution, extracted with dichloromethane (30 mL×3), and the organic phases were combined, washed with saturated saline solution (30 mL), and concentrated under reduced pressure to obtain a crude product. The crude product was purified by column chromatography (PE/EA=1/1) to obtain compound 59d (1.01 g), white solid.

### Step 4 N-(3-(difluoromethyl)-4-(4,4,5,5-tetramethyl-1,3,2-dioxaborolan-2-yl) phenyl) methacrylamide 59e

To a 100 mL round bottom flask, compound 59d (1.01 g, 3.49 mmol), diborate pinacol ester (1.07 g, 4.19 mmol), Pd(dppf)Cl₂ (507 mg, 0.70 mmol), potassium acetate (157 mg, 1.59 mmol) and 1,4-dioxane (20 mL) were added sequentially, the reaction was heated up to 90° C. and reacted for 3 h. After TLC monitored the completion of the reaction, it was cooled down to room temperature, filtered through diatomaceous earth, extracted with ethyl acetate (30 mL×3), and the organic phases were combined, washed with saturated saline (30 mL), and concentrated under reduced pressure to obtain a crude product. The crude product was purified by column chromatography (PE/EA=1/1) to obtain compound 59e (120 mg), light yellow solid.

### Step 5 N-(4-(4-amino-5-(3-fluoro-4-((1-oxotetrahydro-1λ⁶-thiophene-1-ylidene) amino) phenyl)-7-methyl-7H-pyrrolo [2,3-d] pyrimidin-6-yl)-3-(difluoromethyl) phenyl) methacrylamide 59

To a 100 mL round bottom flask, compound 59e (0.91 g, 0.18 mmol), compound 18c (0.100 g, 0.21 mmol), and tetrakis (triphenylphosphine) palladium (20.8 mg, 0.018 mmol), anhydrous potassium phosphate (130 mg, 0.54 mmol), 1,4-dioxane (10 mL), and water (1 mL) were added in sequence, the reaction was heated up to 90° C. and reacted for 2 h. After TLC monitored the completion of the reaction, it was cooled down to room temperature, filtered through diatomaceous earth, extracted with ethyl acetate (30 mL×3), and the organic phases were combined, washed with saturated saline (30 mL), and concentrated under reduced pressure to obtain a crude product. The crude product was purified by column chromatography (DCM/MeOH=6/1) to obtain compound 59 (44 mg), white solid.

MS (ESI) m/z 568.10 [M+H]⁺.

¹H NMR (400 MHz, CDCl₃) δ 8.41 (s, 1H), 7.99 (d, J=8.0 Hz, 1H), 7.77 (d, J=5.0 Hz, 2H), 7.37 (d, J=8.3 Hz, 1H), 7.29 (s, 1H), 7.12 (t, J=8.5 Hz, 1H), 6.90 (d, J=8.0 Hz, 2H), 5.87 (s, 1H), 5.56 (s, 1H), 5.13 (s, 2H), 3.56 (s, 3H), 3.51-3.40 (m, 2H), 3.25 (s, 2H), 2.33 (d, J=7.4 Hz, 4H), 2.12 (s, 3H).

### Example 60 N-(4-(4-amino-7-methyl-5-(4-((1-oxotetrahydro-1λ⁶-thiophene-1-ylidene) amino) phenyl)-7H-pyrrolo [2,3-d] pyrimidin-6-yl) phenyl) but-2-ynamide

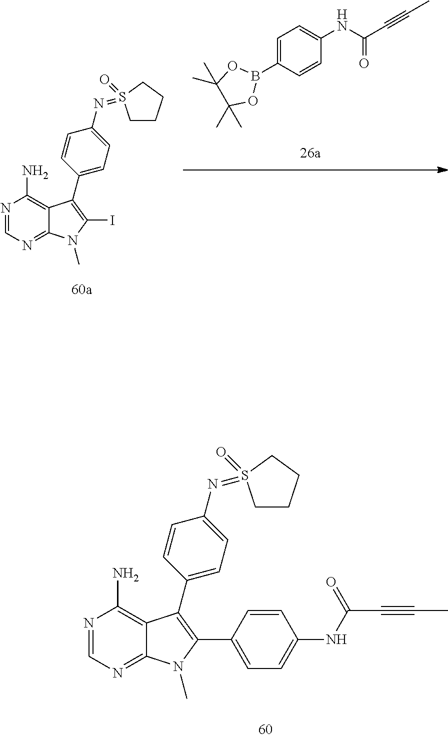

Compound 60 was synthesized with reference to Example 12

MS (ESI) m/z 499.70 [M+H]⁺.

¹H NMR (400 MHz, CDCl₃) δ 8.37 (s, 1H), 7.63-7.50 (m, 4H), 7.40 (d, J=27.0 Hz, 3H), 6.95 (s, 1H), 5.26 (s, 2H), 3.86 (s, 3H), 3.25 (d, J=5.5 Hz, 2H), 3.13 (s, 2H), 2.05 (s, 3H), 2.03 (d, J=21.8 Hz, 4H).

### Example 61 N-(4-(4-amino-5-(2-fluoro-4-((1-oxotetrahydro-1λ⁶-thiophene-1-ylidene) amino) phenyl)-7-methyl-7H-pyrrolo [2,3-d] pyrimidin-6-yl)-3-fluorophenyl) methacrylamide

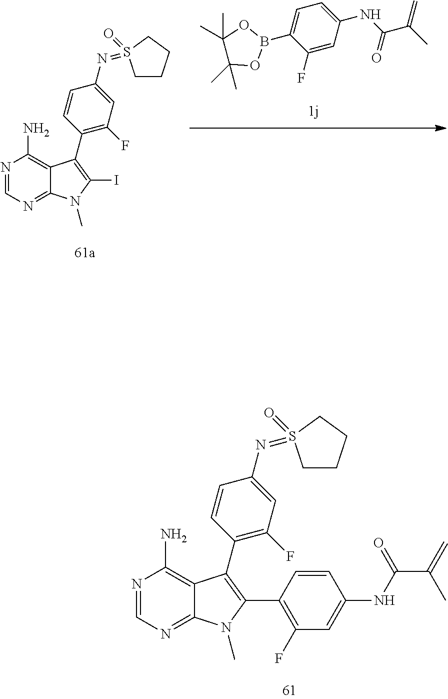 [SMILES: Cn1c(I)c(-c2ccc(N=S3(=O)CCCC3)cc2F)c2c(N)ncnc21 ; C=C(C)C(=O)Nc1ccc(B2OC(C)(C)C(C)(C)O2)c(F)c1 ; C=C(C)C(=O)Nc1ccc(-c2c(-c3ccc(N=S4(=O)CCCC4)cc3F)c3c(N)ncnc3n2C)c(F)c1]

Compound 61 was synthesized with reference to Example 12.

MS (ESI) m/z 537.57 [M+H]⁺.

¹H NMR (400 MHz, CDCl₃) δ 8.36 (s, 1H), 7.81-7.66 (m, 2H), 7.30 (dd, J=8.9, 4.2 Hz, 1H), 7.23 (d, J=11.4 Hz, 1H), 7.15 (dd, J=8.3, 1.7 Hz, 1H), 7.04 (s, 1H), 5.84 (s, 1H), 5.53 (s, 1H), 5.18 (s, 2H), 3.86 (s, 3H), 3.33 (dd, J=12.7, 6.5 Hz, 2H), 3.18 (dd, J=12.9, 6.7 Hz, 2H), 2.38-2.13 (m, 4H), 2.10 (s, 3H).

### Example 62 N-(4-(4-amino-5-(4-(ethyl (isopropyl) (oxo)-1λ⁶-sulfanylidene) phenyl)-7-methyl-7H-pyrrolo [2,3-d] pyrimidin-6-yl)-phenyl) methacrylamide

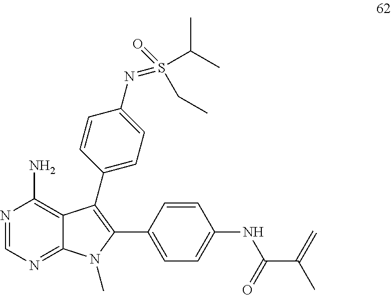 [SMILES: C=C(C)C(=O)Nc1ccc(-c2c(-c3ccc(N=S(=O)(CC)C(C)C)cc3)c3c(N)ncnc3n2C)cc1]

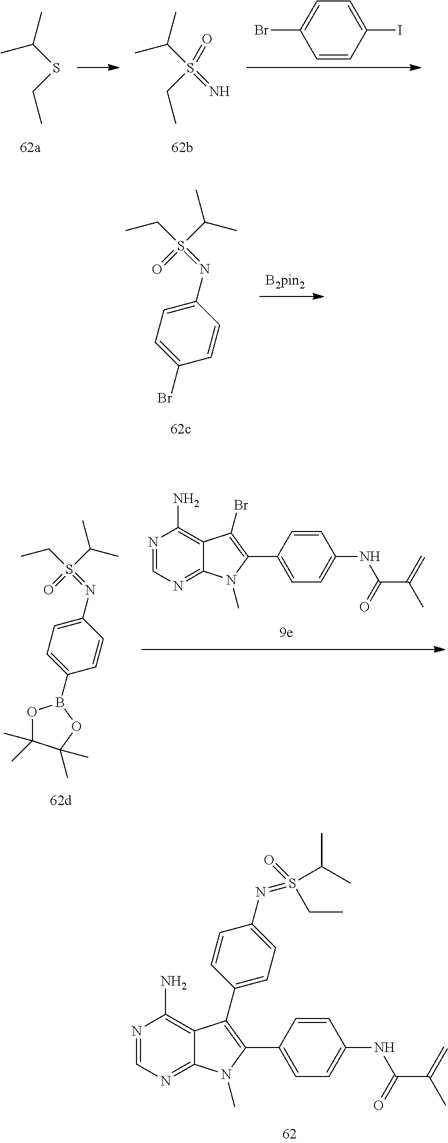

### Step 1 (imino) (isopropyl)-1λ⁶-ethylsulfoxide

Ethyl isopropyl sulfide (3 g, 30 mmol) and ammonium carbamate (3.5 g, 45 mmol) were added to a flask sequentially, and then MeOH (100 mL) and iodobenzene diethyl ester (19.3 g, 60 mmol) were added. The reaction solution was stirred open at room temperature for 30 min, and then concentrated under reduced pressure to remove the solvent to obtain a crude product. The crude product was purified by column chromatography (MeOH/DCM=1/20) to obtain compound 62b (4 g), light brown oil.

¹H NMR (400 MHz, CDCl₃) δ 3.18 (hept, J=6.8 Hz, 1H), 3.03 (q, J=7.5 Hz, 2H), 2.05 (s, 1H), 1.42-1.37 (m, 9H).

### Step 2 ((4-bromophenyl) imino) (isopropyl)-1λ⁶-ethylsulfoxide 62c

Compound 62b (675 mg, 5 mmol), cesium carbonate (2.45 g, 7.5 mmol), 1-bromo-4-iodobenzene (1.7 g, 6 mmol), Pd₂(dba)₃ (137 mg, 0.15 mmol), and Xantphos (260 mg, 0.45 mmol) were added to 1,4-dioxane (10 mL), and the reaction solution was heated up to 100° C. for 16 h. The reaction solution was filtered through diatomaceous earth, the filter cake was washed with ethyl acetate (100 mL), and saturated saline solution (50 mL) was used for washing. The organic phase was dried over anhydrous sodium sulfate and concentrated under reduced pressure to obtain a crude product. The crude product was purified by column chromatography (PE/EtOAc=4/1 to 3/2) to obtain compound 62c (1.3 g), brownish yellow oil.

¹H NMR (400 MHz, CDCl₃) δ 7.27 (d, J=8.7 Hz, 2H), 6.98 (d, J=8.7 Hz, 2H), 3.39 (hept, J=6.9 Hz, 1H), 3.22-3.06 (m, 2H), 1.46 (d, J=6.8 Hz, 3H), 1.41-1.35 (m, 6H).

### Step 3 (isopropyl) ((4-(4,4,5,5-tetramethyl-1,3,2-dioxaborolan-2-yl) phenyl) imino)-1λ⁶-diethylsulfoxide 62d

Compound 62c (1.3 g, 4.5 mmol), bis(pinacolato)diboron (1.4 g, 5.4 mmol), Pd(dppf)Cl₂ (656 mg, 0.9 mmol), potassium acetate (1.3 g, 13.5 mmol), and 1,4-dioxane (12 mL) were added into a sealed tube, after replaced with argon gas three times, sealed, and the reaction solution was heated up to 90° C. and stirred for 16 h. Water (10 mL) was added and extracted with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 62d (1.4 g), light yellow solid.

### Step 4: N-(4-(4-amino-5-(4-(ethyl (isopropyl) (oxo)-1λ⁶-sulfanylidene) phenyl)-7-methyl-7H-pyrrolo [2,3-d] pyrimidin-6-yl)-phenyl) methacrylamide 62

Compound 62d (85 mg, 0.24 mmol), compound 9e (77 mg, 0.2 mmol), Pd(PPh₃)₄ (23 mg, 0.02 mmol), and K₃PO₄ (127 mg, 0.6 mmol) were added into a sealed tube, after replaced with argon gas three times, 1,4-dioxane (2 mL) and H₂O (0.2 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (MeOH/DCM=1/10) to obtain compound 62 (33 mg), white solid.

MS (ESI) m/z 517 [M+H]⁺.

¹H NMR (400 MHz, CDCl₃) δ 8.34 (s, 1H), 7.53 (d, J=8.4, 2H), 7.51 (s, 1H), 7.22 (d, J=8.4, 2H), 7.09-7.03 (m, 4H), 5.79 (s, 1H), 5.48 (q, J=1.6 Hz, 1H), 4.99 (s, 2H), 3.70 (s, 3H), 3.41 (hept, J=6.9 Hz, 1H), 3.26-3.08 (m, 2H), 2.07 (s, 3H), 1.48 (d, J=6.8 Hz, 3H), 1.43 (d, J=7.0 Hz, 3H), 1.40 (t, J=7.4 Hz, 3H).

### Example 63N-(4-(4-amino-5-(4-((3,3-dimethyl-1-oxo-12-thiocyclobutyl-1-ylidene) amino) phenyl)-7-methyl-7H-pyrrolo [2,3-d] pyrimidin-6-yl) phenyl) methacrylamide

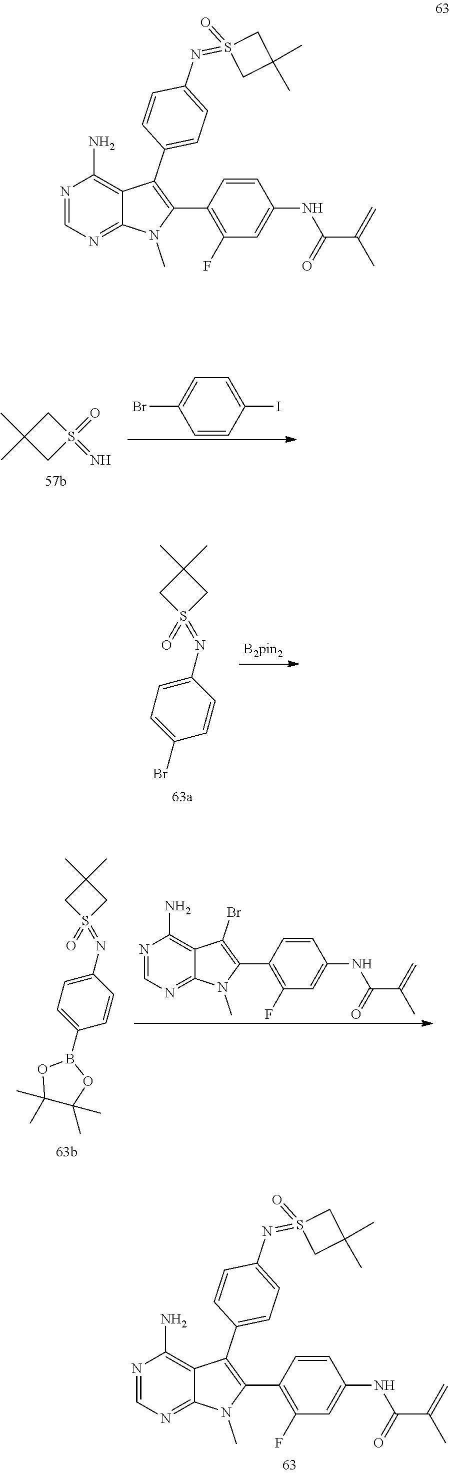

### Step 1 1-((4-bromo-phenyl) imino)-3,3-dimethyl-1λ⁶-thietane-1-oxide 63a

Compound 57b (760 mg, 5.7 mmol), cesium carbonate (2.78 g, 8.6 mmol), 1-bromo-4-iodobenzene (1.94 g, 6.9 mmol), Pd₂(dba)₃ (155 mg, 0.17 mmol), and Xantphos (297 mg, 0.51 mmol) were added to 1,4-dioxane (15 mL), and the reaction solution was heated up to 100° C. for 16 h. The reaction solution was filtered through diatomaceous earth, the filter cake was washed with ethyl acetate (100 mL), and saturated saline solution (50 mL) was used for washing. The organic phase was dried over anhydrous sodium sulfate and concentrated under reduced pressure to obtain a crude product. The crude product was purified by column chromatography (PE/EtOAc=4/1 to 3/2) to obtain compound 63a (1.2 g), light yellow solid.

### Step 3 1-(4-(4,4,5,5-tetramethyl-1,3,2-dioxaborolan-2-yl) phenyl) imino)-3,3-dimethyl-1λ⁶-thietane-1-oxide 63b

Compound 63a (611 mg, 2.1 mmol), bis(pinacolato)diboron ester (700 mg, 2.75 mmol), Pd(dppf)Cl₂ (307 mg, 0.42 mmol), potassium acetate (617 mg, 6.3 mmol), and 1,4-dioxane (10 mL) were added into a sealed tube, after replaced with argon gas three times, sealed, and the reaction solution was heated up to 90° C. and stirred for 16 h. Water (10 mL) was added to extract with DCM (20 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 63b (590 mg), white solid.

¹H NMR (400 MHz, CDCl₃) δ 7.70 (d, J=8.3 Hz, 2H), 7.00 (d, J=8.3 Hz, 2H), 3.95 (d, J=1.6 Hz, 4H), 1.53 (s, 3H), 1.48 (s, 3H), 1.33 (s, 12H).

### Step 4 N-(4-(4-amino-5-(4-(3,3-dimethyl-1-oxo-1λ⁶-thiocyclobutyl-1-ylidene) amino) phenyl)-7-methyl-7H-pyrrolo [2,3-d] pyrimidin-6-yl) phenyl) methacrylamide 63

Compound 63b (144 mg, 0.43 mmol), compound 8e (145 mg, 0.36 mmol), Pd(PPh₃)₄ (42 mg, 0.04 mmol), and K₃PO₄ (229 mg, 1.08 mmol) were added into a sealed tube, after replaced with argon three times, 1,4-dioxane (4 mL) and H₂O (0.4 mL) were added, sealed, and the reaction solution was heated up to 100° C. and stirred for 16 h. Dichloromethane (20 mL) and saturated saline solution (10 mL) were added to extract, and the organic phase was dried over anhydrous sodium sulfate, and concentrated under reduced pressure to obtain a crude product. The crude product was purified by column chromatography (DCM/MeOH=10/1) to obtain compound 63 (90 mg), white solid.

MS (ESI) m/z 533 [M+H]⁺.

¹H NMR (400 MHz, CDCl₃) δ 8.36 (s, 1H), 7.71 (dd, J=11.5, 1.9 Hz, 1H), 7.58 (s, 1H), 7.15-7.03 (m, 4H), 6.94 (d, J=8.1 Hz, 2H), 5.80 (s, 1H), 5.51 (d, J=1.7 Hz, 1H), 5.02 (s, 2H), 4.00 (d, J=12.8 Hz, 2H), 3.94 (d, J=12.8 Hz, 2H), 3.65 (s, 3H), 2.07 (s, 3H), 1.54 (s, 3H), 1.52 (s, 3H).

### Example 64 N-(4-(4-amino-7-cyano-1-methyl-3-(4-((1-oxotetrahydro-2H-12-thiopyran-1-ylidene) amino) phenyl)-1H pyrrolo [3,2-c]pyridine-2-yl)-3-fluorophenyl) methacrylamide

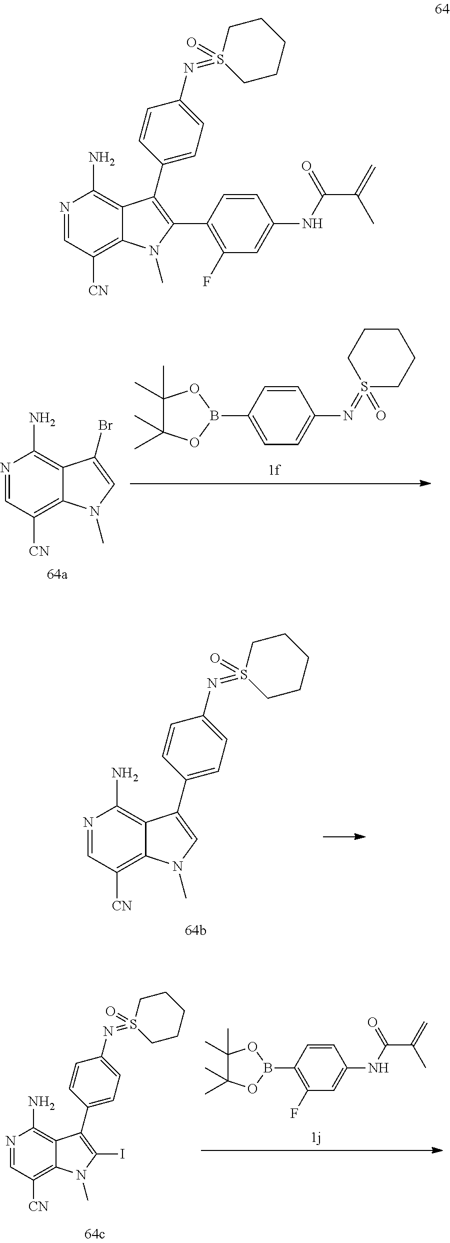

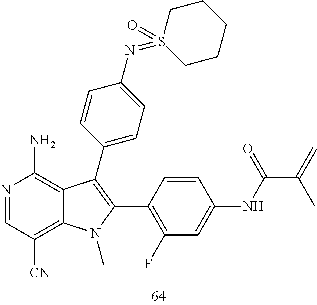 [SMILES: C=C(C)C(=O)Nc1ccc(-c2c(-c3ccc(N=S4(=O)CCCCC4)cc3)c3c(N)ncc(C#N)c3n2C)c(F)c1]

### Step 1 4-amino-1-methyl-3-(4-((1-oxotetrahydro-2H-1λ⁶-thiopyran-1-ylidene) amino) phenyl)-1H-pyrrolo [3,2-c]pyridine-7-nitrile 64b

Compound 64a (45 mg, 0.179 mmol), compound 1f (73 mg, 0.215 mmol), Pd(DtBPF)Cl₂ (12 mg, 0.018 mmol), and cesium fluoride (82 mg, 0.538 mmol) were placed in a 25 mL two necked flask, after replaced with argon gas three times, DMF/H₂O (v/v=8/1, 2.7 mL) was added to react overnight at 90° C. After the reaction was completed, the reaction solution was poured into water (20 mL) and extracted with ethyl acetate (50 mL×3) three times. The organic phase was concentrated, and the crude product was purified by column chromatography (DCM/MeOH=30/1) to obtain compound 64b (40 mg), white solid.

MS (ESI) m/z 380.1 [M+H]⁺.

### Step 2 4-amino-2-iodo-1-methyl-3-(4-((1-oxotetrahydro-2H-1λ⁶-thiopyran-1-ylidene) amino) phenyl)-1H-pyrrolo [3,2-c]pyridine-7-nitrile 64c

Compound 64b (40 mg, 0.105 mmol) was placed in a 25 mL reaction flask, dichloromethane (4.5 mL) and trifluoroacetic acid (25 L) were added in sequence. At 0° C., N-iodosuccinimide (27 mg, 0.116 mmol) was added, and the reaction was carried out at maintained 0° C. for 1 h. After the reaction was completed, the reaction solution was concentrated, and the crude product was purified by column chromatography (DCM/MeOH=30/1) to obtain compound 64c (42 mg), white solid.

MS (ESI) m/z 505.9 [M+H]⁺.

### Step 3 N-(4-(4-amino-7-cyano-1-methyl-3-(4-((1-oxotetrahydro-2H-1λ⁶-thiopyran-1-ylidene) amino) phenyl)-1H pyrrolo [3,2-c]pyridine-2-yl)-3-fluorophenyl) methacrylamide 64

Compound 64c (40 mg, 0.083 mmol), compound 1j (31 mg, 0.100 mmol), Pd(dppf)Cl₂ (7 mg, 0.008 mmol), and potassium phosphate (53 mg, 0.250 mmol) were placed in a 25 mL two necked flask, after replaced with argon gas three times, DMF/H₂O (v/v=8/1, 1.8 mL) was added to dissolve, and the reaction was carried out at 90° C. After the reaction was completed, the reaction solution was poured into water (20 mL) and extracted with ethyl acetate (50 mL×3) three times. The organic phase was concentrated, and the crude product was purified by column chromatography (DCM/MeOH=20/1) to obtain compound 64 (13 mg), white solid.

MS (ESI) m/z 557.0 [M+H]⁺.

¹H NMR (400 MHz, CDCl₃) δ 8.15 (s, 1H), 7.70 (d, J=11.4 Hz, 1H), 7.63 (s, 1H), 7.39 (dd, J=20.0, 8.1 Hz, 2H), 7.33-7.27 (m, 2H), 7.18 (d, J 8.0 Hz, 1H), 6.89 (s, 1H), 5.83 (s, 1H), 5.51 (s, 1H), 5.31 (s, 2H), 4.06 (s, 3H), 3.24 (s, 2H), 3.05 (t, J=11.1 Hz, 2H), 2.08 (s, 3H), 1.99-1.87 (m, 2H), 1.86-1.75 (m, 2H), 1.56-1.42 (m, 2H).

### Example 65 1-(4-(4-amino-7-methyl-5-(4-((1-oxotetrahydro-2H-1λ⁶-thiopyran-1-ylidene) amino) phenyl)-7H-pyrrolo [2,3-d] pyrimidin-6-yl)-3,6-dihydropyridine-1 (2H) yl)-2-fluoropropan-2-enyl-1-one

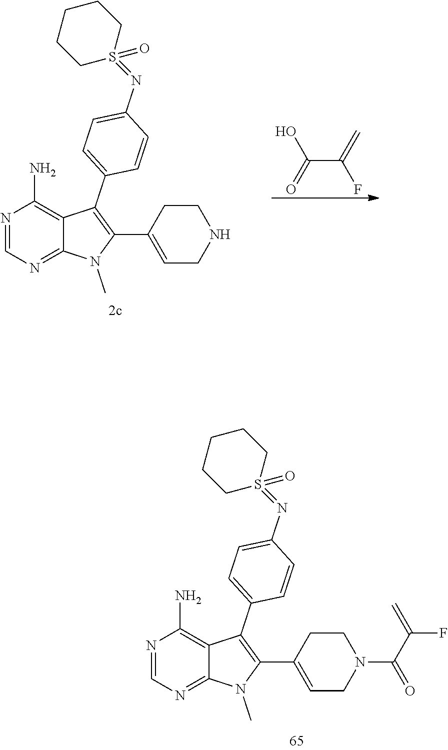

Compound 2c (176 mg, 0.40 mmol), 2-fluoroacrylic acid (36.3 mg, 0.40 mmol), and DIPEA (517 mg, 4.0 mmol) were added into a round bottomed reaction flask, dry DMF (5 mL) was added to dissolve, then HATU (228 mg, 0.60 mmol) was added, and stirred at room temperature for 4 h. After the reaction was completed, it was extracted with EA/H₂O three times, and the organic layers were combined, washed with saturated saline solution, dried over anhydrous sodium sulfate, concentrated under reduced pressure, and purified by column chromatography (DCM/MeOH=30/1) to obtain compound 65 (136 mg), yellow solid.

MS (ESI) m/z 509.2 [M+H]⁺.

¹H NMR (400 MHz, MeOD-d4) δ 8.35 (s, 1H), 7.46-7.33 (m, 4H), 6.05 (s, 1H), 5.36-5.11 (m, 2H), 4.25 (d, J=33.9 Hz, 2H), 3.85 (s, 3H), 3.81-3.73 (m, 2H), 3.70 (t, J=5.5 Hz, 2H), 3.67-3.57 (m, 2H), 2.30-2.19 (m, 2H), 2.18-2.07 (m, 4H), 1.90-1.79 (m, 1H), 1.78-1.68 (m, 1H).

### Example 66 1-(4-(4-amino-7-methyl-5-(4-(1-oxotetrahydro-2H-1λ⁶-thiopyran-1-ylidene) amino) phenyl)-7H-pyrrolo [2,3-d] pyrimidin-6-yl)-3,6-dihydropyridine-1 (2H)-yl) but-2-ynyl-1-one

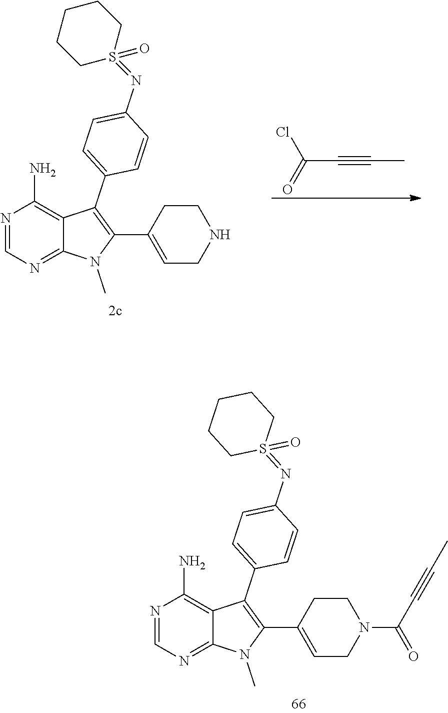

Compound 2c (150 mg, 0.34 mmol) was dissolved in dry DCM (3 mL), and triethylamine (35 mg, 0.34 mmol) was added at 0° C. But-2-ynyl chloride (34.9 mg, 0.34 mmol) was diluted with DCM (2 mL), and added slowly dropwise into the above reaction solution, and stirred at 0° C. for 1 h. After the reaction was completed, it was extracted with DCM/H₂O three times, and the organic layers were combined, washed with saturated saline solution, dried over anhydrous sodium sulfate, concentrated under reduced pressure, and purified by column chromatography (DCM/MeOH=30/1) to obtain compound 66 (73 mg), yellow solid MS (ESI) m/z 503.2 [M+H]⁺.

¹H NMR (400 MHz, MeOD-d4) δ 8.35 (s, 1H), 7.48-7.22 (m, 4H), 6.10-5.96 (m, 1H), 4.43 (q, J 2.8 Hz, 1H), 4.18 (q, J=2.8 Hz, 1H), 3.88 (t, J=5.6 Hz, 1H), 3.84 (d, J=1.2 Hz, 3H), 3.75-3.70 (m, 1H), 3.69-3.63 (m, 2H), 3.61-3.50 (m, 2H), 2.28-2.21 (m, 1H), 2.20-2.09 (m, 5H), 2.07 (s, 1H), 2.03 (s, 2H), 1.88-1.77 (m, 1H), 1.76-1.67 (m, 1H).

### Example 67 (E)-1-(4-(4-amino-7-methyl-5-(4-((1-oxotetrahydro-2H-1λ⁶-thiopyran-1-ylidene) amino) phenyl)-7H-pyrrolo [2,3-d] pyrimidin-6-yl)-3,6-dihydropyridine-1(2H)-yl)-4-(dimethylamino) but-2-enyl-1-one

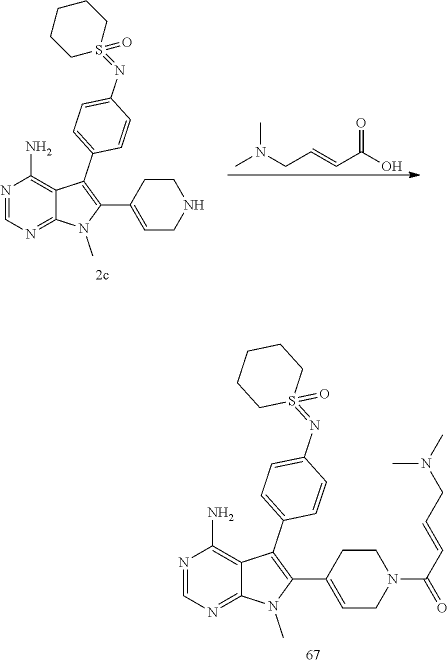

Compound 2c (176 mg, 0.4 mmol) and (E)-4-(dimethylamino) but-2-olefine acid (66 mg, 0.4 mmol) were dissolved in DMF (4 mL), triethylamine (404.8 mg, 4 mmol) and T3P (191 mg, 0.6 mmol) were added in sequence at 0° C. overnight. After the reaction was completed, it was extracted with ethyl acetate/H₂O three times, the organic layers were combined, washed with saturated saline solution, dried over anhydrous sodium sulfate, concentrated under reduced pressure, and perform column chromatography (DCM/MeOH=20/1) to obtain compound 67 (41 mg), yellow solid.

MS (ESI) m/z 548.3 [M+H]⁺.

¹H NMR (400 MHz, MeOD-d4) δ 8.37 (s, 1H), 7.61-7.35 (m, 4H), 7.10-6.91 (m, 1H), 6.82-6.60 (m, 1H), 6.06 (s, 1H), 4.34 (d, J=3.3 Hz, 1H), 4.25 (d, J=3.3 Hz, 1H), 4.04-3.92 (m, 4H), 3.85 (s, 3H), 3.84-3.67 (m, 4H), 2.92 (d, J=6.4 Hz, 6H), 2.32-2.12 (m, 6H), 1.94-1.72 (m, 2H).

### Example 68 N-(4-(4-amino-7-methyl-5-(4-((1-oxotetrahydro-2H-1λ⁶-thiopyran-1-ylidene) amino) phenyl)-7H-pyrrolo [2,3-d] pyrimidin-6-yl) phenyl)-2-fluoroacrylamide

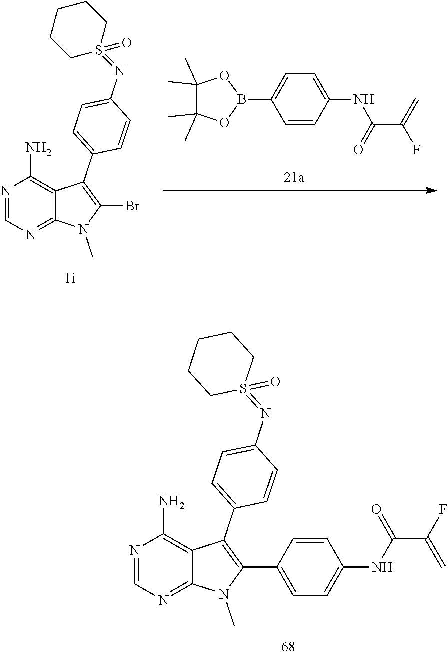

Compound 1i (200 mg, 0.46 mmol) was dissolved in 1,4-dioxane (5 mL) and water (1 mL), compound 21a (161.3 mg, 0.55 mmol), Pd(PPh₃)₄ (106.3 mg, 0.092 mmol), and K₃PO₄ (293 mg, 1.38 mmol) were added in sequence. After the air in the reaction bottle was replaced with nitrogen gas, the reaction was stirred overnight at 100° C. After the reaction was completed, it was filtered with silica gel, and the filtrate was recovered, concentrated under reduced pressure, dissolved in ethyl acetate, washed with saturated saline solution, dried over anhydrous sodium sulfate, concentrated under reduced pressure and subjected to column chromatography (DCM/MeOH=30/1) to obtain compound 68 (118 mg), white solid.

MS (ESI) m/z 519.2 [M+H]⁺.

¹H NMR (400 MHz, MeOD-d4) δ 8.36 (s, 1H), 7.74 (d, J=8.7 Hz, 2H), 7.32 (d, J=8.7 Hz, 2H), 7.15 (d, J=8.5 Hz, 2H), 7.08 (d, J=8.5 Hz, 2H), 5.73 (dd, J=46.5, 3.4 Hz, 1H), 5.32 (dd, J=15.1, 3.5 Hz, 1H), 3.78 (s, 3H), 3.43-3.34 (m, 2H), 3.29-3.19 (m, 2H), 2.09-1.98 (m, 4H), 1.79-1.61 (m, 2H).

### Example 69 N-(4-(4-amino-5-(3-fluoro-4-((1-oxotetrahydro-2H-1λ⁶-thiopyran-1-ylidene) amino) phenyl)-7-methyl-7H-pyrrolo [2,3-d] pyrimidin-6-yl) phenyl) methacrylamide

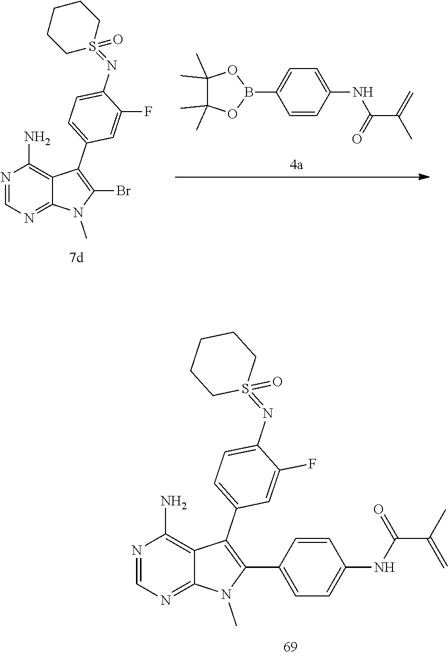

Compound 7d (300 mg, 0.67 mmol) was dissolved in 1,4-dioxane (8 mL) and water (2 mL), and compound 4a (229.2 mg, 0.8 mmol), Pd(PPh₃)₄ (154.8 mg, 0.13 mmol), and K₃PO₄ (426.7 mg, 2.01 mmol) were added in sequence. After the air in the reaction flask was replaced with nitrogen gas, the reaction was stirred overnight at 100° C. After the reaction was completed, it was filtered with silica gel, and the filtrate was recovered, concentrated under reduced pressure, dissolved in ethyl acetate, washed with saturated saline solution, dried over anhydrous sodium sulfate, concentrated under reduced pressure and perform column chromatography (DCM/MeOH=30/1) to obtain compound 69 (116 mg), white solid.

MS (ESI) m/z 533.2 [M+H]⁺.

¹H NMR (400 MHz, DMSO-d₆) δ 9.90 (s, 1H), 8.17 (s, 1H), 7.71 (d, J=8.7 Hz, 2H), 7.26 (d, J=8.7 Hz, 2H), 7.10 (dd, J=9.3, 8.3 Hz, 1H), 6.94-6.79 (m, 2H), 5.99 (s, 1H), 5.80 (s, 1H), 5.53 (s, 1H), 4.08-4.00 (m, 1H), 3.58 (s, 3H), 3.32 (s, 2H), 3.31-3.22 (m, 2H), 1.95 (s, 3H), 1.93-1.83 (m, 4H), 1.68-1.50 (m, 2H).

### Example 70 N-(4-(4-amino-5-(5-fluoro-4-(1-oxo-12′-thiocyclobutan-1-ylidene) amino) cyclohexyl-1,5-dienyl-1-yl)-7-methyl-7H-pyrrolo [2,3-d] pyrimidin-6-yl)-3-methylphenyl) methacrylamide

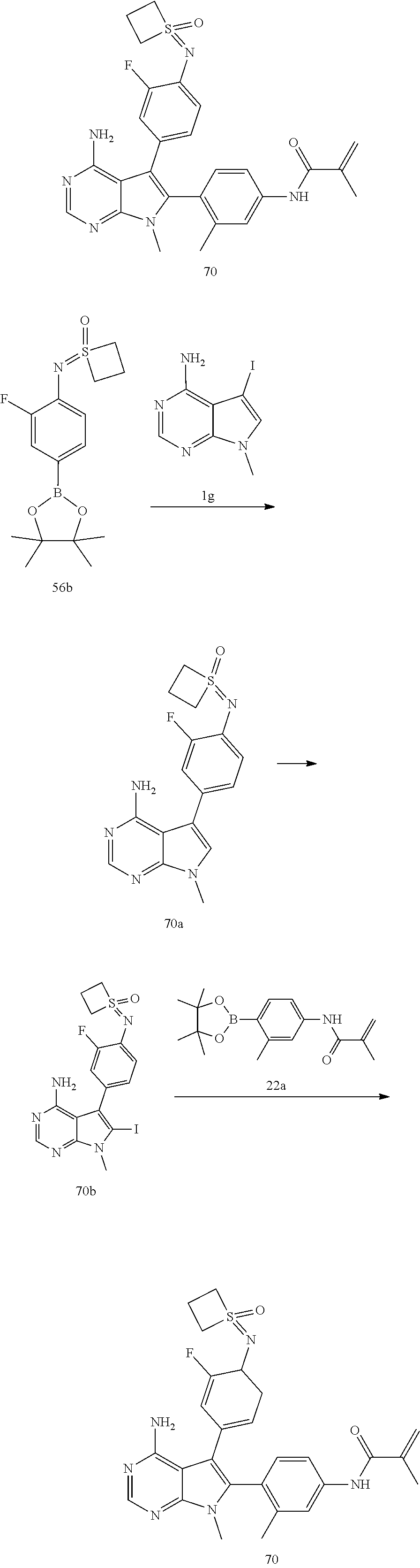

Compound 70 was synthesized with reference to Example 29.

MS (ESI) m/z 519.47 [M+H]⁺.

¹H NMR (400 MHz, CDCl₃) δ 8.30 (s, 1H), 7.43 (d, J=2.1 Hz, 2H), 7.35 (dd, J 8.2, 2.0 Hz, 1H), 7.10 (d, J=8.2 Hz, 1H), 6.92 (t, J=8.6 Hz, 1H), 6.81 (s, 2H), 5.73 (s, 1H), 5.42 (d, J=1.1 Hz, 1H), 4.96 (s, 2H), 4.21 (d, J=8.5 Hz, 4H), 3.45 (s, 1H), 2.26 (s, 2H), 2.01 (s, 3H), 1.94 (s, 1H).

### Example 71 N-(4-(4-amino-5-(4-(((ethylamino) (oxo) (trifluoromethyl)-1λ⁶-sulfaneylidene) amino)-3-fluorophenyl)-7-methyl-7H-pyrrolo [2,3-d] pyrimidin-6-yl) phenyl) methacrylamide KTH021161

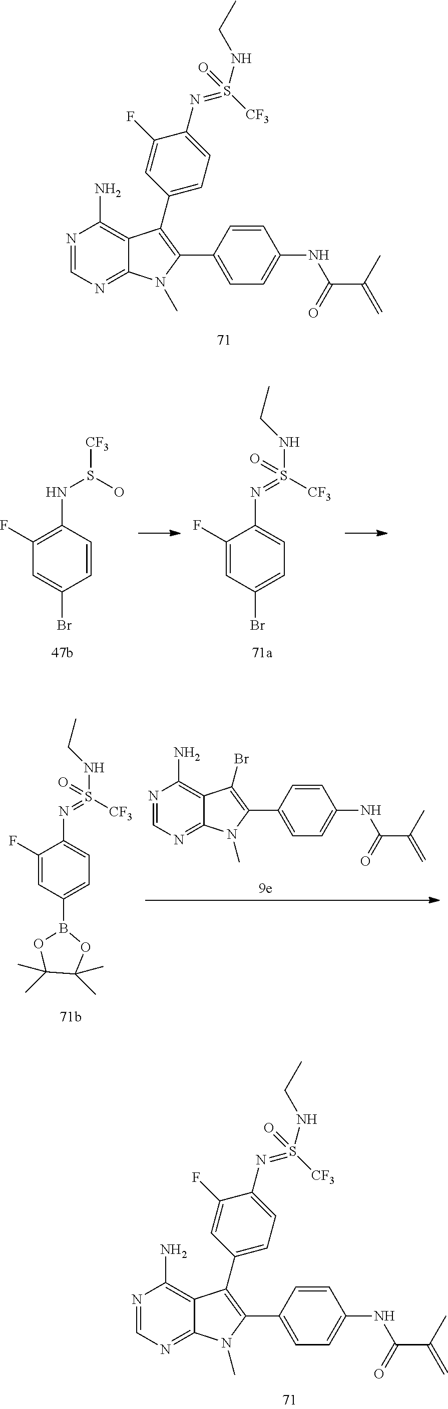

### Step 1 N-ethyl-1,1,1-trifluoro-N′-(4-iodophenyl)-N-methylmethanesulfonimide 71a

Compound 47b (600 mg, 1.96 mmol) and N-chlorosuccinimide (292 mg, 2.16 mmol) were dissolved in acetonitrile (8 mL), tetrabutylammonium fluoride (2.16 mL, 2.16 mmol, 1M) was added at 0° C., and the reaction was carried out at room temperature for 30 min. The reaction solution was concentrated, and then acetonitrile (8 mL) and ethylamine (360 mg, 8.0 mmol) were added, and reacted at room temperature for 2 h. The reaction solution was concentrated, and dried, and the crude product was purified by column chromatography (PE/EtOAc=0-20%) to obtain compound 71a (422 mg).

MS (ESI) m/z 349 [M+H]⁺

### Step 2 N-ethyl-1,1,1-trifluoro-N′-(2-fluoro-4-(4,4,5,5-tetramethyl-1,3,2-dioxaborolan-2-yl) phenyl) methylsulfonyl imine 71b

Compound 71a (260 mg, 0.74 mmol), bis(pinacolato)diboron (227 mg, 0.89 mmol), Pd(dppf)Cl₂ (110 mg, 0.15 mmol), and potassium acetate (220 mg, 2.23 mmol) were placed in a bottle, 1,4-dioxane (6 mL) was added, after replaced N₂ three times, the reaction was carried out at 100° C. for 12 h. The reaction solution was filtered and concentrated, and the crude product was purified by column chromatography (PE/EtOAc=0-20%) to obtain compound 71b (250 mg).

MS (ESI) m/z 396 [M+H]⁺

### Step 3 N-(4-(4-amino-5-(4-(((ethylamino) (oxo) (trifluoromethyl)-1λ⁶-sulfaneylidene) amino)-3-fluorophenyl)-7-methyl-7H-pyrrolo [2,3-d] pyrimidin-6-yl) phenyl) methacrylamide 71

Compound 9e (100 mg, 0.26 mmol), compound 71b (123 mg, 0.31 mmol), Pd(PPh₃)₄ (65 mg, 0.056 mmol), and K₃PO₄ (165 mg, 0.78 mmol) were added into a sealed tube, 1,4-dioxane (5 mL) and H₂O (0.5 mL) were added, after replaced with N₂ three times, the reaction solution was heated up to 100° C. and stirred for 16 h. Dichloromethane (20 mL) and saturated saline solution (20 mL) were added to extract, and the organic phase was dried over anhydrous sodium sulfate, and concentrated under reduced pressure to obtain a crude product. The crude product was purified by column chromatography (MeOH/DCM=1/20) to obtain compound 71 (20 mg), white solid.

MS (ESI) m/z 576.64 [M+H]⁺.

¹H NMR (400 MHz, MeOD-d₄) δ 8.18 (s, 1H), 7.67 (d, J=8.3 Hz, 2H), 7.28 (d, J=8.3 Hz, 2H), 7.17 (t, J=8.4 Hz, 1H), 7.02-6.93 (m, 2H), 5.81 (s, 1H), 5.52 (s, 1H), 3.68 (s, 3H), 2.03 (s, 3H), 1.33-1.29 (m, 2H), 1.15 (t, J=7.2 Hz, 3H).

### Example 72 N-(4-(4-amino-7-methyl-5-(4-(5-oxo-5λ⁶-thiaspiro [2.3]hexane-5-ylidene) amino) phenyl)-7H-pyrrolo [2,3-d] pyrimidin-6-yl)-3-fluorophenyl) methacrylamide

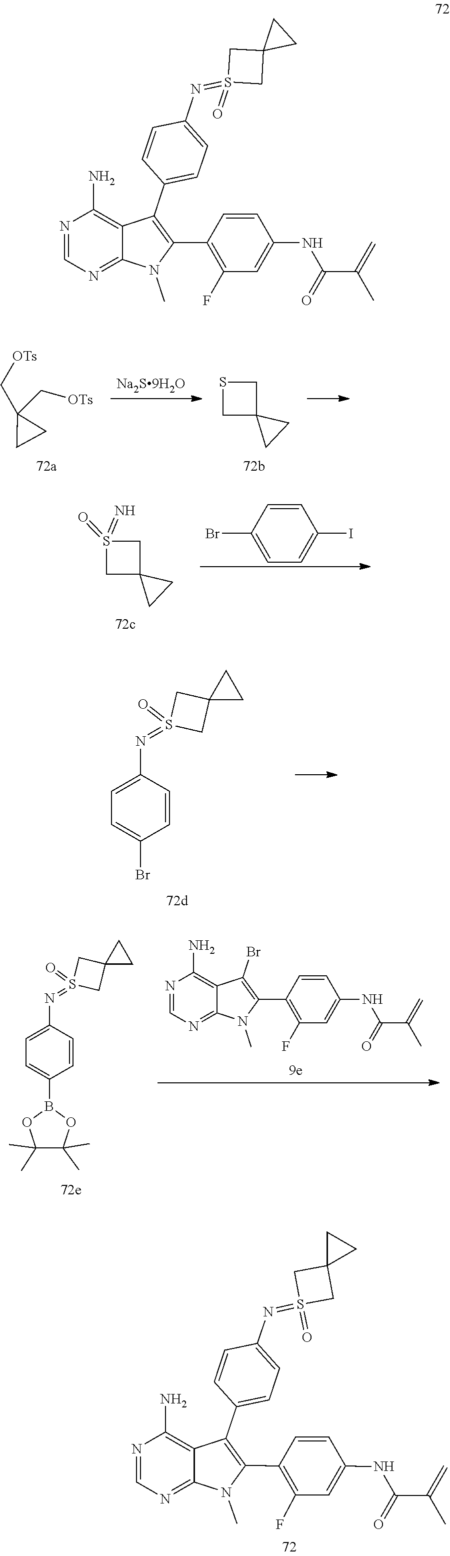

### Step 1 5-thiaclopril [2.3]hexane 72b

Compound 72a was synthesized reference to WO2022152705 A1. Compound 72a (3 g, 7.32 mmol) and sodium sulfide nonahydrate (2.1 g, 8.78 mmol) were placed in a 250 mL thick walled pressure resistant bottle, DMF (15 mL) was added and reacted overnight at 120° C. After the reaction was completed, H₂O (50 mL) was added and extracted with ethyl acetate three times (10 mL×3), and the organic layers were combined, washed with saturated saline solution, and dried over anhydrous sodium sulfate to obtain a ethyl acetate solution of compound 72b, which was directly used in the next step.

### Step 2 5-amino-5λ⁶-thiaspiro [2.3]hexane 5-oxide 72c

The ethyl acetate solution of 72b and ammonium carbamate (856 mg, 11.0 mmol) were added into a flask in sequence, and then MeOH (20 mL) and iodobenzene diethyl ester (5.3 g, 16.47 mmol) were added. The reaction solution was stirred open at room temperature for 30 min, and then concentrated under reduced pressure to remove the solvent to obtain a crude product. The crude product was purified by column chromatography (MeOH/DCM=1/20) to obtain compound 72c (500 mg), light brown oil.

MS (ESI) m/z 131 [M+H]⁺

### Step 3 5-((4-bromophenyl) imino)-5λ⁶-thiaspiro [2.3]hexane 5-oxide 72d

Compound 72c (300 mg, 0.29 mmol), cesium carbonate (1.5 g, 4.58 mmol), 1-bromo-4-iodobenzene (0.65 g, 2.29 mmol), Pd₂(dba)₃ (53 mg, 0.057 mmol), and Xantphos (99 mg, 0.17 mmol) were dissolved in 1,4-dioxane (10 mL), and the reaction solution was heated up to 100° C. to react for 16 h. After the reaction was completed, the reaction solution was filtered through diatomaceous earth, the filter cake was washed with ethyl acetate (100 mL), and saturated saline solution (50 mL) was used for washing. The organic phase was dried over anhydrous sodium sulfate and concentrated under reduced pressure to obtain a crude product. The crude product was purified by column chromatography (PE/EtOAc=4/1 to 3/2) to obtain compound 72d (310 mg).

MS (ESI) m/z 286 [M+H]⁺

### Step 4 5-((4-(4,4,5,5-tetramethyl-1,3,2-dioxaborolan-2-yl) phenyl) imino)-5λ⁶-thiaspiro [2.3]hexane 5-oxide 72e

Compound 72d (310 mg, 1.1 mmol), bis(pinacolato)diboron (330 mg, 1.3 mmol), Pd(dppf)Cl₂ (160 mg, 0.22 mmol), potassium acetate (330 mg, 3.3 mmol), and 1,4-dioxane (10 mL) were added into a round bottom flask, after replaced with argon gas three times, the reaction solution was heated up to 100° C. and stirred for 16 h. After the reaction was completed, it was cooled down to room temperature, water (20 mL) was added and extracted with ethyl acetate (15 mL×3). The organic phase was washed with saturated saline solution (20 mL), dried over anhydrous sodium sulfate, and concentrated under reduced pressure to obtain a crude product. The crude product was purified by column chromatography (PE/EtOAc=3/2) to obtain compound 72e (280 mg), light yellow solid.

MS (ESI) m/z 333 [M+H]⁺

### Step 5 N-(4-(4-amino-7-methyl-5-(4-(5-oxo-5λ⁶-thiaspiro [2.3]hex-5-ylidene) amino) phenyl)-7H-pyrrolo [2,3-d] pyrimidin-6-yl)-3-fluorophenyl) methacrylamide 72

Compound 72e (50 mg, 0.15 mmol), compound 9e (50 mg, 0.12 mmol), Pd(PPh₃)₄ (28 mg, 0.025 mmol), K₃PO₄ (80 mg, 0.37 mmol), 1,4-dioxane (2 mL), and H₂O (0.2 mL) were added into a round bottom flask, after replaced with argon gas three times, and the reaction solution was heated up to 100° C. and stirred for 16 h. After the reaction was completed, dichloromethane (20 mL) and saturated saline solution (10 mL) were added and extracted with dichloromethane. The organic phase was dried over anhydrous sodium sulfate and concentrated under reduced pressure to obtain a crude product. The crude product was purified by column chromatography (MeOH/DCM=1/15) to obtain compound 72 (15 mg), white solid.

MS (ESI) m/z 531.6 [M+H]⁺

¹H NMR (400 MHz, MeOD-d₄) δ 8.21 (s, 1H), 7.74 (dd, J=12.1, 2.1 Hz, 1H), 7.38 (dd, J=8.5, 2.1 Hz, 1H), 7.18 (dt, J=8.3, 4.3 Hz, 3H), 7.10-6.89 (m, 2H), 5.83 (s, 1H), 5.57 (d, J=1.6 Hz, 1H), 4.44-4.28 (m, 2H), 4.23-4.11 (m, 2H), 3.64 (s, 3H), 2.04 (t, J=1.2 Hz, 3H), 1.09-0.81 (m, 4H).

### Example 73 N-(4-(4-amino-5-(4-(diethyl (oxo)-λ⁶-sulfaneylidene) amino) phenyl)-7-methyl-7H-pyrrolo [2,3-d] pyrimidin-6-yl)-3-(difluoromethyl) phenyl) methacrylamide

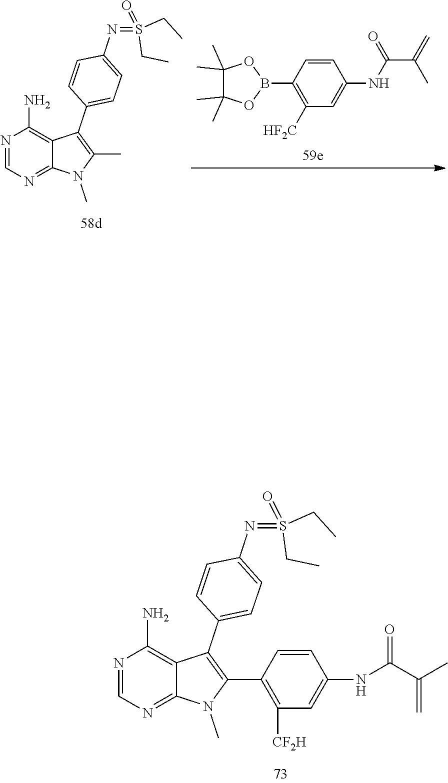

Compound 73 was synthesized with reference to the similar synthetic route and steps of compound 12, wherein compound 12a was replaced with compound 58d, compound 12b was replaced with compound 59e.

MS (ESI) m/z 553 [M+H]⁺.

¹H NMR (400 MHz, DMSO-d₆) δ 10.1 (s, 1H), 8.19 (s, 1H), 8.05 (s, 1H), 7.95 (d, J=8.0 Hz, 1H), 7.48 (d, J=8.0 Hz, 1H), 6.99 (d, J=8.0 Hz, 2H), 6.86 (d, J=8.0 Hz, 2H), 6.46 (t, J=8.0 Hz, 1H), 6.05 (s, 2H), 5.85 (s, 1H), 5.57 (s, 1H), 3.41 (s, 3H), 3.26-3.23 (m, 4H), 1.96 (s) 3H), 1.23 (s, 6H).

### Example 74 N-(4-(4-amino-5-(3-fluoro-4-(1-oxo-1λ⁶-thietan-1-imino) phenyl)-7-methyl-7H-pyrrolo [2,3-d] pyrimidin-6-yl)-3-chlorophenyl) methacrylamide

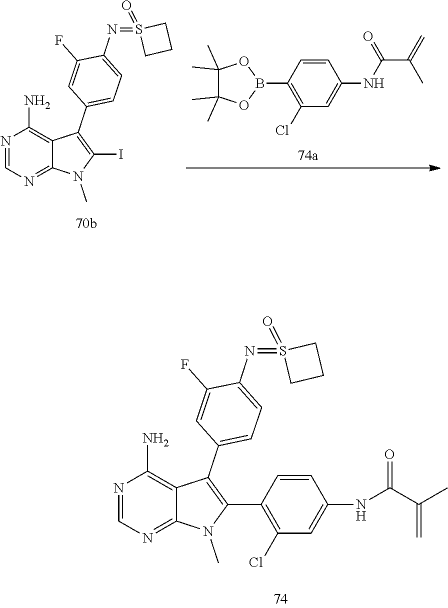

Compound 74 was synthesized with reference to the similar synthetic route and steps of compound 12, wherein, compound 12a was replaced with compound 70b, compound 12b was replaced with compound 74a.

MS (ESI) m/z 539 [M+H]⁺.

¹H NMR (400 MHz, DMSO-d₆) δ 10.08 (s, 1H), 8.21 (s, 1H), 8.03 (s, 1H), 7.67 (d, J 8.0 Hz, 1H), 7.59-7.56 (m, 3H), 7.35 (t, J=12.0 Hz, 1H), 6.14 (s, 2H), 5.84 (s, 1H), 5.58 (s, 1H), 4.29-4.26 (m, 4H), 3.46 (s, 3H), 2.28-2.18 (m, 2H), 1.96 (s, 3H).

### Example 75 N-(4-(4-amino-5-(3-fluoro-4-(1-oxo-1λ⁶-thiocyclobutan-1-ylidene) amino) phenyl)-7-methyl-7H-pyrrolo [2,3-d] pyrimidin-6-yl) fluorophenyl] methacrylamide

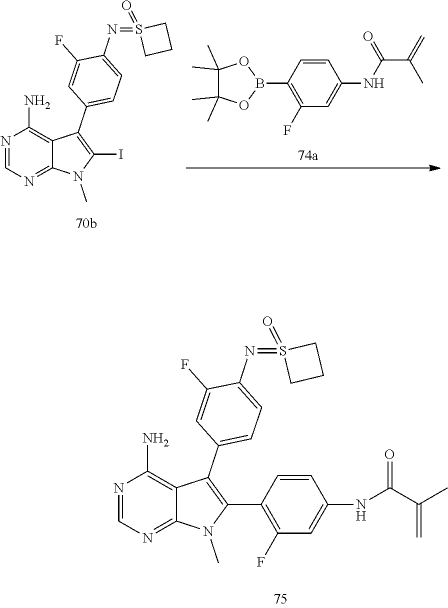

Compound 75 was synthesized with reference to the similar synthetic route and steps of compound 12, wherein, compound 12a was replaced with compound 70b, compound 12b was replaced with compound 1j.

MS (ESI) m/z 523 [M+H]⁺.

¹H NMR (400 MHz, DMSO-d₆) δ 10.12 (s, 1H), 8.21 (s, 1H), 7.79 (d, J=8.0 Hz, 1H), 7.52 (d, J=8.0 Hz, 1H), 7.32 (t, J=12.0 Hz, 2H), 7.00-6.89 (m, 2H), 6.12 (s, 2H), 5.84 (s, 1H), 5.59 (s, 1H), 4.31-4.27 (m, 4H), 3.53 (s, 3H), 2.22-2.19 (m, 2H), 1.96 (s, 3H).

### Example 76 N-(4-(4-amino-5-(4-(diethyl (oxo)-λ⁶-sulfaneyliden) amino)-2,5-difluorophenyl)-7-methyl-7H-pyrrolo [2,3-d] pyrimidin-6-yl)-3-fluorophenyl) methacrylamide KTH021179

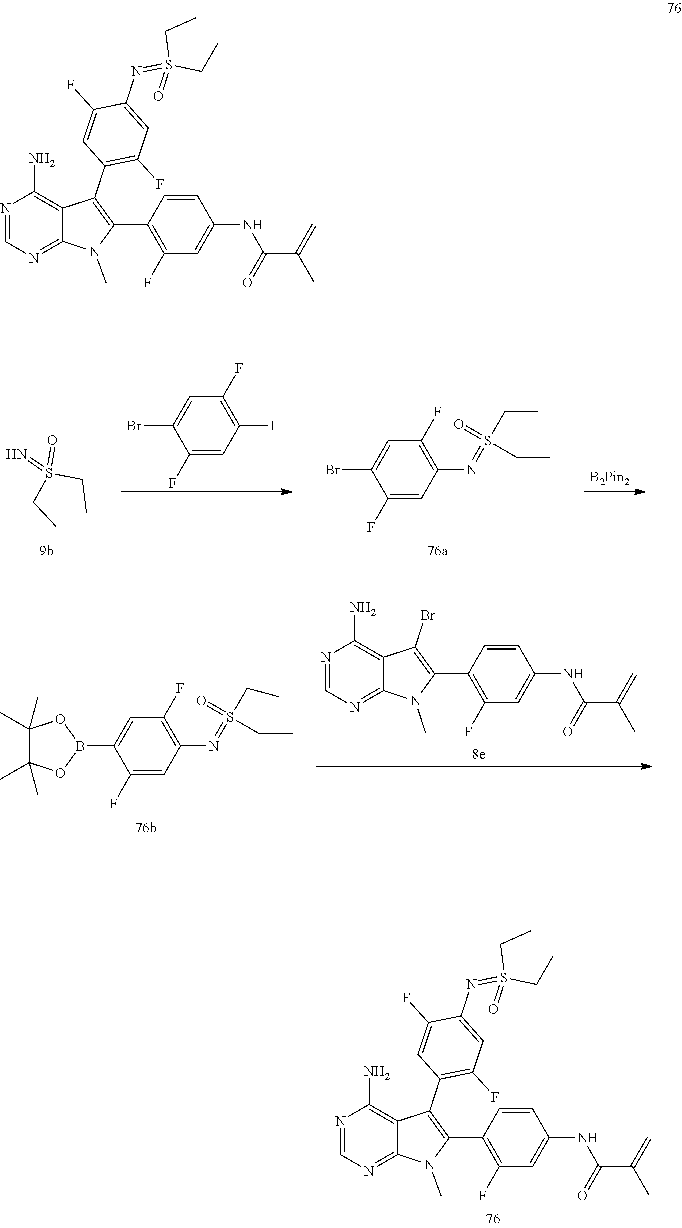

### Step 1 ((4-bromo-2,5-difluorophenyl) imino) diethyl-λ⁶-sulfone 76a

At room temperature, compound 9b (1 g, 8.25 mmol), 5-bromo-1,4-difluoro-2-iodobenzene (3.16 g, 9.91 mmol), tris (dibenzylideneacetone) dipalladium (183.14 mg, 0.19 mmol), 4,5-diphenylphosphine-9,9-dimethyloxanthracene (324.17 mg, 0.56 mmol), and cesium carbonate (3.76 g, 11.55 mmol) were added sequentially to a round bottom flask, 1,4-dioxane (20 mL) was added, followed by vacuum pumping and nitrogen gas purging three times, and heated up to 100° C. and stirred for 3 h. After the reaction was completed, the mixture was cooled down to room temperature and concentrated under reduced pressure to obtain a crude product. The crude product was purified by column chromatography (PE/EtOAc=1/3) to obtain compound 76a (800 mg), yellow solid.

MS (ESI) m/z 311 [M+H]⁺.

### Step 2 (2,5-difluoro-4-(4,4,5,5-tetramethyl-1,3,2-dioxaborolan-2-yl) phenyl) imino diethyl-λ⁶-sulfone 76b

At room temperature, compound 76a (800 mg, 2.05 mmol), bis(pinacolato)diboron (782.54 mg, 3.08 mmol), potassium acetate (197.49 mg, 54.75 mmol), 1,1′-biphenylphosphine ferrocene palladium chloride (197.49 mg, 0.26 mmol), and 1,4-dioxane (10 mL) were added to a round bottom flask sequentially, followed by vacuum pumping and nitrogen gas purging three times, and heated up to 100° C. and stirred for 1 h. After the reaction was completed, the mixture was cooled down to room temperature and filtered under reduced pressure, the filter cake was washed three times with dichloromethane, and the filtrate was concentrated under reduced pressure to obtain a crude product. The crude product was purified by column chromatography (PE/EtOAc=1/5) to obtain compound 76b (700 mg), light yellow solid.

MS (ESI) m/z 360 [M+H]⁺.

### Step 3 N-(4-(4-amino-5-(4-(diethyl (oxo)-λ⁶-sulfaneylidene) amino)-2,5-difluorophenyl)-7-methyl-7H-pyrrolo [2,3-d] pyrimidin-6-yl)-3-fluorophenyl) methacrylamide 76

At room temperature, compound 76b (106 mg, 0.30 mmol), compound 8e (100 mg, 0.25 mmol), tetrakis (triphenylphosphine) palladium (28.65 mg, 0.025 mmol), potassium phosphate (157.93 mg, 0.74 mmol), 1,4-dioxane (2 mL), and water (0.4 mL) were added to a round bottom flask sequentially, followed by vacuum pumping and nitrogen gas purging three times, and heated up to 90° C. and stirred for 2 h. After the reaction was completed, the mixture was cooled down to room temperature and concentrated under reduced pressure to obtain a crude product. The crude product was purified by column chromatography (DCM/MeOH=15/1) to obtain compound 76 (9.1 mg), white solid.

MS (ESI) m/z 557 [M+H]⁺.

¹H NMR (400 MHz, DMSO-d₆) δ 10.1 (s, 1H), 8.20 (s, 1H), 7.76 (d, J=8.0 Hz, 1H), 7.49 (d, J=8.0 Hz, 1H), 7.23-7.04 (m, 2H), 6.89 (s, 1H), 6.05 (s, 2H), 5.83 (s, 1H), 5.58 (s, 1H), 3.56 (s, 3H), 3.37-3.34 (m, 4H), 1.96 (s, 3H), 1.27 (s, 6H).

### Example 77 N-(4-(4-amino-5-(2,5-difluoro-4-((1-oxo-12-thiocyclobutan-1-ylidene) amino) phenyl)-7-methyl-7H-pyrrolo [2,3-d] pyrimidin-6-yl)-3-fluorophenyl) methacrylamide

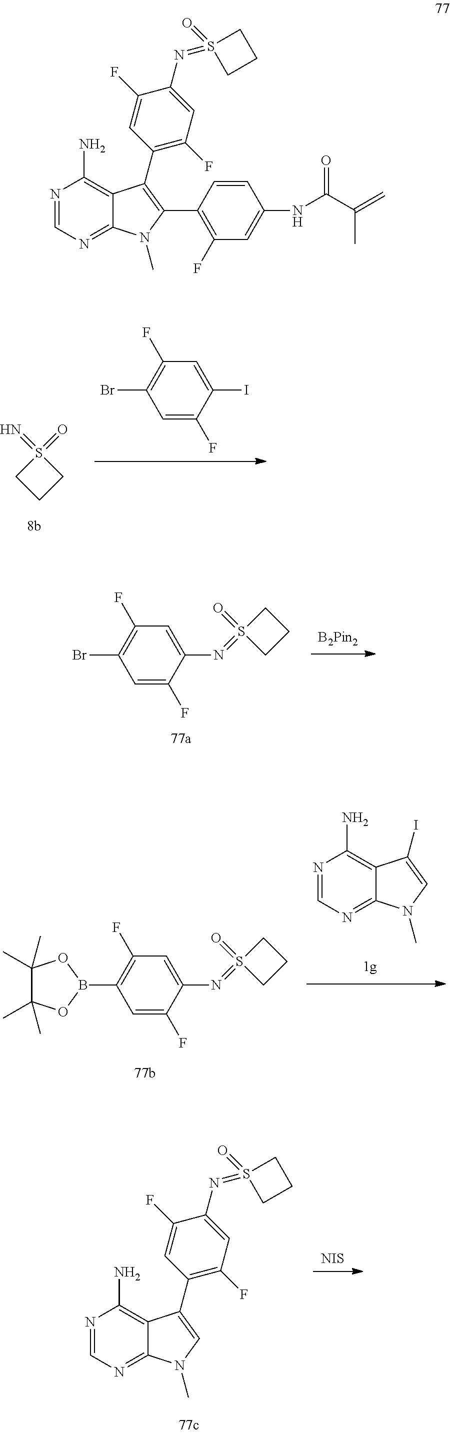

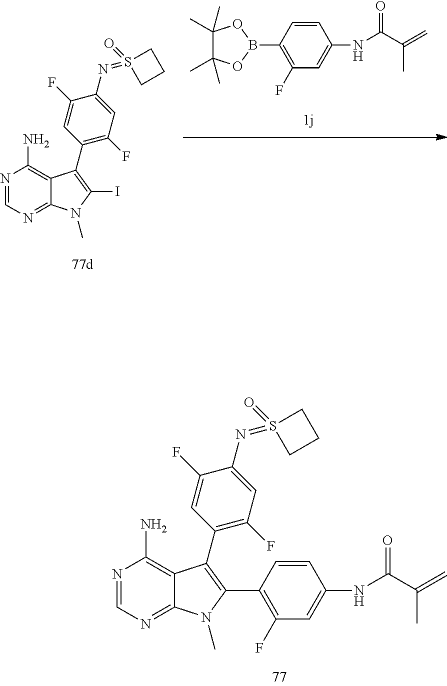

### Step 1 1-((4-bromo-2,5-difluorophenyl) imino)-1λ⁶-thietane-1-oxide 77a

At room temperature, compound 8b (1 g, 9.51 mmol), 1-bromo-2,5-difluoro-4-iodobenzene (3.63 g, 11.41 mmol), tris (dibenzylideneacetone) dipalladium (219.77 mg, 0.24 mmol), 4,5-diphenylphosphine-9,9-dimethyloxanthracene (410.82 mg, 0.71 mmol), cesium carbonate (4.34 g, 13.31 mmol), and 1,4-dioxane (20 mL) were added to a round bottom flask sequentially, followed by vacuum pumping and nitrogen gas purging three times, and heated up to 90° C. and stirred for 16 h. After TLC monitored the completion of the reaction, the mixture was cooled down to room temperature and concentrated under reduced pressure to obtain a crude product. The crude product was purified by column chromatography (PE/EtOAc=3/1) to obtain compound 77a (630 mg), light yellow solid.

MS (ESI) m/z 295 [M+H]⁺.

### Step 2 1-(2,5-difluoro-4-(4,4,5,5-tetramethyl-1,3,2-dioxaborolan-2-yl) phenyl) imino)-1λ⁶-thietane-1-oxide 77b

At room temperature, compound 77a (630 mg, 2.14 mmol), diboronic acid pinal ester (652.63 mg, 2.57 mmol), 1,1′-biphenylphosphine ferrocene palladium chloride (156.58 mg, 0.214 mmol), potassium acetate (525.05 mg, 5.35 mmol), and 1,4-dioxane (6 mL) were added to a round bottom flask sequentially, followed by vacuum pumping and nitrogen gas purging three times, and heated up to 100° C. and stirred for 1 h. After the reaction was completed, the mixture was concentrated under reduced pressure to obtain a crude product. The crude product was purified by column chromatography (PE/EtOAc=3/1) to obtain compound 77b (500 mg), brown solid.

MS (ESI) m/z 344[M+H]⁺.

### Step 3 1-(4-(4-amino-7-methyl-7H-pyrrolo [2,3-d] pyrimidin-5-yl)-2,5-difluorophenyl) imino)-1λ⁶-thietane-1-oxide 77c

At room temperature, compound 77b (500 mg, 1.45 mmol), compound 1g (40.20 mg, 0.14 mmol), tetrakis (triphenylphosphine) palladium (167.55 mg, 0.145 mmol), potassium phosphate (923.37 mg, 4.35 mmol), 1,4-dioxane (10 mL), and water (2 mL) were added to a round bottom flask sequentially, followed by vacuum pumping and nitrogen gas purging three times, and heated up to 90° C. and stirred for 3 h. After TLC monitored the completion of the reaction, the mixture was cooled down to room temperature and concentrated under reduced pressure to obtain a crude product. The crude product was purified by column chromatography (DCM/MeOH=15/1) to obtain compound 77c (300 mg), reddish brown solid.

MS (ESI) m/z 364 [M+H]⁺.

### Step 4 1-(4-(4-amino-6-iodo-7-methyl-7H-pyrrolo [2,3-d] pyrimidin-5-yl)-2,5-difluorophenyl) imino)-1λ⁶-thietane-1-oxide 77d

At room temperature, compound 77c (300 mg, 0.83 mmol) was dissolved in dichloromethane (5 mL), and then trifluoroacetic acid (188.36 mg, 1.65 mmol) was added. After it was cooled down to 0° C., NIS (278.75 mg, 1.24 mmol) was added in batches, and the reaction was carried out at room temperature for 2 h. After the reaction was completed, saturated sodium bicarbonate solution was added and extracted with dichloromethane. The organic phase was concentrated under reduced pressure to obtain a crude product. The crude product was purified by column chromatography (DCM/MeOH=15/1) to obtain compound 77d (50 mg), light yellow solid.

MS (ESI) m/z 489 [M+H]⁺.

### Step 5 N-(4-(4-amino-5-(2,5-difluoro-4-(1-oxo-1λ⁶-thiocyclobutan-1-ylidene) amino) phenyl)-7-methyl-7H-pyrrolo [2,3-d] pyrimidin-6-yl)-3-fluorophenyl) methacrylamide 77

At room temperature, compound 77d (50 mg, 0.10 mmol), compound 1j (37.23 mg, 0.12 mmol), tetrakis (triphenylphosphine) palladium (11.56 mg, 0.01 mmol), potassium phosphate (64.95 mg, 0.31 mmol), 1,4-dioxane (2 mL), and water (0.4 mL) were added to a round bottom flask sequentially, followed by vacuum pumping and nitrogen gas purging three times, and heated up to 90° C. and stirred for 2 h. After the reaction was completed, the mixture was cooled down to room temperature and concentrated under reduced pressure to obtain a crude product. The crude product was purified by column chromatography (DCM/MeOH=15/1) to obtain compound 77 (20 mg), white solid.

MS (ESI) m/z 541 [M+H]⁺.

¹H NMR (400 MHz, DMSO-d₆) δ 10.10 (s, 1H), 8.19 (s, 1H), 8.16 (s, 1H), 7.77 (d, J=8.0 Hz, 1H), 7.48 (d, J=8.0 Hz, 2H), 7.21 (t, J=12.0 Hz, 1H), 6.02 (s, 2H), 5.82 (s, 1H), 5.57 (s, 1H), 4.37-4.28 (m, 4H), 3.51 (s, 3H), 2.27-2.21 (m, 2H), 1.98 (s, 3H).

### Example 78 N-(4-(4-amino-7-(2-(dimethylamino) ethyl)-5-(4-((1-hydroperoxide-1λ⁶-thiophene-1-ylidene) amino) phenyl)-7H-pyrrolo [2,3-d] pyrimidin-6-yl)-3-fluorophenyl) methacrylamide

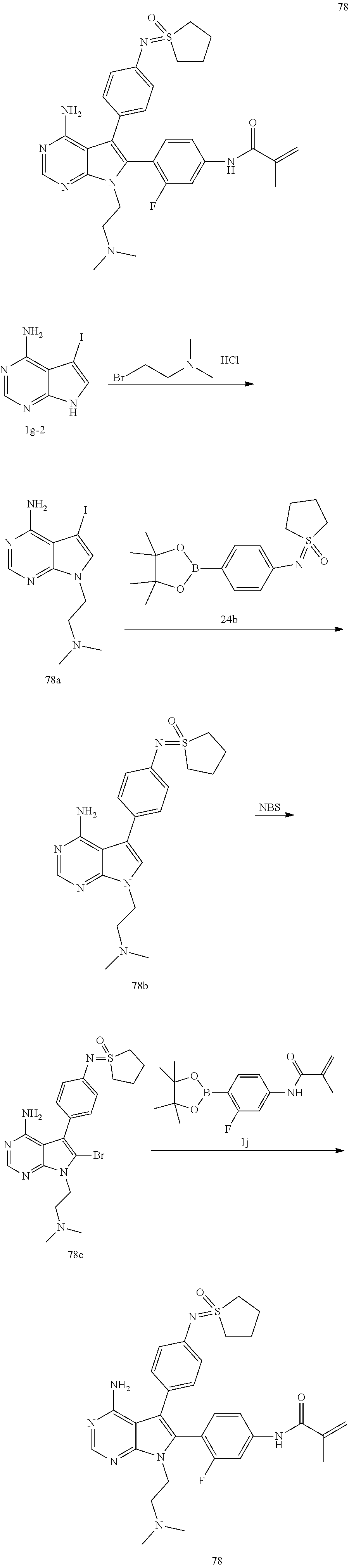

### Step 1 7-(2-(dimethylamino) ethyl)-5-iodo-7H-pyrrolo [2,3-d] pyrimidin-4-amine 78a

At room temperature, compound 1g-2 (1 g, 3.85 mmol), (2-bromomethyl) dimethylamine hydrochloride (872.78 mg, 5.78 mmol), cesium carbonate (2.51 g, 7.7 mmol), and N,N-dimethylformamide (10 mL) were added to a round bottom flask sequentially, and the reaction was stirred at room temperature for 16 h. After TLC monitored the completion of the reaction, the mixture was diluted with water, extracted with ethyl acetate (30 mL×3), and washed with saturated saline (30 mL), and the organic phases were combined, dried over anhydrous sodium sulfate and concentrated under reduced pressure to obtain a crude product. The crude product was purified by column chromatography (DCM/MeOH=15/1) to obtain compound 78a (400 mg), gray solid.

MS (ESI) m/z 332 [M+H]⁺.

### Step 2 1-(4-(4-amino-7-(2-(dimethylamino) ethyl)-7H-pyrrolo [2,3-d] pyrimidin-5-yl) phenyl) imino) tetrahydro-1H-1λ⁶-thiophene-1-oxide 78b

At room temperature, compound 78a (400 mg, 1.2 mmol), compound 24b (423.72 mg, 0.13 mmol), tetrakis (triphenylphosphine) palladium (138.67 mg, 0.12 mmol), potassium phosphate (764.17 mg, 3.6 mmol), 1,4-dioxane (10 mL), and water (2 mL) were added to a round bottom flask sequentially, followed by vacuum pumping and nitrogen gas purging three times, and heated up to 90° C. and stirred for 3 h. After TLC monitored the completion of the reaction, the mixture was cooled down to room temperature and concentrated under reduced pressure to obtain a crude product. The crude product was purified by column chromatography (DCM/MeOH=15/1) to obtain compound 78b (210 mg), reddish brown solid.

MS (ESI) m/z 399 [M+H]⁺.

### Step 3 1-(4-(4-amino-6-bromo-7-(2-(dimethylamino) ethyl)-7H-pyrrolo [2,3-d]pyrimidin-5-yl) phenyl) imino) tetrahydro-1H-1λ⁶-thiophene-1-oxide 78c

At room temperature, compound 78b (210 mg, 0.53 mmol) was dissolved in DMF (5 mL), and it was cooled down to 0° C., and a solution of NBS in DMF (98.55 mg NBS dissolved in 2 mL DMF) was added dropwise, and then temperature was restored to room temperature to react for additional 2 h. After the reaction was completed, the reaction was quenched by added saturated sodium bisulfite aqueous solution, extracted with dichloromethane, and the organic phase was concentrated to obtain a crude product. The crude product was purified by column chromatography (DCM/MeOH=15/1) to obtain compound 78c (100 mg), light yellow solid.

MS (ESI) m/z 477 [M+H]⁺.

### Step 4 N-(4-(4-amino-7-(2-(dimethylamino) ethyl)-5-(4-((1-hydroperoxide-1λ⁶-thiophene-1-ylidene) amino) phenyl)-7H-pyrrolo [2,3-d] pyrimidin-6-yl)-3-fluorophenyl) methacrylamide 78

At room temperature, compound 78c (100 mg, 0.21 mmol), compound 1j (76.9 mg, 0.25 mmol), tetrakis (triphenylphosphine) palladium (24.26 mg, 0.02 mmol), potassium phosphate (133.62 mg, 0.63 mmol), 1,4-dioxane (2 mL), and water (0.4 mL) were added to a round bottom flask sequentially, followed by vacuum pumping and nitrogen gas purging three times, and heated up to 90° C. and stirred for 2 h. After TLC monitored the completion of the reaction, the mixture was cooled down to room temperature and concentrated under reduced pressure to obtain a crude product. The crude product was purified by column chromatography (DCM/MeOH=15/1) to obtain compound 78 (7.7 mg), white solid.

MS (ESI) m/z 576 [M+H]⁺.

¹H NMR (400 MHz, DMSO-d₆) δ 10.10 (s, 1H), 8.17 (s, 1H), 7.74 (d, J=8.0 Hz, 1H), 7.51 (d, J=8.0 Hz, 1H), 7.31 (t, J=12.0 Hz, 1H), 7.05 (d, J=8.0 Hz, 2H), 6.65 (d, J=8.0 Hz, 2H), 6.02 (s, 2H), 5.82 (s, 1H), 5.57 (s, 1H), 4.16-3.99 (m, 2H), 3.35-3.21 (m, 4H), 2.38-2.30 (m, 2H), 2.21-2.08 (m, 4H), 1.98 (s, 6H), 1.94 (s, 3H).

### Example 79 N-(4-(4-amino-7-(2-hydroxy-2-methylpropyl)-5-(4-((1-hydroperoxid-1λ⁶-thiophene-1-ylidene) amino) phenyl)-7H-pyrrolo [2,3-d] pyrimidin-6-yl)-3-fluorophenyl) methacrylamide

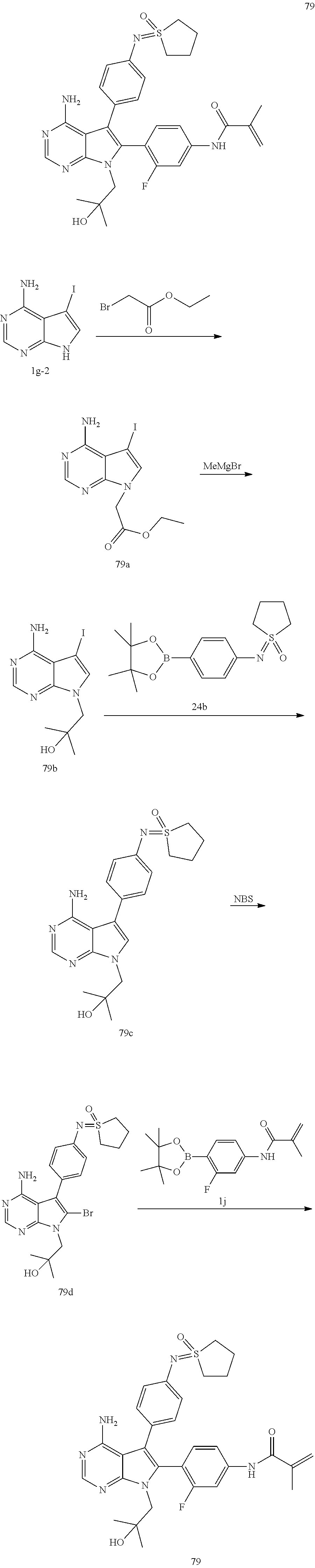

### Step 1 ethyl 2-(4-amino-5-iodo-7H-pyrrolo [2,3-d] pyrimidin-7-yl) acetate 79a

Compound 1g-2 (1 g, 3.85 mmol), ethyl bromoacetate (963.59 mg, 5.77 mmol), and cesium carbonate (2.51 g, 7.7 mmol) were dissolved in N,N-dimethylformamide (10 mL) and the reaction was carried out at room temperature for 16 h. After the reaction was completed, water (100 mL) was added and extracted with ethyl acetate (30 mL×3), and washed with saturated saline (30 mL). The organic phases were combined, dried over anhydrous sodium sulfate and concentrated under reduced pressure to obtain a crude product. The crude product was purified by column chromatography (DCM/MeOH=15/1) to obtain compound 79a (600 mg), gray solid.

MS (ESI) m/z 347 [M+H]⁺.

### Step 2 1-(4-amino-5-iodo-7H-pyrrolo [2,3-d] pyrimidin-7-yl)-2-methylpropyl-2-ol 79b

Compound 79a (600 mg, 1.67 mmol) was dissolved in tetrahydrofuran (10 mL), followed by vacuum pumping and nitrogen gas purging three times. After it was cooled down to −56° C., 1.0 M methyl magnesium bromide-tetrahydrofuran solution (8.4 ml, 8.34 mmol) was added slowly dropwise in batches, and the reaction was stirred at −56° C. for additional 2 h. After the reaction was completed, the mixture was warmed up to room temperature naturally, diluted with water, extracted with ethyl acetate (30 mL×3), and washed with saturated saline (30 mL), and the organic phases were combined, dried over anhydrous sodium sulfate and concentrated under reduced pressure to obtain a crude product. The crude product was purified by column chromatography (DCM/MeOH=15/1) to obtain compound 79b (400 mg), light yellow solid.

MS (ESI) m/z 333 [M+H]⁺.

### Step 3 1-(4-(4-amino-7-(2-hydroxy-2-methylpropyl)-7H-pyrrolo [2,3-d] pyrimidin-5-yl) phenyl) imino) tetrahydro-1H-1λ⁶-thiophene-1-oxide 79c

To a round bottom flask, compound 79b (400 mg, 1.2 mmol), compound 24b (423.72 mg, 0.13 mmol), tetrakis (triphenylphosphine) palladium (138.67 mg, 0.12 mmol), potassium phosphate (764.17 mg, 3.6 mmol), 1,4-dioxane (10 mL), and water (2 mL) were added in sequence, followed by vacuum pumping and nitrogen gas purging three times, and heated up to 90° C. and stirred for 3 h. After the reaction was completed, the mixture was cooled down to room temperature and concentrated under reduced pressure to obtain a crude product. The crude product was purified by column chromatography (DCM/MeOH=15/1) to obtain compound 79c (150 mg), reddish brown solid.

MS (ESI) m/z 400 [M+H]⁺.

### Step 4 1-(4-(4-amino-6-bromo-7-(2-hydroxy-2-methylpropyl)-7H-pyrrolo [2,3-d]pyrimidin-5-yl) phenyl) imino) tetrahydro-1H-1λ⁶-thiophene-1-oxide 79d

Compound 79c (150 mg, 0.37 mmol) was dissolved in N,N-dimethylformamide (5 mL). After it was cooled down to 0° C., N-bromosuccinimide (69.93 mg, 0.39 mmol) was added in batches, and the reaction was stirred at room temperature for 2 h. After the reaction was completed, the mixture was concentrated under reduced pressure to obtain a crude product. The crude product was purified by column chromatography (DCM/MeOH=15/1) to obtain compound 79d (100 mg), light yellow solid.

MS (ESI) m/z 478 [M+H]⁺.

### Step 5 N-(4-(4-amino-7-(2-hydroxy-2-methylpropyl)-5-(4-((1-hydroperoxid-1λ⁶-thiophene-1-ylidene) amino) phenyl)-7H-pyrrolo [2,3-d] pyrimidin-6-yl)-3-fluorophenyl) methacrylamide 79

To a round bottom flask, compound 79d (100 mg, 0.21 mmol), compound 1j (70.19 mg, 0.23 mmol), tetrakis (triphenylphosphine) palladium (24.26 mg, 0.02 mmol), potassium phosphate (133.62 mg, 0.63 mmol), 1,4-dioxane (2 mL), and water (0.4 mL) were added in sequence, followed by vacuum pumping and nitrogen gas purging three times, and heated up to 90° C. and reacted for 2 h. After the reaction was completed, the mixture was cooled down to room temperature and concentrated under reduced pressure to obtain a crude product. The crude product was purified by column chromatography (DCM/MeOH=15/1) to obtain compound 79 (13.4 mg), white solid.

MS (ESI) m/z 577 [M+H]⁺.

¹H NMR (400 MHz, DMSO-d₆) δ 10.06 (s, 1H), 8.17 (s, 1H), 7.71 (d, J=8.0 Hz, 1H), 7.47 (d, J=8.0 Hz, 1H), 7.27 (t, J=12.0 Hz, 1H), 7.28-7.03 (m, 2H), 6.88-6.84 (m, 2H), 6.02 (s, 2H), 5.80 (s, 1H), 5.56 (s, 1H), 4.91 (s, 1H), 4.16-3.99 (m, 2H), 3.28-3.21 (m, 4H), 2.22-2.02 4 (m, 4H), 1.94 (s, 3H), 0.88 (s, 6H).

### Example 80 N-(4-(4-amino-5-(3-methoxy-4-((1-hydroperoxid-12-thiophene-1-ylidene) amino) phenyl)-7-methyl-7H-pyrrolo [2,3-d] pyrimidin-6-yl)-3-fluorophenyl) methacrylamide

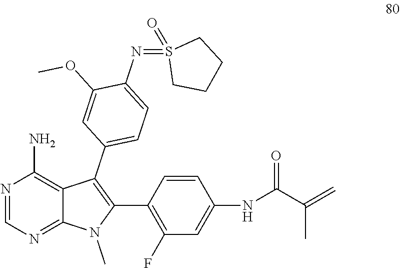 [SMILES: C=C(C)C(=O)Nc1ccc(-c2c(-c3ccc(N=S4(=O)CCCC4)c(OC)c3)c3c(N)ncnc3n2C)c(F)c1]

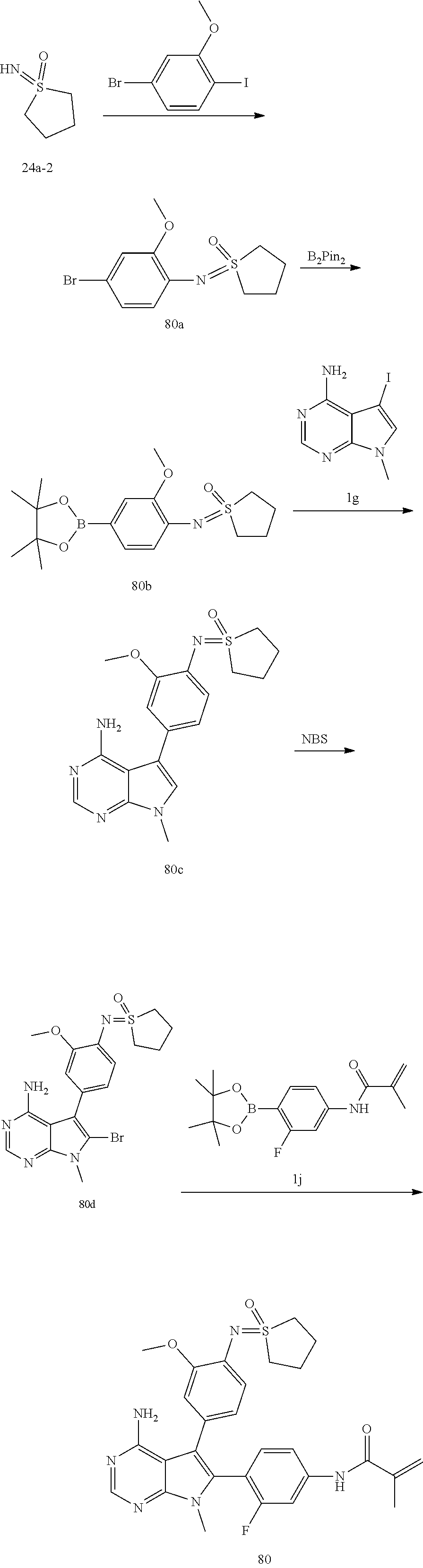

### Step 1 1-((4-bromo-2-methoxyphenyl) imino) tetrahydro-1H-1λ⁶-thiophene-1-oxide 80a

At room temperature, compound 24a-2 (1 g, 8.4 mmol), 4-bromo-1-iodo-2-methoxybenzene (3.14 g, 10.08 mmol), tris (dibenzylideneacetone) dipalladium (192.3 mg, 0.21 mmol), 4,5-diphenylphosphine-9,9-dimethyloxaanthracene (364.53 mg, 0.63 mmol), cesium carbonate (3.83 g, 11.76 mmol), and 1,4-dioxane (20 mL) were added to a round bottom flask sequentially, followed by vacuum pumping and nitrogen gas purging three times, and heated up to 90° C. and stirred for 3 h. After TLC monitored the completion of the reaction, the mixture was cooled down to room temperature and concentrated under reduced pressure to obtain a crude product. The crude product was purified by column chromatography (PE/EtOAc=1/3) to obtain compound 80a (1.2 g), yellow solid.

MS (ESI) m/z 303 [M+H]⁺.

### Step 2 1-((2-methoxy-4-(4,4,5,5-tetramethyl-1,3,2-dioxaborolan-2-yl) phenyl) imino) tetrahydro-1H-1λ⁶-thiophene-1-oxide 80b

To a round bottom flask, compound 80a (1.2 g, 3.96 mmol), bis(pinacolato)diboron (1.21 g, 4.75 mmol), potassium acetate (971.59 mg, 9.9 mmol), 1,1′-biphenylphosphine ferrocene palladium chloride (285.36 mg, 0.39 mmol), and 1,4-dioxane (20 mL) were added in sequence, followed by vacuum pumping and nitrogen gas purging three times, and heated up to 100° C. and stirred for 1 h. After the reaction was completed, the mixture was cooled down to room temperature and filtered under reduced pressure, the filter cake was washed with dichloromethane three times, and all filtrates were combined, concentrated under reduced pressure to obtain a crude product. The crude product was purified by column chromatography (PE/EtOAc=1/5) to obtain compound 80b (1 g), light yellow solid.

MS (ESI) m/z 352[M+H]⁺.

### Step 3 1-(4-(4-amino-7-methyl-7H-pyrrolo [2,3-d] pyrimidin-5-yl)-2-methoxyphenyl) imino) tetrahydro-1H-1λ⁶-thiophene-1-oxide 80c

To around bottom flask, compound 80b (1 g, 2.84 mmol), compound 1g (654.10 mg, 2.37 mmol), tetrakis (triphenylphosphine) palladium (273.87 mg, 0.237 mmol), potassium phosphate (1.51 g, 7.11 mmol), 1,4-dioxane (20 mL), and water (4 mL) were added in sequence, followed by vacuum pumping and nitrogen gas purging three times, and heated up to 90° C. and reacted for 3 h. After TLC monitored the completion of the reaction, the mixture was cooled down to room temperature and concentrated under reduced pressure to obtain a crude product. The crude product was purified by column chromatography (DCM/MeOH=15/1) to obtain compound 80c (430 mg), reddish brown solid.

MS (ESI) m/z 372 [M+H]⁺.

### Step 4 1-(4-(4-amino-6-bromo-7-methyl-7H-pyrrolo [2,3-d] pyrimidin-5-yl)-2-methoxyphenyl) imino) tetrahydro-1H-1λ⁶-thiophene-1-oxide 80d

Compound 80c (430 mg, 1.16 mmol) was dissolved in N,N-dimethylformamide (5 mL), and it was cooled down down to 0° C., and then N-bromosuccinimide (217.13 mg, 1.22 mmol) was added in batches, and heated up to room temperature and reacted for 2 h. After the reaction was completed, the mixture was concentrated under reduced pressure to obtain a crude product. The crude product was purified by column chromatography (DCM/MeOH=15/1) to obtain compound 80d (300 mg), light yellow solid.

MS (ESI) m/z 450 [M+H]⁺.

### Step 5 N-(4-(4-amino-5-(3-methoxy-4-((1-hydroperoxid-1λ⁶-thiophene-1-ylidene) amino) phenyl)-7-methyl-7H-pyrrolo [2,3-d] pyrimidin-6-yl)-3-fluorophenyl) methacrylamide 80

To a round bottom flask, compound 80d (100 mg, 0.22 mmol), compound 1j (77.08 mg, 0.24 mmol), tetrakis (triphenylphosphine) palladium (25.42 mg, 0.022 mmol), potassium phosphate (140.1 mg, 0.66 mmol), 1,4-dioxane (2 mL), and water (0.4 mL) were added in sequence, followed by vacuum pumping and nitrogen gas purging three times, and heated up to 90° C. and reacted for 2 h. After the reaction was completed, the mixture was cooled down to room temperature and concentrated under reduced pressure to obtain a crude product. The crude product was purified by column chromatography (DCM/MeOH=15/1) to obtain compound 80 (17.4 mg), white solid.

MS (ESI) m/z 549 [M+H]⁺.

¹H NMR (400 MHz, DMSO-d₆) δ 10.11 (s, 1H), 8.19 (s, 1H), 7.78 (d, J=8.0 Hz, 1H), 7.48 (d, J=8.0 Hz, 1H), 7.29 (t, J=12.0 Hz, 1H), 6.85 (d, J=8.0 Hz, 1H), 6.73-6.65 (m, 2H), 6.12 (s, 2H), 5.82 (s, 1H), 5.57 (s, 1H), 3.60 (s, 3H), 3.53 (s, 3H), 3.31-3.16 (m, 4H), 2.16-2.09 (m, 4H), 1.95 (s, 3H).

### Example 81 N-(4-(4-amino-5-(3-methoxy-4-((1-hydroperoxid-1λ⁶-thiophene-1-ylidene) amino) phenyl)-7-methyl-7H-pyrrolo [2,3-d] pyrimidin-6-yl)-3-chlorophenyl) methacrylamide

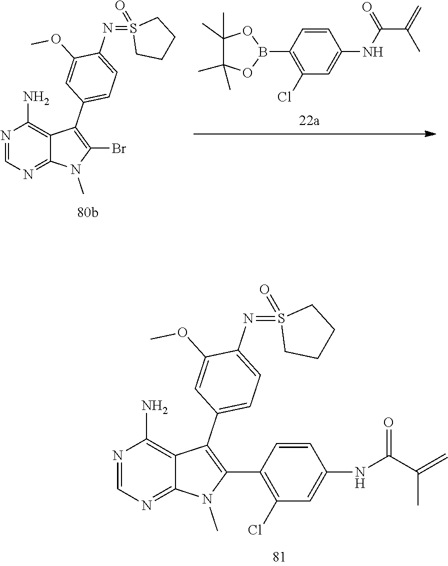

Compound 81 was synthesized with reference to the similar synthetic route and steps of compound 12, wherein, compound 12a was replaced with compound 80b, compound 12b was replaced with compound 74a.

MS (ESI) m/z 566 [M+H]⁺.

¹H NMR (400 MHz, DMSO-d₆) δ 10.08 (s, 1H), 8.19 (s, 1H), 8.02 (d, J=8.0 Hz, 1H), 7.64 (d, J=8.0 Hz, 1H), 7.35 (t, J=20.0 Hz, 2H), 6.84 (d, J=8.0 Hz, 2H), 6.72-6.66 (m, 4H), 6.12 (s, 2H), 5.83 (s, 1H), 5.57 (s, 1H), 4.31-4.27 (m, 4H), 3.59 (s, 3H), 3.46 (s, 3H), 1.94 (s, 3H) 3H).

### Example 82 N-(4-(4-amino-5-(3-chloro-4-((1-oxide tetrahydro-1λ⁶-thiophene-1-ylidene) amino) phenyl)-7-(methyl-d₃)-7H-pyrrolo [2,3-d] pyrimidin-6-yl)-3-fluorophenyl) methacrylamide

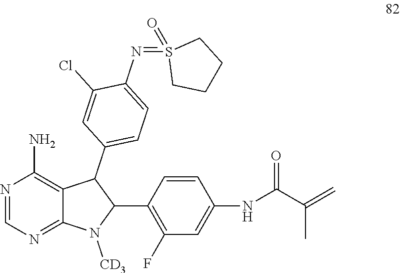 [SMILES: [2H]C([2H])([2H])N1c2ncnc(N)c2C(c2ccc(N=S3(=O)CCCC3)c(Cl)c2)C1c1ccc(NC(=O)C(=C)C)cc1F]

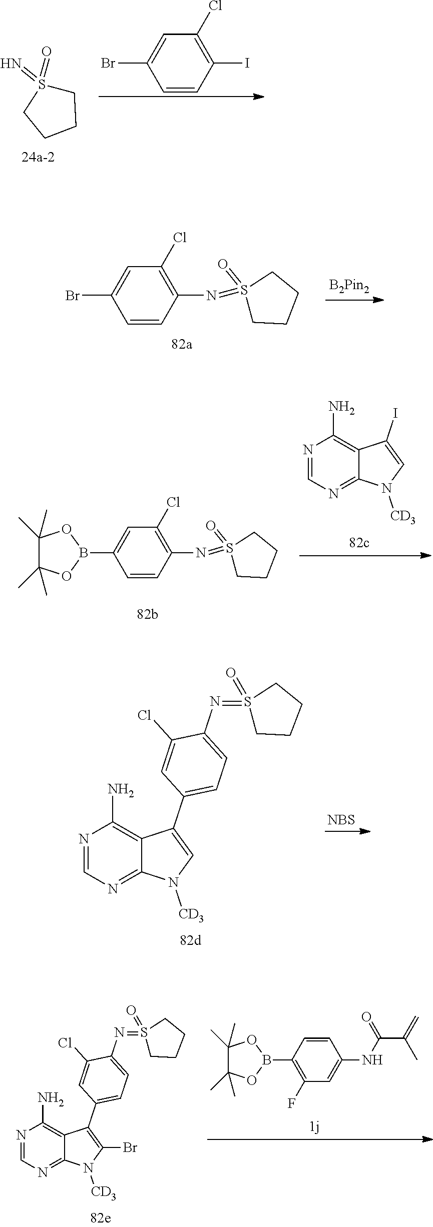

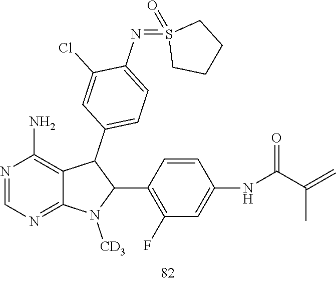 [SMILES: [2H]C([2H])([2H])N1c2ncnc(N)c2C(c2ccc(N=S3(=O)CCCC3)c(Cl)c2)C1c1ccc(NC(=O)C(=C)C)cc1F]

### Step 1 1-((4-bromo-2-chlorophenyl) imino) tetrahydro-1H-1λ⁶-thiophene-1-oxide 82a

To a round bottom flask, compound 24a-2 (1 g, 8.4 mmol), 4-bromo-2-chloro-1-iodobenzene (3.18 g, 10.08 mmol), tris (dibenzylideneacetone) dipalladium (153.84 mg, 0.17 mmol), 4,5-diphenylphosphine-9,9-dimethyloxanthracene (364.53 mg, 0.50 mmol), cesium carbonate (3.56 g, 10.92 mmol), and 1,4-dioxane (10 mL) were added in sequence, followed by vacuum pumping and nitrogen gas purging three times, and heated up to 90° C. and reacted for 3 h. After the reaction was completed, it was cooled down to room temperature, and the mixture was concentrated under reduced pressure to obtain a crude product. The crude product was purified by column chromatography (PE/EtOAc=1/3) to obtain compound 82a (1.2 g), yellow solid.

MS (ESI) m/z 307 [M+H]⁺.

### Step 2 1-((2-chloro-4-(4,4,5,5-tetramethyl-1,3,2-dioxaborolan-2-yl) phenyl) imino) tetrahydro-1H-1λ⁶-thiophene-1-oxide 82b

To a round bottom flask, 82a (1.2 g, 3.9 mmol), bis(pinacolato)diboron (1.19 g, 4.68 mmol), potassium acetate (956.86 mg, 9.75 mmol), 1,1′-biphenylphosphine ferrocene palladium chloride (285.36 mg, 0.39 mmol), and 1,4-dioxane (20 mL) were added sequentially, followed by vacuum pumping and nitrogen gas purging three times, and heated up to 100° C. and reacted for 1 h. After the reaction was completed, the mixture was cooled down to room temperature and filtered under reduced pressure, the filter cake was washed with dichloromethane three times, and all filtrates were combined and concentrated under reduced pressure to obtain a crude product. The crude product was purified by column chromatography (PE/EtOAc=1/5) to obtain compound 82b (1 g), light yellow solid.

MS (ESI) m/z 356 [M+H]⁺.

### Step 3 1-(4-(4-amino-7-(methyl-d₃)-7H-pyrrolo [2,3-d] pyrimidin-5-yl)-2-chlorophenyl) imino) tetrahydro-1H-1λ⁶-thiophene-1-oxide 82d

To a round bottom flask, compound 82b (1 g, 2.82 mmol), compound 82c (709.09 mg, 2.56 mmol), tetrakis (triphenylphosphine) palladium (325.87 mg, 0.282 mmol), potassium phosphate (1.80 g, 8.46 mmol), 1,4-dioxane (10 mL), and water (2 mL) were added in sequence, followed by vacuum pumping and nitrogen gas purging three times, and heated up to 90° C. and reacted for 3 h. After the reaction was completed, the mixture was cooled down to room temperature and concentrated under reduced pressure to obtain a crude product. The crude product was purified by column chromatography (DCM/MeOH=15/1) to obtain compound 82d (530 mg), reddish brown solid.

MS (ESI) m/z 379 [M+H]⁺.

### Step 4 1-(4-(4-amino-6-bromo-7-(methyl-d₃)-7H-pyrrolo [2,3-d] pyrimidin-5-yl)-2-chlorophenyl) imino) tetrahydro-1H-1λ⁶-thiophene-1-oxide 82e

Compound 82d (530 mg, 1.40 mmol) was dissolved in N,N-dimethylformamide (6 mL). After it was cooled down to 0° C., NBS (261.63 mg, 1.47 mmol) was added in batches, and it was heated up to room temperature and reacted for 2 h. After the reaction was completed, the mixture was concentrated under reduced pressure to obtain a crude product. The crude product was purified by column chromatography (DCM/MeOH=15/1) to obtain compound 82e (460 mg), yellow solid.

MS (ESI) m/z 457 [M+H]⁺.

### Step 5 N-(4-(4-amino-5-(3-chloro-4-((1-hydroperoxid-1λ⁶-thiophene-1-ylidene) amino) phenyl)-7-(methyl-d₃)-7H-pyrrolo [2,3-d] pyrimidin-6-yl)-3-fluorophenyl) methacrylamide 82

To a round bottom flask, compound 82e (100 mg, 0.22 mmol), compound 1j (73.85 mg, 0.24 mmol), tetrakis (triphenylphosphine) palladium (25.42 mg, 0.022 mmol), potassium phosphate (140.1 mg, 0.66 mmol), 1,4-dioxane (2 mL), and water (0.4 mL) were added in sequence, followed by vacuum pumping and nitrogen gas purging three times, and heated up to 90° C. and reacted for 2 h. After the reaction was completed, the mixture was cooled down to room temperature and concentrated under reduced pressure to obtain a crude product. The crude product was purified by column chromatography (DCM/MeOH=15/1) to obtain compound 82 (15.7 mg), white solid.

MS (ESI) m/z 556 [M+H]⁺.

¹H NMR (400 MHz, DMSO-d₆) δ 10.11 (s, 1H), 8.19 (s, 1H), 7.78 (d, J=8.0 Hz, 1H), 7.51 (d, J=8.0 Hz, 1H), 7.29 (t, J=20.0 Hz, 1H), 7.17 (s, 1H), 7.07 (s, 1H), 7.01 (s, 1H), 6.11-5.95 (m, 2H), 5.83 (s, 1H), 5.58 (s, 1H), 3.40-3.37 (m, 4H), 2.22-2.12 (m, 4H), 1.95 (s, 3H).

### Example 83 N-(4-(4-amino-7-(methyl-d₃)-5-(4-(1-oxotetrahydro-1λ⁶-thiophene-1-imino) phenyl)-7H-pyrrolo [2,3-d] pyrimidin-6-yl)-3-fluorophenyl) methacrylamide

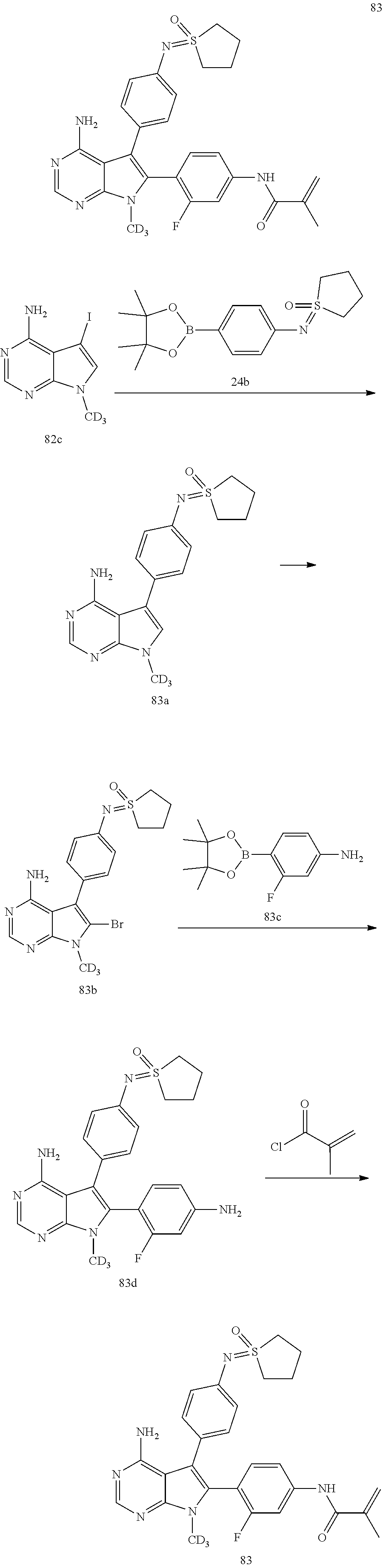

### Step 1 1-(4-(4-amino-7-(methyl-d₃)-7H-pyrrolo [2,3-d] pyrimidin-5-yl) phenyl) imino) tetrahydro-1H-1λ⁶-thiophene-1-oxide 83a

To a reaction flask, compound 82c (18.9 g, 62 mmol), compound 24b (16.8 g, 52 mmol), Pd(PPh₃)₄ (6.0 g, 5.2 mmol), K₃PO₄ (33 g, 156 mmol), 1,4-dioxane (300 mL), and H₂O (30 mL) were added, after replaced with argon gas three times, the reaction solution was heated up to 70° C. and stirred for 16 h. Dioxane was removed by distillated under reduced pressure, water (50 mL) and DCM (200 mL) were added, and it was stirred, filtered through diatomaceous earth, extracted, and concentrated under reduced pressure to obtain crude product. The crude product was purified by column chromatography (MeOH/DCM=1/20) to obtain compound 83a (9.3 g), light yellow solid.

¹H NMR (400 MHz, CDCl₃) δ 8.33 (s, 1H), 7.34 (d, J=8.4 Hz, 2H), 7.14 (d, J=8.4 Hz, 2H), 6.88 (s, 1H), 5.12 (s, 2H), 3.50-3.40 (m, 2H), 3.25-3.18 (m, 2H), 2.40-2.20 (m, 4H).

### Step 2 1-(4-(4-amino-6-bromo-7-(methyl-d₃)-7H-pyrrolo [2,3-d] pyrimidin-5-yl) phenyl) imino) tetrahydro-1H-1λ⁶-thiophene-1-oxide 83b

Compound 83a (7.0 g, 20 mmol) was dissolved in DMF (30 mL), and a solution of NBS (3.8 g, 21 mmol) in DMF (30 mL) was added dropwise at 0° C. The reaction solution was stirred at 0° C. for 1 h, and then quenched with saturated Na₂S₂O₃ aqueous solution (30 mL). Water (20 mL) was added, and it was stirred for half an hour to precipitate a solid, filtered, washed with water, washed with ethyl acetate, and dried to obtain compound 83b (5.8 g), yellow solid.

¹H NMR (400 MHz, CDCl₃) δ 8.29 (s, 1H), 7.33 (d, J=8.4 Hz, 2H), 7.16 (d, J=8.4 Hz, 2H), 4.98 (s, 2H), 3.52-3.43 (m, 2H), 3.28-3.20 (m, 2H), 2.42-2.22 (m, 4H).

### Step 3 1-(4-(4-amino-6-(4-amino-2-fluorophenyl)-7-(methyl-d₃)-7H-pyrrolo [2,3-d]pyrimidin-5-yl) phenyl) imino) tetrahydro-1H-1λ⁶-thiophene-1-oxide 83d

To a reaction flask, compound 83b (5.0 g, 12 mmol), 83c (4.27 g, 18 mmol), Pd(PPh₃)₄ (1.39 g, 1.2 mmol), K₃PO₄ (7.6 g, 36 mmol), dioxane (60 mL), and water (6 mL) were added, after replaced with argon gas three times, the reaction solution was heated up to 90° C. and stirred for 16 h, and then Dichloromethane (40 mL) and H₂O (40 mL) were added, stirred for half an hour, filtered, washed with water, and washed with dichloromethane to obtain compound 83d (4.0 g), gray solid.

¹H NMR (400 MHz, CDCl₃) δ 8.32 (s, 1H), 7.13 (d, J=8.4 Hz, 2H), 6.98 (d, J=8.4 Hz, 2H), 6.85 (t, J=8.2 Hz, 1H), 6.42 (dd, J=11.4, 2.4 Hz, 1H), 6.35 (dd, J=8.2, 2.4 Hz, 1H), 5.01 (s, 2H), 3.89 (s, 2H), 3.46-3.36 (m, 2H), 3.24-3.14 (m, 2H), 2.40-2.18 (m, 4H).

### Step 4 N-(4-(4-amino-7-(methyl-d₃)-5-(4-(1-oxotetrahydro-1λ⁶-thiophene-1-imino) phenyl)-7H-pyrrolo [2,3-d] pyrimidin-6-yl)-3-fluorophenyl) methacrylamide 83

Compound 83d (4.2 g, 9.3 mmol), pyridine (5 mL), and DMF (20 mL) were added to a reaction flask, a solution of methacryloyl chloride (1.36 g, 13 mmol) in DMF (20 mL) was added dropwise at 0° C., and it was heated up to room temperature to react for 1 h. The reaction was quenched with added water, extracted with ethyl acetate (200 mL×3), washed with saturated saline solution, and concentrated under reduced pressure to obtain a crude product. The crude product was purified by column chromatography (MeOH/DCM=1/20) to obtain compound 83 (2.9 g), white solid.

MS (ESI) m/z 522 [M+H]⁺.

¹H NMR (400 MHz, CDCl₃) δ 8.36 (s, 1H), 7.70 (dd, J=11.8, 1.8 Hz, 1H), 7.57 (s, 1H), 7.17-6.93 (m, 6H), 5.80 (s, 1H), 5.51 (d, J=1.6 Hz, 1H), 5.00 (s, 2H), 3.49-3.35 (m, 2H), 3.24-3.14 (m, 2H), 2.40-2.19 (m, 4H), 2.07 (s, 3H).

### Example 84 N-(4-(4-amino-5-(3-fluoro-4-(1-oxo-1λ⁶-thietan-1-imino) phenyl)-7-(methyl-d₃)-7H-pyrrolo [2,3-d] pyrimidin-6-yl)-3-chlorophenyl) methacrylamide

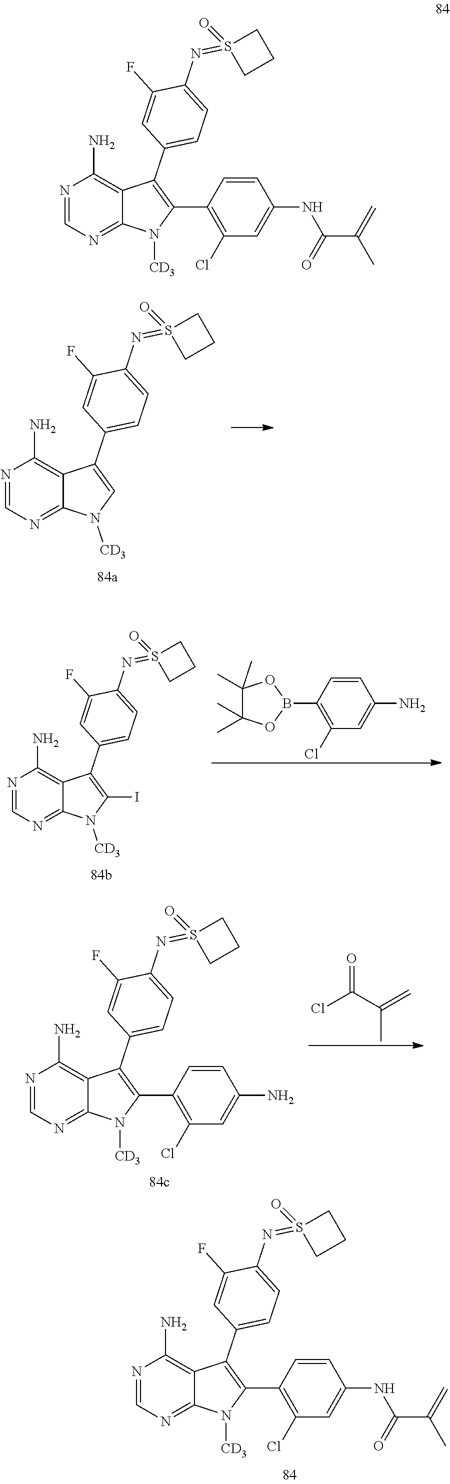

### Step 1 1-(4-(4-amino-6-iodo-7-(methyl-d₃)-7H-pyrrolo [2,3-d] pyrimidin-5-yl)-2-fluorophenyl) imino)-1λ⁶-thietane-1-oxide 84b

Compound 84a (216 mg, 0.6 mmol) was dissolved in dichloromethane (6 mL), trifluoroacetic acid (0.14 mL, 1.8 mmol) was added at 0° C., and NIS (168 mg, 0.72 mmol) was added in batches. The reaction solution was heated up to room temperature and stirred for 1.5 h, and then quenched with added Na₂S₂O₃ (sat. 3 mL), extracted with water (10 mL), dichloromethane, and concentrated under reduced pressure to obtain a crude product. The crude product was purified by column chromatography (MeOH/DCM=1/20) to obtain compound 84b (light yellow solid, 183 mg, 64%).

¹H NMR (400 MHz, CDCl₃) δ 8.24 (s, 1H), 7.21-7.06 (m, 3H), 5.11 (s, 2H), 4.41-4.26 (m, 4H), 2.43-2.32 (m, 2H).

### Step 2 1-(4-(4-amino-6-(4-amino-2-chlorophenyl)-7-(methyl-d₃)-7H-pyrrolo [2,3-d]pyrimidin-5-yl)-2-fluorophenyl) imino)-1λ⁶-thietane-1-oxide 84c

To a reaction flask, compound 84b (183 mg, 0.4 mmol), 3-chloro-4-(4,4,5,5-tetramethyl-1,3,2-dioxaborolan-2-yl) aniline (150 mg, 0.6 mmol), Pd(PPh₃)₄ (45 mg, 0.04 mmol), K₃PO₄ (0.25 g, 1.2 mmol), dioxane (4 mL), and water (0.4 mL) were added, after replaced with argon gas three times, the reaction solution was heated up to 80° C. and stirred for 16 h, and then extracted with dichloromethane (40 mL) and H₂O (10 mL). The crude product was concentrated under reduced pressure and purified by column chromatography (MeOH/DCM=1/20) to obtain compound 84c (light yellow solid, 106 mg, 58%).).

¹H NMR (400 MHz, CDCl₃) δ 8.36 (s, 1H), 7.05-6.88 (m, 4H), 6.77 (d, J=2.4 Hz, 1H), 6.50 (dd, J=8.3, 2.4 Hz, 1H), 5.02 (s, 2H), 4.38-4.20 (m, 4H), 3.86 (s, 2H), 2.41-2.28 (m, 2H).

### Step 3 N-(4-(4-amino-5-(3-fluoro-4-(1-oxo-1λ⁶-thietan-1-imino) phenyl)-7-(methyl-d₃)-7H-pyrrolo [2,3-d] pyrimidin-6-yl)-3-chlorophenyl) methacrylamide 84

Compound 84c (50 mg, 0.1 mmol), pyridine (0.5 mL), and DMF (2 mL) were added to a reaction flask, a solution of methacryloyl chloride (14 mg, 0.13 mmol) in DMF (2 mL) was added dropwise at 0° C., and heated up to room temperature to react for 1 h. The reaction was quenched with added water, extracted with ethyl acetate (100 mL×3), washed with saturated saline solution, and concentrated under reduced pressure to obtain a crude product. The crude product was purified by column chromatography (MeOH/DCM=1/20) to obtain compound 84 (38 mg), white solid.

MS (ESI) m/z 542 [M+H]⁺.

¹H NMR (400 MHz, CDCl₃) δ 8.35 (s, 1H), 7.89 (d, J=2.2 Hz, 1H), 7.73 (s, 1H), 7.37 (dd, J=8.4, 2.2 Hz, 1H), 7.11 (d, J=8.4 Hz, 1H), 6.99 (t, J=8.4 Hz, 1H), 6.92 (d, J=10.0 Hz, 2H), 5.80 (s, 1H), 5.50 (d, J=1.6 Hz, 1H), 5.13 (s, 2H), 4.34-4.20 (m, 4H), 2.37-2.27 (m, 2H).

### Example 85 N-(4-(4-amino-5-(4-(diethyl (oxo)-λ⁶-sulfaneylidene) amino)-2-fluorophenyl)-7-methyl-7H-pyrrolo [2,3-d] pyrimidin-6-yl)-3-fluorophenyl) methacrylamide

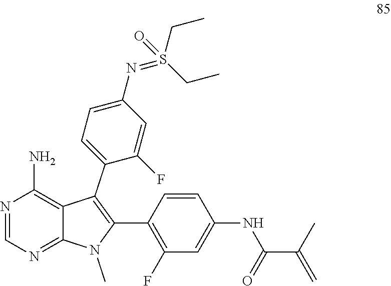 [SMILES: C=C(C)C(=O)Nc1ccc(-c2c(-c3ccc(N=S(=O)(CC)CC)cc3F)c3c(N)ncnc3n2C)c(F)c1]

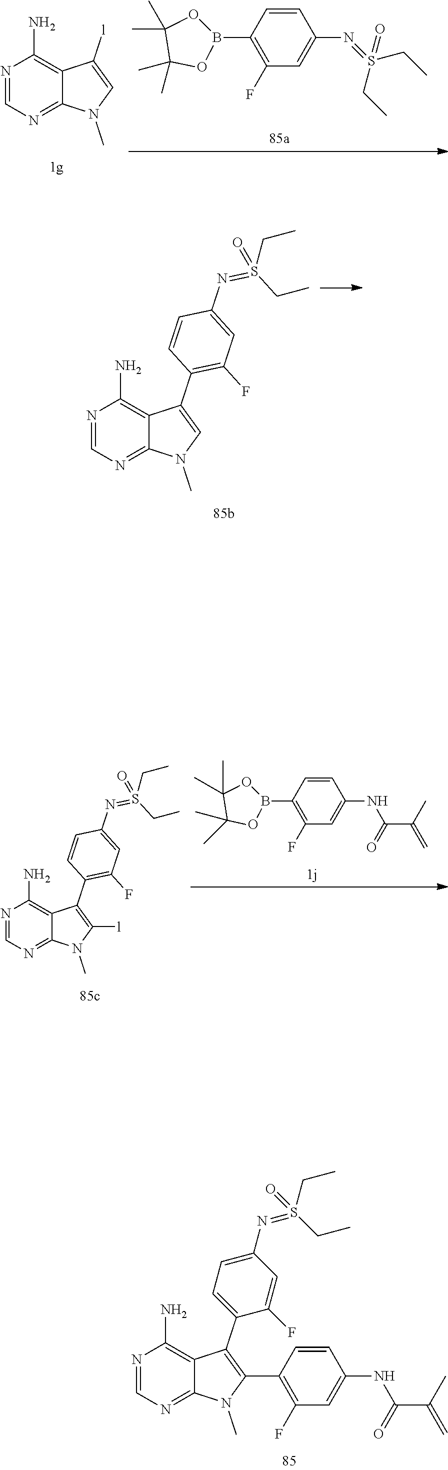

### Step 1 1 (4-(4-amino-7-methyl-7H-pyrrolo [2,3-d] pyrimidin-5-yl)-3-fluorophenyl) imino) diethyl-λ⁶-sulfone 85b

To the reaction flask, compound 85a (500 mg, 1.54 mmol), 1,4-dioxane (5 mL), H₂O (1 mL), compound 1g (383 mg, 1.40 mmol), potassium phosphate (890 mg, 4.2 mmol), and Pd(PPh₃)₄ (162 mg, 0.14 mmol) were added in sequence, after replaced with nitrogen gas three times, heated up to 90° C. and reacted for 3 h. New spots were observed in TLC monitoring, and the reaction solution was diluted with ethyl acetate and H₂O. After filtrated through diatomaceous earth, it was extracted with ethyl acetate, the organic phase was washed with saturated saline solution, dried over anhydrous sodium sulfate, concentrated under reduced pressure, and purified by column chromatography (DCM/MeOH=10/1) to obtain compound 85b (262 mg), yellow solid.

MS (ESI) m/z 362 [M+H]⁺

### Step 2 (4-(4-amino-6-iodo-7-methyl-7H-pyrrolo [2,3-d] pyrimidin-5-yl)-3-fluorophenyl) imino) diethyl-λ⁶-sulfone 85c

Compound 85b (262 mg, 0.72 mmol) was dissolved in anhydrous DCM (3 mL), TFA (246 mg, 2.16 mmol) and NIS (163 mg, 0.72 mmol) were added at 0° C. for 1 h, and then the reaction was quenched with added sodium thiosulfate solution, extracted with saturated sodium bicarbonate aqueous solution and dichloromethane, the organic layers were combined, and washed with saturated salt water once. The organic phase was dried over anhydrous sodium sulfate, concentrated under reduced pressure, and purified by column chromatography (PE/EA=1:1) to obtain compound 85c (176 mg), yellow solid.

MS (ESI) m/z 488 [M+H]⁺

### Step 3 N-(4-(4-amino-5-(4-(diethyl (oxo)-λ⁶-sulfaneylidene) amino)-2-fluorophenyl)-7-methyl-7H-pyrrolo [2,3-d] pyrimidin-6-yl)-3-fluorophenyl) methacrylamide 85

To a reaction flask, compound 85c (176 mg, 0.36 mmol), 1,4-dioxane (4 mL), water (1 mL), compound 1j (132 mg, 0.433 mmol), Pd(PPh₃)₄ (42 mg, 0.036 mmol), and potassium phosphate (82.8 mg, 0.39 mmol) were added, after replaced with nitrogen gas three times, the reaction was carried out at 90° C. for 3 h. The reaction solution was diluted with added Ethyl acetate (10 mL) and H₂O (10 mL), filtered through diatomaceous earth, and extracted with ethyl acetate, and the organic phase was washed with saturated saline solution, dried over anhydrous sodium sulfate, concentrated under reduced pressure, and purified by column chromatography (DCM/MeOH=10/1) to obtain compound 85 (57.5 mg), white solid.

MS (ESI) m/z 539.2 [M+H]⁺.

¹H NMR (400 MHz, MeOD-d4) δ 8.17 (s, 1H), 7.64 (dd, J=12.2, 1.9 Hz, 1H), 7.42-7.34 (m, 2H), 7.29-7.19 (m, 3H), 5.83 (s, 1H), 5.56 (s, 1H), 3.84 (s, 3H), 3.23 (m, 4H), 2.05 (s, 3H), 1.29 (t, J=7.4 Hz, 6H).

### Example 86 N-(4-(4-amino-5-(4-(diethyl (oxo)-λ⁶-sulfaneylidene) amino) phenyl)-7-(2-methoxyethyl)-7H-pyrrolo [2,3-d] pyrimidin-6-yl)-3-fluorophenyl) methacrylamide

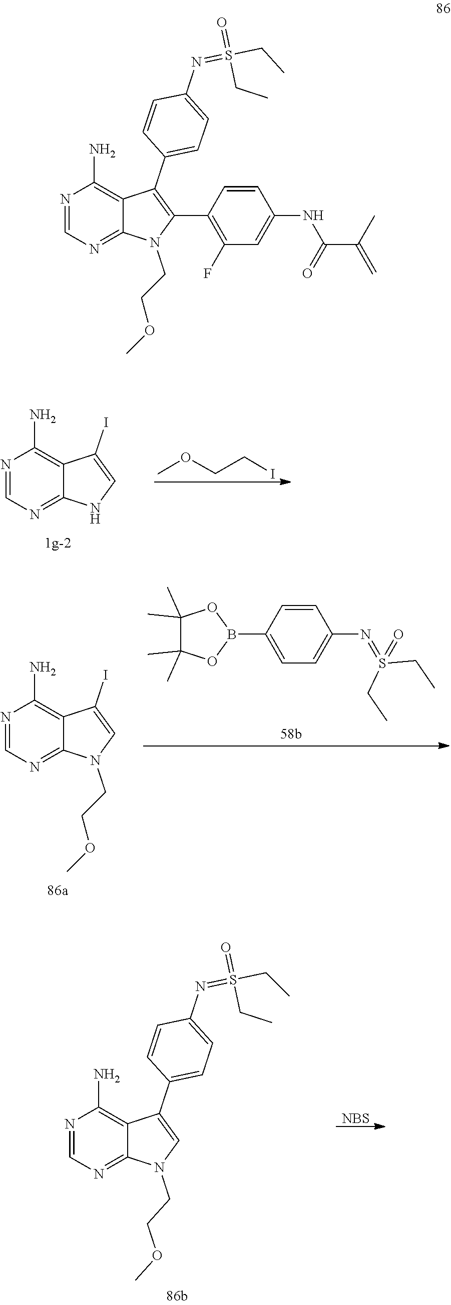

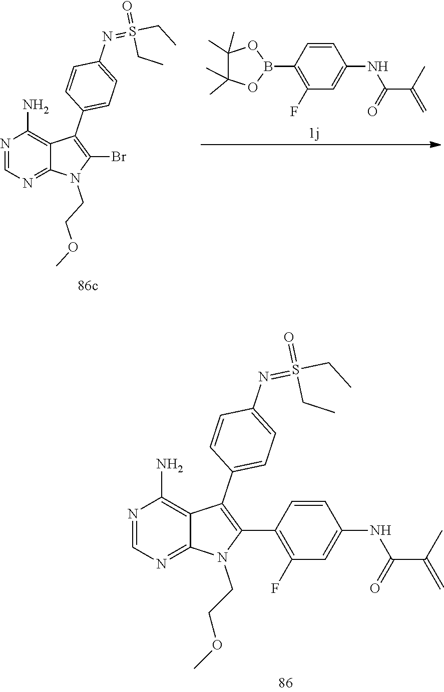

### Step 1 5-iodo-7-(2-methoxyethyl)-7H-pyrrolo [2,3-d] pyrimidin-4-amine 86a

To a reaction flask, compound 1g-2 (500 mg, 1.92 mmol), DMF (5 mL), cesium carbonate (1.25 g, 3.84 mmol) and 1-iodo-2-methoxyethane (429 mg, 2.31 mmol) were added sequentially, the reaction was carried out at room temperature for 2 h. H₂O (50 mL) was added and extracted with ethyl acetate, and the organic phase was washed with saturated saline solution, dried over anhydrous sodium sulfate, concentrated under reduced pressure, and purified by column chromatography (DCM/MeOH=10/1) to obtain compound 86a (464 mg).

MS (ESI) m/z 319 [M+H]⁺

### Step 2 (4-(4-amino-7-(2-methoxyethyl)-7H-pyrrolo [2,3-d] pyrimidin-5-yl) phenyl) imino) diethyl-λ⁶-sulfone 86b

To a reaction flask, compound 86a (440 mg, 1.38 mmol), 1,4-dioxane (5 mL), water (1 mL), compound 58b (537 mg, 1.66 mmol), Pd(PPh₃)₄ (160 mg, 0.138 mmol), and potassium phosphate (292 mg, 1.38 mmol) were added in sequence, after replaced with nitrogen gas three times, the reaction was carried out at 90° C. for 2 h. After the reaction was completed, it was cooled down to room temperature, and the reaction solution was diluted with added ethyl acetate and H₂O. After filtrated through diatomaceous earth to extract with EA, and the organic phase was washed with saturated saline solution, dried over anhydrous sodium sulfate, concentrated under reduced pressure, and purified by column chromatography (DCM/MeOH=10/1) to obtain compound 86b (140 mg), white solid.

MS (ESI) m/z 388 [M+H]⁺

### Step 3 (4-(4-amino-6-bromo-7-(2-methoxyethyl)-7H-pyrrolo [2,3-d] pyrimidin-5-yl) phenyl) imino) diethyl-6-sulfone 86c

Compound 86b (92 mg, 0.24 mmol) was dissolved in anhydrous DMF (4 mL), NBS (44 mg, 0.25 mmol) was added at 0° C., and the reaction was carried out at 0° C. for 1 h, and then the reaction was quenched with added sodium thiosulfate solution, extracted with added H₂O (40 mL) and ethyl acetate 3 times, and the organic phase was dried over anhydrous sodium sulfate, concentrated under reduced pressure, and purified by column chromatography (DCM/MEOH=10/1) to obtain compound 86c (87 mg), yellow oil.

MS (ESI) m/z 467 [M+H]⁺

### Step 4 N-(4-(4-amino-5-(4-(diethyl (oxo)-λ⁶-sulfaneylidene) amino) phenyl)-7-(2-methoxyethyl)-7H-pyrrolo [2,3-d] pyrimidin-6-yl)-3-fluorophenyl) methacrylamide 86

To a reaction flask, compound 86c (87 mg, 0.19 mmol), 1,4-dioxane (2.4 mL), water (0.6 mL), compound 1j (68 mg, 0.22 mmol), potassium phosphate (119 mg, 0.56 mmol), and Pd(PPh₃)₄ (22 mg, 0.019 mmol) were added in sequence, after replaced with nitrogen gas three times, the reaction was carried out at 90° C. for 3 h. After the reaction was completed, it was cooled down to room temperature, and the reaction solution was diluted with ethyl acetate and H₂O. After filtered through diatomaceous earth, it was extracted with ethyl acetate, and the organic phase was washed with saturated saline solution, dried over anhydrous sodium sulfate, concentrated under reduced pressure, and purified by column chromatography (DCM/MeOH=10/1) to obtain compound 86 (9.1 mg), white solid.

MS (ESI) m/z 565.38 [M+H]⁺.

¹H NMR (400 MHz, MeOD-d4) δ 8.19 (s, 1H), 7.69 (dd, J=12.0, 2.0 Hz, 1H), 7.39 (dd, J=8.4, 2.0 Hz, 1H), 7.25 (t, J=8.3 Hz, 1H), 7.14 (d, J=8.2 Hz, 2H), 7.05 (d, J=8.6 Hz, 2H), 5.83 (s, 1H), 5.57 (s, 1H), 4.38 (s, 1H), 4.21 (s, 1H), 3.59 (s, 1H), 3.50 (s, 1H), 3.29 (d, J=7.4 Hz, 4H), 3.16 (d, J=8.9 Hz, 3H), 2.04 (s, 3H), 1.46-1.29 (m, 6H).

### Example 87 N-(4-(4-amino-7-methyl-5-(3-methyl-4-((1-tetrahydro-1λ⁶-thiophene-1-ylidene) amino) phenyl)-7H-pyrrolo [2,3-d] pyrimidin-6-yl)-3-fluorophenyl) methacrylamide

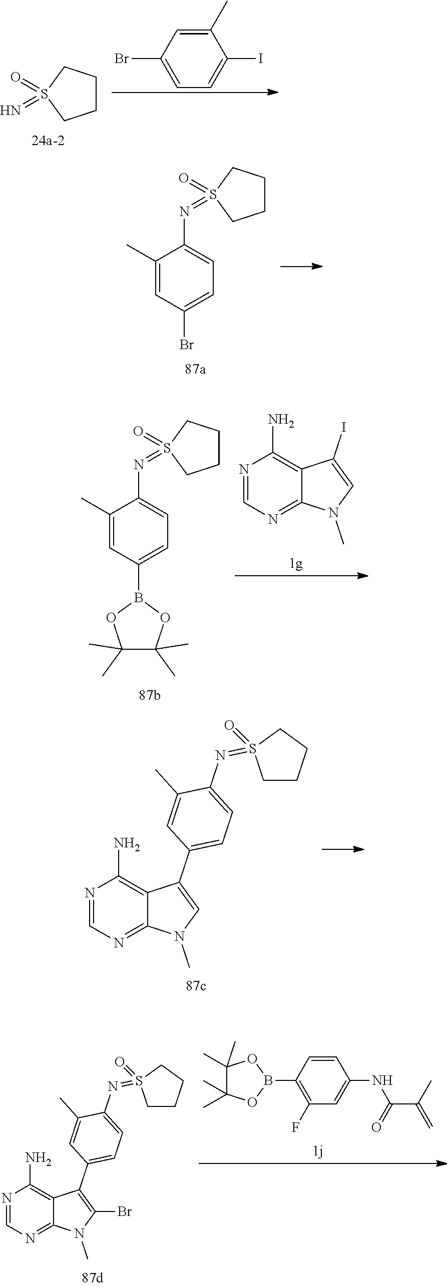

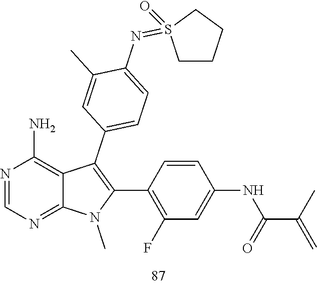 [SMILES: C=C(C)C(=O)Nc1ccc(-c2c(-c3ccc(N=S4(=O)CCCC4)c(C)c3)c3c(N)ncnc3n2C)c(F)c1]

Compound 87 was synthesized with reference to the similar synthetic route and steps of compound 82, wherein, 4-bromo-2-chloroiodobenzene was replaced with 4-bromo-1-iodo-2-methylbenzene.

MS (ESI) m/z 533.35[M+H]⁺.

¹H NMR (400 MHz, DMSO-d₆) δ 10.03 (s, 1H), 8.11 (s, 1H), 7.69 (dd, J=12.4, 1.9 Hz, 1H), 7.41 (dd, J=8.4, 2.0 Hz, 1H), 7.20 (t, J=8.4 Hz, 1H), 7.02-6.76 (m, 3H), 5.75 (s, 1H), 5.50 (s, 1H), 3.44 (s, 3H), 3.18 (m, 4H), 2.18-1.99 (m, 7H), 1.88 (s, 3H).

### Example 88 N-(4-(4-amino-5-(3-chloro-4-((1-oxide tetrahydro-1λ⁶-thiophene-1-ylidene) amino) phenyl)-7-methyl-7H-pyrrolo [2,3-d] pyrimidin-6-yl)-3-fluorophenyl) methacrylamide

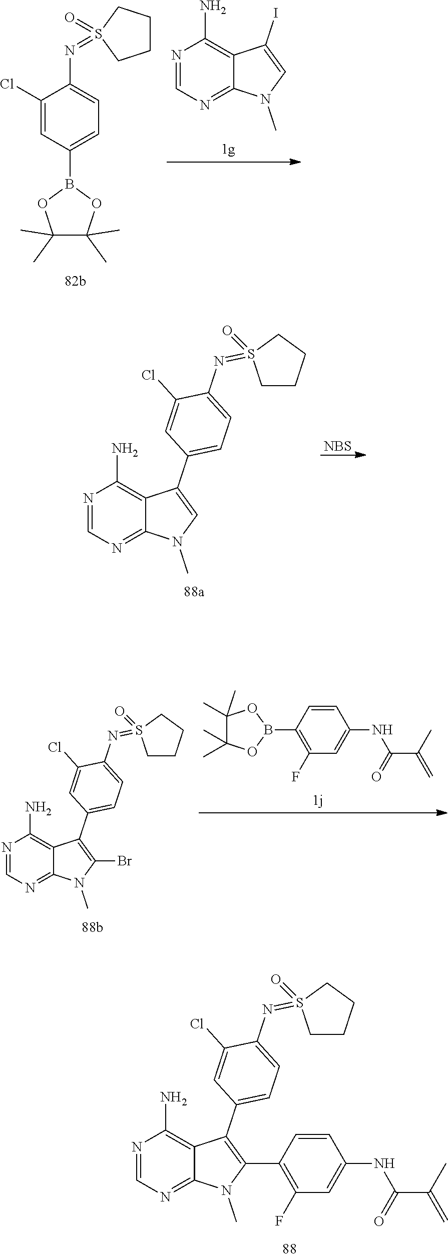

Compound 88 was synthesized with reference to the similar synthetic route and steps of compound 87, wherein, compound 87b was replaced with compound 82b.

MS (ESI) m/z 553.3 [M+H]⁺.

¹H NMR (400 MHz, DMSO-d₆) δ 10.12 (s, 1H), 8.19 (s, 1H), 7.77 (dd, J=12.4, 1.8 Hz, 1H), 7.50 (dd, J=8.5, 1.9 Hz, 1H), 7.29 (t, J=8.4 Hz, 1H), 7.16 (d, J=1.9 Hz, 1H), 7.07 (d, J=8.2 Hz, 1H), 7.02 (dd, J=8.2, 1.9 Hz, 1H), 5.83 (s, 1H), 5.58 (s, 1H), 3.51 (s, 3H), 3.28 (m, 4H), 2.17 (m, 4H), 1.95 (s, 3H).

### Example 89 N-(4-(4-amino-5-(3-(difluoromethyl)-4-((1-tetrahydro-12-thiophene-1-ylidene) amino) phenyl)-7-methyl-7H-pyrrolo [2,3-d] pyrimidin-6-yl)-3-fluorophenyl) methacrylamide

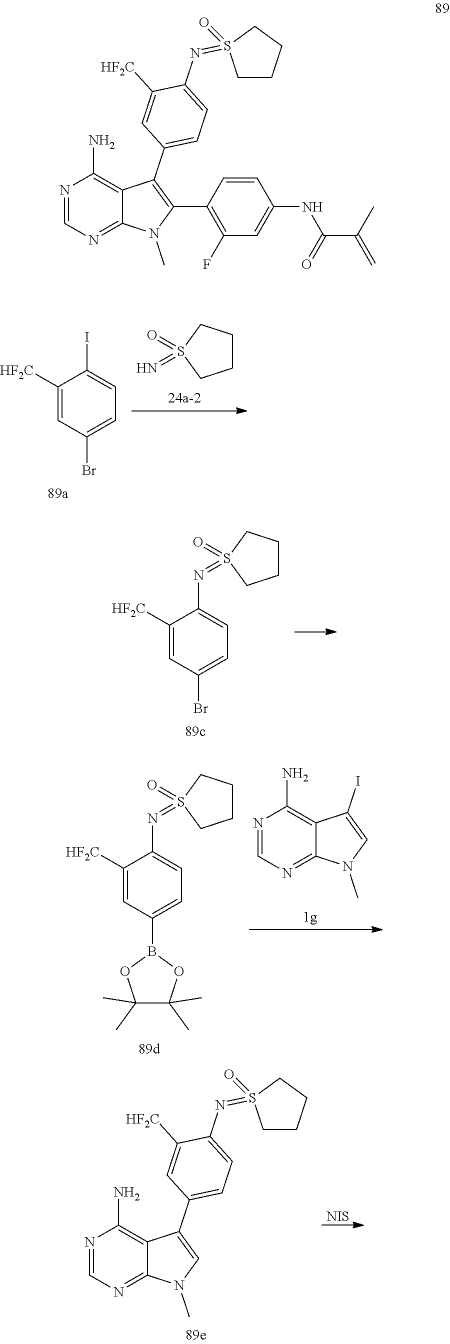

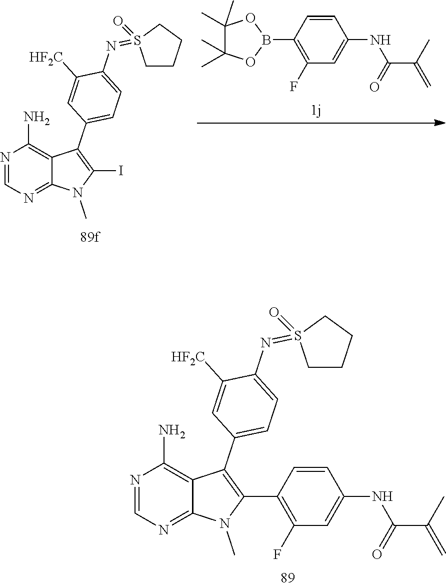

Compound 89 was synthesized with reference to the similar synthetic route and steps of compound 77, wherein, 1-bromo-2,5-difluoro-4-iodobenzene was replaced with compound 89a. Compound 89a was synthesized with reference to WO 2023/001794A1.

MS (ESI) m/z 569.15 [M+H]⁺.

¹H NMR (400 MHz, MeOD-d4) δ 8.19 (s, 1H), 7.72 (dd, J=12.1, 1.8 Hz, 1H), 7.44-7.34 (m, 2H), 7.24-6.83 (m, 4H), 5.81 (s, 1H), 5.55 (s, 1H), 3.62 (s, 3H), 3.46-3.39 (m, 2H), 3.27-3.19 (m, 2H), 2.27 (m, 4H), 2.02 (s, 3H).

### Example 90 N-(4-(4-amino-5-(3-fluoro-4-((1-tetrahydro-1λ⁶-thiophene-1-ylidene) amino) phenyl)-7-(methyl-d₃)-7H-pyrrolo [2,3-d] pyrimidin-6-yl)-5-chloro-2-methylphenyl) methacrylamide

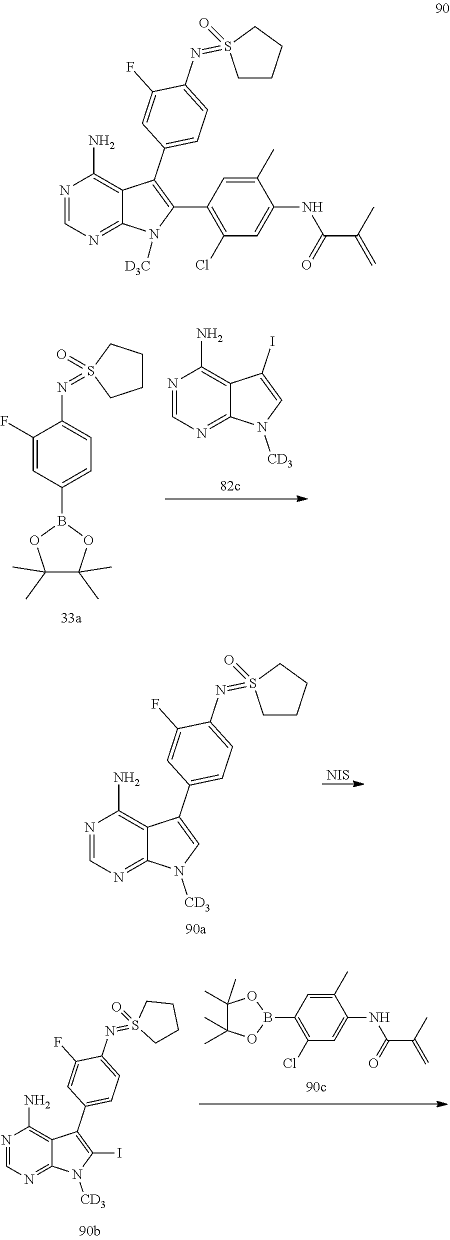

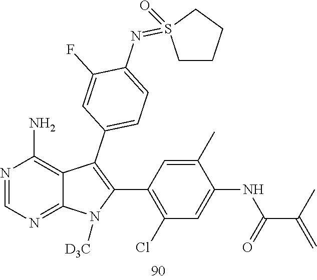 [SMILES: [2H]C([2H])([2H])n1c(-c2cc(C)c(NC(=O)C(=C)C)cc2Cl)c(-c2ccc(N=S3(=O)CCCC3)c(F)c2)c2c(N)ncnc21]

Compound 90 was synthesized with reference to the similar synthetic route and steps of compound 85, wherein, compound 85a was replaced with compound 33a, and compound 1g was replaced with compound 82c in step 1, and compound 1j was replaced with compound 90c in step 3.

MS (ESI) m/z 570.0 [M+H]⁺.

¹H NMR (400 MHz, DMSO-d₆) δ 9.39 (s, 1H), 8.20 (s, 1H), 7.65 (s, 1H), 7.32 (s, 1H), 7.11-6.81 (m, 3H), 5.88 (s, 1H), 5.55 (s, 1H), 3.45-3.36 (m, 2H), 3.27 (dd, J=13.2, 6.6 Hz, 2H), 2.23-2.07 (m, 7H), 1.97 (s, 3H).

### Example 91 N-(4-(4-amino-5-(3-fluoro-4-((1-tetrahydro-1λ⁶-thiophene-1-ylidene) amino) phenyl)-7-(methyl-d₃)-7H-pyrrolo [2,3-d] pyrimidin-6-yl)-5-fluoro-2-methylphenyl) methacrylamide

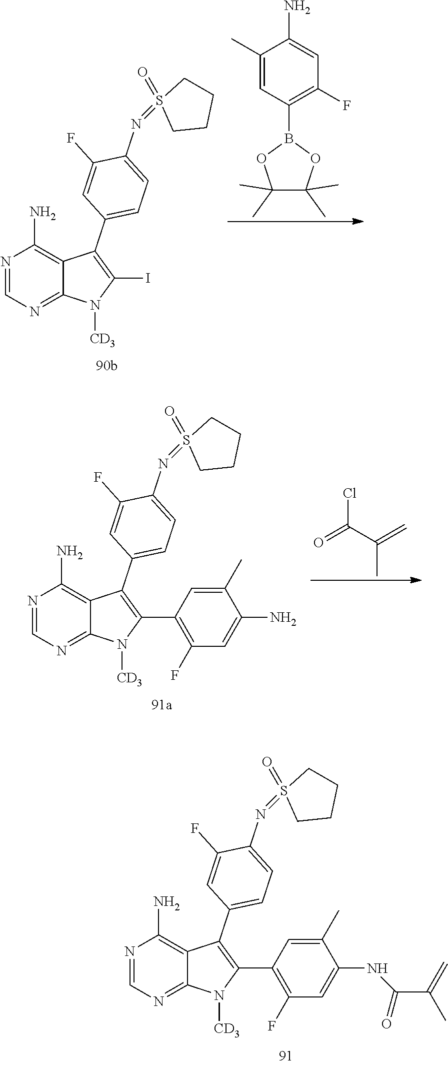

### Step 1 1-(4-(4-amino-6-(4-amino-2-fluoro-5-methylphenyl)-7-(methyl-d₃)-7H-pyrrolo [2,3-d] pyrimidin-5-yl)-2-fluorophenyl) imino) tetrahydro-1H-1λ⁶-thiophene-1-oxide 91a

Compound 90b (210 mg, 0.43 mmol), 5-fluoro-2-methyl-4-(4,4,5,5-tetramethyl-1,3,2-dioxaborolan-2-yl)aniline (160 mg, 0.63 mmol), Pd(PPh₃)₄ (49 mg, 0.04 mmol), and potassium phosphate (270 mg, 1.29 mmol) were dissolved in 1,4-dioxane (8 mL) and water (2 mL), after replaced with nitrogen gas three times, the reaction was refluxed at 90° C. for 12 h. The reaction solution was cooled down to room temperature, filtered through diatomaceous earth, concentrated under reduced pressure to remove the solvent, extracted with DCM/H₂O three times, and the organic layers were combined, washed with saturated saline solution, dried over anhydrous sodium sulfate, concentrated under reduced pressure, purified by column chromatography (DCM/MEOH=40/1) to obtain compound 91a (100 mg), light yellow solid.

MS (ESI) m/z 485.90 [M+H]⁺.

### Step 2 N-(4-(4-amino-5-(3-fluoro-4-((1-tetrahydro-1λ⁶-thiophene-1-ylidene) amino) phenyl)-7-(methyl-d₃)-7H-pyrrolo [2,3-d] pyrimidin-6-yl)-5-fluoro-2-methylphenyl) methacrylamide 91

Compound 91a (44 mg, 0.09 mmol) was dissolved with anhydrous DMF (10 mL), and pyridine (0.5 mL) was added dropwise. Methacryloyl chloride (11 mg, 0.11 mmol) was dissolved in anhydrous DMF (1 mL), and the solution of methacryloyl chloride in DMF was added dropwise to the DMF solution of compound 91a at 0° C., and the reaction was carried out for 1 h, quenched with water, extracted with DCM/H₂O three times, and the organic layers were combined, washed with saturated saline solution, dried over anhydrous sodium sulfate, concentrated under reduced pressure, and purified by reverse column chromatography to obtain compound 91 (13 mg), white solid.

MS (ESI) m/z 553.88 [M+H]⁺.

¹H NMR (400 MHz, CDCl₃) δ 8.39 (s, 1H), 8.15 (d, J=11.6 Hz, 1H), 7.48 (s, 1H), 7.13 (t, J=8.5 Hz, 1H), 7.01-6.86 (m, 3H), 5.88 (s, 1H), 5.55 (d, J=1.8 Hz, 1H), 5.06 (s, 2H), 3.46 (dt, J=13.3, 6.8 Hz, 2H), 3.25 (dt, J=13.0, 6.6 Hz, 2H), 2.34 (q, J=7.7 Hz, 4H), 2.20 (s, 3H), 2.12 (s, 3H).

### Example 92 N-(4-(4-amino-5-(3-fluoro-4-((1-tetrahydro-1λ⁶-thiophene-1-ylidene) amino) phenyl)-7-methyl-7H-pyrrolo [2,3-d] pyrimidin-6-yl) phenyl)-2-(dimethylamino) methyl) acrylamide

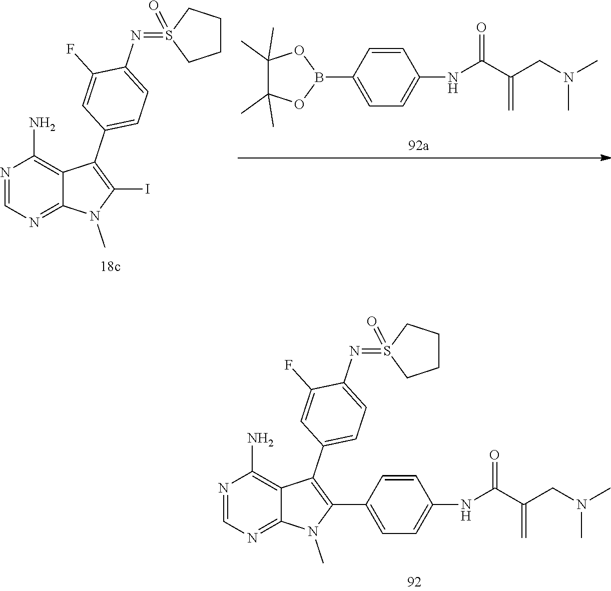

Compound 92 was synthesized with reference to the similar synthetic route and steps of compound 12, wherein, compound 12a was replaced with compound 18c, compound 12b was replaced with compound 92a.

MS (ESI) m/z 562 [M+H]⁺.

¹H NMR (400 MHz, DMSO-d₆) δ 11.25 (s, 1H), 8.19 (s, 1H), 7.67 (d, J=8.0 Hz, 2H), 7.32 (d, J=8.0 Hz, 2H), 7.05 (t, J=12.0 Hz, 1H), 6.99-6.91 (m, 2H), 6.12 (s, 2H), 6.03 (s, 1H), 5.59 (s, 1H), 4.22 (s, 2H), 3.41-3.23 (m, 4H), 2.51-2.47 (m, 4H), 2.25 (s, 3H), 0.88 (s, 6H).

### Example 93 N-(4-(4-amino-5-(4-((dicyclopropyl (oxo)-λ⁶-sulfaneylidene) amino) phenyl)-7-methyl-7H-pyrrolo [2,3-d] pyrimidin-6-yl) phenyl) methacrylamide

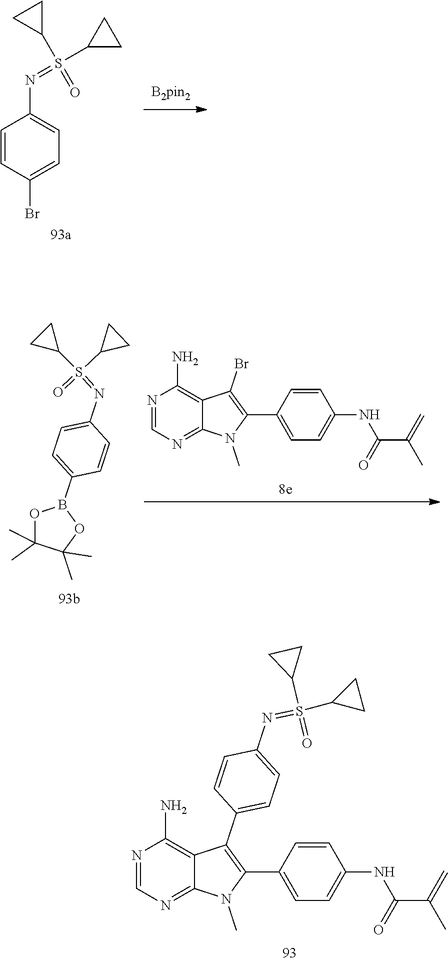

Compound 93 was synthesized with reference to the similar synthetic route and steps of compound 11, wherein, compound 11c was replaced with compound 93a.

MS (ESI) m/z 527.2 [M+H]⁺.

¹H NMR (400 MHz, CDCl₃) δ 8.34 (s, 1H), 7.61-7.50 (m, 3H), 7.23 (d, J=8.46, 2H), 7.10-6.94 (m, 2H), 5.81 (s, 1H), 5.50 (s, 1H), 5.17 (br. s., 2H), 3.77-3.69 (s, 3H), 2.57-2.45 (m, 2H), 2.08 (s, 3H), 1.39-1.16 (m, 4H), 1.11-0.97 (m, 4H)

### Example 94 N-(4-amino-5-(3-fluoro-4-(1-oxotetrahydro-1λ⁶-thiophene-1-ylidene) amino) phenyl)-7-(methyl-d₃)-7H-pyrrolo [2,3-d] pyrimidin-6-yl)-3-fluorophenyl) methacrylamide

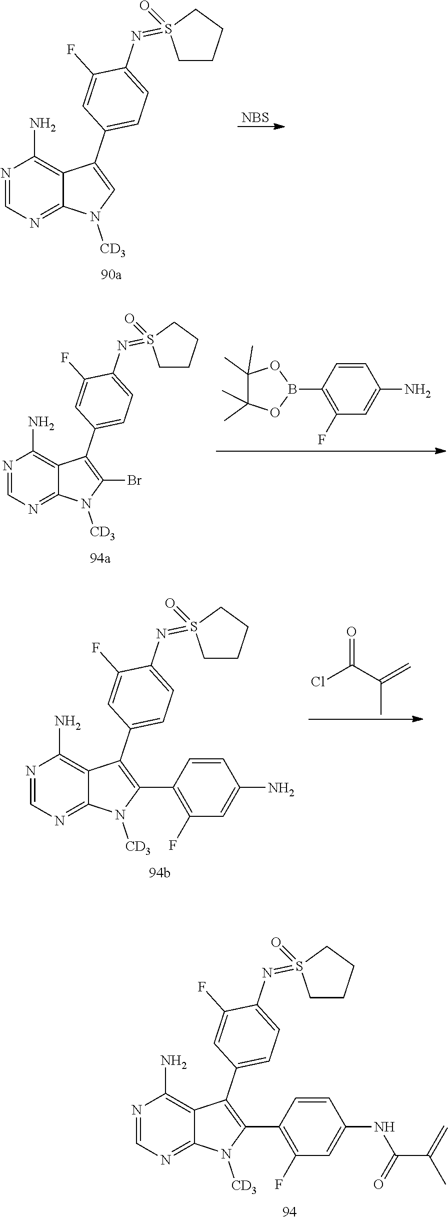

### Step 1 1-(4-(4-amino-6-bromo-7-(methyl-d₃)-7H-pyrrolo [2,3-d] pyrimidin-5-yl)-2-fluorophenyl) imino) tetrahydro-1H-1λ⁶-thiophene-1-oxide 94a

Compound 90a (2.1 g, 5.79 mmol) was dissolved with anhydrous DMF (10 mL), NBS (1.08 g, 6.08 mmol) was added at 0° C., and the reaction was carried out for 2 h, and then quenched with added sodium thiosulfate solution, and extracted with dichloromethane, and the organic layers were combined, and washed with saturated salt water once. The organic phase was dried over anhydrous sodium sulfate, concentrated under reduced pressure, and purified by column chromatography (DCM/MEOH=30/1) to obtain compound 94a (1.4 g), yellow solid.

MS (ESI) m/z 441 [M+H]⁺.

### Step 2 1-(4-(4-amino-6-(4-amino-2-fluorophenyl)-7-(methyl-d₃)-7H-pyrrolo [2,3-d]pyrimidin-5-yl)-2-fluorophenyl) imino) tetrahydro-1H-1λ⁶-thiophene-1-oxide 94b

Compound 94a (1.4 g, 3.17 mmol), 3-fluoro-4-(4,4,5,5-tetramethyl-1,3,2-dioxaborolan-2-yl) aniline (1.13 g, 4.76 mmol), Pd(PPh₃)₄ (366 mg, 0.32 mmol), and potassium phosphate (2.02 g, 9.52 mmol) were dissolved in 1,4-dioxane (20 mL) and water (2 mL), after replaced with nitrogen three times, the reaction was refluxed at 100° C. for 12 h. The reaction solution was cooled down to room temperature, filtered through diatomaceous earth, concentrated under reduced pressure to remove the solvent, extracted with DCM/H₂O three times, and the organic layers were combined, washed with saturated saline solution and dried over anhydrous sodium sulfate, concentrated under reduced pressure, purified by column chromatography (DCM/MEOH=40/1) to obtain compound 94b (1.33 mg), white solid.

MS (ESI) m/z 472 [M+H]⁺.

### Step 3 N-(4-(4-amino-5-(3-fluoro-4-((1-tetrahydro-1λ⁶-thiophene-1-ylidene) amino) phenyl)-7-(methyl-d₃)-7H-pyrrolo [2,3-d] pyrimidin-6-yl)-3-fluorophenyl) methacrylamide 94

Compound 94b (610 mg, 1.29 mmol) was dissolved with anhydrous DMF (10 mL), methacryloyl chloride (162 mg, 4.76 mmol) was added at 0° C., the reaction was carried out for 1 h, and then quenched with water, extracted with dichloromethane, and the organic layers were combined, and washed with saturated salt once. The organic phase was dried over anhydrous sodium sulfate, concentrated under reduced pressure, and purified by column chromatography (DCM/MEOH=20/1) to obtain compound 94 (140 mg), white solid.

MS (ESI) m/z 540.2[M+H]⁺.

¹H NMR (400 MHz, CDCl₃) δ 8.30 (s, 1H), 7.65 (dd, J=11.6, 2.1 Hz, 1H), 7.54 (s, 1H), 7.04 (ddd, J=8.6, 5.4, 3.3 Hz, 2H), 6.98 (t, J=8.0 Hz, 1H), 6.86 (d, J=9.7 Hz, 2H), 5.74 (s, 1H), 5.45 (d, J=1.6 Hz, 1H), 4.96 (s, 2H), 3.36 (d, J=6.3 Hz, 2H), 3.18 (s, 2H), 2.24 (d, J=8.1 Hz, 4H), 2.00 (t, J=1.2 Hz, 3H).

### Example 95 N-(4-(4-amino-5-(3-chloro-4-((1-oxo-1λ⁶-thietan-1-ylidene) amino) phenyl)-7-(methyl-d₃)-7H-pyrrolo [2,3-d] pyrimidin-6-yl)-3-chlorophenyl) methacrylamide

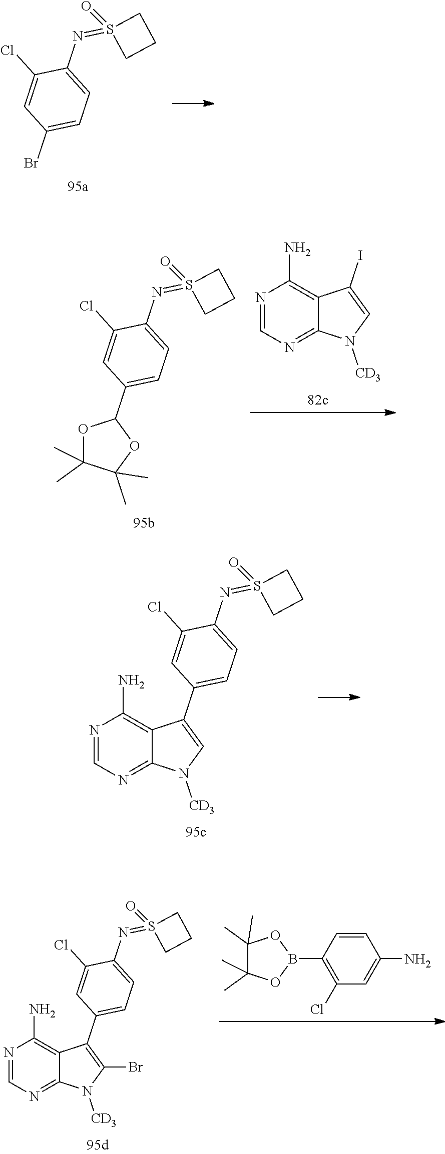

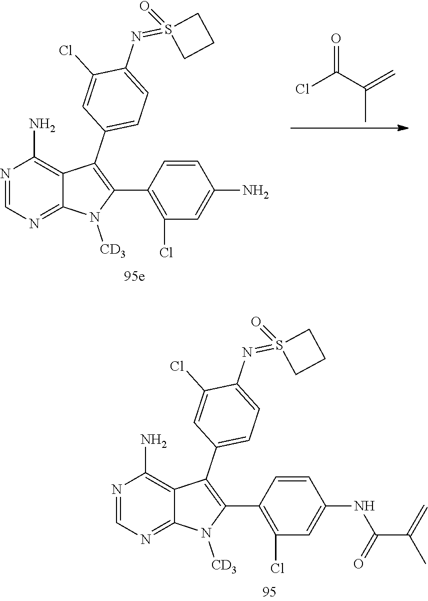

### Step 1 1-(2-chloro-4-(4,4,5,5-tetramethyl-1,3,2-dioxaborolan-2-yl) phenyl) imino)-1λ⁶-thietane-1-oxide 95b

Compound 95a (4 g, 13.58 mmol), bis(pinacolato)diboron (4.14 g, 16.29 mmol), Pd(dppf)Cl₂ (1.99 g, 2.72 mmol), and potassium acetate (3.99 g, 40.73 mmol) were added to 1,4-dioxane (50 mL), after replaced with argon gas three times, the reaction solution was heated up to 90° C. and stirred for 16 h. Water (100 mL) was added and extracted with DCM (100 mL×3). The organic phase was washed with saturated saline solution (200 mL), dried over anhydrous sodium sulfate, and concentrated under reduced pressure to obtain a crude product. The crude product was purified by column chromatography (PE/EtOAc=3/2) to obtain compound 95b (4.17 g), light yellow solid.

MS (ESI) m/z 342 [M+H]⁺

### Step 2 1-(4-(4-amino-7-(methyl-d₃)-7H-pyrrolo [2,3-d] pyrimidin-5-yl)-2-chlorophenyl) imino)-1λ⁶-thietane-1-oxide 95c

Compound 95b (3.57 g, 10.45 mmol), compound 82c (3.76 g, 13.58 mmol), tetrakis (triphenylphosphine) palladium (1.21 g, 1.04 mmol), and potassium phosphate (6.65 g, 31.35 mmol) were dissolved in 1,4-dioxane (30 mL) and water (3 mL), after replaced with nitrogen three times, the reaction was carried out at 70° C. overnight. After the reaction was completed, it was cooled down to room temperature, filtered through diatomaceous earth, concentrated under reduced pressure to remove the solvent, extracted with DCM/H₂O three times, and the organic layers were combined, and washed with saturated sodium chloride once, and the organic phase was dried over anhydrous sodium sulfate, concentrated under reduced pressure, and purified by column chromatography (DCM/MEOH=30/1) to obtain compound 95c (2.91 g), yellow solid.

MS (ESI) m/z 365 [M+H]⁺.

### Step 3 1-(4-(4-amino-6-bromo-7-(methyl-d₃)-7H-pyrrolo [2,3-d] pyrimidin-5-yl)-2-chlorophenyl) imino)-1λ⁶-thietane-1-oxide 95d

Compound 95c (1 g, 2.74 mmol) was dissolved with anhydrous DMF (10 mL), NBS (512 mg, 2.88 mmol) was added at 0° C., and the reaction was carried out for 2 h, and then quenched with sodium thiosulfate solution, extracted with EA/H₂O 4 times, and the organic layers were combined, washed with saturated salt water once, dried over anhydrous sodium sulfate, concentrated under reduced pressure, and purified by column chromatography (DCM/MEOH=30/1) to obtain compound 95d (800 mg), yellow solid.

MS (ESI) m/z 443 [M+H]⁺.

### Step 4 1-((4-(4-amino-6-(4-amino-2-chlorophenyl)-7-(methyl-d₃)-7H-pyrrolo [2,3-d]pyrimidin-5-yl)-2-chlorophenyl) imino)-1λ⁶-thietane-1-oxide 95e

Compound 95d (800 mg, 1.80 mmol), 3-chloro-4-(4,4,5,5-tetramethyl-1,3,2-dioxaborolan-2-yl) aniline (686 mg, 2.70 mmol), tetrakis (triphenylphosphine) palladium (208 mg, 0.18 mmol), and potassium phosphate (1.15 g, 5.41 mmol) were dissolved in 1,4-dioxane (20 mL) and water (2 mL), after replaced with nitrogen gas three times, the reaction was refluxed at 100° C. for 12 h. The reaction solution was cooled down to room temperature, filtered through diatomaceous earth, concentrated under the reduced pressure to remove the solvent, extracted with DCM/H₂O three times, and the organic layers were combined, washed with saturated saline solution, dried over anhydrous sodium sulfate. concentrated under the reduced pressure, and purified by column chromatography (DCM/MEOH=40/1) to obtain compound 95e (376 mg), white solid.

MS (ESI) m/z 490 [M+H]⁺.

### Step 5 N-(4-(4-amino-5-(3-chloro-4-((1-oxo-1λ⁶-thietan-1-ylidene) amino) phenyl)-7-(methyl-d₃)-7H-pyrrolo [2,3-d] pyrimidin-6-yl)-3-chlorophenyl) methacrylamide 95

Compound 95e (376 mg, 0.77 mmol) was dissolved with anhydrous DMF (10 mL), methacryloyl chloride (96 mg, 0.92 mmol) was added at 0° C., the reaction was carried out for 1 h, and then quenched with water, extracted with dichloromethane, and the organic layers were combined, washed with saturated salt once, and the organic phase was dried over anhydrous sodium sulfate, concentrated under reduced pressure, and purified by column chromatography (DCM/MEOH=20/1) to obtain compound 95 (60 mg), white solid.

MS (ESI) m/z 558 [M+H]⁺.

¹H NMR (400 MHz, CDCl₃) δ 8.37 (s, 1H), 7.89 (d, J=2.1 Hz, 1H), 7.55 (s, 1H), 7.38 (dd, J=8.3, 2.2 Hz, 1H), 7.29 (s, 1H), 7.14 (d, J=8.3 Hz, 1H), 7.06 (d, J=8.1 Hz, 1H), 7.02 (s, 1H), 5.81 (s, 1H), 5.52 (d, J=1.6 Hz, 1H), 5.02 (s, 2H), 4.33-4.19 (m, 4H), 2.44-2.31 (m, 2H), 2.07 (t, J=1.3 Hz, 3H).

### Example 96 N-(4-(4-amino-5-(3-fluoro-4-((methyl (oxo) (phenyl)-λ⁶-sulfaneylidene) amino) phenyl)-7-(methyl-d₃)-7H-pyrrolo [2,3-d] pyrimidin-6-yl)-3-fluorophenyl) methacrylamide

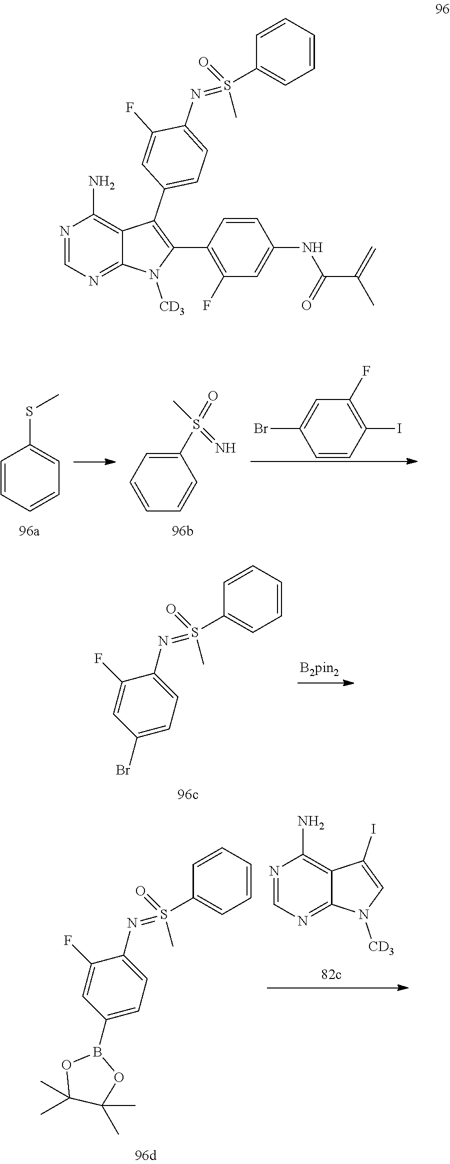

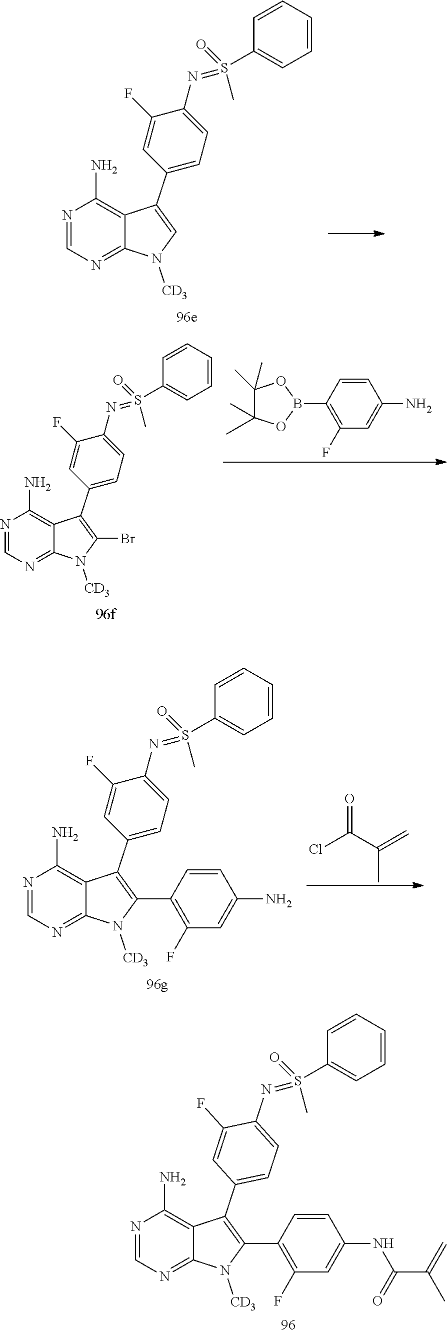

### Step 1 imino (methyl) phenyl)-λ⁶-sulfone 96b

Compound 96a (10 g, 81 mmol) and ammonium carbamate (9.43 g, 120 mmol) were added to a flask sequentially, and then MeOH (200 mL) and iodobenzene diethyl ester (52 g, 161 mmol) were added. The reaction solution was stirred open at room temperature for 30 min, and then concentrated under reduced pressure to remove the solvent to obtain a crude product. The crude product was purified by column chromatography (MeOH/DCM=1/20) to obtain compound 96b (5.76 g), colorless oil.

MS (ESI) m/z 156 [M+H]⁺

### Step 2 ((4-bromo-2-fluorophenyl) imino) (methyl) phenyl-λ⁶-sulfone 96c

Compound 96b (5.76 g, 37.11 mmol), cesium carbonate (18.15 g, 55.66 mmol), 1-bromo-3-fluoro-4-iodobenzene (11.17 g, 37.11 mmol), Pd₂(dba)₃ (1.02 g, 1.11 mmol), and Xantphos (1.29 g, 2.23 mmol) were added to 1,4-dioxane (100 mL), the reaction solution was heated up to 100° C. to react for 16 h. The reaction solution was filtered through diatomaceous earth, and the filter cake was washed with ethyl acetate (300 mL), and saturated saline solution (500 mL) was used for washing. The organic phase was dried over anhydrous sodium sulfate and concentrated under reduced pressure to obtain a crude product. The crude product was purified by column chromatography (PE/EtOAc=4/1 to 3/2) to obtain compound 96c (11.14 g), light yellow solid.

MS (ESI) m/z 328 [M+H]⁺

### Step 3 (2-fluoro-4-(4,4,5,5-tetramethyl-1,3,2-dioxaborolan-2-yl) phenyl) imino) (methyl) phenyl-λ⁶-sulfone 96d

Compound 96c (2 g, 6.09 mmol), bis(pinacolato)diboron (1.86 mg, 7.31 mmol), Pd(dppf)Cl₂ (892 mg, 1.22 mmol), and potassium acetate (1.79 g, 18.28 mmol) were added to 1,4-dioxane (30 mL), after replaced with argon gas three times, and the reaction solution was heated up to 90° C. and stirred for 16 h. Water (100 mL) as added and extracted with DCM (50 mL×3). The organic phase was washed with saturated saline solution (200 mL), dried over anhydrous sodium sulfate, and concentrated under reduced pressure to obtain a crude product. The crude product was purified by column chromatography (PE/EtOAc=3/2) to obtain compound 96d (2.08 g), light yellow solid.

MS (ESI) m/z 376 [M+H]⁺

### Step 4 (4-(4-amino-7-methyl-d₃)-7H-pyrrolo [2,3-d] pyrimidin-5-yl)-2-fluorophenyl) imino) (methyl) phenyl-λ⁶-sulfone 96e

Compound 96d (2.08 g, 5.54 mmol), compound 82c (2.00 g, 7.21 mmol), tetrakis (triphenylphosphine) palladium (640 mg, 0.55 mmol), and potassium phosphate (4.20 g, 19.83 mmol) were added in 1,4-dioxane (30 mL) and water (3 mL), after replaced with nitrogen gas three times, the reaction was carried out at 70° C. overnight. After the reaction was completed, it was cooled down to room temperature, filtered through diatomaceous earth, concentrated under reduced pressure to remove the solvent, extracted with DCM/H₂O three times, and the organic layers were combined, washed with saturated sodium chloride once, dried over anhydrous sodium sulfate, concentrated under reduced pressure, and purified by column chromatography (DCM/MEOH=30/1) to obtain compound 96e (1.26 g), yellow solid.

MS (ESI) m/z 399 [M+H]⁺.

### Step 5 (4-(4-amino-6-bromo-7-methyl-d₃)-7H-pyrrolo [2,3-d] pyrimidin-5-yl)-2-fluorophenyl) imino) (methyl) phenyl-λ⁶-sulfone 96f

Compound 96e (1.26 g, 3.16 mmol) was dissolved with anhydrous DMF (15 mL), NBS (591 mg, 3.32 mmol) was added at 0° C., the reaction was carried out for 2 h, and then quenched with sodium thiosulfate solution, extracted with dichloromethane, and the organic layers were combined, and washed with saturated salt water once, and the organic phase was dried over anhydrous sodium sulfate, concentrated under reduced pressure, and purified by column chromatography (DCM/MEOH=30/1) to obtain compound 96f (726 g), yellow solid.

MS (ESI) m/z 477 [M+H]⁺.

### Step 6 (4-(4-amino-6-(4-amino-2-fluorophenyl)-7-methyl-d₃)-7H-pyrrolo [2,3-d]pyrimidin-5-yl)-2-fluorophenyl) imino) (methyl) phenyl-λ⁶-sulfone 96g

Compound 96f (726 mg, 1.52 mmol), 3-fluoro-4-(4,4,5,5-tetramethyl-1,3,2-dioxaborolan-2-yl) aniline (578 mg, 2.28 mmol), tetrakis (triphenylphosphine) palladium (176 mg, 0.15 mmol), and potassium phosphate (967 mg, 4.56 mmol) were added in 1,4-dioxane (20 mL) and water (2 mL), after replaced with nitrogen gas three times, the reaction was refluxed at 100° C. for 12 h. The reaction solution was cooled down to room temperature, and filtered through diatomaceous earth, concentrated under reduced pressure to remove the solvent, extracted with DCM/H₂O three times, and the organic layers were combined, washed with saturated saline solution, dried over anhydrous sodium sulfate, concentrated under reduced pressure, and purified by column chromatography (DCM/MEOH=40/1) to obtain compound 96g (550 mg), white solid.

MS (ESI) m/z 508 [M+H]⁺.

### Step 7 N-(4-(4-amino-5-(3-fluoro-4-((methyl (oxo) (phenyl)-λ⁶-sulfaneylidene) amino) phenyl)-7-(methyl-d₃)-7H-pyrrolo [2,3-d] pyrimidin-6-yl)-3-fluorophenyl) methacrylamide 96

Compound 96g (550 mg, 1.08 mmol) was dissolved with anhydrous DMF (10 mL), methacryloyl chloride (136 mg, 1.30 mmol) was added at 0° C., and the reaction was carried out for 1 h, and then quenched with water, extracted with dichloromethane, and the organic layers were combined, washed with saturated salt once, and the organic phase was dried over anhydrous sodium sulfate, concentrated under reduced pressure, and purified by column chromatography (DCM/MEOH=20/1) to obtain compound 96 (25 mg), white solid.

MS (ESI) m/z 576 [M+H]⁺.

¹H NMR (400 MHz, CDCl₃) δ 8.34 (s, 1H), 8.00 (dd, J=7.2, 1.7 Hz, 2H), 7.64 (d, J=7.3 Hz, 2H), 7.60-7.54 (m, 3H), 7.05 (d, J=8.3 Hz, 1H), 6.98 (d, J=8.0 Hz, 1H), 6.87 (d, J=11.3 Hz, 1H), 6.77 (s, 1H), 5.82 (s, 1H), 5.53 (d, J=1.6 Hz, 1H), 4.95 (s, 2H), 3.29 (s, 3H), 2.09 (d, J=0.6 Hz, 3H).

### Example 97 N-(4-(4-amino-5-(3-fluoro-4-((1-oxo-1λ⁶-thiophene-1-ylidene) amino) phenyl)-7-(methyl-d₃)-7H-pyrrole [2,3-d] pyrimidin-6-yl)-3-fluorophenyl) methacrylamide

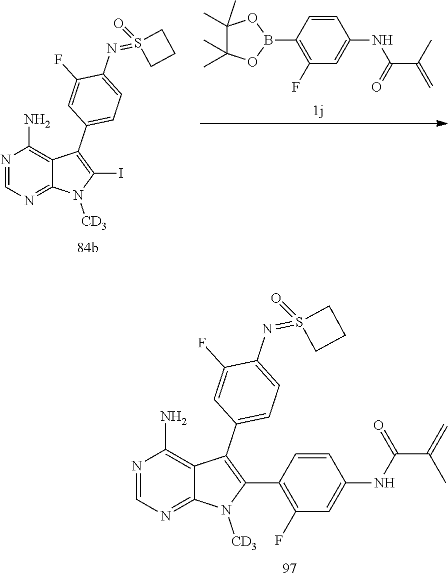

Compound 97 was synthesized with reference to the similar synthetic route and steps of compound 12, wherein, compound 12a was replaced with compound 84b, compound 12b was replaced with compound 1j.

MS (ESI) m/z 526.10 [M+H]⁺.

¹H NMR (400 MHz, CDCl₃) δ 8.37 (s, 1H), 7.73 (dd, J=11.5, 2.0 Hz, 1H), 7.58 (s, 2H), 7.07-6.92 (m, 3H), 6.94 (d, J=9.6 Hz, 1H), 5.81 (s, 1H), 5.52 (s, 1H), 5.00 (s, 2H), 4.42-4.21 (m, 4H), 2.41-2.28 (m, 2H), 2.07 (s, 3H).

### Example 98 N-(4-(4-amino-7-(methyl-d₃)-5-(4-((1-oxo-1λ⁶-thiophene-1-ylidene) amino) phenyl)-7H-pyrrolo [2,3-d] pyrimidin-6-yl)-3-fluorophenyl) methacrylamide

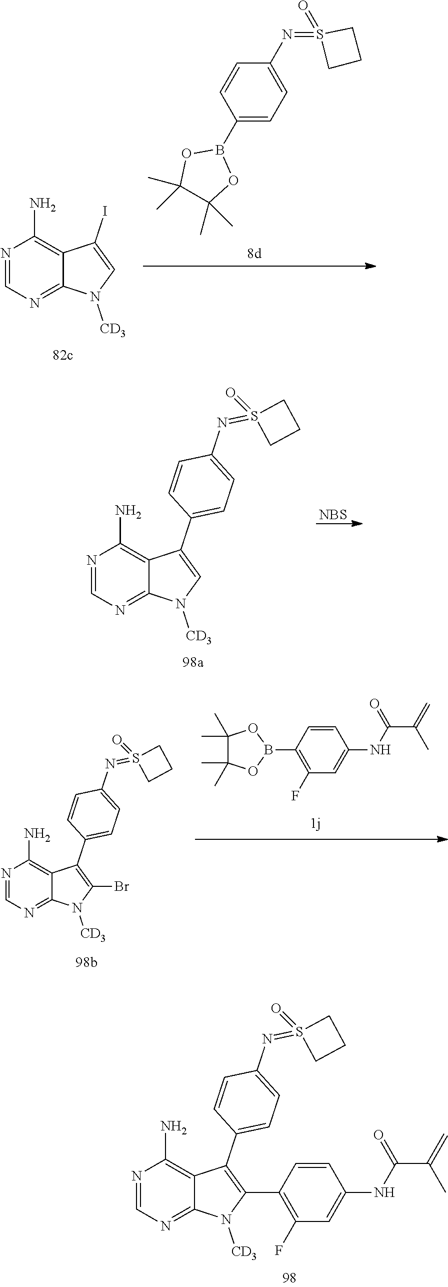

### Step 1 1-(4-(4-amino-7-(methyl-d₃)-7H-pyrrolo [2,3-d] pyrimidin-5-ylphenyl) imino)-1λ⁶-thiophene-1-oxide 98a

Compound 82c (2.0 g, 7.22 mmol) was dissolved in 1,4-dioxane (20 mL) and H₂O (4 mL), compound 8d (2.44 g, 7.94 mmol), potassium phosphate (4.70 g, 22.0 mmol), and tetratriphenylphosphine palladium (0.80 g, 0.70 mmol) were added, and the reaction was carried out at 90° C. for 14 h under N₂ protection. After TLC monitored the completion of the reaction, it was extracted with ethyl acetate (50 mL×3), and the organic phases were combined and concentrated under reduced pressure to obtain a crude product. The crude product was purified by column chromatography (DCM/MeOH=10/1) to obtain compound 98a (1300 mg) MS (ESI) m/z 331.10 [M+H]⁺.

### Step 2 1-(4-(4-amino-6-bromo-7-(methyl-d₃)-7H-pyrrolo [2,3-d] pyrimidin-5-yl) phenyl) imino)-1λ⁶-thiophene-1-oxide 98b

Compound 98a (0.30 g, 0.91 mmol) was dissolved in DMF (5 mL), and it was cooled down to 0° C., NBS (0.18 g, 1.00 mmol) was added, and the reaction was carried out at room temperature for 1 h. After TLC monitored the completion of the reaction, it was extracted with dichloromethane (30 mL×3), and the organic phases were combined and concentrated under reduced pressure to obtain crude product compound 98b (0.20 g), which was directly used in the next step.

MS (ESI) m/z 409.10 [M+H]⁺.

### Step 3 N-(4-(4-amino-7-(methyl-d₃)-5-(4-((1-oxo-1λ⁶-thiophene-1-ylidene) amino) phenyl)-7H-pyrrolo [2,3-d] pyrimidin-6-yl)-3-fluorophenyl) methacrylamide 98

Compound 98b (0.20 g, 0.49 mmol) was dissolved in 1,4-dioxane (5 mL) and H₂O (1 mL), compound 1j (0.17 g, 0.54 mmol), potassium phosphate (320 mg, 1.50 mmol), and tetrakis (triphenylphosphine) palladium (0.036 g, 0.05 mmol) were added, and the reaction was carried out at 90° C. for 14 h under N₂ protection. After TLC monitored the completion of the reaction, it was extracted with ethyl acetate (50 mL×3), and the organic phases were combined and concentrated under reduced pressure to obtain a crude product. The crude product was purified by column chromatography (DCM/MeOH=10/1) to obtain compound 98 (22.0 mg).

MS (ESI) m/z 508.10 [M+H]⁺.

¹H NMR (400 MHz, CDCl₃) δ 8.37 (d, J=7.8 Hz, 1H), 7.73 (dd, J=11.5, 2.0 Hz, 1H), 7.61 (s, 1H), 7.23-7.04 (m, 4H), 6.97 (d, J=8.1 Hz, 2H), 5.83 (s, 1H), 5.54 (d, J=1.6 Hz, 1H), 5.10 (s, 2H), 4.24 (dd, J=9.3, 7.2 Hz, 4H), 2.55-2.30 (m, 2H), 2.09 (d, J=1.3 Hz, 3H).

### Example 99 N-(4-(4-amino-5-(3-fluoro-4-((1-oxo-1λ⁶-thiophene-1-ylidene) amino) phenyl)-7-methyl-7H-pyrrolo [2,3-d] pyrimidin-6-yl)-3-methylphenyl) methacrylamide

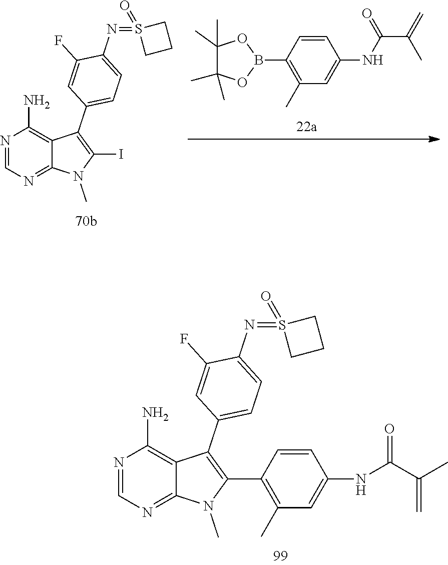

Compound 99 was synthesized with reference to the similar synthetic route and steps of compound 12, wherein, compound 12a was replaced with compound 70b, compound 12b was replaced with compound 22a.

MS (ESI) m/z 519.2 [M+H]⁺.

¹H NMR (400 MHz, CDCl₃) δ 8.30 (s, 1H), 7.43 (d, J=2.8 Hz, 2H), 7.35 (dd, J=8.2, 2.3 Hz, 1H), 7.10 (d, J=8.3 Hz, 1H), 6.91 (d, J=8.3 Hz, 1H), 6.85-6.77 (m, 2H), 5.73 (s, 1H), 5.42 (d, J 1.6 Hz, 1H), 4.95 (s, 2H), 4.20 (q, J=8.4, 8.0 Hz, 4H), 3.45 (s, 3H), 2.26 (t, J=8.3 Hz, 2H), 2.02-1.90 (m, 6H).

### Example 100 N-(4-(4-amino-5-(4-(diethyl (oxo)-1λ⁶-sulfaneylidene) amino)-3-fluorophenyl)-7-methyl-7H-pyrrolo [2,3-d] pyrimidin-6-yl)-3-fluorophenyl) methacrylamide

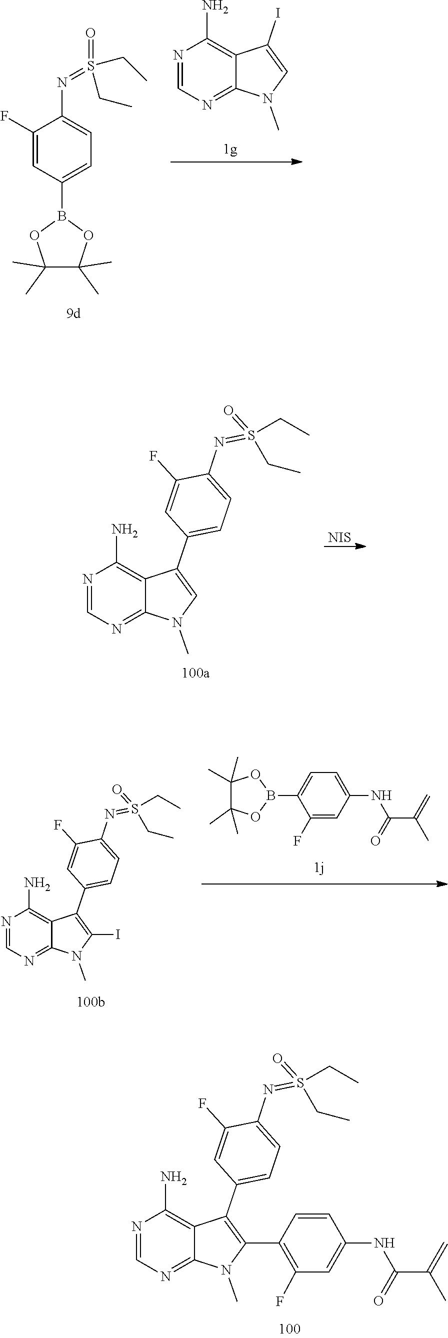

Compound 100 was synthesized with reference to the similar synthetic route and steps of compound 85, wherein, compound 85a was replaced with compound 9d.

MS (ESI) m/z 538.2 [M+H]⁺.

¹H NMR (400 MHz, CDCl₃) δ 8.30 (s, 1H), 7.64 (dd, J=11.6, 2.1 Hz, 1H), 7.50 (s, 1H), 7.13 (t, J=8.5 Hz, 1H), 7.06-6.96 (m, 2H), 6.85 (d, J 10.4 Hz, 2H), 5.74 (s, 1H), 5.45 (q, J=1.6 Hz, 1H), 4.95 (s, 2H), 3.58 (d, J=1.1 Hz, 3H), 3.15 (tq, J=14.3, 7.2 Hz, 4H), 1.97 (s, 3H), 1.37 (t, J=7.5 Hz, 6H).

### Example 101 N-(4-(4-amino-5-(4-(diethyl (oxo)-1λ⁶-sulfaneylidene) amino)-3-fluorophenyl)-7-methyl-7H-pyrrolo [2,3-d] pyrimidin-6-yl)-3-methylphenyl) methacrylamide

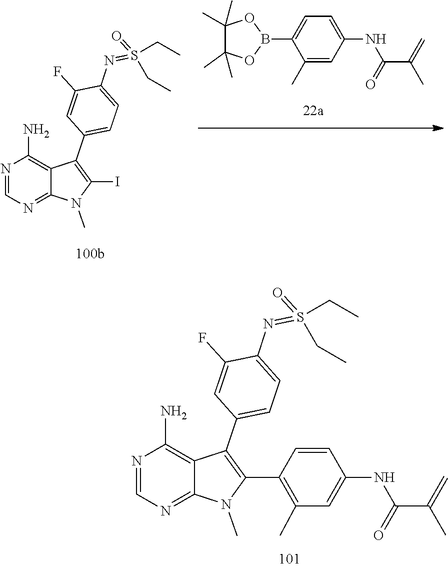

Compound 101 was synthesized with reference to the similar synthetic route and steps of compound 12, wherein, compound 12a was replaced with compound 100b, compound 12b was replaced with compound 22a.

MS (ESI) m/z 535.3 [M+H]⁺.

¹H NMR (400 MHz, CDCl₃) δ 8.36 (s, 1H), 7.50 (d, J=2.5 Hz, 2H), 7.41 (dd, J=8.1, 2.2 Hz, 1H), 7.17 (dt, J=8.4, 4.3 Hz, 2H), 6.85 (d, J=9.5 Hz, 2H), 5.80 (s, 1H), 5.49 (d, J=1.8 Hz, 1H), 5.09 (s, 2H), 3.52 (s, 3H), 3.20 (m, 4H), 2.08 (t, J=1.2 Hz, 3H), 2.00 (s, 3H), 1.43 (t, J=7.5 Hz, 6H).

### Example 102 N-(4-(4-amino-5-(4-(diethyl (oxo)-1λ⁶-sulfaneylidene) amino) phenyl)-7-methyl-7H-pyrrolo [2,3-d] pyrimidin-6-yl)-3-chlorophenyl) methacrylamide

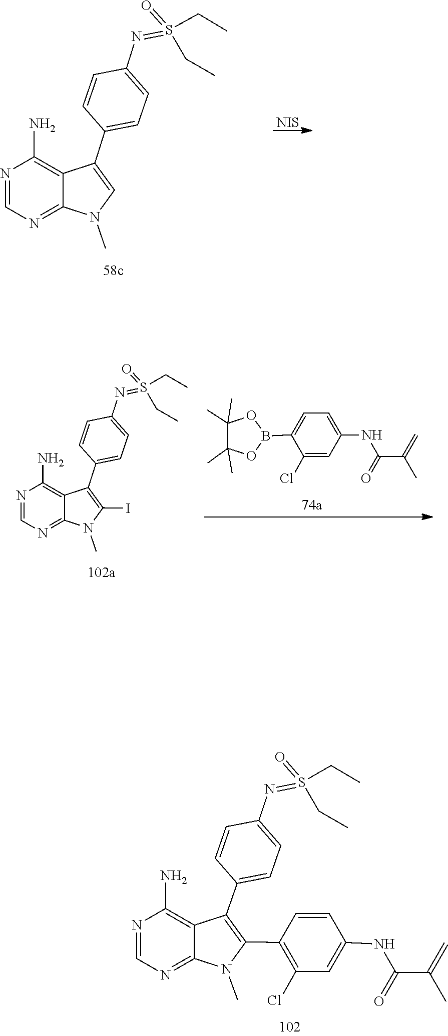

Compound 102 was synthesized with reference to the similar synthetic route and steps of compound 77, wherein, compound 12a was replaced with compound 58c, compound 1j was replaced with compound 74a.

MS (ESI) m/z 537.3 [M+H]⁺.

¹H NMR (400 MHz, CDCl₃) δ 8.39 (s, 1H), 7.89 (d, J=2.2 Hz, 1H), 7.61 (s, 1H), 7.37 (dd, J=8.3, 2.2 Hz, 1H), 7.14 (m, 3H), 7.03 (d, J=8.0 Hz, 2H), 5.83 (s, 1H), 5.54 (s, 1H), 5.08 (s, 2H), 3.61 (s, 3H), 3.31-3.10 (m, 4H), 2.10 (s, 3H), 1.43 (t, J=7.4 Hz, 6H).

### Example 103 N-(4-(4-amino-5-(4-(diethyl (oxo)-1λ⁶-sulfaneylidene) amino) phenyl)-7-(2,2,2-trifluoroethyl)-7H-pyrrolo [2,3-d] pyrimidin-6-yl)-3-fluorophenyl) methacrylamide

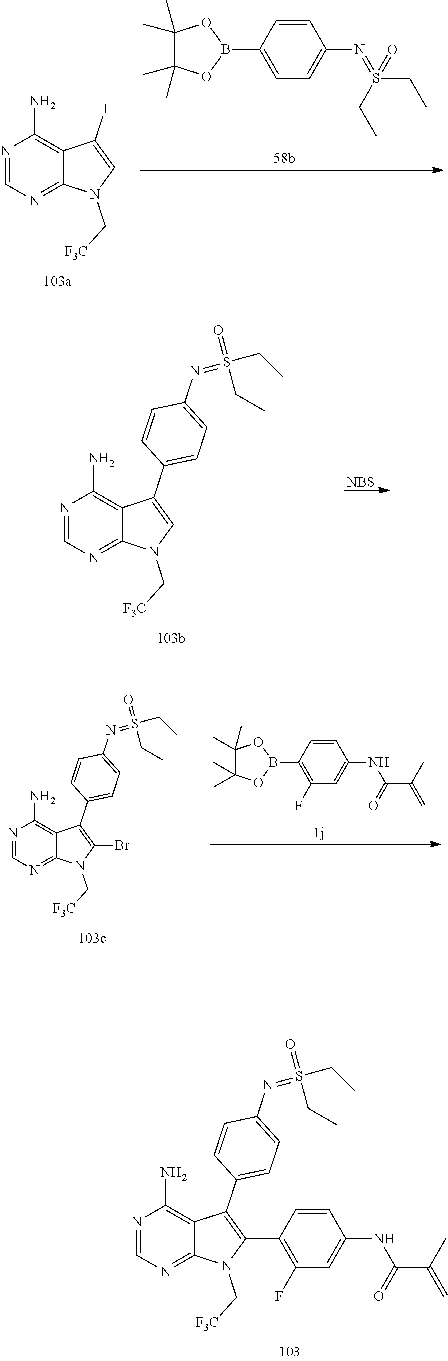

Compound 103 was synthesized with reference to the similar synthetic route and steps of compound 85, wherein, in step 1, compound 1g was replaced with compound 103a, compound 58d was replaced with compound 85a.

MS (ESI) m/z 589.3 [M+H]⁺.

¹H NMR (400 MHz, CDCl₃) δ 8.41 (s, 1H), 7.77 (d, J=11.5 Hz, 1H), 7.62 (s, 1H), 7.23-6.94 (m, 6H), 5.83 (s, 1H), 5.55 (s, 1H), 5.11 (s, 2H), 3.24 (m, 4H), 2.10 (s, 3H), 1.65 (s, 2H), 1.44 (t, J=7.5 Hz, 6H).

### Example 104 N-(4-(4-amino-5-(3-chloro-4-((1-oxo-1λ⁶-thiophene-1-ylidene) amino) phenyl)-7-methyl-7H-pyrrolo [2,3-d] pyrimidin-6-yl)-3-fluorophenyl) methacrylamide

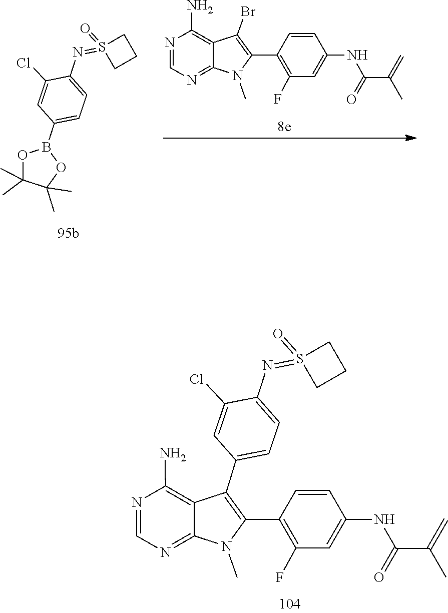 [SMILES: CC1(C)OB(c2ccc(N=S3(=O)CCC3)c(Cl)c2)OC1(C)C ; C=C(C)C(=O)Nc1ccc(-c2c(Br)c3c(N)ncnc3n2C)c(F)c1 ; C=C(C)C(=O)Nc1ccc(-c2c(-c3ccc(N=S4(=O)CCC4)c(Cl)c3)c3c(N)ncnc3n2C)c(F)c1]

Compound 104 was synthesized with reference to the similar synthetic route and steps of compound 31, wherein, compound 24b was replaced with compound 95b, compound 9e was replaced with compound 8e.

MS (ESI) m/z 539.1 [M+H]⁺.

¹H NMR (400 MHz, CDCl₃) δ 8.37 (s, 1H), 7.73 (dd, J=11.6, 2.0 Hz, 1H), 7.58 (s, 1H), 7.29 (s, 3H), 7.14-6.97 (m, 3H), 5.81 (s, 1H), 5.52 (q, J=1.6 Hz, 1H), 5.00 (s, 1H), 4.42-4.15 (m, 3H), 2.47-2.28 (m, 3H), 1.59 (s, 6H).

### Example 105 N-(4-(4-amino-7-methyl-5-(4-(4-oxo-1,4λ⁶-oxathiohexane-4-ylidene) amino) phenyl)-7H-pyrrolo [2,3-d] pyrimidin-6-yl)-3-fluorophenyl) methacrylamide

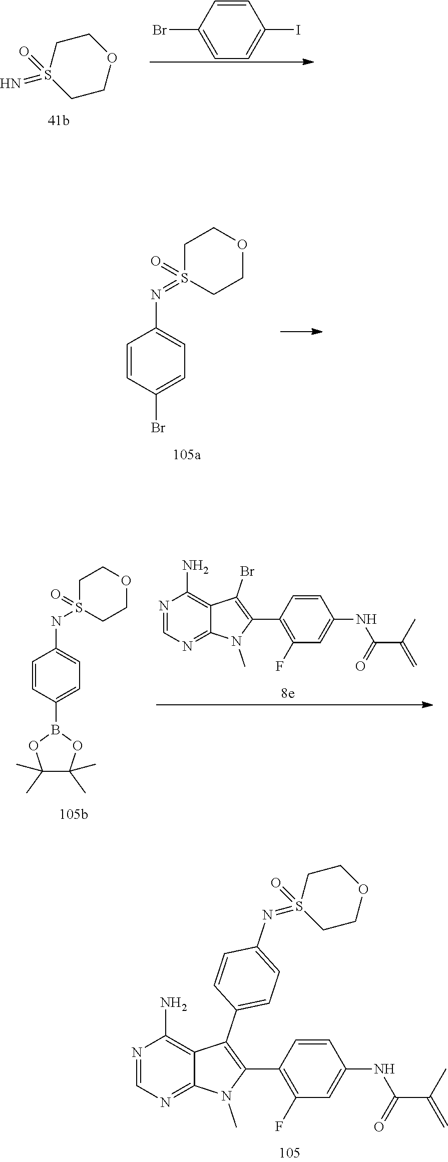

Compound 105 was synthesized with reference to the similar synthetic route and steps of compound 8, wherein, compound 8b was replaced with compound 41b.

MS (ESI) m/z 535.2 [M+H]⁺.

¹H NMR (400 MHz, CDCl₃) δ 8.39 (s, 1H), 7.71 (dd, J=11.5, 2.0 Hz, 1H), 7.59 (s, 1H), 7.19-7.05 (m, 6H), 5.83 (s, 1H), 5.55 (q, J=1.5 Hz, 1H), 5.04 (s, 2H), 4.22 (dt, J=12.8, 4.3 Hz, 2H), 4.12 (m, 2H), 3.68 (d, J=1.1 Hz, 4H), 3.48-3.36 (m, 3H), 3.30 (m, 3H).

### Example 106 N-(4-(4-amino-5-(4-((3,3-dimethyl-1-oxo-1λ⁶-thiocyclobutan-1-ylidene) amino)-3-fluorophenyl)-7-methyl-7H-pyrrolo [2,3-d] pyrimidin-6-yl)-3-fluorophenyl) methacrylamide

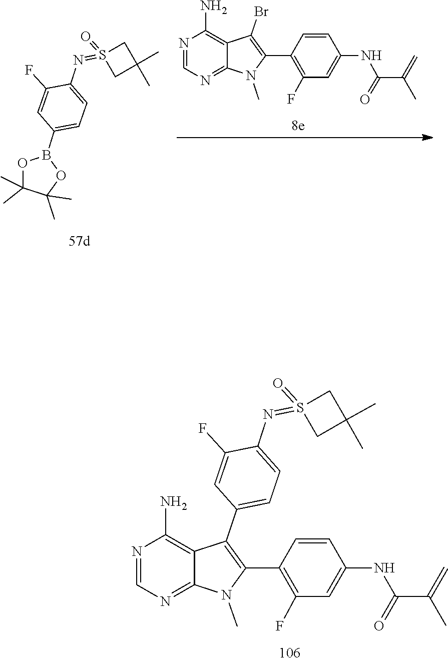

Compound 106 was synthesized with reference to the similar synthetic route and steps of compound 8, wherein, compound 8d was replaced with compound 57d.

MS (ESI) m/z 551.1 [M+H]⁺.

¹H NMR (400 MHz, CDCl₃) δ 8.39 (s, 1H), 7.76 (dd, J=11.6, 2.1 Hz, 1H), 7.60 (s, 1H), 7.17-7.02 (m, 3H), 6.96 (d, J=9.9 Hz, 2H), 5.84 (s, 1H), 5.55 (d, J=1.8 Hz, 1H), 5.04 (s, 2H), 4.20-4.01 (m, 4H), 3.68 (d, J=1.1 Hz, 2H), 2.10 (s, 2H), 1.56 (m, 4H), 1.30 (d, J=9.4 Hz, 4H).

### Example 107 N-(4-(4-amino-5-(4-(dimethyl (oxo)-1λ⁶-sulfaneylidene) amino) phenyl)-7-methyl-7H-pyrrolo [2,3-d] pyrimidin-6-yl)-3-fluorophenyl) methacrylamide

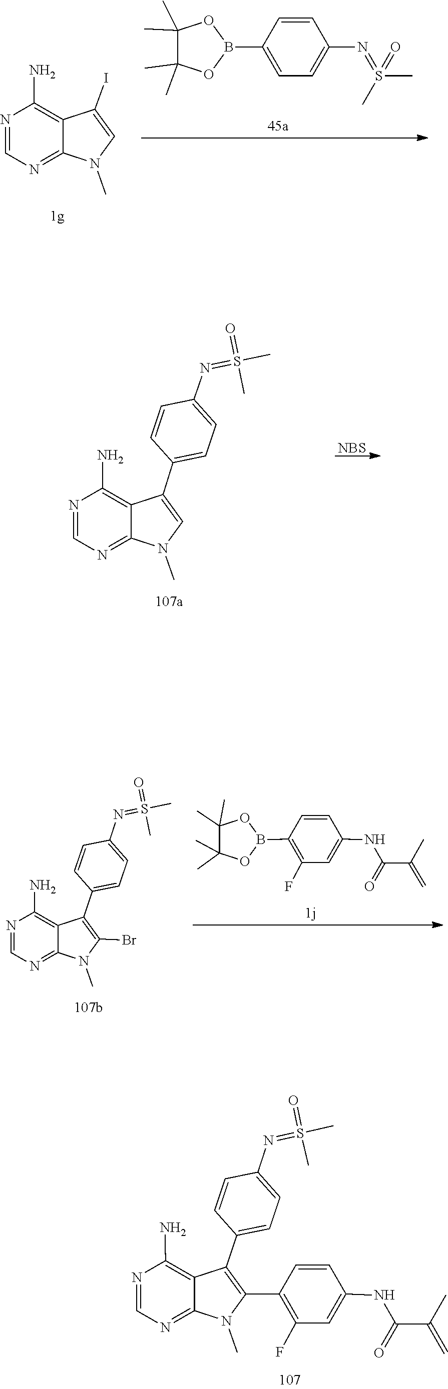

It was synthesized with reference to the similar synthetic route and steps of compound 85. MS (ESI) m/z 493.2 [M+H]⁺.

¹H NMR (400 MHz, CDCl₃) δ 8.29 (s, 1H), 7.63 (d, J=11.7 Hz, 1H), 7.49 (s, 1H), 7.11-6.85 (m, 5H), 5.73 (s, 1H), 5.45 (s, 1H), 5.28 (s, 1H), 4.96 (s, 2H), 3.59 (s, 3H), 2.00 (s, 3H), 1.19 (s, 6H).

### Example 108 N-(4-(4-amino-5-(4-((3,3-dimethyl-1-oxo-1λ⁶-thiophene-1-ylidene) amino) phenyl)-7-(methyl-d₃)-7H-pyrrolo [2,3-d] pyrimidin-6-yl)-3-fluorophenyl) methacrylamide

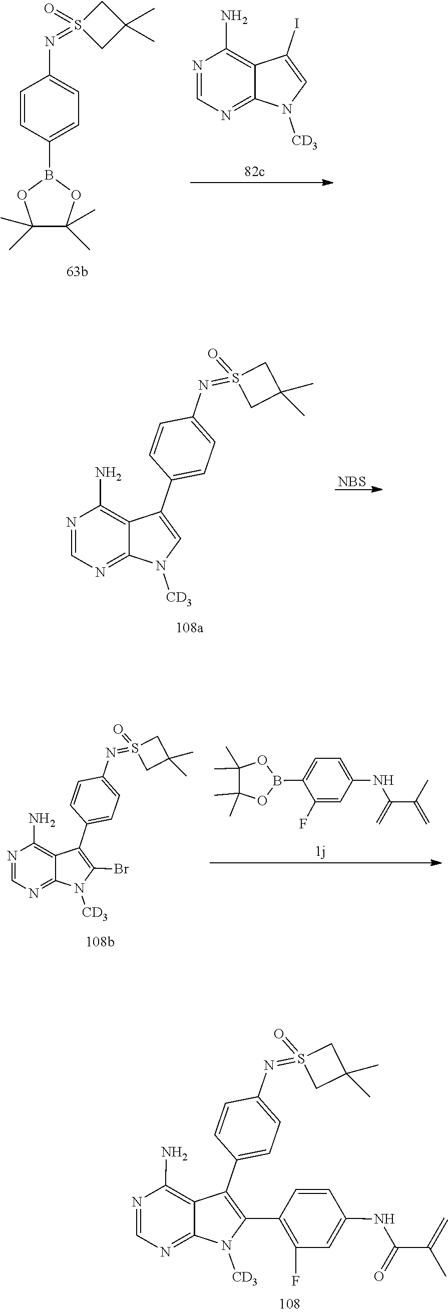

Compound 108 was synthesized with reference to the similar synthetic route and steps of compound 85, wherein, in step 1, compound 85a was replaced with compound 63b, compound 1g was replaced with compound 82c.

MS (ESI) m/z 536.9 [M+H]⁺.

¹H NMR (400 MHz, CDCl₃) δ 8.36 (s, 1H), 7.71 (dd, J=11.8, 1.8 Hz, 1H), 7.56 (s, 1H), 7.17-7.03 (m, 4H), 6.94 (d, J=8.1 Hz, 2H), 5.80 (s, 1H), 5.52 (d, J=1.6 Hz, 1H), 5.02 (s, 2H), 4.05-3.90 (m, 4H), 2.05 (d, J=10.4 Hz, 3H), 1.54 (s, 3H), 1.52 (s, 3H).

### Example 109 N-(4-(4-amino-5-(4-(diethyl (oxo)-λ⁶-sulfaneylidene) amino) phenyl)-7-(methyl-d₃)-7H-pyrrolo [2,3-d] pyrimidin-6-yl)-3-fluorophenyl) methacrylamide

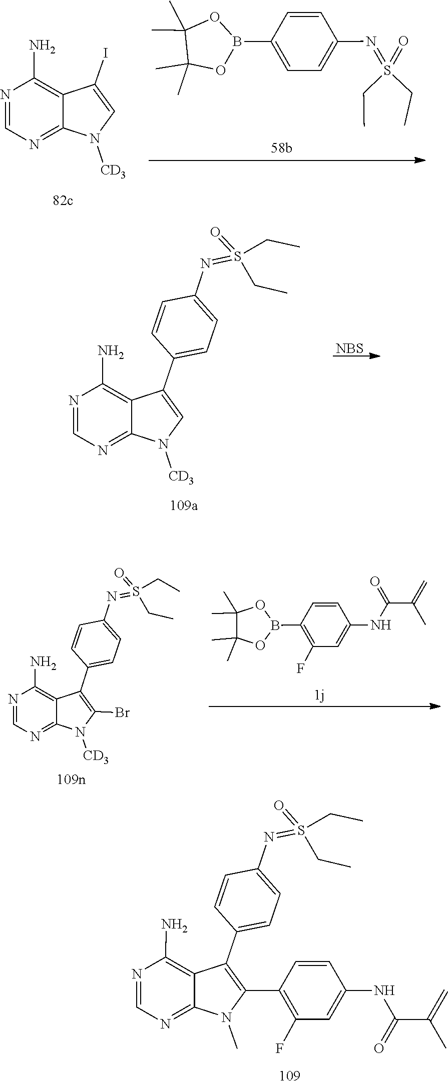

Compound 109 was synthesized with reference to the similar synthetic route and steps of compound 58, wherein, in step 1, compound 1g was replaced with compound 82c.

MS (ESI) m/z 542.10 [M+H]⁺.

¹H NMR (400 MHz, CDCl₃) δ 8.27 (s, 1H), 7.94-7.82 (m, 1H), 7.57-7.42 (m, 2H), 7.38-7.24 (m, 3H), 5.79 (s, 1H), 5.72 (s, 1H), 5.00 (s, 2H), 2.61 (q, 4H), 2.07 (s, 3H), 1.22 (t, 6H).

### Example 110 N-(4-(4-amino-7-methyl-5-(4-(1-tetrahydro-1λ⁶-thiophene-1-ylidene) amino) phenyl)-7H-pyrrolo [2,3-d] pyrimidin-6-yl)-3-fluorophenyl) acrylamide

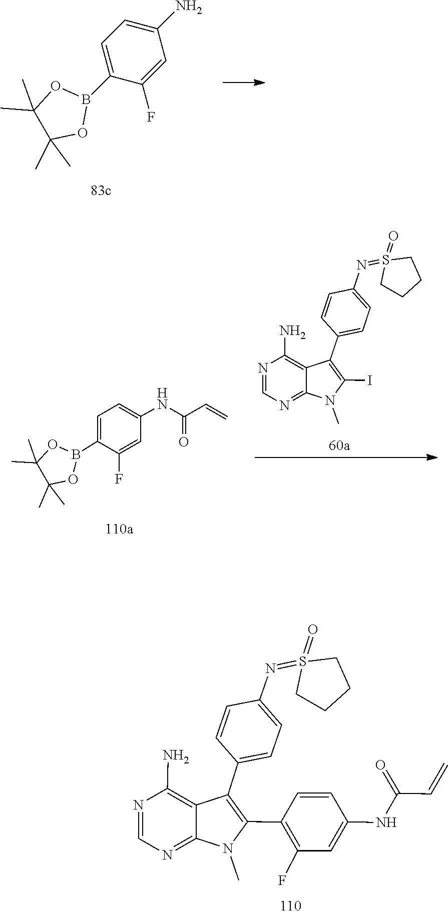

### Step 1 N-(3-fluoro-4-(4,4,5,5-tetramethyl-1,3,2-dioxaborolan-2-yl) phenyl) acrylamide 110a

Compound 83c (100 mg, 0.42 mmol), acrylic acid (45 mg, 0.63 mmol), and triethylamine (127 mg, 1.26 mmol) were dissolved in THE (5 mL), and then T3P (50% DMF solution) (0.63 mmol) was added, and the reaction was carried out at room temperature for 2 h, then concentrated under reduced pressure to remove the solvent, extracted with DCM/H₂O three times, and the organic layers were combined, and washed once with saturated sodium chloride, and the organic phase was dried over anhydrous sodium sulfate, concentrated under reduced pressure, and purified by column chromatography (PE/EA=5/1) to obtain compound 110a (80 mg), yellow solid.

MS (ESI) m/z 292.02 [M+H]⁺.

### Step 2 N-(4-(4-amino-7-methyl-5-(4-(1-tetrahydro-1λ⁶-thiophene-1-ylidene) amino) phenyl)-7H-pyrrolo [2,3-d] pyrimidin-6-yl)-3-fluorophenyl) acrylamide 110

Compound 110a (80 mg, 0.17 mmol), compound 60a (59 mg, 0.20 mmol), Pd(dppf)Cl₂ (25 mg, 0.034 mmol), and potassium phosphate (108 mg, 0.51 mmol) were dissolved in 1,4-dioxane (2 mL) and water (0.5 mL), after replaced with nitrogen gas three times, the reaction was refluxed at 90° C. for 6 h. The reaction solution was cooled down to room temperature, filtered through diatomaceous earth, concentrated under reduced pressure to remove the solvent, extracted with DCM/H₂O three times, and the organic layers were combined and washed with saturated saline solution, and the organic phase was dried over anhydrous sodium sulfate, concentrated under reduced pressure, and purified by column chromatography (DCM/MEOH=40/1) to obtain compound 110 (40 mg), yellow solid.

MS (ESI) m/z 505.03 [M+H]⁺.

¹H NMR (400 MHz, CDCl₃) δ 8.28 (s, 1H), 7.66 (d, J=11.3 Hz, 1H), 7.48 (s, 1H), 7.08-7.01 (m, 3H), 6.99 (d, J=7.8 Hz, 1H), 6.91 (d, J=8.1 Hz, 2H), 6.39 (d, J=16.8 Hz, 1H), 6.18 (dd, J=16.9, 10.2 Hz, 1H), 5.75 (d, J=10.2 Hz, 1H), 4.97 (s, 2H), 3.58 (s, 3H), 3.36 (dd, J=13.0, 6.6 Hz, 2H), 3.12 (dt, J=12.7, 6.6 Hz, 2H), 2.33-2.13 (m, 4H).

### Example 111 N-(4-(4-amino-7-methyl-5-(4-(1-tetrahydro-1λ⁶-thiophene-1-ylidene) amino) phenyl)-7H-pyrrolo [2,3-d] pyrimidin-6-yl)-3-fluorophenyl)-2-fluoroacrylamide

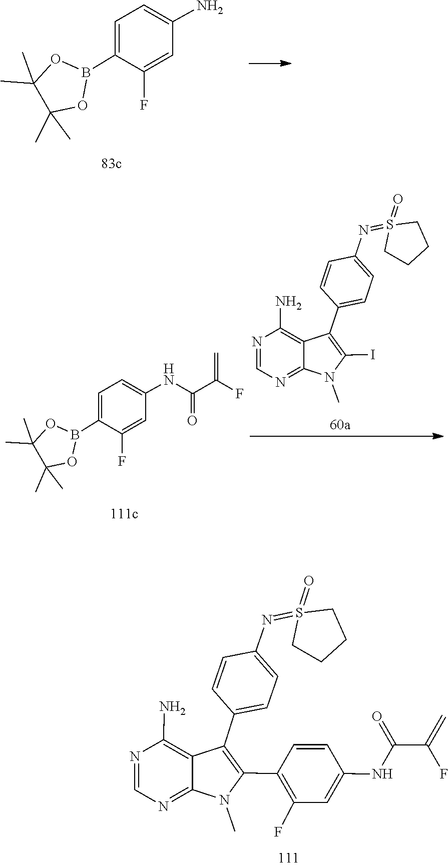

### Step 1 2-fluoro-N-(3-fluoro-4-(4,4,5,5-tetramethyl-1,3,2-dioxaborolan-2-yl) phenyl) acrylamide 111c

Compound 83c (100 mg, 0.42 mmol), 2-fluoroacrylic acid (57 mg, 0.63 mmol), and triethylamine (127 mg, 1.26 mmol) was dissolved in THE (5 mL), and then T3P (50% DMF solution) (0.63 mmol) was added, and the reaction was carried out at room temperature for 2 h, concentrated under reduced pressure to remove the solvent, extracted with DCM/H₂O three times, and the organic layers were combined, and washed with saturated salt twice, and the organic phase was dried over anhydrous sodium sulfate, concentrated under reduced pressure, and purified by column chromatography (PE/EA=5/1) to obtain compound 111c (85 mg), yellow solid.

MS (ESI) m/z 310.02 [M+H]⁺.

### Step 2 N-(4-(4-amino-7-methyl-5-(4-(1-hydroperoxide-1λ⁶-thiophene-1-ylidene) amino) phenyl)-7H-pyrrolo [2,3-d] pyrimidin-6-yl)-3-fluorophenyl)-2-fluoroacrylamide 111

Compound 111c (30 mg, 0.097 mmol), compound 60a (56 mg, 0.12 mmol), Pd(dppf)Cl₂ (14 mg, 0.019 mmol), and potassium phosphate (61 mg, 0.29 mmol) were dissolved in 1,4-dioxane (2 mL) and water (0.5 mL), after replaced with nitrogen gas three times, the reaction was refluxed at 90° C. for 6 h. The reaction solution was cooled down to room temperature, filtered through diatomaceous earth, concentrated under reduced pressure to remove the solvent, extracted with DCM/H₂O three times, and the organic layers were combined, washed with saturated saline solution, dried over anhydrous sodium sulfate, concentrated under reduced pressure, purified by column chromatography (DCM/MEOH=40/1) to obtain compound 111 (4 mg), white solid.

MS (ESI) m/z 523.10 [M+H]⁺.

¹H NMR (400 MHz, CDCl₃) δ 8.29 (s, 1H), 7.93 (d, J=5.4 Hz, 1H), 7.70-7.59 (m, 1H), 7.12-6.97 (m, 4H), 6.91 (d, J=8.1 Hz, 2H), 5.79 (dd, J=47.8, 3.6 Hz, 1H), 5.23 (dd, J=15.1, 3.5 Hz, 1H), 4.99 (s, 2H), 3.59 (s, 3H), 3.35 (dt, J=12.6, 6.4 Hz, 2H), 3.12 (dt, J=12.9, 6.6 Hz, 2H), 2.38-2.11 (m, 4H).

### Example 112 N-(4-(4-amino-7-methyl-5-(4-((1-tetrahydro-1λ⁶-thiophene-1-ylidene) amino) phenyl)-7H-pyrrolo [2,3-d] pyrimidin-6-yl)-3-fluorophenyl) but-2-ynamide

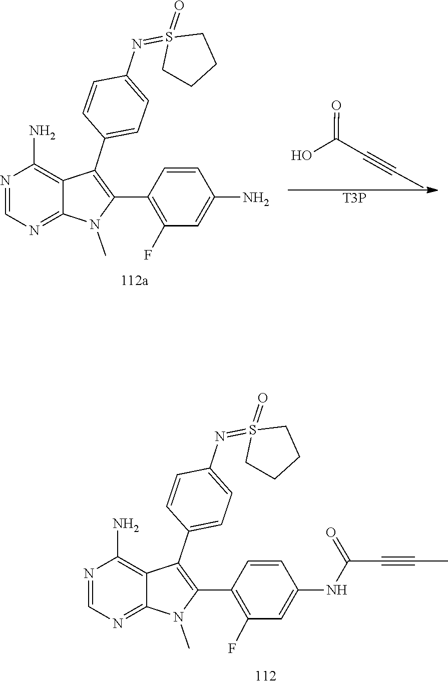

Compound 112a (100 mg, 0.22 mmol), 2-butyne acid (27 mg, 0.33 mmol), and triethylamine (67 mg, 0.66 mmol) were dissolved in THE (5 mL), and then T3P (50% DMF solution) (0.33 mmol) was added, and the reaction was carried out at room temperature for 2 h, then concentrated under reduced pressure to remove the solvent, extracted with DCM/H₂O three times, and the organic layers were combined, washed with saturated sodium chloride once, dried over anhydrous sodium sulfate, concentrated under reduced pressure, and purified by column chromatography (DCM/MeOH=40/1) to obtain compound 112 (10 mg), white solid.

MS (ESI) m/z 517.20 [M+H]⁺.

¹H NMR (400 MHz, CDCl₃) δ 8.28 (s, 1H), 7.53 (d, J=10.3 Hz, 2H), 7.04 (d, J 7.8 Hz, 2H), 7.00-6.95 (m, 2H), 6.91 (d, J=8.0 Hz, 2H), 4.97 (s, 2H), 3.59-3.56 (m, 3H), 3.35 (dt, J=13.2, 6.5 Hz, 2H), 3.13 (dt, J=12.9, 6.6 Hz, 2H), 2.32-2.09 (m, 5H).

### Example 113 N-(4-(4-amino-7-methyl-5-(4-(1-tetrahydro-1λ⁶-thiophene-1-ylidene) amino) phenyl-3,5-d2)-7H-pyrrolo [2,3-d] pyrimidin-6-yl)-3-fluorophenyl) methacrylamide

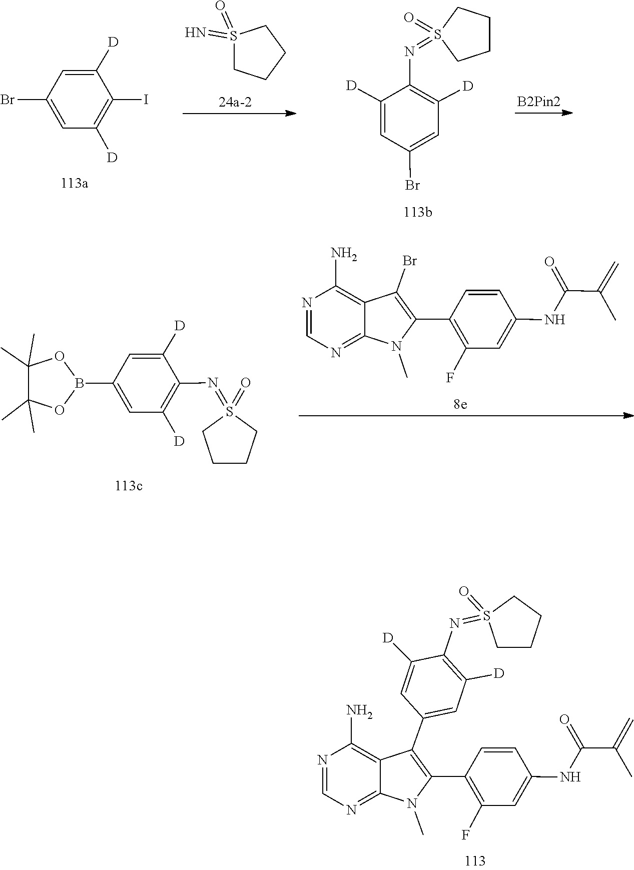 [SMILES: [2H]c1cc(Br)cc([2H])c1I ; N=S1(=O)CCCC1 ; [2H]c1cc(Br)cc([2H])c1N=S1(=O)CCCC1 ; [2H]c1cc(B2OC(C)(C)C(C)(C)O2)cc([2H])c1N=S1(=O)CCCC1 ; C=C(C)C(=O)Nc1ccc(-c2c(Br)c3c(N)ncnc3n2C)c(F)c1 ; [2H]c1cc(-c2c(-c3ccc(NC(=O)C(=C)C)cc3F)n(C)c3ncnc(N)c23)cc([2H])c1N=S1(=O)CCCC1]

Compound 113 was synthesized with reference to the similar synthetic route and steps of compound 105, wherein, in step 1, p-bromoiodobenzene was replaced with compound 113a.

MS (ESI) m/z 521.2 [M+H]⁺

¹H NMR (400 MHz, MeOD-d4) δ 8.18 (s, 1H), 7.71 (dd, J=12.1, 2.0 Hz, 1H), 7.35 (dd, J=8.4, 2.0 Hz, 1H), 7.15 (t, J=4.1 Hz, 3H), 5.81 (s, 1H), 5.54 (d, J 1.1 Hz, 1H), 3.61 (s, 3H), 3.42 (m, 2H), 3.25-3.20 (m, 2H), 2.37-2.30 (m, 2H), 2.22 (m, 2H), 2.02 (s, 3H).

### Example 114 N-(4-(4-amino-7-methyl-5-(4-((1-oxide tetrahydro-1λ⁶-thiophene-1-ylidene) amino) phenyl-2,3,5,6-d4)-7H-pyrrolo [2,3-d] pyrimidin-6-yl)-3-fluorophenyl) methacrylamide

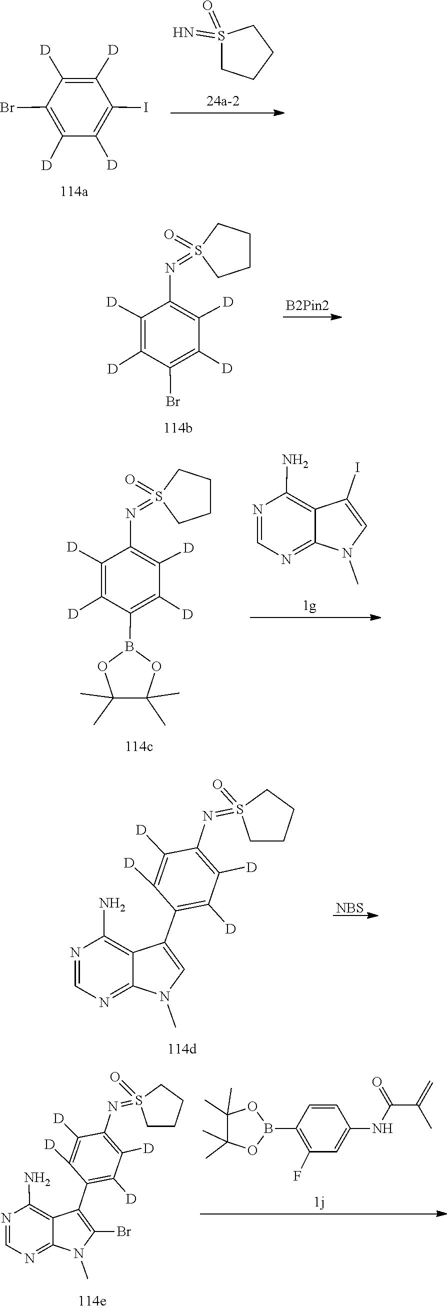

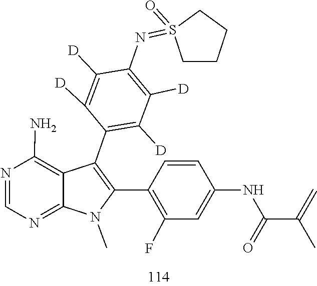 [SMILES: [2H]c1c([2H])c(-c2c(-c3ccc(NC(=O)C(=C)C)cc3F)n(C)c3ncnc(N)c23)c([2H])c([2H])c1N=S1(=O)CCCC1]

### Step 1 1-((4-bromophenyl-2,3,5,6-d4) imino) tetrahydro-1H-1λ⁶-thiophene-1-oxide 114b

Compound 114a was synthesized with reference to Organic Letters (2021), 23 (5), 1554-1560. To the reaction flask, compound 114a (1.13 g, 3.95 mmol), anhydrous 1,4-dioxane (11 mL), compound 24a-2 (471 mg, 3.95 mmol), Xantphos (171 mg, 0.30 mmol), cesium carbonate (1.80 g, 5.53 mmol), and Pd₂(dba)₃ (90 mg, 0.099 mmol) were added in sequence, after replaced with nitrogen gas three times, the reaction was refluxed at 100° C. for 3 h. TLC showed the formation of new dots, the reaction was diluted with ethyl acetate and H₂O, filtered through diatomaceous earth, extracted with ethyl acetate, and the organic phase was washed with saturated saline solution, dried over anhydrous sodium sulfate, concentrated under reduced pressure, and purified by silica gel column chromatography (PE/EA=1/1) to obtain compound 114b (775 mg), light yellow oil.

MS (ESI) m/z 278 [M+H]⁺.

### Step 2 1-(4-(4,4,5,5-tetramethyl-1,3,2-dioxaborolan-2-yl) phenyl-2,3,5,6-d4) imino) tetrahydro-1H-1λ⁶-thiophene-1-oxide 114c

To the reaction flask, compound 114b (725 mg, 2.61 mmol), anhydrous 1,4-dioxane (8 mL), bis(pinacolato)diboron (993 mg, 3.91 mmol), potassium acetate (768 mg, 7.83 mmol), and Pd(dppf)Cl₂ (189 mg, 0.261 mmol) were added in sequence, after replaced with nitrogen gas three times, the reaction was heated up to 90° C. for 4 h. TLC showed the formation of new dots, and after the reaction was completed, the reaction solution was cooled down to room temperature, diluted with ethyl acetate and H₂O, filtered through diatomaceous earth, extracted with ethyl acetate, and the organic phase was washed with saturated saline solution, dried over anhydrous sodium sulfate, concentrated under reduced pressure, and purified by silica gel column chromatography (PE/EA=1/1) to obtain compound 114c (532 mg), yellow solid.

MS (ESI) m/z 326 [M+H]⁺.

### Step 3 1-(4-(4-amino-7-methyl-7H-pyrrolo [2,3-d] pyrimidin-5-yl) phenyl-2,3,5,6-d4) imino) tetrahydro-1H-1λ⁶-thiophene-1-oxide 114d

To a reaction flask, compound 114c (467 mg, 1.44 mmol), 1,4-dioxane (5 mL), H₂O (1 mL), compound 1g (359 mg, 1.31 mmol), potassium phosphate (1.66 g, 7.8 mmol), and Pd(PPh₃)₄ (300 mg, 0.26 mmol) were added in sequence, after replaced with nitrogen gas three times, the reaction was carried at 90° C. for 5 h. TLC showed the formation of new dots, and after the reaction was completed, the reaction solution was cooled down to room temperature, diluted with dichloromethane and H₂O, filtered through diatomaceous earth, extracted with dichloromethane, and the organic phase was washed with saturated saline solution, dried over anhydrous sodium sulfate, concentrated under reduced pressure, and purified by silica gel column chromatography (DCM/MeOH=10/1) to obtain compound 114d (144 mg), yellow solid.

MS (ESI) m/z 346 [M+H]⁺.

### Step 4 1-(4-(4-amino-6-bromo-7-methyl-7H-pyrrolo [2,3-d] pyrimidin-5-yl) phenyl-2,3,5,6-d4) imino) tetrahydro-1H-1λ⁶-thiophene-1-oxide 114e

Compound 114d (94 mg, 0.27 mmol) was dissolved in DMF (2 mL), and a solution of NBS in DMF (51 mg of NBS dissolved in 2 mL of DMF) was added dropwise in an ice water bath, and reacted under ice water bath for 1 h, and TLC showed the formation of new spots. The reaction was quenched with added saturated NaHSO₃ solution (10 mL) and H₂O (20 mL), extracted with dichloromethane (20 mL×3), and the organic phases were combined, washed with saturated saline solution, dried over anhydrous sodium sulfate, concentrated under reduced pressure, and purified by silica gel column chromatography to obtain 114e (70 mg), yellow solid.

MS (ESI) m/z 425 [M+H]⁺.

### Step 5 N-(4-(4-amino-7-methyl-5-(4-((1-oxide tetrahydro-1λ⁶-thiophene-1-ylidene) amino) phenyl-2,3,5,6-d4)-7H-pyrrolo [2,3-d] pyrimidin-6-yl)-3-fluorophenyl) methacrylamide 114

To a reaction flask, compound 114c (70 mg, 0.16 mmol), 1,4-dioxane (4 mL), water (1 mL), compound 1j (60 mg, 0.20 mmol), potassium phosphate (104 mg, 0.49 mmol), and Pd(PPh₃)₄ (9 mg, 0.016 mmol) were added in sequence, after replaced with nitrogen gas three times, the reaction was refluxed at 90° C. for 5 h. TLC showed the formation of new spots, and after the reaction was completed, it was cooled down to room temperature, and the reaction solution was diluted with dichloromethane and H₂O. After filtered through diatomaceous earth, it was extracted with dichloromethane, and the organic phase was washed with saturated saline solution, dried over anhydrous sodium sulfate, concentrated under reduced pressure, and purified by silica gel column chromatography (DCM/MeOH=10/1) to obtain compound 114 (5.0 mg), white solid.

MS (ESI) m/z 523.2 [M+H]⁺.

¹H NMR (400 MHz, DMSO-d₆) δ 10.11 (s, 1H), 8.20 (s, 1H), 7.77 (dd, J=12.4, 1.9 Hz, 1H), 7.50 (dd, J=8.5, 1.9 Hz, 1H), 7.28 (t, J=8.4 Hz, 1H), 5.84 (s, 1H), 5.59 (s, 1H), 3.54 (s, 3H), 3.38 (d, J 6.7 Hz, 2H), 3.28-3.22 (m, 2H), 2.26-2.06 (m, 4H), 1.97 (s, 3H).

### Internal Control Compound BGJ398

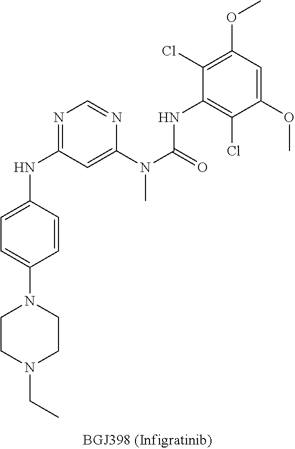 [SMILES: CCN1CCN(c2ccc(Nc3cc(N(C)C(=O)Nc4c(Cl)c(OC)cc(OC)c4Cl)ncn3)cc2)CC1]

This compound was purchased commercially, manufacturer: MCE, item number: HY-13311

### Control Compound I

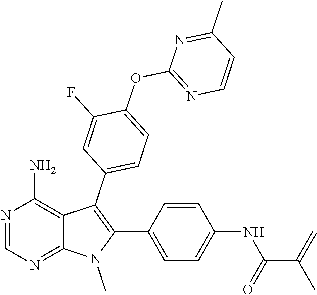 [SMILES: C=C(C)C(=O)Nc1ccc(-c2c(-c3ccc(Oc4nccc(C)n4)c(F)c3)c3c(N)ncnc3n2C)cc1]

It was synthesized with reference to WO2020231990A1.

### Test Example 1: the test of compounds on cell viability of Ba/F3 Cells

In this experiment, the inhibitory effect of compounds on cell proliferation was studied by detecting their effects on in vitro cell viability of Ba/F3 cell lines (Ba/F3-TL-FGFR1, Ba/F3-TL-FGFR2, Ba/F3-TL-FGFR3). BJG398 is an internal control compound in this project, a cell line stably expresses the fusion protein of the intracellular kinase domain of TEL-FGFR subtype obtained by transfection into Ba/F3 cells (Hefei PreceDo Biomedical Technology Co., Ltd.) for testing.

The cell line was cultured in a incubator under the culture condition of 37° C. and 5% CO₂, regularly passaged, and cells in logarithmic growth phase were taken for plating. The test compounds and the internal control compound BGJ398 were prepared into a 10 mM solution using DMSO.

Preparation of compound stock plate (tube): It was diluted with DMSO from the highest concentration to the lowest concentration in a 4-fold gradient, for a total of 9 concentrations, and the internal control compound was diluted with 4-fold diluent, for a total of 9 concentrations. Then, the preparation of the compound working solution: 98 μL cell culture medium was added to a flat bottomed 96 well transparent drug plate, and 2 μL compound was aspirated from the compound stock plate and added to the cell culture medium in 96 well transparent drug plate. 2 μL DMSO was added to the solvent control. After the compound or DMSO was added, and then mixed well by blowed with a pipette.

Cell plating and administration: Suspension cells were plated according to the following steps: 1) cells were stained with trypan blue, and live cells were counted to ensure a cell viability rate of over 90%; 2) the cell concentration was adjusted to an appropriate level; 3) 95 μL cell suspension (2000 cells/cell) was added to each well of the detection cell plate of compounds, and culture medium without cells (containing 0.1%) was added to the Min control well; 4) administration in cell plate for compounds detection: 5 μL of 20× compound working solution was taken and added to the cell culture plate as shown in Table 1. 5 μL DMSO cell culture medium mixture was added to the Max control. The final concentration of DMSO was 0.1%; 5) the culture plate was cultured in a 37° C., 5% CO₂ incubator for 72 h.

It was performed according to the instructions of the PromegaCellTiter Glo luminescent cell viability assay kit (Promega-G7573): 1) the CellTiter Glo buffer was thawed and placed to room temperature; 2) the CellTiter Glo substrate was placed to room temperature; 3) CellTiter Glo buffer was added to a bottle of CellTiter Glo substrate to dissolve the substrate and prepare CellTiter Glo working solution; 4) slow vortex oscillation for sufficient dissolution; 5) the cell culture plate was taken out and placed for 10 min for equilibrated to room temperature; 6) 50 L (equivalent to half the volume of cell culture medium in each well) of CellTiter Glo working solution was added to each well; 7) the culture plate was shaken on the track shaker for 2 min to induce cell lysis; 8) the culture plate was placed at room temperature for 10 min to stabilize the luminescence signal; 9) the luminous signal was detected on the SpectraMaxParadigm reader.

The data on inhibition rate of cell proliferation was processed using the following formula:

$$Inhibition Rate ( Inh \% ) = 100 - ( {RLU}_{Drug} - {RLU}_{Min} ) / ( {RLU}_{Max} - {RLU}_{Min} ) * 100 \% .$$

The inhibition rates of compounds at different concentrations were calculate in EXCEL, then GraphPad Prism software was used to make inhibition rate curves and calculate relevant parameters, including maximum and minimum cell inhibition rates, IC₅₀ values.

TABLE 1
Results of proliferation inhibition of test compounds on Ba/F3 cell lines
(Ba/F3-TEL-FGFR₁, Ba/F3-TEL-FGFR2, Ba/F3-TEL-FGFR3)

| | Ba/F3 cell | | |
|---|---|---|---|
| | FGFR2 | FGFR1 | FGFR3 |
| Compound number | (IC₅₀ nM) | (IC₅₀ nM) | (IC₅₀ nM) |
| BGJ398 | 4.2 | 11.3 | 6.7 |
| Control compound I | 2.5 | 1323 | 1615 |
| 1 | 2.3 | 656.1 | 869.2 |
| 2 | 11.2 | 1509 | 846.5 |
| 3 | 699.7 | — | — |
| 4 | 9.9 | 2371 | 3361 |
| 5 | 29.3 | — | — |
| 6 | 9.3 | — | — |
| 7 | 2.4 | 626.8 | 544.9 |
| 8 | 0.7 | 600.3 | — |
| 9 | 14.9 | — | — |
| 10 | 106.6 | — | — |
| 11 | 171.8 | — | — |
| 12 | 275.7 | — | — |
| 13 | 0.6 | — | — |
| 14 | 9.9 | — | — |
| 15 | 15.4 | — | — |
| 16 | 50.1 | — | — |
| 17 | 4616 | — | — |
| 18 | 21.5 | — | — |
| 19 | 2982 | — | — |
| 20 | 86.7 | — | — |
| 21 | 0.8 | — | — |
| 22 | 2.7 | 716.7 | — |
| 23 | 0.7 | 568.2 | — |
| 24 | 2.1 | 1909 | — |
| 25 | 38.7 | — | — |
| 26 | 4.5 | — | 109.6 |
| 27 | 26.0 | — | — |
| 28 | 0.9 | — | — |
| 29 | 1209 | — | — |
| 30 | 1442 | — | — |
| 31 | 8.3 | 2094 | — |
| 32 | 0.4 | — | — |
| 33 | 11.2 | 2310 | 1292 |
| 34 | 17.9 | — | — |
| 35 | 4.4 | — | — |
| 36 | 53.5 | — | — |
| 37 | 32.0 | — | — |
| 38 | 246.9 | — | — |
| 39 | 10.2 | — | — |
| 40 | 7.7 | — | — |
| 41 | 123.6 | — | — |
| 42 | 32.9 | — | — |
| 43 | 14.2 | — | — |
| 44 | 216.4 | — | — |
| 45 | 83.1 | — | — |
| 46 | 161.7 | — | — |
| 47 | 2.7 | — | — |
| 48 | 0.9 | 625.1 | 577.3 |
| 49 | 12.8 | — | — |
| 50 | 7.6 | — | — |
| 51 | 19.1 | — | — |
| 52 | 2.3 | — | — |
| 53 | 10.2 | — | — |
| 54 | 2.8 | — | — |
| 55 | 3.0 | — | — |
| 56 | 12.6 | — | — |
| 57 | 17.4 | — | — |
| 58 | 3.0 | 2026 | 441.7 |
| 59 | 7.8 | 1316 | — |
| 60 | 13.1 | — | — |
| 61 | 150.2 | — | — |
| 62 | 9.2 | — | — |
| 63 | 1.1 | 395.9 | — |
| 64 | 658.3 | — | — |
| 65 | 120.9 | — | — |
| 66 | 27.8 | — | — |
| 67 | 105.0 | — | — |
| 68 | 0.5 | — | — |
| 69 | 11.4 | — | — |
| 70 | 2.7 | 774.6 | — |
| 71 | 2.5 | — | — |
| 72 | 2.2 | — | — |
| 73 | 11.1 | — | — |
| 74 | 0.5 | 299.6 | — |
| 75 | 0.8 | 700.9 | — |
| 76 | 6.1 | — | — |
| 77 | 4.7 | 883.7 | — |
| 78 | 585.1 | — | — |
| 79 | 61.1 | — | — |
| 80 | 46.0 | — | — |
| 81 | 18.1 | — | — |
| 82 | 0.8 | 484.1 | — |
| 83 | 1.1 | 660.7 | 319.2 |
| 84 | 0.8 | 250.4 | — |
| 85 | 907.3 | — | — |
| 86 | 67.4 | — | — |
| 87 | 2.1 | 665.7 | — |
| 88 | 1.5 | 561.1 | 215.9 |
| 89 | 6.5 | 685.9 | — |
| 90 | 3.6 | — | 173.8 |
| 91 | 23.9 | — | — |
| 92 | 0.8 | — | — |
| 93 | 31.7 | — | — |
| 94 | 1.1 | 625.4 | 204.7 |
| 95 | 0.7 | 483.6 | — |
| 96 | 29.8 | — | — |
| 97 | 1.9 | 479.8 | — |
| 98 | 0.6 | 416.1 | — |
| 99 | 2.7 | — | — |
| 100 | 1.9 | 823.2 | 414.4 |
| 101 | 4.7 | — | — |
| 102 | 0.6 | — | — |
| 103 | 13.9 | — | — |
| 104 | 0.9 | 640.1 | — |
| 105 | 20.9 | — | — |
| 106 | 2.2 | — | — |
| 107 | 15.6 | 3629 | — |
| 108 | 2.5 | — | — |
| 109 | 4.5 | — | — |
| 110 | 2.0 | — | — |
| 111 | <0.15 | — | — |
| 112 | 2.0 | — | — |
| 113 | 1.8 | — | — |
| 114 | 1.2 | — | — |

—: Not tested

As can be seen from the above table, the compounds of the present invention have a good inhibitory effect on the proliferation in Ba/F3-TEL-FGFR2 cells, and exhibit high selectivity on Ba/F3-TEL-FGFR1 and Ba/F3-TEL-FGFR3.

### Test Example 2: Human Liver Microsomes Stability Test

Human liver microsomes stability test was performed by co-incubating compounds with human liver microsomes in vitro. Firstly, a 10 mM stock solution of the test compound in DMSO solvent was prepared, and then the compound was diluted with methanol to a 0.15 mM working solution. A certain amount PBS, 12 mM MgCl₂, human liver microsomes (Corning), and compound working solution were mixed to achieve a compound concentration of 1.0 μM, a human liver microsome concentration of 0.5 mg/mL, and the MgCl₂ concentration of 3 mM in the reaction system. A deep well plate was taken, 270 μL microsomal buffer mixture solution was added to each well, and then 30 μL preheated 10 mM NADPH solution was added to initiate the reaction, and it was incubated at 37° C. At 0, 5, 15, 30, and 60 min of incubation, 200 μL methanol:acetonitrile=1:1 containing an internal standard was added to the corresponding well to terminate the reaction. After the reaction was terminated at the last 60 min, the deep well plate was subjected to vortex vibration for 5 min (600 rpm/min), and then centrifugated for 10 min. After centrifugation, the supernatant was taken, and purified water was added at a 1:1 ratio for LC-MS/MS detection, and the peak area ratio of the compound to the internal standard at each time point was obtained, and the peak area ratio of the compound at 5, 15, 30, and 60 min was compared with the peak area ratio at 0 minute to calculate the remaining percentage of the compound at each time point, and T_{1/2} was calculated using Graphpad 7 software.

TABLE 2
Results of human liver microsomes stability test

| | Remaining percentage | | |
|---|---|---|---|
| | of compound after 60 | | CLᵢₙₜ |
| Compound | min of incubation (%) | T_{1/2} (min) | (mL/min/kg) |
| Control | 36.4 | 42.0 | 29.7 |
| compound I | | | |
| 2 | 54.0 | 69.3 | 18.0 |
| 4 | 61.6 | 85.6 | 14.6 |

The experimental results showed that compared with the control compound I, the compound of the present invention exhibited better liver metabolic stability and better druggability.

### Test Example 3: Pharmacokinetic Test in Rats

Rat pharmacokinetic experiments were performed using male SD rats, 180-220g, provided by Beijing Vital River Laboratory Animal Technology Co., Ltd. 3 rats that were fasted overnight were taken, and administered orally by gavage (10 mg/kg). Blood samples were collected before administration, as well as 5, 15, 30 min and 1, 2, 4, 6, 8, 24 h after administration; another 3 rats were taken and administered intravenously (1 mg/kg), blood samples were collected before administration, as well as 5, 15, 30 min and 1, 2, 4, 6, 8, 24 h after administration. Blood sample was centrifuged at 6800g, 2-8° C. for 6 min, and plasma was collected, and stored at −80° C. Plasma of each time point was taken, 10 times the amount of 50% methanol acetonitrile solution containing internal standard was added, and mixed by vortex for 5 min, centrifuged at 18000 rpm at 4° C. for 10 min, and the supernatant was taken directly for LC-MS/MS analysis. The main pharmacokinetic parameters were analyzed using WinNonlin8.2 software for non compartmental model analysis.

TABLE 3
pharmacokinetic test in rats results of test compounds

| | Pharmacokinetic parameters in rats | | | |
|---|---|---|---|---|
| | (Oral gavage administration) (10 mg/kg) | | | |
| | Cmax | Tmax | AUC₀₋ₜ | T_{1/2} |
| Compound | (ng/mL) | (hr) | (h * ng/mL) | (hr) |
| Control compound I | 141 | 0.67 | 606 | 0.85 |
| 8 | 352 | 0.25 | 630 | 1.81 |
| 24 | 270 | 0.25 | 871 | 1.43 |
| 33 | 306 | 0.42 | 778 | 1.32 |
| 48 | 212 | 0.83 | 883 | 3.44 |
| 58 | 604 | 0.50 | 1423 | 3.18 |
| 63 | 556 | 0.5 | 1696 | 2.59 |
| 83 | 414 | 0.33 | 1483 | 1.87 |

The results of pharmacokinetic test in rats showed that the compounds in the present invention have high oral exposure, good pharmacokinetic properties, and good druggability.

### Test Example 4: Manual Patch Clamp Technique for Detecting the Effect of Test Compounds on Stable Overexpressed hERG Channel Current

### 4.1. Preparation of Test Compound

A small amount of compound 24 was weighed, and prepared into a 31.346 mM stock solution using DMSO. The test compound stock solution was diluted with DMSO to 10 mM, 3.33 mM, 1 mM, 0.33 mM, and 0.1 mM diluent in sequence. The test compound diluents were diluted with extracellular fluid containing 0.5% F68 in sequence to prepare working solutions of 30 μM, 10 μM, 3 μM, 1 μM and 0.3 μM, and 0.3% DMSO was ensured. All concentrations were dissolved without visible precipitation.

A small amount of control compound I was weighed, and prepared into a 31.311 mM stock solution using DMSO. The test compound stock solution was diluted with DMSO to 10 mM, 3.33 mM, 1 mM, 0.33 mM, and 0.1 mM diluent in sequence. The test compound diluents were diluted with extracellular fluid containing 0.5% F68 in sequence to prepare working solutions with concentrations of 30 μM, 10 μM, 3 μM, 1 μM and 0.3 μM, and 0.3% DMSO was ensured. All concentrations were dissolved without visible precipitation.

The positive control Cisapride was prepared into a 10 mM stock solution using DMSO. Cisapride stock solution was diluted with DMSO to 1 mM, 333.33 μM, 33.33 μM, 3.33 μM, 0.33 μM, and 0.033 μM diluent in sequence. Cisapride diluent was diluted with extracellular fluid in sequence to prepare working solutions of 1000 nM, 100 nM, 10 nM, 1 nM and 0.1 nM, and 0.3% DMSO was ensured. All concentrations were dissolved without visible precipitation.

### 4.2 Cell Culture

The HEK-293 cell line stably expressing hERG potassium channels was cultured in DMEM medium containing 10% fetal bovine serum and 0.8 mg/mL G418 at a temperature of 37° C. and a carbon dioxide concentration of 5%. Cells were inoculated into 6 cm cell culture dishes, with a total of 2.5×10⁵ cells per dish (final volume: 5 mL). To maintain the electrophysiological activity of cells, the cell density must not exceed 80%. Before patch clamp detection, cells were isolated with TrypLE™ Express, 4×10³ cells were spread onto cover glass, and cultured in a 24 well plate (final volume: 500 μL) for 18 h, and then experimental test was performed.

### 4.3. Patch Clamp Detection

The voltage stimulation protocol for recording hERG currents using whole cell patch clamp was as follows: when a whole cell seal was formed, the cell membrane voltage was clamped at −80 mV. The clamping voltage was reduced from −80 mV to −50 mV and maintained for 0.5 s (as a leakage current detection), and then stepped up to 30 mV and maintained for 2.5 s, and then quickly restored to −50 mV and maintained for 4 s to excite the tail current of the hERG channel. Data was collected repeatedly every 10 s to observe the effect of the drug on hERG tail current. The experimental data was collected by IPA amplifier (Sutter Instrument) and stored in SutterPatch (with Igor Pro) software.

When the hERG current recorded by the whole cell was stabilized, administration began, and each drug concentration acted for about 5 min (or until the current was stabilized), and then the next concentration was detected, multiple concentrations were detected for each test compound. The cover glass covered with cells was placed in the recording bath under an inverted microscope, the blank control solution and the test compound working solution sequentially flowed through the recording bath from low concentration to high concentration using gravity perfusion to act on the cells, liquid exchange was performed using a peristaltic pump during recording. The current of each cell detected in the extracellular fluid without the compound serves as its own control group. Each concentration should be independently tested twice using at least two cells. All electrophysiological tests were performed at room temperature.

### 4.4 Test Results

The inhibitory effects of compound 24 and control compound I on hERG channels were detected in two independent replicate tests, and the IC₅₀ values of the test compounds on hERG current were calculated by fitting the concentration effect curve. The test results were as follows:

TABLE 4
Inhibition rates and IC₅₀ results of compound I and compound 24 on hERG
current

| | HERG current inhibition rate % (Mean ± SE) | | | | | | |
|---|---|---|---|---|---|---|---|
| Compound | 0.3 μM | 1 μM | 3 μM | 10 μM | 30 μM | n | IC₅₀ |
| Control | 6.28% ± | 16.23% ± | 28.98% ± | 64.51% ± | 89.42% ± | 2 | 6.046 μM |
| compound I | 0.31% | 0.51% | 0.56% | 1.31% | 0.20% | | |
| 24 | −1.24% ± | 7.73% ± | 10.17% ± | 32.17%± | 14.21% ± | 2 | >10 μM |
| | 1.48% | 0.87% | 0.62% | 2.92% | 2.95% | | |

The experimental results show that compared with the control compound I, the compound of the present invention have weak inhibition on hERG and have good safety.

### Test Example 5: In Vitro Evaluation of The Inhibitory Effect of The Test Compound on Cytochrome P450 Enzyme

### 5.1 Experimental Materials

### 5.1.1 Human Liver Microsomes

| Vendor | CAS | Cat No. | Lot No. | Concentration |
|---|---|---|---|---|
| CORNING | NA | 452161 | 1151001 | 20 mg/mL |

### 5.1.2 Positive Control

| | | | | Concentration | |
|---|---|---|---|---|---|
| | | | | of stock | |
| Name | Vendor | Cat No. | Lot No. | solution | Solvent |
| α-naphthoflavone | SIGMA | 604-59-1 | SLBN2386V | 0.3 mM | DMSO |
| Sulfamethoxazole | J&K | 178125 | LP20Q121 | 10 mM | DMSO |
| Omeprazole | TCI | 73590-58-6 | X5JEB | 100 mM | DMSO |
| Quinidine | Energy | 56-54-2 | 990XROHQ | 10 mM | DMSO |
| | Chemical | | | | |
| Ketoconazole | J&K | 973125 | LI30P36 | 2.5 mM | DMSO |

### 5.1.3 Probe Substrate

| | | | | Concentration | |
|---|---|---|---|---|---|
| | | | | of stock | |
| Name | Vendor | Cat No. | Lot No. | solution | Solvent |
| Phenacetin | aladdin | NA | F2101248 | 30 mM | DMSO |
| Diclofenac acid | TC1 | D3748 | U5XOJ | 10 mM | DMSO |
| S-methomyl | MCE | HY-B1184 | 117020 | 35 mM | DMSO |
| Dextromethorphan | National | 6700-34-1 | 100201- | 5 mM | DMSO |
| hydrobromide | Institute for | | 201204 | | |
| | Food and Drug | | | | |
| | Control | | | | |
| Testosterone | aladdin | NA | F2126139 | 80 mM | DMSO |
| Midazolam | National | 59467-70-8 | 171265- | 5 mM | DMSO |
| | Institute for | | 201402 | | |
| | Food and Drug | | | | |
| | Control | | | | |

### 5.1.4 Internal Standard

| Name | Vendor | Cat No. | Lot No. |
|---|---|---|---|
| Toluene sulfonamide | Supelco | T0891 | SLCK4822 |
| Verapamil | TCI | V0118 | RFMWJ |

### 5.1.5 Other Reagents

| Name | Vendor | Cat No. | Lot No. |
|---|---|---|---|
| NADPH | MCE | HY-F0003 | 139299 |
| Phosphate buffered | Yuanpei | B320KJ | K211021 |
| saline (PBS) | | | |

Termination solution containing internal standard: toluene sulfonamide concentration of 20.0 ng/mL, verapamil concentration of 10.0 ng/mL

### 5.2 Experimental Preparation

Preparation of positive control inhibitor and compound working solution: 16 μL inhibitor stock solution or compound stock solution for each CYP subtype were taken and added to 24 L DMSO. Then, they were diluted gradually three times with diluent to obtain different concentrations of test sample/positive control inhibitor working solution (7 concentrations each except for blank)

Preparation of substrates for each CYP subtype: Preparation of substrates for each CYP subtype: for CYP1A2, CYP2C9, CYP3A4 (midazolam), and CYP2D6, 8 μL substrate stock solution was added to 1992 μL PBS; for CYP2C19, 40 μL substrate stock solution and 160 L human liver microsomes (20.0 mg/mL) were added to 1800 μL of PBS; for CYP3A4 (testosterone), 40 μL substrate stock solution was added to 1960 μL PBS.

Preparation of liver microsomal working solution: 300 μL human liver microsomes (20.0 mg/mL) was taken and added to 29700 μL PBS and mixed well, and the concentration of the prepared human liver microsomal working solution was 0.200 mg/mL.

Preparation of 4×NADPH working solution: A certain weight of NADPH tetrasodium salt was weighted and dissolved to a suitable volume of PBS to obtain NADPH working solution, with a concentration of 8.00 mM.

Preparation of test sample group working solution: The human liver microsomal working solution was divided into 96 well plates at 796 μL per well, and then 4 μL test sample working solution was added.

Preparation of positive control group working solution: The human liver microsomal working solution was divided into 96 well plates at 199 μL per well, and then 1 μL positive control inhibitor working solution was added.

Blank solvent working solution preparation: The human liver microsomal working solution was divided into 96 well plates at 199 μL per well, and then 1 μL DMSO was added.

### 5.3 Experimental Steps

(a) 30 μL test sample/positive control inhibitor working solution or 30 μL blank solvent working solution per well were transferred into a 96 well plate, and 15 μL substrate working solution of different enzyme subtypes transferred into each well, and mixed evenly.
(b) The above incubation plates and 4×NADPH working solution were preheated at 37° C. for 5 min.
(c) 15 μL preheated 4×NADPH working solution per well was transferred into the incubation plate, and the reaction initiated, and continued to be incubated at 37° C. The incubation time is as follows:

| | | | | | Liver | |
|---|---|---|---|---|---|---|
| | | Final | | Final highest | microsomal | Incubation |
| | | concentration | | concentration | concentration | time |
| CYP | Substrate | (μM) | Inhibitor | (μM) | (mg/mL) | (min) |
| 1A2 | Phenacetin | 30.0 | α-naphthoflavone | 0.300 | 0.100 | 10 |
| 2C9 | Diclofenac acid | 10.0 | Sulfamethoxazole | 10.0 | 0.100 | 10 |
| 2C19 | S-meifentuin | 35.0 | Omeprazole | 100 | 0.500 | 45 |
| 2D6 | Dextromethorphan | 5.00 | Quinidine | 10.0 | 0.100 | 20 |
| 3A4 | Testosterone | 80.0 | Ketoconazole | 2.50 | 0.100 | 5 |
| 3A4 | Midazolam | 5.00 | Ketoconazole | 2.50 | 0.1 | 5 |

(d) After the reaction time was reached, 180 μL termination solution was added to terminate the reaction.
(e) The incubation plate was vibrated at 600 rpm for 10 min, and then centrifuged at 6000 rpm for 15 min.
(f) After centrifugation, 80 μL supernatant was taken and added to 120 μL ultrapure water, and after it was mixed evenly, LC-MS/MS was used to analyze the metabolites of the probe substrate.

TABLE 5
Inhibition tests results of control compound I and the compounds
of the present invention on CYP subtypes

| | IC₅₀ (μM) | | | | | |
|---|---|---|---|---|---|---|
| | | | | | 3A4 | 3A4 |
| Compound | 1A2 | 2C9 | 2C19 | 2D6 | (Testosterone) | (Testosterone) |
| Control | >10 | 1.68 | 7.78 | 1.50 | 11.84 | 10.85 |
| compound I | | | | | | |
| 9 | — | — | — | 24.61 | — | 13.52 |
| 24 | >10 | 29 | 19.37 | 14.27 | 10.68 | — |
| 33 | — | — | — | 9.52 | — | 19.19 |
| 56 | — | — | — | 5.54 | — | 13.23 |
| 58 | >10 | 29.68 | 5.42 | 9.81 | >10 | — |
| 83 | >10 | 14.40 | 14.13 | 8.77 | 11.57 | — |

—: Not tested

The experimental results indicate that compared with the control compound I, the compounds of the present invention have weaker inhibitory effects on CYP subtypes and have lower potential drug interaction risks.

### Test Example 6: In Vitro Evaluation of Rat Liver Cells Metabolic Stability of Test Compound

### 6.1 Rat Liver Cells and Reagents

| Species | Strain | Cat No. | Lot No. | Gender | Supplier |
|---|---|---|---|---|---|
| Rat | SD | M00005 | DVB | Male | BioIVT |

| Reagent | Vendor | Cat No. |
|---|---|---|
| Isotonic percoll | Cytiva | 10303640 |
| HEPES | Sigma | RNBH8969 |
| DPBS | Gibco | 2224922 |
| FBS | Corning | 35-076-CV |
| Human-insulin | Gibco | 12585-014 |
| Dexamethasone | NICPBP | — |
| Williams E Medium | Gibco | 2336217 |
| GlutaMAX (100x) | Gibco | 22777239 |

### 6.2 Experimental Design

### 6.2.1 Preparation of Compound Working Solution

Test compound and control drug verapamil powder were prepared into 10 mm stock solution using DMSO, and they were diluted with 50% acetonitrile/50% water to 10 μM working solution before use.

### 6.2.2 Preparation of Liver Cells

1) 49.5 mL Williams E Medium and 0.5 mL glutaMAX were mixed as incubation medium. The liver cell resuscitation solution and incubation solution were preheated in a 37° C. water bath for at least 15 min before use.
2) A tube of liver cells stored at ultra-low temperature was taken and ensured that they remain in a low-temperature frozen state before resuscitation. The liver cells were placed quickly in a 37° C. water bath and shaken gently until all ice crystals were dispersed.
3) The contents of the liver cells tubes were poured into a centrifuge tube containing 50 mL of resuscitation medium, and they were centrifuged at 100 g for 10 min. After centrifugation, the resuscitation medium was aspirated, and sufficient incubation medium was added to obtain a cell suspension with a cell density of approximately 1.0×10⁶ cells/mL.
4) AO/PI staining was used to count liver cells and determine viable cell density, the survival rate of liver cells must be greater than 75%. The liver cell suspension was diluted with incubation medium to a viable cell density of 0.5×10⁶ cells/mL.

### 6.3 Experimental Methods

1) 198 μL live cell suspension was transferred to a 96 well deep well plate, and the deep well plate was placed on a vortex and preheat in the incubator for 10 min.
2) 2 μL 10 μM test compound or positive control compound was added to each well for reaction initiation, and the deep well plate was placed back on the incubator vortex.
3) The incubation plate was placed into the incubator for incubation. 25 μL suspension was taken at 0.5, 15, 30, 60, 90, and 120 min, and 6-fold (150 μL) acetonitrile containing internal standard quencher (100 nM alprazolam, 200 nM caffeine, and 100 nM toluenesulfonylbutamide) was added to a new 96 well plate to terminate the reaction. The plate was vortexed for 5 min and centrifuged at 3220 rpm and 4° C. for 45 min to precipitate the protein. 100 μL of supernatant was transferred to the sample plate, and 100 μL pure water was added and mixed well for UPLC-MS/MS analysis. All incubation systems were conducted in a dual parallel manner.

### 6.4 Data Analysis and Experimental Results

All data was calculated using Microsoft Excel software. Peak area was detected by extracting ion chromatogram. In vitro half-life (T_{1/2}) and in vitro clearance rate (in μL/min/10⁶ cells) of the parent drug were detected by linearly fitting of the natural logarithm of the elimination percentage of the parent drug versus time.

TABLE 6
Results of rat liver cell stability test

| | Remaining percentage | in vitro | |
|---|---|---|---|
| | of compound after 60 | t_{1/2} | in vitro CLᵢₙₜ |
| Compound | min of incubation (%) | (min) | (μL/min/10⁶ cells) |
| Control | 18.14 | 24.52 | 56.66 |
| compound I | | | |
| 23 | 24.45 | 28.65 | 48.40 |
| 24 | 37.55 | 37.34 | 37.13 |
| 83 | 35.86 | 37.15 | 37.31 |

The experimental results showed that compared with the control compound I, the compounds in the present invention exhibited better liver cell metabolic stability and better druggability.

### Test Example 7: The Test of The Proliferation Inhibitory Activity of Compounds on SNU16 and A204 Cells

CCK-8 (Bimake/B34304) colorimetric method was used to detect the in vitro proliferation inhibitory effect of compounds on suspended or adherent cells in this experiment. The specific experimental steps were as follows:
1) Cell plating: SNU16 and A204 tumor cells (cells from Shanghai Institute of Organic Chemistry, Chinese Academy of Sciences) in the logarithmic growth phase were digested and centrifuged for trypan blue counting, and the cells were seeded into a transparent 96 well plate at a density of 4000 cells/100 μl/well. Blank control group: only culture medium was added.
2) Preparation of working solution: The test compound and internal control compound BGJ398 were both prepared into 10 mM stock solutions using DMSO. The test compound and control compound stock solution were diluted with the corresponding culture medium required for cell culture (with or without solvent DMSO) to obtain a series of working solutions, with concentrations of twice the final concentration. The solvent DMSO contents in each concentration group were consistent with that of the solvent control group. The final concentration of A204 cells started at 1 μM and was diluted twice. The final concentration of SNU-16 cells started at 1 μM and was diluted 4-fold.
3) Co-incubation: After 24 h of plate plating, 100 μl/well mother solution of compound with series concentration was added to a 96 well plate and mixed well, then co-cultured for 72 h. All groups should have at least 4 replicates, with 10 concentrations of each compound. Blank control group: Only culture medium was added, and no cells or drugs were added, to eliminate interference from the contrast color of the culture medium.
4) Measurement of absorbance: 30 μl of CCK8/culture medium (1:2) mixed system was added to each well, and co-cultured for 2 h, then shaken thoroughly to make it uniform, and the absorbance values at A450 and A650 were measured on a BioTek Synergy Hi enzyme-linked immunosorbent assay reader.
5) Process of data: The raw data of A450-A650 was obtained, the cell survival rates of each treatment well were obtained, and a nonlinear regression model was used to calculate the IC₅₀ values of the compound on different cells.

Calculation of cell survival rate: Cell survival rate (0%)=[(As−Ac)/(Ab−Ac)]×1000%] (As: experimental well; Ab: Solvent control well; Ac: Blank well).

TABLE 7
Results of proliferation inhibition of test
compounds on SNU16 and A204 cell lines

| | Cells | |
|---|---|---|
| Compound number | SNU16 (IC₅₀ nM) | A204 (IC₅₀ nM) |
| BGJ398 | 4.3 | 100 |
| Control compound I | 13.2 | 3220 |
| 1 | 5.57 | 1314 |
| 7 | 5.43 | 582.7 |
| 8 | 1.34 | 618 |
| 21 | 1.72 | 332.7 |
| 22 | 4.97 | 1606 |
| 23 | 2.4 | 617.6 |
| 24 | 1.59 | 678.9 |
| 26 | 3.36 | 285.4 |
| 32 | 9.56 | 2250 |
| 35 | 4.91 | 406.4 |
| 48 | 13.8 | 1350 |
| 58 | 9.52 | 1271 |
| 63 | 8.71 | 459.7 |
| 68 | 1.65 | 267.4 |
| 74 | 1.19 | 595 |
| 83 | 1.89 | 627.1 |
| 87 | 7.74 | 3595 |
| 88 | 5.01 | 858.4 |
| 97 | 2.66 | 763.7 |
| 98 | 1.81 | 845.4 |
| 100 | 4.78 | 642.2 |
| 102 | 2.63 | 502 |
| 104 | 5.51 | 1018 |
| 109 | 9.38 | 1045 |

As can be seen from the above table, the compounds of the present invention have a good proliferation inhibitory effect on SNU-16 cells and weak proliferation inhibitory activity on A204 cells, which showed high selectivity.

All references mentioned in the present invention are cited as references in this application, as if each reference were cited separately. In addition, it should be understood that after reading the above teaching content of the present invention, those skilled in the art can make various changes or modifications to the present invention, and these equivalent forms also fall within the scope of the claims attached to this application.

1. A compound as shown in formula (I), a tautomer, a stereoisomer, a hydrate, a solvate, or a pharmaceutically acceptable salt thereof: 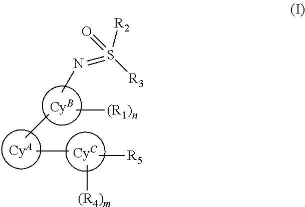
wherein, n and m are each independently 0, 1, 2, or 3;
Cy^{A} is selected from the group consisting of: 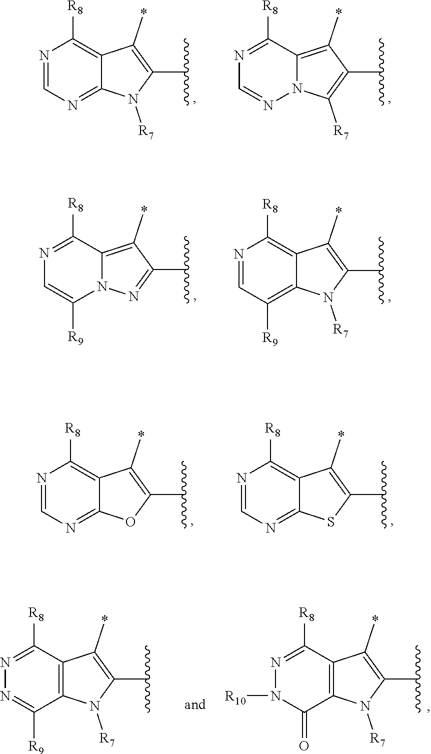
wherein [symbol] denotes a bond connected to Cy^{B}, and 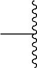
denotes a bond connected to Cy^{C};
Cy^{B} is selected from the group consisting of: C₆₋₁₀ aryl and 5-12 membered heteroaryl;
Cy^{C} is selected from the group consisting of: C₆₋₁₀ aryl, 5-12-membered heteroaryl, saturated or partially unsaturated C₃₋₆ carbocyclyl and saturated or partially unsaturated 4-12-membered heterocyclyl;
R₁ is selected from the group consisting of: H, D, halogen, CN, OH, NH₂, C₁₋₆ alkyl, C₁₋₆ alkoxy, C₁₋₆ haloalkyl, C₁₋₆ haloalkoxy, C₁₋₆ alkylamino, C₂-4 alkenyl, C₁₋₄ haloalkenyl, C₂₋₄ alkynyl, C₁₋₄ haloalkynyl, C₃₋₆ saturated or partially unsaturated carbocyclyl, C₃₋₆ halogenated saturated or partially unsaturated carbocyclyl, C₃₋₆ saturated or partially unsaturated carbocyclyl-O—, SF₅ and 4-12 membered heterocyclyl;
R₂ and R₃ are each independently selected from the group consisting of: C₁₋₆ alkyl, C₁₋₆ alkoxy, C₁₋₆ haloalkyl, C₁₋₆ haloalkoxy, C₁₋₆ alkylamino, C₃₋₆ saturated or partially unsaturated carbocyclyl and 4-12-membered heterocyclyl; wherein R₂ and R₃ can be the same or different;
or R₂ and R₃ together with the sulfur atom to which they are connected form a 4-12-membered heterocyclyl;
R₄ is selected from the group consisting of: H, D, halogen, CN, OH, NH₂, C₁₋₆ alkyl, C₁₋₆ alkoxy, C₁₋₆ haloalkyl, C₁₋₆ haloalkoxy, C₁₋₆ alkylamino, C₂₋₄ alkenyl, C₁₋₄ haloalkenyl, C₂₋₄ alkynyl, C₁₋₄ haloalkynyl, C₃₋₆ saturated or partially unsaturated carbocyclyl, C₃₋₆ halogenated saturated or partially unsaturated carbocyclyl, C₃₋₆ carbocyclyl-O—, SF₅ and 4-12-membered heterocyclyl;
R₅ is -L-Rw, wherein L is selected from the group consisting of: covalent bond, —C₁₋₄ alkyl-, —NR₆—, —C₁₋₄ alkyl NR₆—;
R₆ is selected from the group consisting of: H, C₁₋₆ alkyl, C₁₋₆ alkoxy, C₁₋₆ haloalkyl and C₁₋₆ haloalkoxy;
Rw is selected from the group consisting of: halogen, cyano, 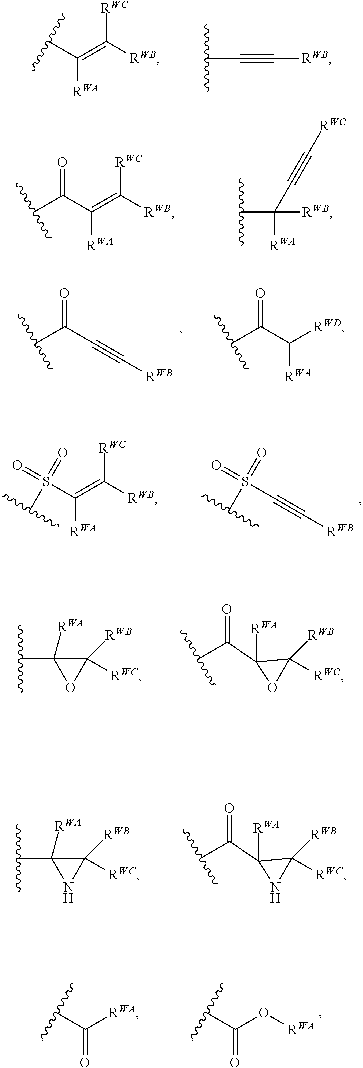
and 5-12-membered nitrogen-containing heterocyclyl;
R^{WA}, R^{WB}, and R^{WC} are each independently selected from the group consisting of: H, D, halogen, CN, C(O)Ra, C(O)ORa, NRaRb, C(O)NRaRb, C(O)NRaORb, or selected from the group consisting of: C₁₋₆ alkyl, C₁₋₆ alkoxy, C₁₋₆ haloalkyl, C₁₋₆ haloalkoxy, phenyl, 3 to 7-membered saturated or partially unsaturated heterocyclyl with 1 to 2 heteroatoms independently selected from nitrogen, oxygen and sulfur, and 5 to 6-membered heteroaryl with 1 to 4 heteroatoms independently selected from nitrogen, oxygen and sulfur; and the R^{WA}, R^{WB}, and R^{WC} can each be independently substituted with 1, 2, or 3 substituents selected from the group consisting of: C₁₋₆ alkylamino and C₁₋₆ alkoxy;
or R₆ and Rw together form a ring; and the ring is selected from the group consisting of: 5-12 membered heteroaryl and 4-12 membered heterocyclyl;
R₇ is selected from the group consisting of: H, C₁₋₆ alkyl, C₁₋₆ alkoxy, C₁₋₆ haloalkyl, C₁₋₆ haloalkoxy, C₁₋₆ alkylamino, C₂₋₄ alkenyl, C₁₋₄ haloalkenyl, C₂₋₄ alkynyl, C₁₋₄ haloalkynyl, C₃₋₆ saturated or partially unsaturated carbocyclyl, C₃₋₆ halogenated saturated or partially unsaturated carbocyclyl, C₆₋₁₀ aryl, 5-12 membered heteroaryl, 4-12-membered heterocyclyl; or R₄ and R₇, as well as the atoms between them, together form a ring, and the ring is selected from the group consisting of: C₆₋₈ saturated or partially unsaturated carbocyclyl, C₆₋₁₀ aryl, 6-12 membered heteroaryl and 6-12 membered saturated or partially unsaturated heterocyclyl;
R₈ is selected from the group consisting of: H, OH, NH₂, C₁₋₆ alkyl, C₁₋₆ alkoxy, C₁₋₆ haloalkyl, C₁₋₆ haloalkoxy and C₁₋₆ alkylamino;
R₉ is selected from the group consisting of: H, D, halogen, CN, NH₂, C₁₋₆ alkyl, C₁₋₆ alkoxy, C₁₋₆ haloalkyl, C₁₋₆ haloalkoxy and C₁₋₆ alkylamino;
unless otherwise specified, each of the above-mentioned alkyl, alkoxy, haloalkyl, haloalkoxy, alkylamino, alkenyl, haloalkenyl, alkynyl, haloalkynyl, saturated or partially unsaturated carbocyclyl, halogenated saturated or partially unsaturated carbocyclyl, saturated or partially unsaturated heterocyclyl, aryl and heteroaryl can be substituted with one or more Ra;
Ra and Rb are each independently selected from H, D, halogen, CN, oxo (═O), OH, NH₂, C₁₋₆ alkyl, C₁₋₆ alkoxy, C₁₋₆ haloalkyl, C₁₋₆ haloalkoxy, C₁₋₆ alkylamino, C₂₋₄ alkyenyl, C₁₋₄ haloalkenyl, C₂₋₄ alkynyl, C₁₋₄ haloalkynyl, C₃₋₆ saturated or partially unsaturated carbocyclyl, C₃₋₆ halogenated saturated or partially unsaturated carbocyclyl, C₃₋₆ saturated or partially unsaturated carbocyclyl-O—, 4-12 membered heterocyclyl;
wherein, the heterocyclyl is a saturated or partially unsaturated non-aromatic group; the carbocyclyl or heterocyclyl can be optionally in the form of a monocycle, bridged ring, spirocycle, or fused ring.

2. The compound according to claim 1, or a tautomer, a stereoisomer, a hydrate, a solvate, or a pharmaceutically acceptable salt thereof, wherein, the compound has a structure as shown in formula (I-1): 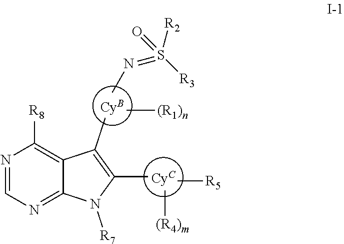
wherein, R₁, R₂, R₃, R₄, R₅, R₇, R₈, Cy^{B} and Cy^{C} are as defined in claim 1;
or R₇, R₄, and atoms to which they connected together form a 5-12 membered carbocyclyl or heterocyclyl.

3. The compound according to claim 1, or a tautomer, a stereoisomer, a hydrate, a solvate, or a pharmaceutically acceptable salt thereof, wherein, the compound has a structure as shown in formula (I-2):

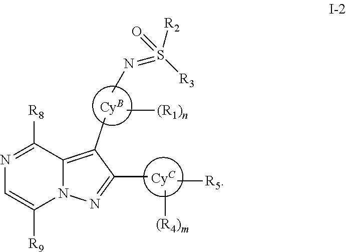

4. The compound according to claim 1, or a tautomer, a stereoisomer, a hydrate, a solvate, or a pharmaceutically acceptable salt thereof, wherein, Cy^{B} is selected from the group consisting of: phenyl and 5-7 membered heteroaryl;
preferably, Cy^{B} is selected from the group consisting of: phenyl, pyridyl, pyrimidinyl, pyridazinyl, pyrrolyl, furanyl, thienyl, imidazolyl, pyrazolyl, thiazolyl, triazolyl, and oxazolyl.

5. The compound according to claim 1, or a tautomer, a stereoisomer, a hydrate, a solvate, or a pharmaceutically acceptable salt thereof, wherein, Cy^{C} is selected from the group consisting of: phenyl, 5-7 membered heteroaryl, saturated or partially unsaturated C₃-6 saturated or partially unsaturated carbocyclyl, and saturated or partially unsaturated 4-7 membered heterocyclyl;
preferably, Cy^{C} is selected from the group consisting of: phenyl, pyridyl, pyrimidinyl, pyridazinyl, pyrrolyl, furanyl, thiophenyl, imidazolyl, pyrazolyl, thiazolyl, triazolyl, oxazolyl, and 5-7 membered saturated or partially unsaturated heterocyclyl.

6. The compound according to claim 1, or a tautomer, a stereoisomer, a hydrate, a solvate, or a pharmaceutically acceptable salt thereof, wherein, R₅ is -L-Rw, wherein L is selected from the group consisting of: covalent bonds, —C₁₋₄ alkyl, and —NR₆—; R₆ is selected from the group consisting of: H, and C₁₋₆ alkyl;
Rw is selected from the group consisting of: cyano, 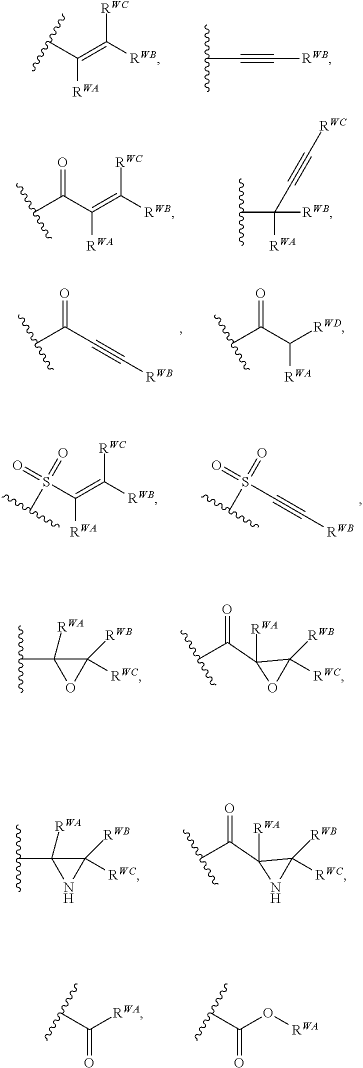
and 5-7 membered nitrogen-containing heterocyclyl;
R^{WA}, R^{WB}, and R^{WC} are each independently selected from the group consisting of: H, D, halogen, C₁₋₆ alkyl, and C₁₋₆ haloalkyl; and R^{WA}, R^{WB}, and R^{WC} can each be independently substituted with 1, 2, or 3 substituents selected from the group consisting of: C₁₋₆ alkylamino and C₁₋₆ alkoxy;
or R₆ and Rw together form a ring; and the ring is selected from the group consisting of: 5-7 membered heteroaryl and 4-7 membered heterocyclyl.

7. The compound according to claim 1, or a tautomer, a stereoisomer, a hydrate, a solvate, or a pharmaceutically acceptable salt thereof, wherein,
R₁ is selected from the group consisting of: H, D, halogen, CN, OH, NH₂, C₁₋₆ alkyl, C₁₋₆ alkoxy, C₁₋₆ haloalkyl, C₁₋₆ haloalkoxy, C₁₋₆ alkylamino, C₂₋₄ alkenyl, C₁₋₄ haloalkenyl, C₂₋₄ alkynyl, C₁₋₄haloalkynyl and SFs;
R₂ and R₃ are each independently selected from the group consisting of: C₁₋₆ alkyl, C₁₋₆ alkoxy, C₁₋₆ haloalkyl, C₁₋₆ haloalkoxy and C₁₋₆ alkylamino; wherein, R₂ and R₃ can be the same or different;
or R₂ and R₃ together with the sulfur atom to which they are connected form a 3-7 membered heterocyclyl (preferably a 4-6 membered heterocyclyl);
R₄ is selected from the group consisting of: H, D, halogen, CN, OH, NH₂, C₁₋₆ alkyl, C₁₋₆ alkoxy, C₁₋₆ haloalkyl, C₁₋₆ haloalkoxy and C₁₋₆ alkylamino;
preferably, R₁ is selected from the group consisting of: H, D, halogen, C₁₋₆ alkyl, C₁₋₆ alkoxy, C₁₋₆ haloalkyl and C₁₋₆ haloalkoxy;
R₂ and R₃ are each independently selected from the group consisting of: C₁₋₆ alkyl, C₁₋₆ haloalkyl, C₁₋₆ alkylamino and C₁₋₆ haloalkylamino; wherein, R₂ and R₃ can be the same or different; or R₂ and R₃ together with the sulfur atom to which they are connected form a 4-8 membered heterocyclyl;
R₄ is selected from the group consisting of: H, D, halogen, C₁₋₆ alkyl, C₁₋₆ alkoxy, C₁₋₆ haloalkyl, and C₁₋₆ haloalkoxy.

8. The compound according to claim 1, or a tautomer, a stereoisomer, a hydrate, a solvate, or a pharmaceutically acceptable salt thereof, wherein the compound is selected from the group consisting of:

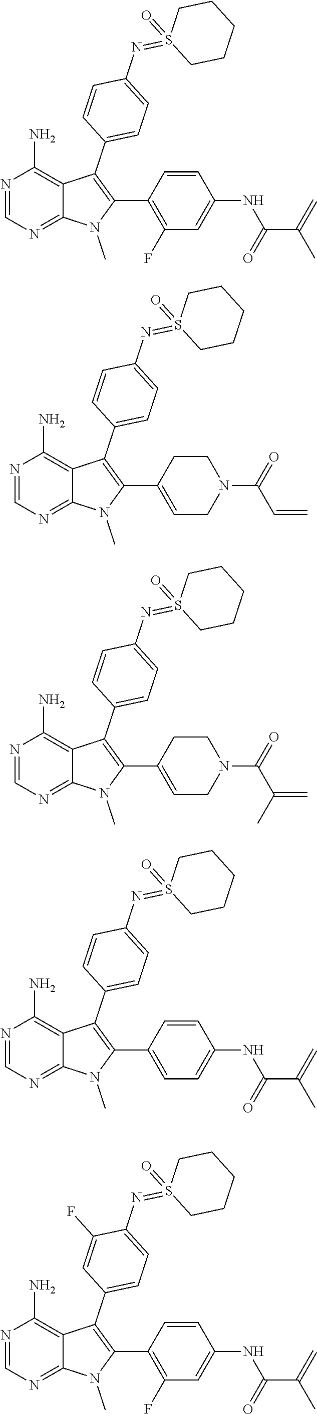

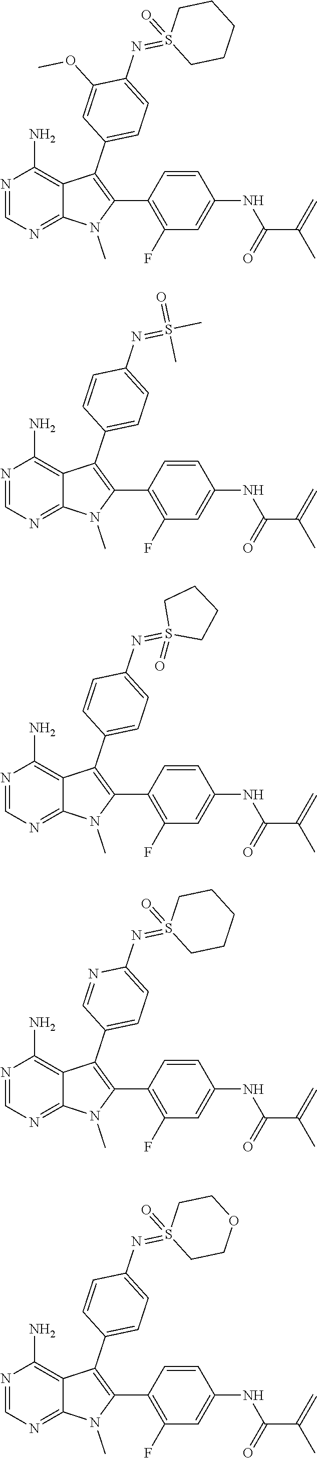

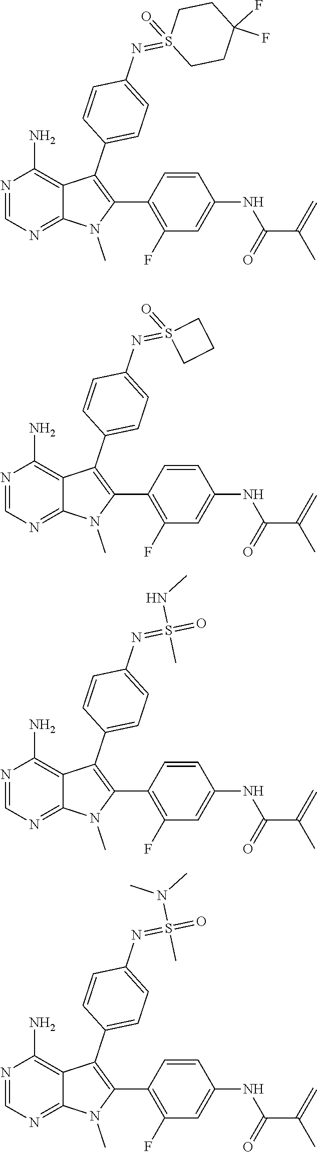

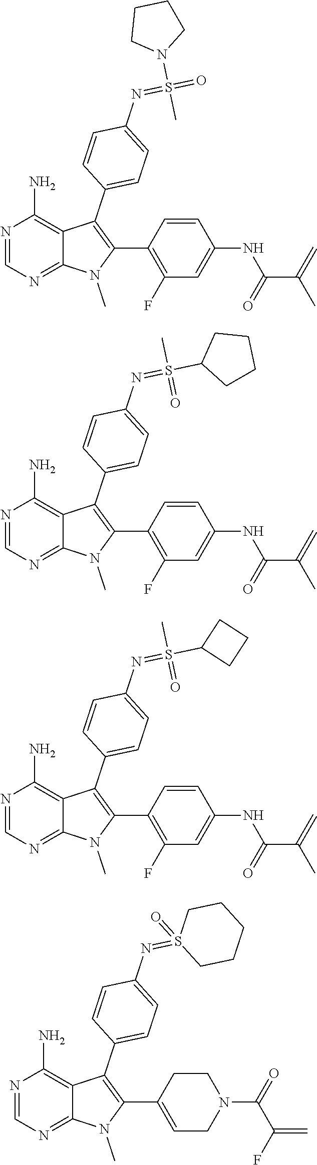

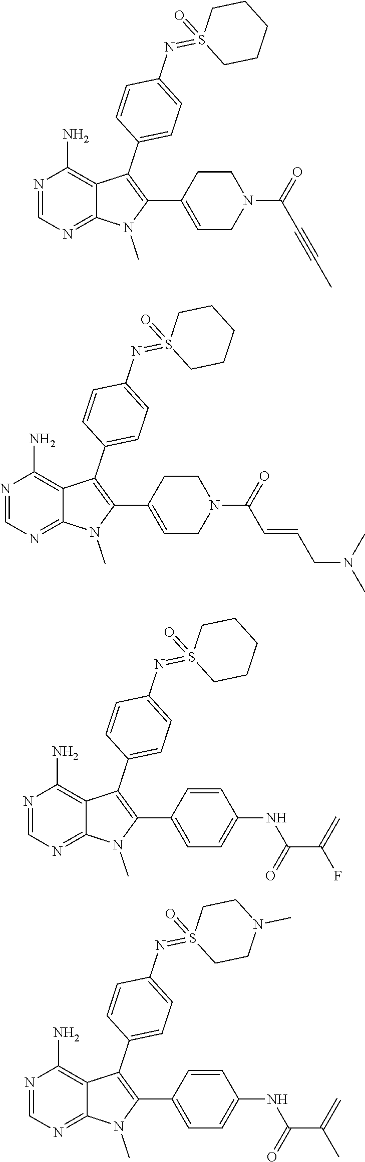

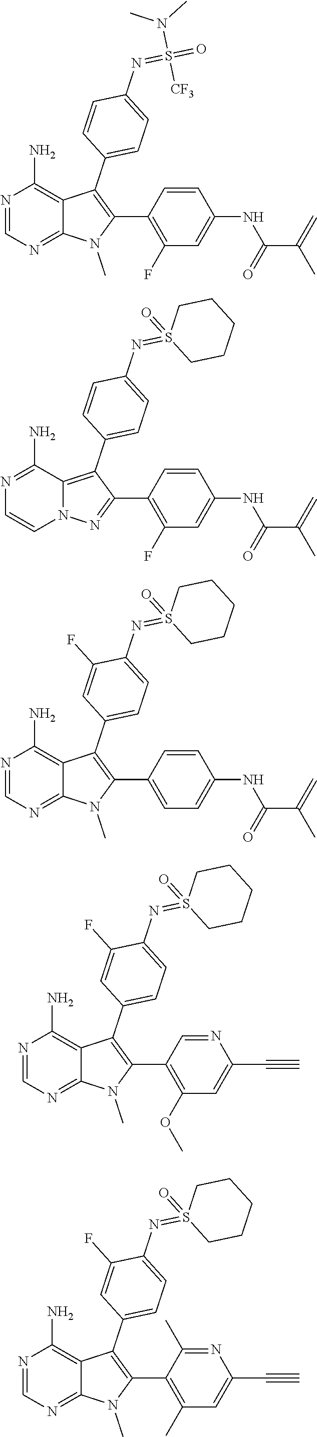

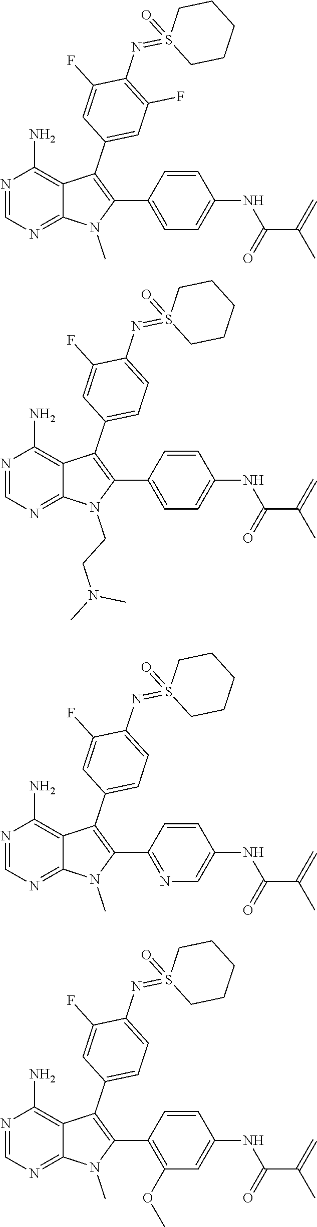

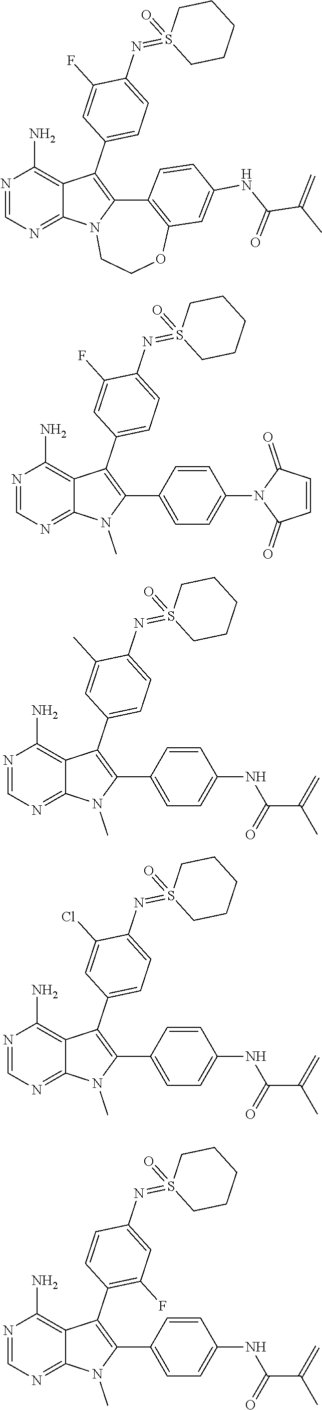

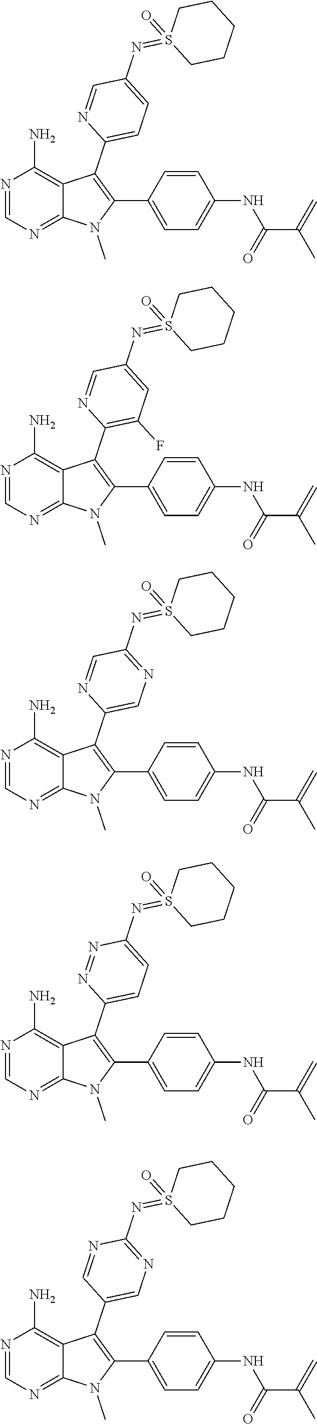

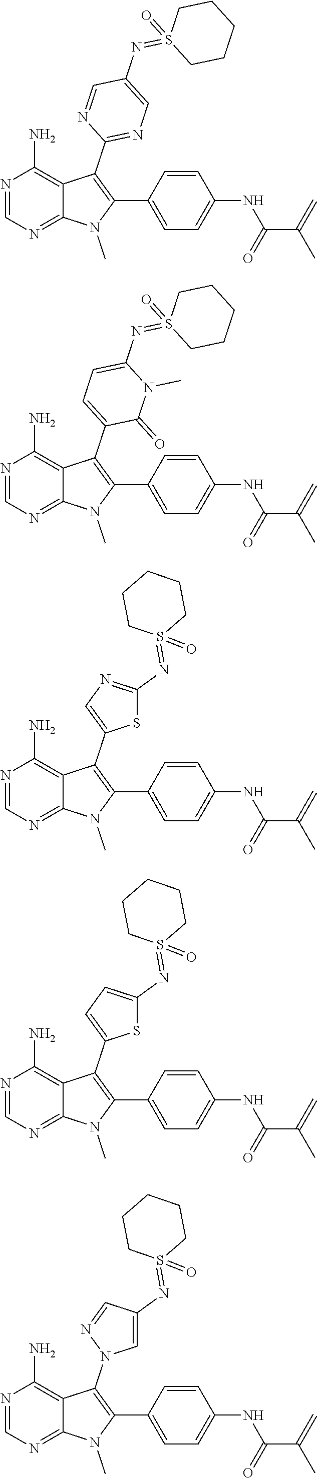

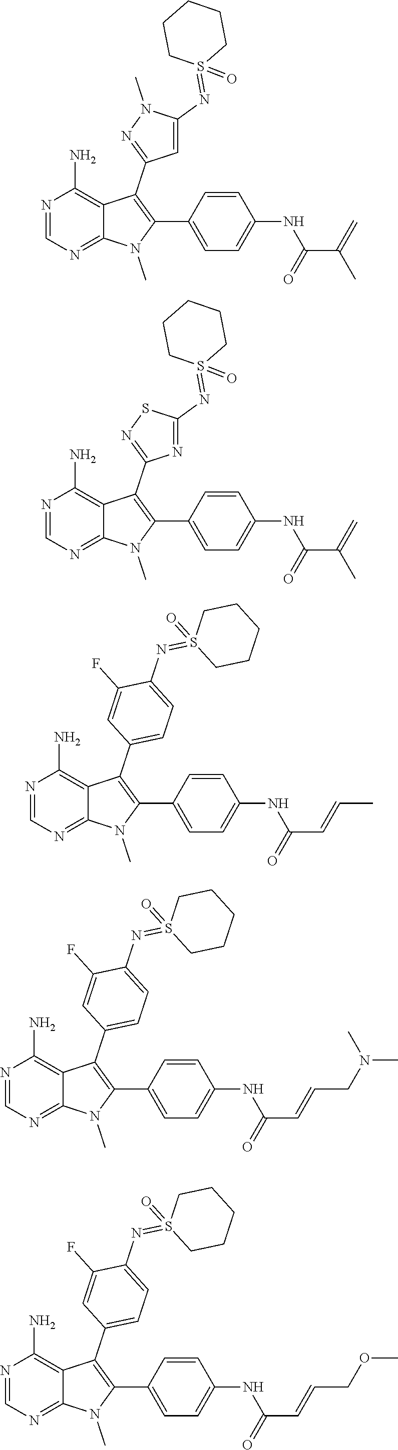

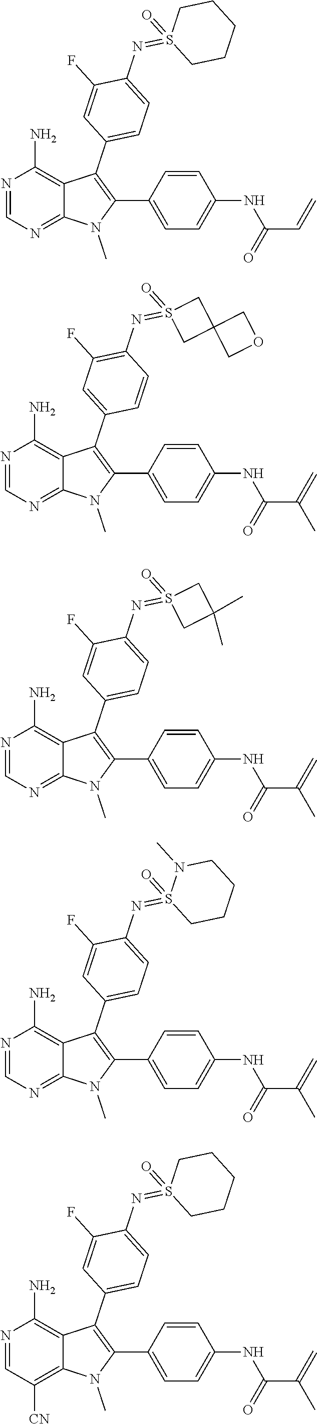 [SMILES: C=CC(=O)Nc1ccc(-c2c(-c3ccc(N=S4(=O)CCCCC4)c(F)c3)c3c(N)ncnc3n2C)cc1 ; C=C(C)C(=O)Nc1ccc(-c2c(-c3ccc(N=S4(=O)CC5(COC5)C4)c(F)c3)c3c(N)ncnc3n2C)cc1 ; C=C(C)C(=O)Nc1ccc(-c2c(-c3ccc(N=S4(=O)CC(C)(C)C4)c(F)c3)c3c(N)ncnc3n2C)cc1 ; C=C(C)C(=O)Nc1ccc(-c2c(-c3ccc(N=S4(=O)CCCCN4C)c(F)c3)c3c(N)ncnc3n2C)cc1 ; C=C(C)C(=O)Nc1ccc(-c2c(-c3ccc(N=S4(=O)CCCCC4)c(F)c3)c3c(N)ncc(C#N)c3n2C)cc1]

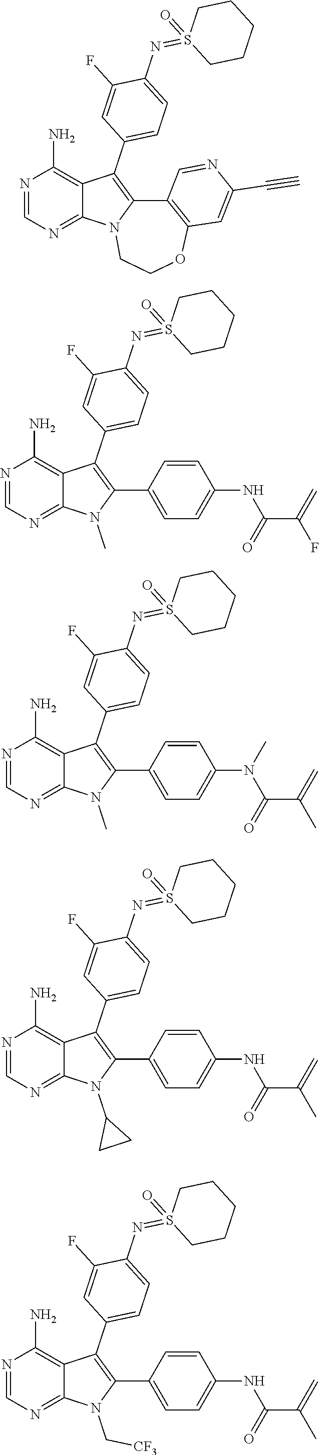

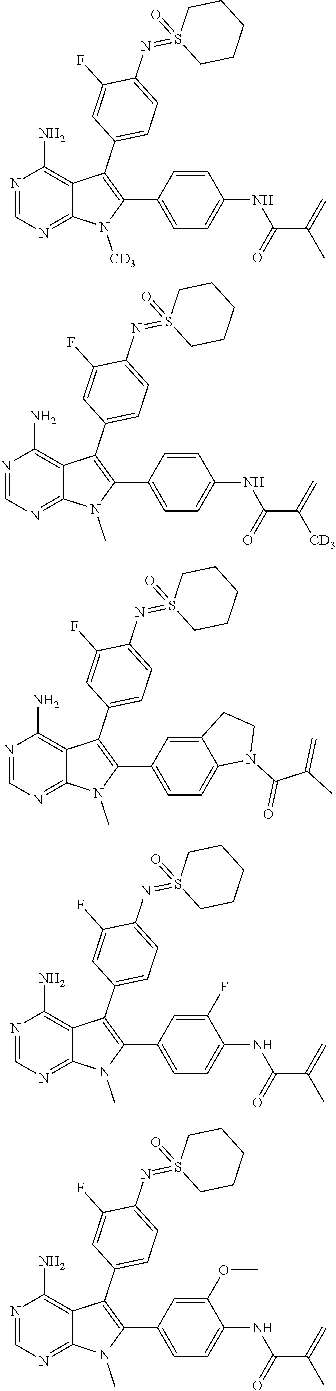

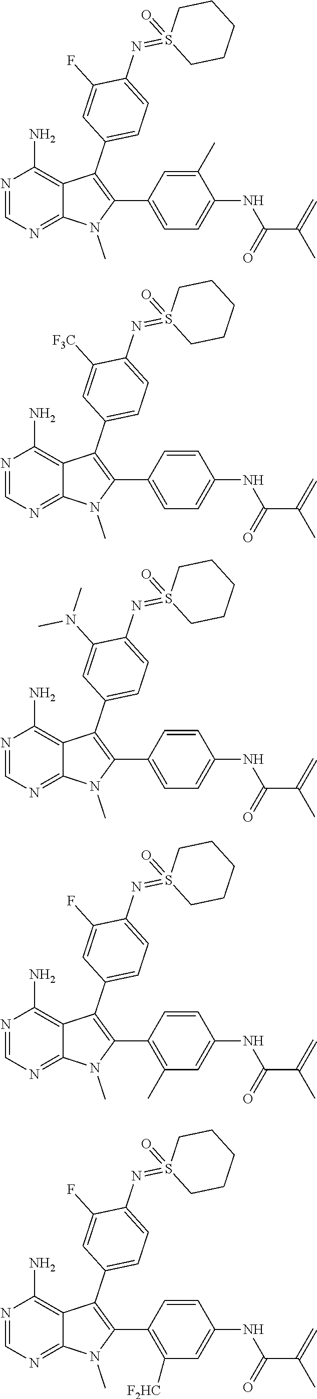

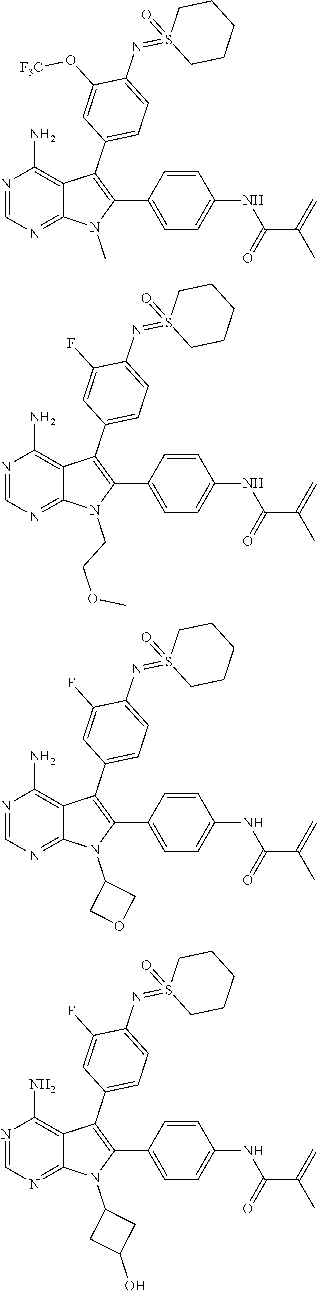

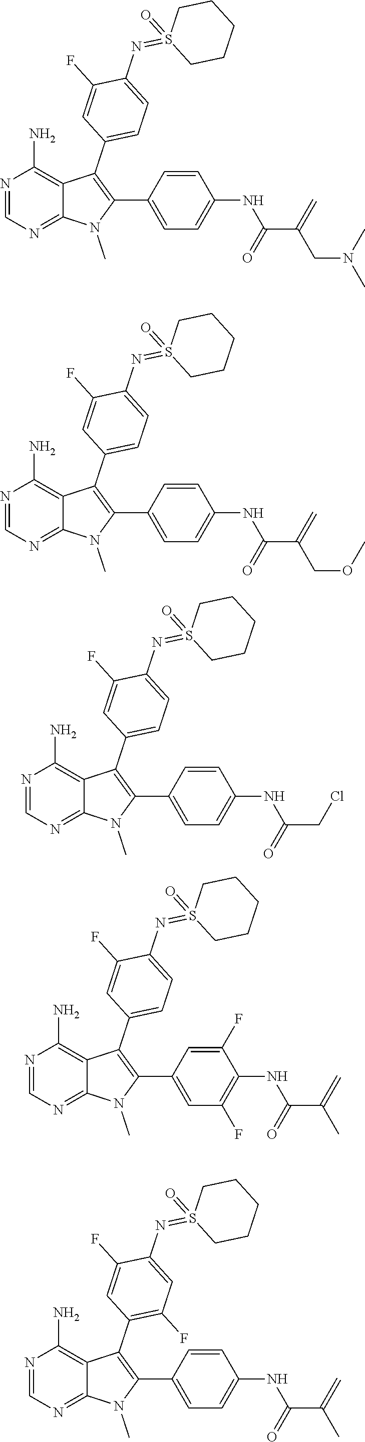

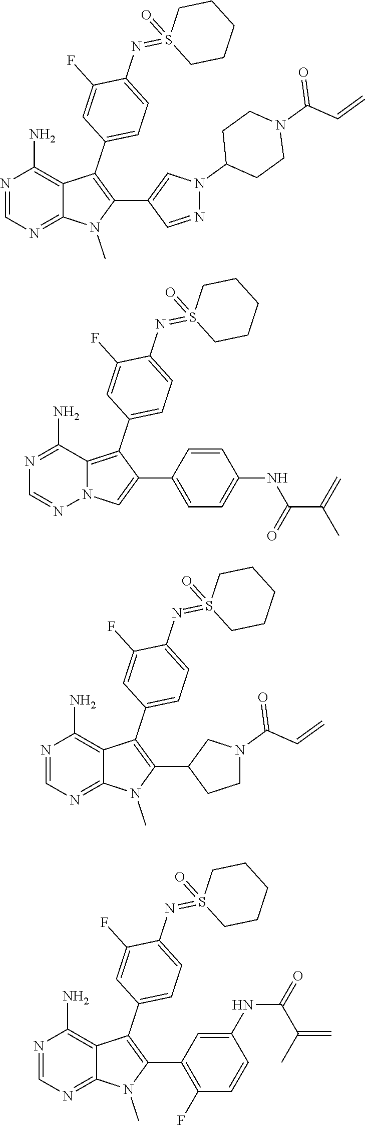

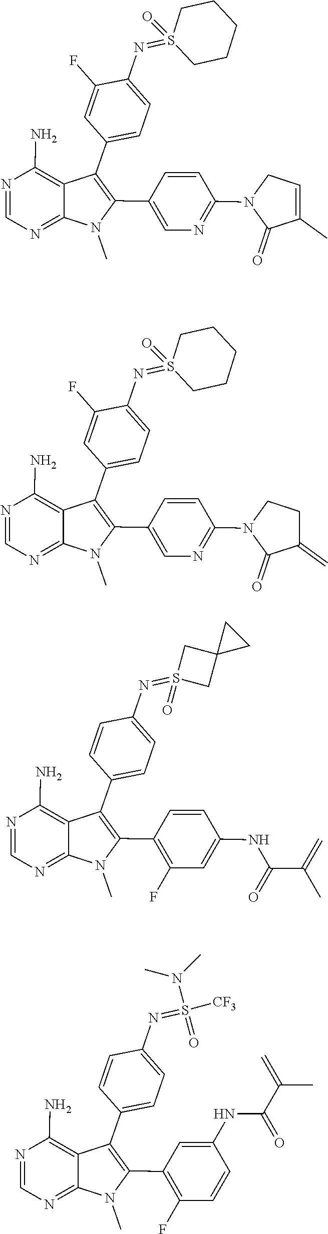 [SMILES: CC1=CCN(c2ccc(-c3c(-c4ccc(N=S5(=O)CCCCC5)c(F)c4)c4c(N)ncnc4n3C)cn2)C1=O ; C=C1CCN(c2ccc(-c3c(-c4ccc(N=S5(=O)CCCCC5)c(F)c4)c4c(N)ncnc4n3C)cn2)C1=O ; C=C(C)C(=O)Nc1ccc(-c2c(-c3ccc(N=S4(=O)CC5(CC5)C4)cc3)c3c(N)ncnc3n2C)c(F)c1 ; C=C(C)C(=O)Nc1ccc(F)c(-c2c(-c3ccc(N=S(=O)(N(C)C)C(F)(F)F)cc3)c3c(N)ncnc3n2C)c1]

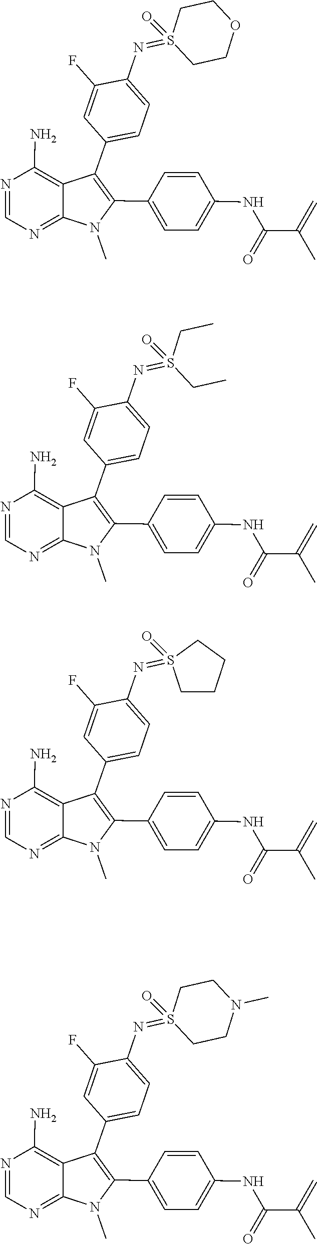 [SMILES: C=C(C)C(=O)Nc1ccc(-c2c(-c3ccc(N=S4(=O)CCOCC4)c(F)c3)c3c(N)ncnc3n2C)cc1 ; C=C(C)C(=O)Nc1ccc(-c2c(-c3ccc(N=S(=O)(CC)CC)c(F)c3)c3c(N)ncnc3n2C)cc1 ; C=C(C)C(=O)Nc1ccc(-c2c(-c3ccc(N=S4(=O)CCCC4)c(F)c3)c3c(N)ncnc3n2C)cc1 ; C=C(C)C(=O)Nc1ccc(-c2c(-c3ccc(N=S4(=O)CCN(C)CC4)c(F)c3)c3c(N)ncnc3n2C)cc1]

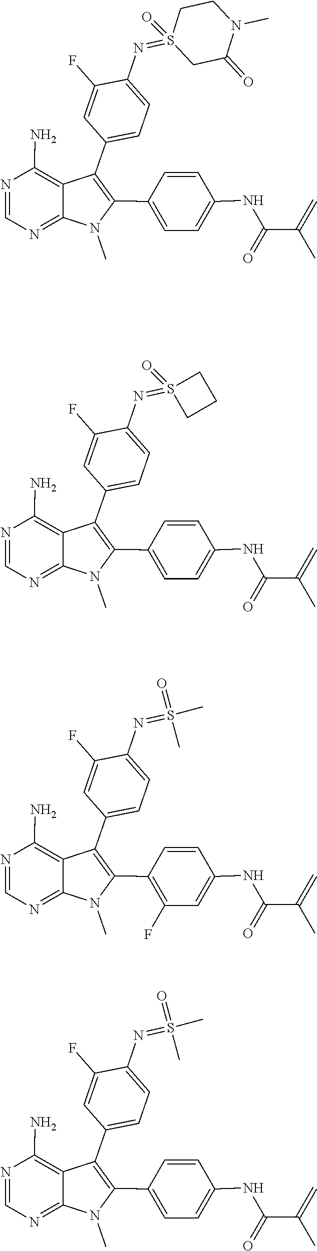 [SMILES: C=C(C)C(=O)Nc1ccc(-c2c(-c3ccc(N=S4(=O)CCN(C)C(=O)C4)c(F)c3)c3c(N)ncnc3n2C)cc1 ; C=C(C)C(=O)Nc1ccc(-c2c(-c3ccc(N=S4(=O)CCC4)c(F)c3)c3c(N)ncnc3n2C)cc1 ; C=C(C)C(=O)Nc1ccc(-c2c(-c3ccc(N=S(C)(C)=O)c(F)c3)c3c(N)ncnc3n2C)c(F)c1 ; C=C(C)C(=O)Nc1ccc(-c2c(-c3ccc(N=S(C)(C)=O)c(F)c3)c3c(N)ncnc3n2C)cc1]

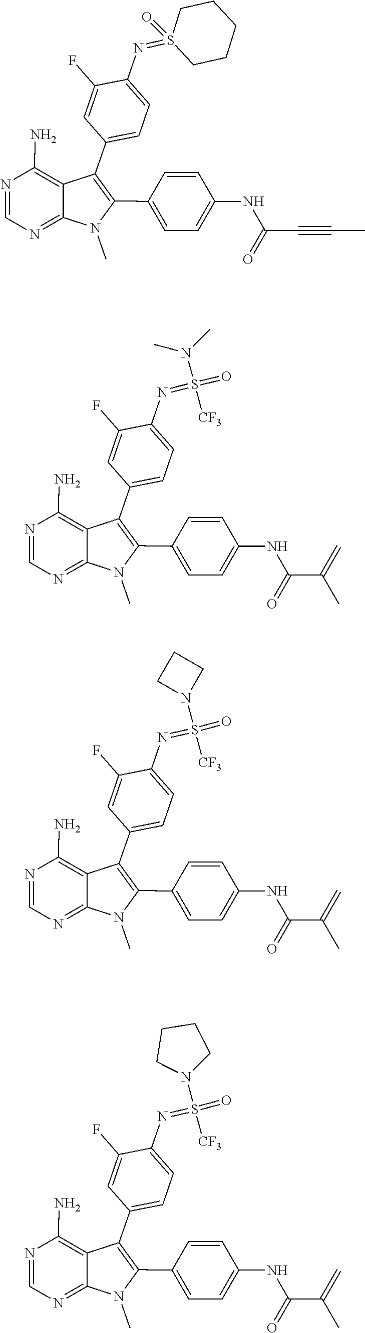 [SMILES: CC#CC(=O)Nc1ccc(-c2c(-c3ccc(N=S4(=O)CCCCC4)c(F)c3)c3c(N)ncnc3n2C)cc1 ; C=C(C)C(=O)Nc1ccc(-c2c(-c3ccc(N=S(=O)(N(C)C)C(F)(F)F)c(F)c3)c3c(N)ncnc3n2C)cc1 ; C=C(C)C(=O)Nc1ccc(-c2c(-c3ccc(N=S(=O)(N4CCC4)C(F)(F)F)c(F)c3)c3c(N)ncnc3n2C)cc1 ; C=C(C)C(=O)Nc1ccc(-c2c(-c3ccc(N=S(=O)(N4CCCC4)C(F)(F)F)c(F)c3)c3c(N)ncnc3n2C)cc1]

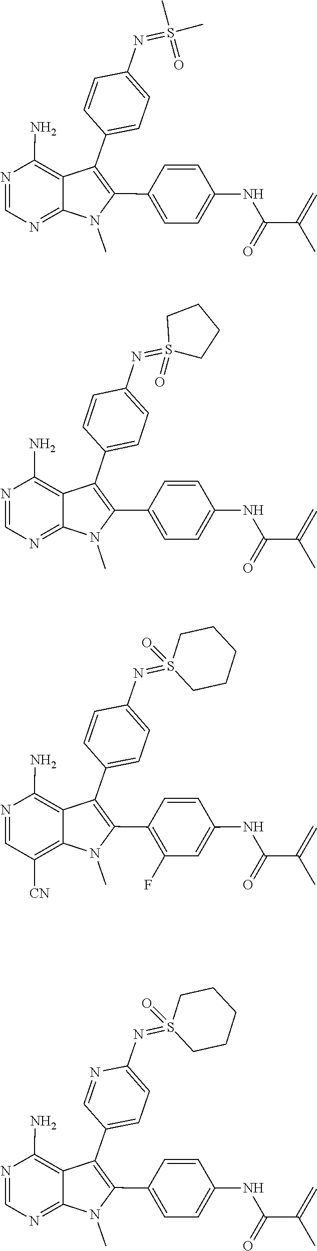 [SMILES: C=C(C)C(=O)Nc1ccc(-c2c(-c3ccc(N=S(C)(C)=O)cc3)c3c(N)ncnc3n2C)cc1 ; C=C(C)C(=O)Nc1ccc(-c2c(-c3ccc(N=S4(=O)CCCC4)cc3)c3c(N)ncnc3n2C)cc1 ; C=C(C)C(=O)Nc1ccc(-c2c(-c3ccc(N=S4(=O)CCCCC4)cc3)c3c(N)ncc(C#N)c3n2C)c(F)c1 ; C=C(C)C(=O)Nc1ccc(-c2c(-c3ccc(N=S4(=O)CCCCC4)nc3)c3c(N)ncnc3n2C)cc1]

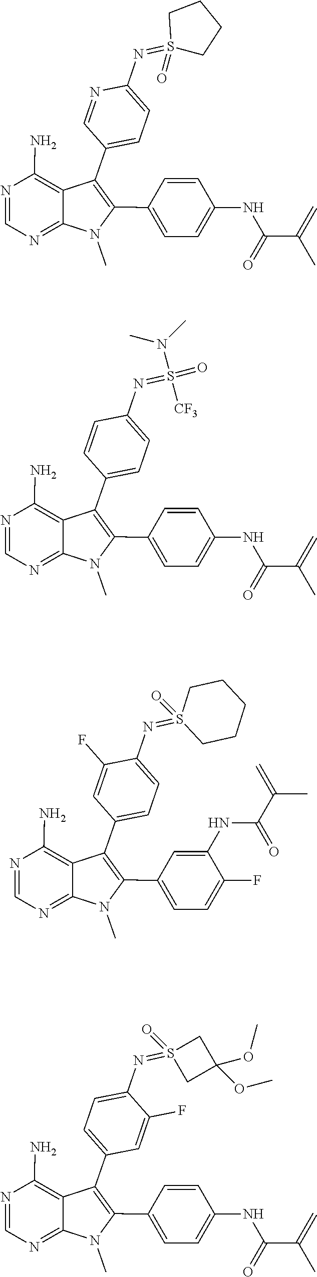 [SMILES: C=C(C)C(=O)Nc1ccc(-c2c(-c3ccc(N=S4(=O)CCCC4)nc3)c3c(N)ncnc3n2C)cc1 ; C=C(C)C(=O)Nc1ccc(-c2c(-c3ccc(N=S(=O)(N(C)C)C(F)(F)F)cc3)c3c(N)ncnc3n2C)cc1 ; C=C(C)C(=O)Nc1cc(-c2c(-c3ccc(N=S4(=O)CCCCC4)c(F)c3)c3c(N)ncnc3n2C)ccc1F ; C=C(C)C(=O)Nc1ccc(-c2c(-c3ccc(N=S4(=O)CC(OC)(OC)C4)c(F)c3)c3c(N)ncnc3n2C)cc1]

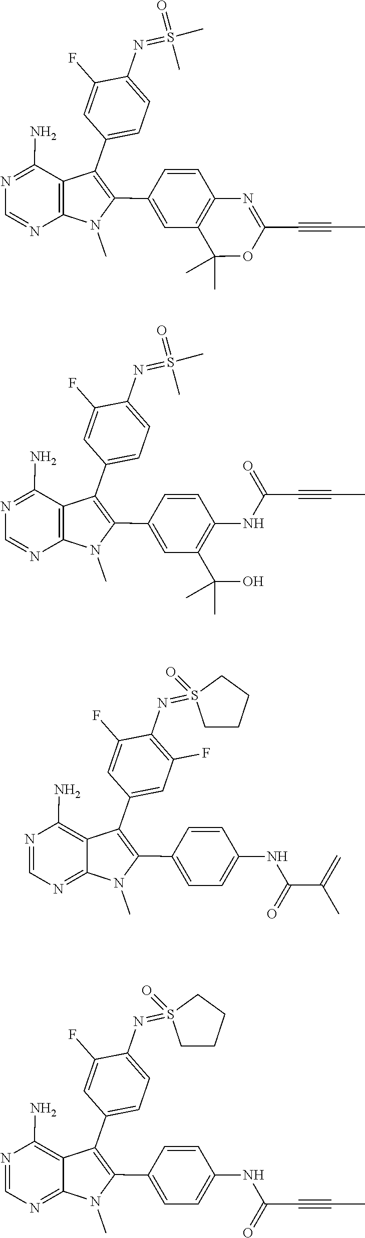 [SMILES: CC#CC1=Nc2ccc(-c3c(-c4ccc(N=S(C)(C)=O)c(F)c4)c4c(N)ncnc4n3C)cc2C(C)(C)O1 ; CC#CC(=O)Nc1ccc(-c2c(-c3ccc(N=S(C)(C)=O)c(F)c3)c3c(N)ncnc3n2C)cc1C(C)(C)O ; C=C(C)C(=O)Nc1ccc(-c2c(-c3cc(F)c(N=S4(=O)CCCC4)c(F)c3)c3c(N)ncnc3n2C)cc1 ; CC#CC(=O)Nc1ccc(-c2c(-c3ccc(N=S4(=O)CCCC4)c(F)c3)c3c(N)ncnc3n2C)cc1]

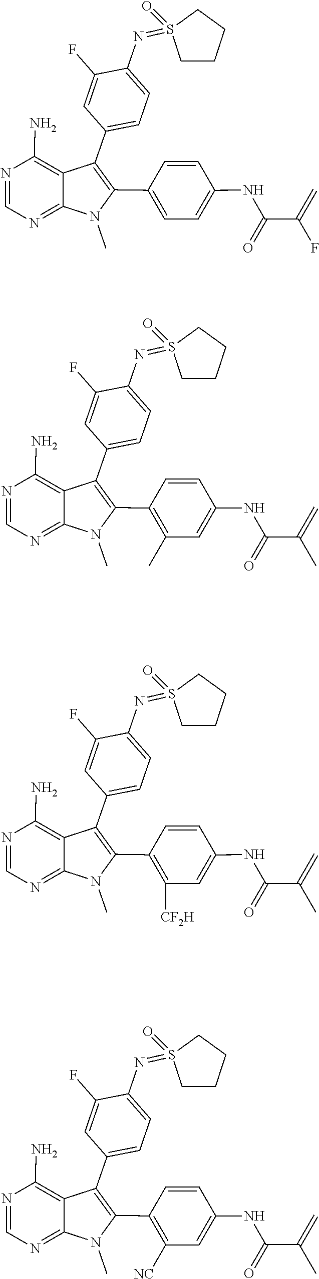 [SMILES: C=C(F)C(=O)Nc1ccc(-c2c(-c3ccc(N=S4(=O)CCCC4)c(F)c3)c3c(N)ncnc3n2C)cc1 ; C=C(C)C(=O)Nc1ccc(-c2c(-c3ccc(N=S4(=O)CCCC4)c(F)c3)c3c(N)ncnc3n2C)c(C)c1 ; C=C(C)C(=O)Nc1ccc(-c2c(-c3ccc(N=S4(=O)CCCC4)c(F)c3)c3c(N)ncnc3n2C)c(C(F)F)c1 ; C=C(C)C(=O)Nc1ccc(-c2c(-c3ccc(N=S4(=O)CCCC4)c(F)c3)c3c(N)ncnc3n2C)c(C#N)c1]

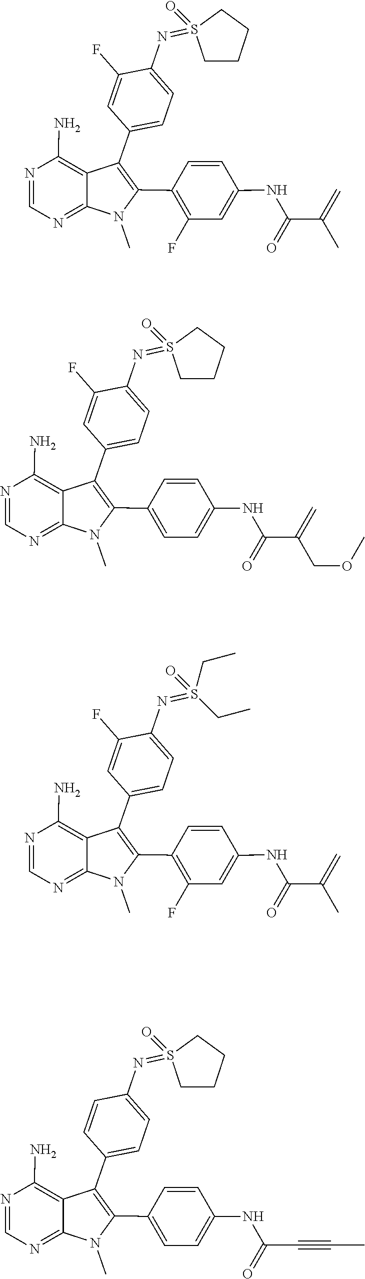 [SMILES: C=C(C)C(=O)Nc1ccc(-c2c(-c3ccc(N=S4(=O)CCCC4)c(F)c3)c3c(N)ncnc3n2C)c(F)c1 ; C=C(COC)C(=O)Nc1ccc(-c2c(-c3ccc(N=S4(=O)CCCC4)c(F)c3)c3c(N)ncnc3n2C)cc1 ; C=C(C)C(=O)Nc1ccc(-c2c(-c3ccc(N=S(=O)(CC)CC)c(F)c3)c3c(N)ncnc3n2C)c(F)c1 ; CC#CC(=O)Nc1ccc(-c2c(-c3ccc(N=S4(=O)CCCC4)cc3)c3c(N)ncnc3n2C)cc1]

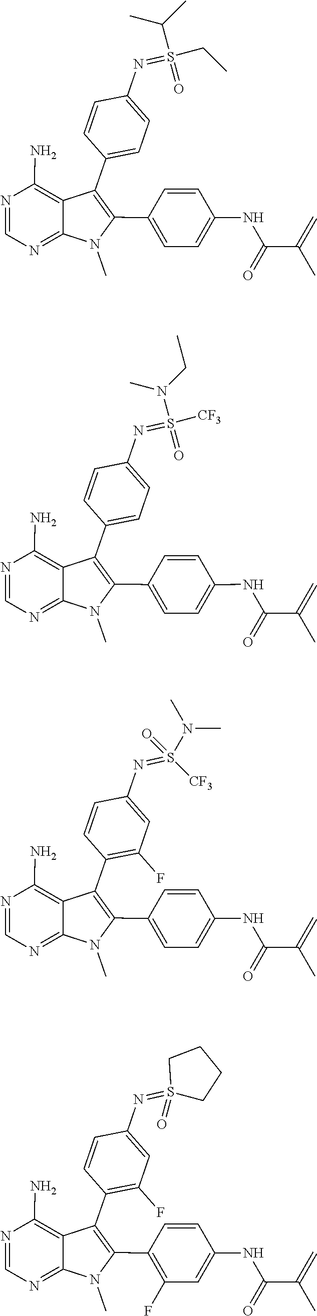 [SMILES: C=C(C)C(=O)Nc1ccc(-c2c(-c3ccc(N=S(=O)(CC)C(C)C)cc3)c3c(N)ncnc3n2C)cc1 ; C=C(C)C(=O)Nc1ccc(-c2c(-c3ccc(N=S(=O)(N(C)CC)C(F)(F)F)cc3)c3c(N)ncnc3n2C)cc1 ; C=C(C)C(=O)Nc1ccc(-c2c(-c3ccc(N=S(=O)(N(C)C)C(F)(F)F)cc3F)c3c(N)ncnc3n2C)cc1 ; C=C(C)C(=O)Nc1ccc(-c2c(-c3ccc(N=S4(=O)CCCC4)cc3F)c3c(N)ncnc3n2C)c(F)c1]

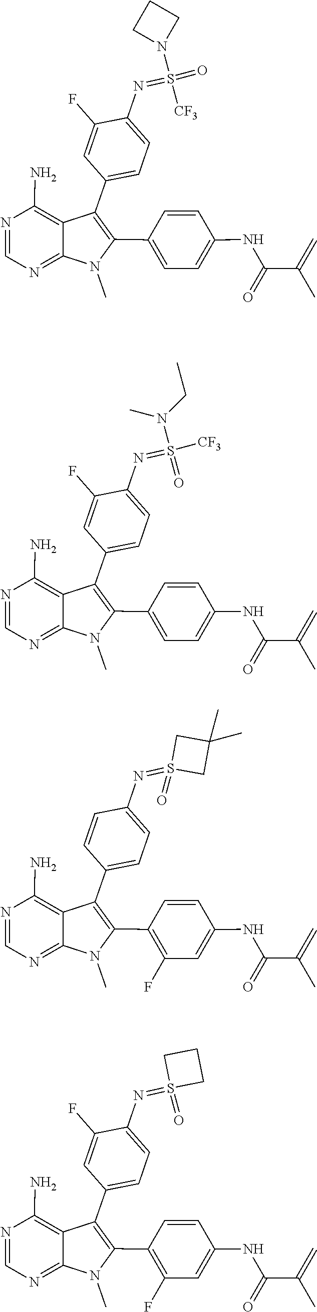 [SMILES: C=C(C)C(=O)Nc1ccc(-c2c(-c3ccc(N=S(=O)(N4CCC4)C(F)(F)F)c(F)c3)c3c(N)ncnc3n2C)cc1 ; C=C(C)C(=O)Nc1ccc(-c2c(-c3ccc(N=S(=O)(N(C)CC)C(F)(F)F)c(F)c3)c3c(N)ncnc3n2C)cc1 ; C=C(C)C(=O)Nc1ccc(-c2c(-c3ccc(N=S4(=O)CC(C)(C)C4)cc3)c3c(N)ncnc3n2C)c(F)c1 ; C=C(C)C(=O)Nc1ccc(-c2c(-c3ccc(N=S4(=O)CCC4)c(F)c3)c3c(N)ncnc3n2C)c(F)c1]

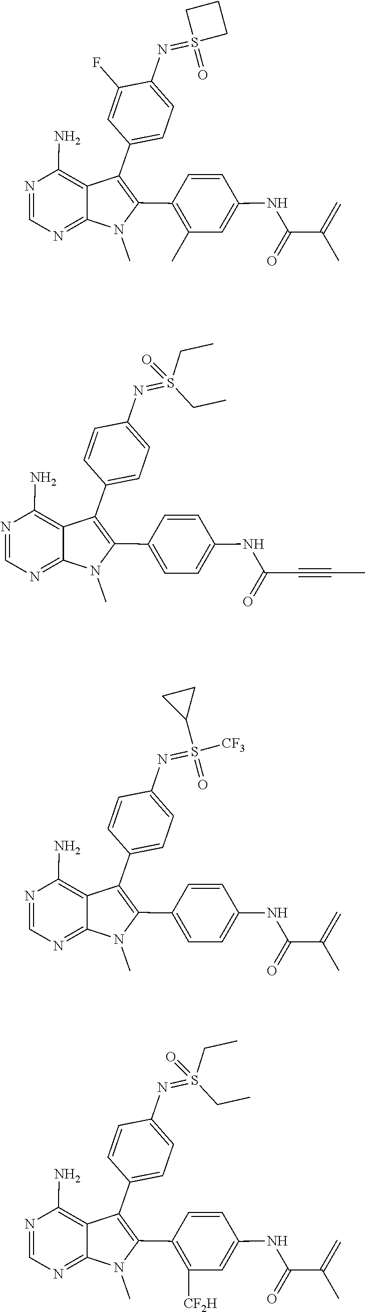 [SMILES: C=C(C)C(=O)Nc1ccc(-c2c(-c3ccc(N=S4(=O)CCC4)c(F)c3)c3c(N)ncnc3n2C)c(C)c1 ; CC#CC(=O)Nc1ccc(-c2c(-c3ccc(N=S(=O)(CC)CC)cc3)c3c(N)ncnc3n2C)cc1 ; C=C(C)C(=O)Nc1ccc(-c2c(-c3ccc(N=S(=O)(C4CC4)C(F)(F)F)cc3)c3c(N)ncnc3n2C)cc1 ; C=C(C)C(=O)Nc1ccc(-c2c(-c3ccc(N=S(=O)(CC)CC)cc3)c3c(N)ncnc3n2C)c(C(F)F)c1]

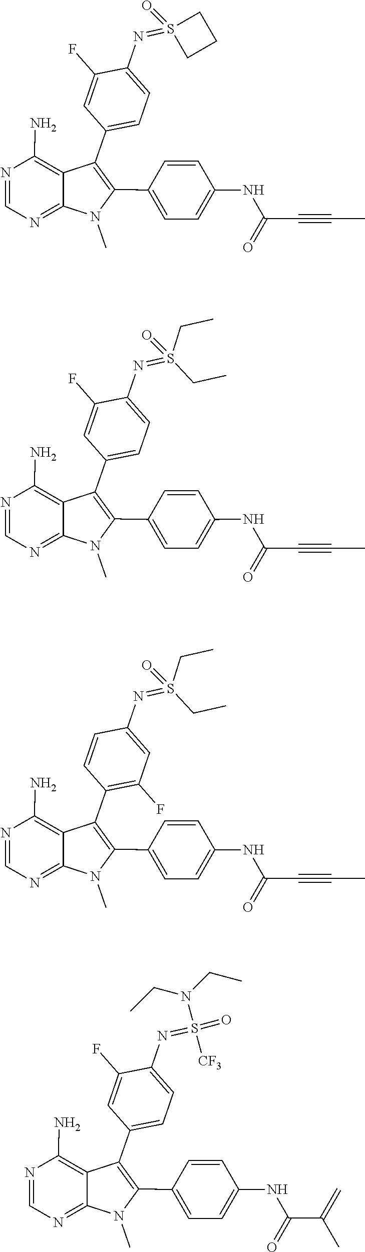 [SMILES: CC#CC(=O)Nc1ccc(-c2c(-c3ccc(N=S4(=O)CCC4)c(F)c3)c3c(N)ncnc3n2C)cc1 ; CC#CC(=O)Nc1ccc(-c2c(-c3ccc(N=S(=O)(CC)CC)c(F)c3)c3c(N)ncnc3n2C)cc1 ; CC#CC(=O)Nc1ccc(-c2c(-c3ccc(N=S(=O)(CC)CC)cc3F)c3c(N)ncnc3n2C)cc1 ; C=C(C)C(=O)Nc1ccc(-c2c(-c3ccc(N=S(=O)(N(CC)CC)C(F)(F)F)c(F)c3)c3c(N)ncnc3n2C)cc1]

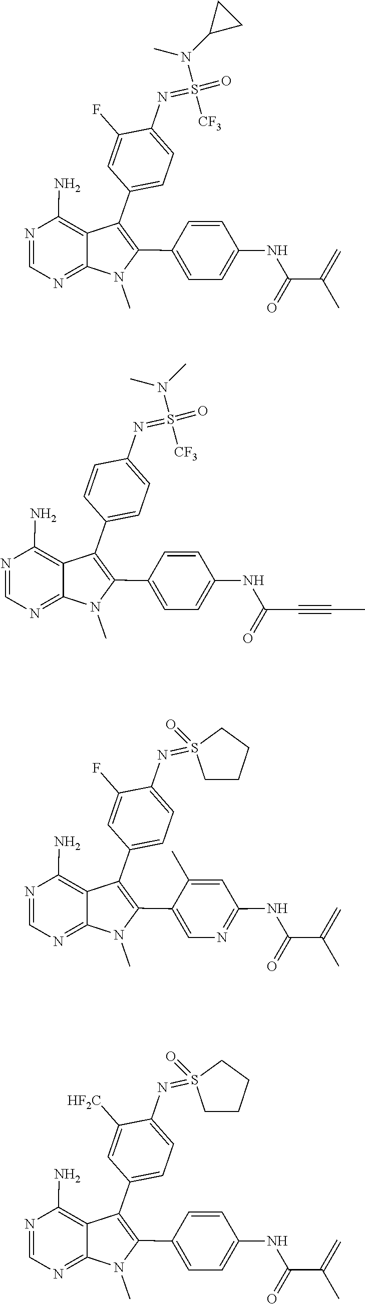

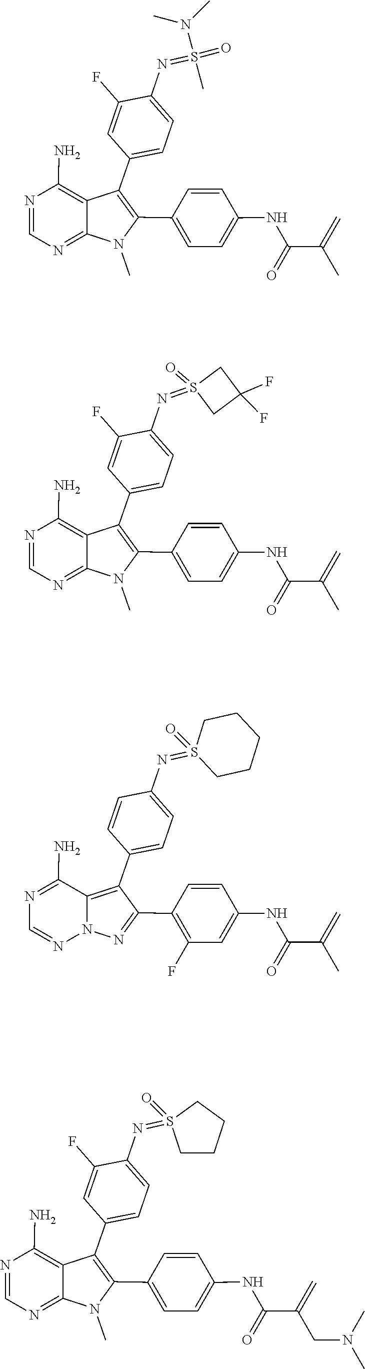 [SMILES: C=C(C)C(=O)Nc1ccc(-c2c(-c3ccc(N=S(C)(=O)N(C)C)c(F)c3)c3c(N)ncnc3n2C)cc1 ; C=C(C)C(=O)Nc1ccc(-c2c(-c3ccc(N=S4(=O)CC(F)(F)C4)c(F)c3)c3c(N)ncnc3n2C)cc1 ; C=C(C)C(=O)Nc1ccc(-c2nn3ncnc(N)c3c2-c2ccc(N=S3(=O)CCCCC3)cc2)c(F)c1 ; C=C(CN(C)C)C(=O)Nc1ccc(-c2c(-c3ccc(N=S4(=O)CCCC4)c(F)c3)c3c(N)ncnc3n2C)cc1]

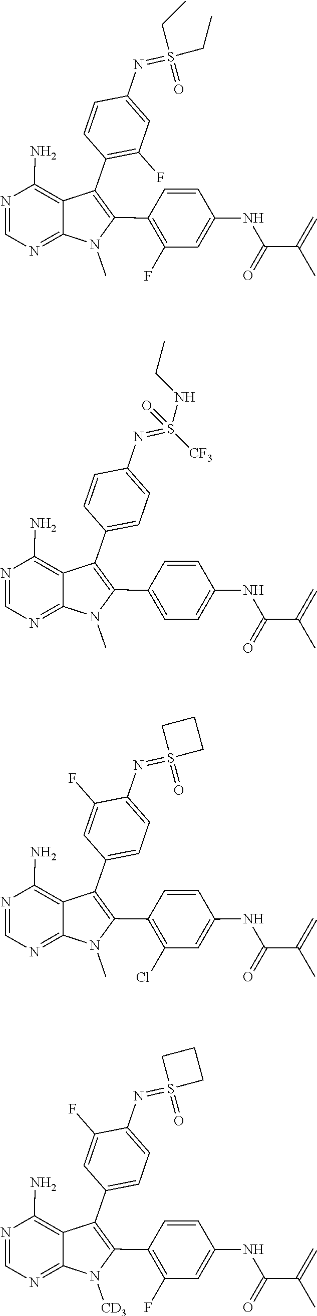 [SMILES: C=C(C)C(=O)Nc1ccc(-c2c(-c3ccc(N=S(=O)(CC)CC)cc3F)c3c(N)ncnc3n2C)c(F)c1 ; C=C(C)C(=O)Nc1ccc(-c2c(-c3ccc(N=S(=O)(NCC)C(F)(F)F)cc3)c3c(N)ncnc3n2C)cc1 ; C=C(C)C(=O)Nc1ccc(-c2c(-c3ccc(N=S4(=O)CCC4)c(F)c3)c3c(N)ncnc3n2C)c(Cl)c1 ; [2H]C([2H])([2H])n1c(-c2ccc(NC(=O)C(=C)C)cc2F)c(-c2ccc(N=S3(=O)CCC3)c(F)c2)c2c(N)ncnc21]

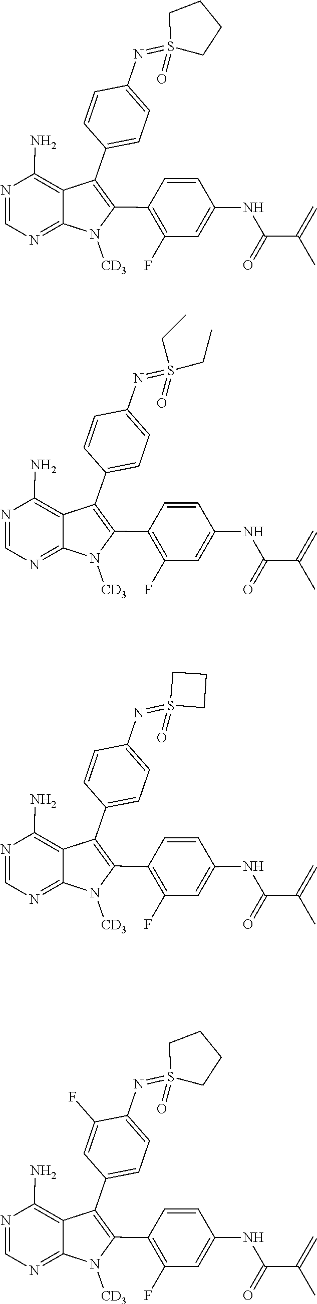 [SMILES: [2H]C([2H])([2H])n1c(-c2ccc(NC(=O)C(=C)C)cc2F)c(-c2ccc(N=S3(=O)CCCC3)cc2)c2c(N)ncnc21 ; [2H]C([2H])([2H])n1c(-c2ccc(NC(=O)C(=C)C)cc2F)c(-c2ccc(N=S(=O)(CC)CC)cc2)c2c(N)ncnc21 ; [2H]C([2H])([2H])n1c(-c2ccc(NC(=O)C(=C)C)cc2F)c(-c2ccc(N=S3(=O)CCC3)cc2)c2c(N)ncnc21 ; [2H]C([2H])([2H])n1c(-c2ccc(NC(=O)C(=C)C)cc2F)c(-c2ccc(N=S3(=O)CCCC3)c(F)c2)c2c(N)ncnc21]

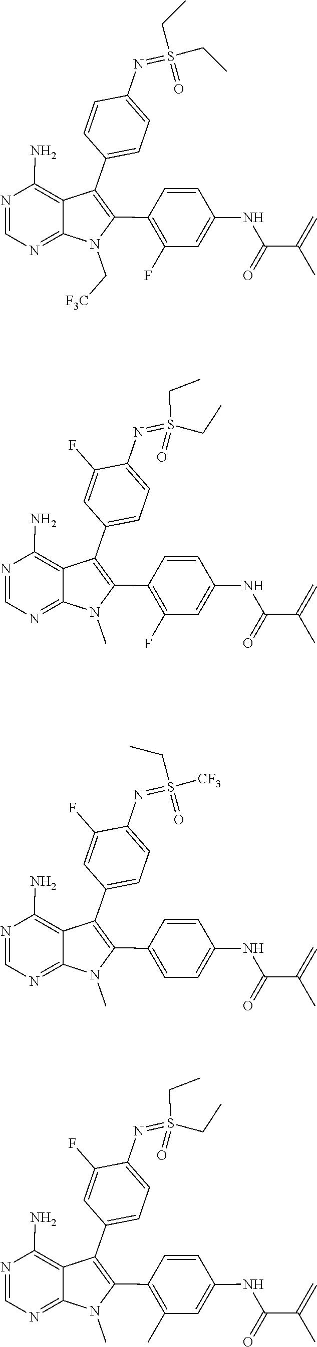 [SMILES: C=C(C)C(=O)Nc1ccc(-c2c(-c3ccc(N=S(=O)(CC)CC)cc3)c3c(N)ncnc3n2CC(F)(F)F)c(F)c1 ; C=C(C)C(=O)Nc1ccc(-c2c(-c3ccc(N=S(=O)(CC)CC)c(F)c3)c3c(N)ncnc3n2C)c(F)c1 ; C=C(C)C(=O)Nc1ccc(-c2c(-c3ccc(N=S(=O)(CC)C(F)(F)F)c(F)c3)c3c(N)ncnc3n2C)cc1 ; C=C(C)C(=O)Nc1ccc(-c2c(-c3ccc(N=S(=O)(CC)CC)c(F)c3)c3c(N)ncnc3n2C)c(C)c1]

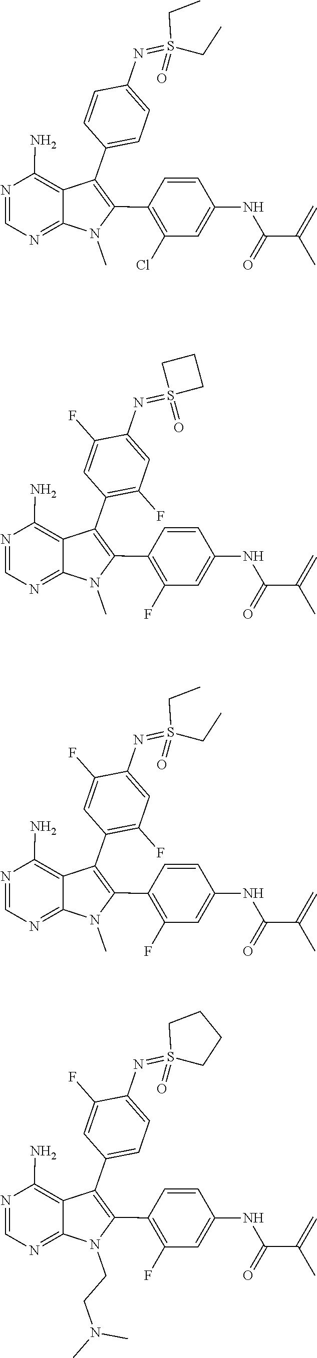 [SMILES: C=C(C)C(=O)Nc1ccc(-c2c(-c3ccc(N=S(=O)(CC)CC)cc3)c3c(N)ncnc3n2C)c(Cl)c1 ; C=C(C)C(=O)Nc1ccc(-c2c(-c3cc(F)c(N=S4(=O)CCC4)cc3F)c3c(N)ncnc3n2C)c(F)c1 ; C=C(C)C(=O)Nc1ccc(-c2c(-c3cc(F)c(N=S(=O)(CC)CC)cc3F)c3c(N)ncnc3n2C)c(F)c1 ; C=C(C)C(=O)Nc1ccc(-c2c(-c3ccc(N=S4(=O)CCCC4)c(F)c3)c3c(N)ncnc3n2CCN(C)C)c(F)c1]

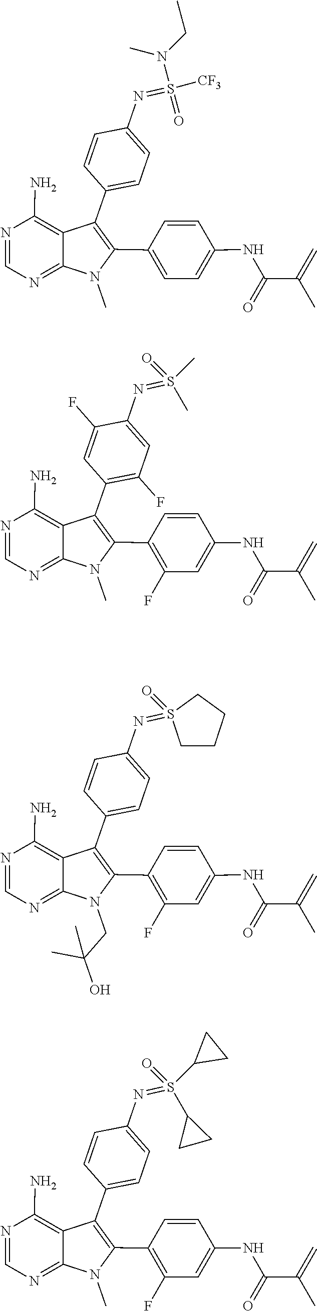 [SMILES: C=C(C)C(=O)Nc1ccc(-c2c(-c3ccc(N=S(=O)(N(C)CC)C(F)(F)F)cc3)c3c(N)ncnc3n2C)cc1 ; C=C(C)C(=O)Nc1ccc(-c2c(-c3cc(F)c(N=S(C)(C)=O)cc3F)c3c(N)ncnc3n2C)c(F)c1 ; C=C(C)C(=O)Nc1ccc(-c2c(-c3ccc(N=S4(=O)CCCC4)cc3)c3c(N)ncnc3n2CC(C)(C)O)c(F)c1 ; C=C(C)C(=O)Nc1ccc(-c2c(-c3ccc(N=S(=O)(C4CC4)C4CC4)cc3)c3c(N)ncnc3n2C)c(F)c1]

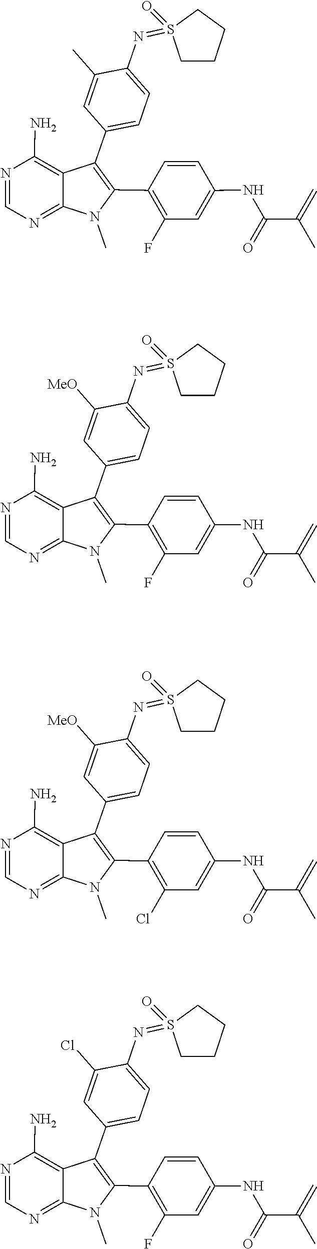 [SMILES: C=C(C)C(=O)Nc1ccc(-c2c(-c3ccc(N=S4(=O)CCCC4)c(C)c3)c3c(N)ncnc3n2C)c(F)c1 ; C=C(C)C(=O)Nc1ccc(-c2c(-c3ccc(N=S4(=O)CCCC4)c(OC)c3)c3c(N)ncnc3n2C)c(F)c1 ; C=C(C)C(=O)Nc1ccc(-c2c(-c3ccc(N=S4(=O)CCCC4)c(OC)c3)c3c(N)ncnc3n2C)c(Cl)c1 ; C=C(C)C(=O)Nc1ccc(-c2c(-c3ccc(N=S4(=O)CCCC4)c(Cl)c3)c3c(N)ncnc3n2C)c(F)c1]

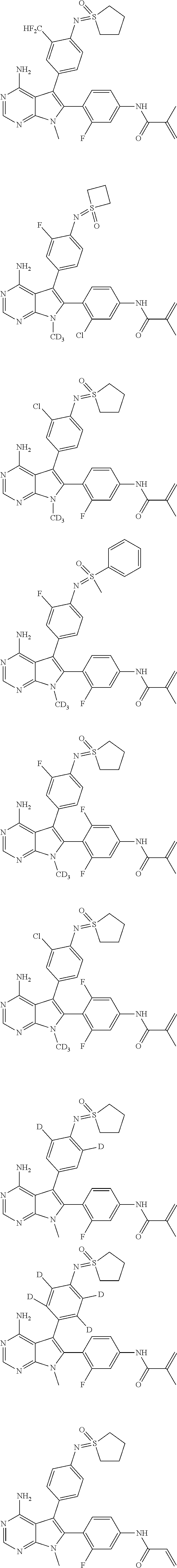

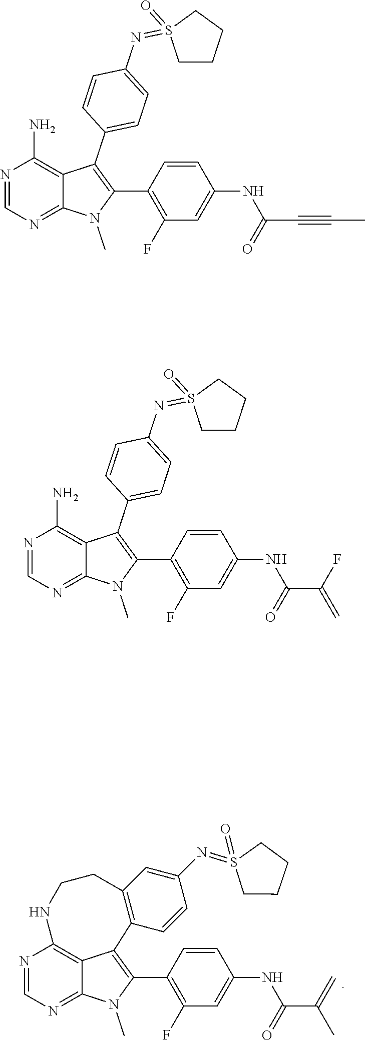 [SMILES: CC#CC(=O)Nc1ccc(-c2c(-c3ccc(N=S4(=O)CCCC4)cc3)c3c(N)ncnc3n2C)c(F)c1 ; C=C(F)C(=O)Nc1ccc(-c2c(-c3ccc(N=S4(=O)CCCC4)cc3)c3c(N)ncnc3n2C)c(F)c1 ; C=C(C)C(=O)Nc1ccc(-c2c3c4c(ncnc4n2C)NCCc2cc(N=S4(=O)CCCC4)ccc2-3)c(F)c1]

9. A pharmaceutical composition, comprising:
(1) one or more of the compound according to claim 1, or a tautomer, a stereoisomer, a hydrate, a solvate, or a pharmaceutically acceptable salt thereof;
(2) one or more pharmaceutically acceptable carriers, excipients, adjuvants, excipients, and/or diluents.

10. The use of the compound according to claim 1, or a tautomer, a stereoisomer, a hydrate, a solvate, or a pharmaceutically acceptable salt thereof, or mixtures thereof in the preparation a pharmaceutical composition for treating or preventing diseases or conditions associated with abnormal activity or expression levels of FGFR
preferably, the disease or condition is specifically associated with a subtype of FGFR;
more preferably, the FGFR subtype is selected from the group consisting of: FGFR2.

11. The compound according to claim 1, or a tautomer, a stereoisomer, a hydrate, a solvate, or a pharmaceutically acceptable salt thereof, wherein, R₈ is selected from the group consisting of H, NH₂, C₁₋₆ alkyl, and C₁₋₆ alkylamino.

12. The compound according to claim 1, or a tautomer, a stereoisomer, a hydrate, a solvate, or a pharmaceutically acceptable salt thereof, wherein, R₉ is selected from the group consisting of: H, D, halogen, CN, C₁₋₆ alkyl and C₁₋₆ haloalkyl.

13. The use of the pharmaceutical composition in treating or preventing diseases or condition associated with abnormal activity or expression levels of FGFR;
preferably, the disease or condition is selected from the group consisting of:
cholangiocarcinoma, hepatic carcinoma, breast carcinoma, prostate carcinoma, lung carcinoma, thyroid carcinoma, gastric carcinoma, ovarian carcinoma, rectal carcinoma, endometrial carcinoma and uroepithelial carcinoma;
more preferably, the cholangiocarcinoma is intrahepatic cholangiocarcinoma;
further more preferably, the hepatic carcinoma is hepatocellular carcinoma;
the most preferably, the lung carcinoma is squamous cell carcinoma or non-small cell lung carcinoma.
